SEQUENCE LISTING (204 sequences)

SEQ ID NO: 1 - protein, 124 aa, synthetic construct
  REGION 1..124: note = Synthetic Construct
EVQLVESGGGLVQPGGSLRLSCAASGYTFSNYWIHWVRQAPGKGLEWIGIIYPSSGSTKYAQKFQGRVTISRDNSKNTLYLQLNSLRAEDTAVYYCARDRAPGSSGYYDGFDYWGQGTLVTVSS

SEQ ID NO: 2 - protein, 112 aa, synthetic construct
  REGION 1..112: note = Synthetic Construct
DIQLTQSPSSLSASVGDRVTITCRASQSVDFHGISFLHWYQQKPGKAPKLLIYDASSLESGVPSRFSGSGSGTDFTLTISSLQPEDFATYYCHQRSTTPLTFGQGTKVEIKR

SEQ ID NO: 3 - protein, 118 aa, synthetic construct
  REGION 1..118: note = Synthetic Construct
EVQLVESGGGLVQPGGSLRLSCAASGYSITSGYYWSWIRQAPGKGLEWIGEIYHSGNTYYNPSLKSRVTISRDNSKNTLYLQLNSLRAEDTAVYYCARRVPLGAFDYWGQGTLVTVSS

SEQ ID NO: 4 - protein, 109 aa, synthetic construct
  REGION 1..109: note = Synthetic Construct
DIQLTQSPSSLSASVGDRVTITCRASQTVISRYLAWYQQKPGKAPKLLIYDASNLETGVPSRFSGSGSGTDFTLTISSLQPEDFATYYCQQYYAWPLTFGQGTKVEIKR

SEQ ID NO: 5 - protein, 121 aa, synthetic construct
  REGION 1..121: note = Synthetic Construct
EVQLVESGGGLVQPGGSLRLSCAASGYSITSGYHWGWIRQAPGKGLEWIGRIYPSSGSTKYAQKFQGRVTISRDNSKNTLYLQLNSLRAEDTAVYYCARGVRGSYGWFDVWGQGTLVTVSS

SEQ ID NO: 6 - protein, 109 aa, synthetic construct
  REGION 1..109: note = Synthetic Construct
DIQLTQSPSSLSASVGDRVTITCSASSRVSYVYWYQQKPGKAPKLLIYAASTLQSGVPSRFSGSGSGTDFTLTISSLQPEDFATYYCQHYVSSPRVYTFGQGTKVEIKR

SEQ ID NO: 7 - protein, 118 aa, synthetic construct
  REGION 1..118: note = Synthetic Construct
EVQLVESGGGLVQPGGSLRLSCAASGFTFTSYGIHWVRQAPGKGLEWVSAISGSGGSTYYADSVKGRFTISRDNSKNTLYLQLNSLRAEDTAVYYCARSGHAYDFDYWGQGTLVTVSS

SEQ ID NO: 8 - protein, 112 aa, synthetic construct
  REGION 1..112: note = Synthetic Construct
DIQLTQSPSSLSASVGDRVTITCRASQSVDFYGISFLDWYQQKPGKAPKLLIYAASTLQSGVPSRFSGSGSGTDFTLTISSLQPEDFATYYCQQGGTSPWTFAQGTKVEIKR

SEQ ID NO: 9 - protein, 118 aa, synthetic construct
  REGION 1..118: note = Synthetic Construct
QVQLVQSGAEVKKPGSSVKVSCKASGFTFTSYAIHWVRQAPGQGLEWIGIINPNFGSTNYAQKFQGRVTITADKSTSTAYMELSSLRSEDTAVYYCARYYYGASFDVWGQGTLVTVSS

SEQ ID NO: 10 - protein, 115 aa, synthetic construct
  REGION 1..115: note = Synthetic Construct
DIQLTQSPSSLSASVGDRVTITCKSGQSLLHGDVKTYLYWYQQKPGKAPKLLIYDASNRATGIPSRFSGSGSGTDFTLTISSLQPEDFATYYCQQYASWPPGFTFGQGTKVEIKR

SEQ ID NO: 11 - protein, 120 aa, synthetic construct
  REGION 1..120: note = Synthetic Construct
EVQLVESGGGLVQPGGSLRLSCAASGYSITSGHYWNWIRQAPGKGLEWIGIISPSGGGTKYAQKFQGRVTISRDNSKNTLYLQLNSLRAEDTAVYYCARGARGYYALDYWGQGTLVTVSS

SEQ ID NO: 12 - protein, 109 aa, synthetic construct
  REGION 1..109: note = Synthetic Construct
EIVLTQSPATLSLSPGERATLSCRASQSVRGRLLAWYQQKPGQAPRLLIYAASSLQSGVPARFSGSGSGTDFTLTISSLEPEDFAVYYCAQYLELPYTFGQGTKVEIKR

SEQ ID NO: 13 - protein, 116 aa, synthetic construct
  REGION 1..116: note = Synthetic Construct
EVQLVESGGGLVQPGGSLRLSCAASGYSISSGYHWDWIRQAPGKGLEWLARIYWDGDKRYSPSLKSRLTISRDNSKNTLYLQLNSLRAEDTAVYYCARFSDGFDPWGQGTLVTVSS

SEQ ID NO: 14 - protein, 112 aa, synthetic construct
  REGION 1..112: note = Synthetic Construct
EIVLTQSPATLSLSPGERATLSCRASQSVDFVGISFLHWYQQKPGQAPRLLIYDASNRATGIPARFSGSGSGTDFTLTISSLEPEDFAVYYCVQGLQWPHTFGQGTKVEIKR

SEQ ID NO: 15 - protein, 118 aa, synthetic construct
  REGION 1..118: note = Synthetic Construct
QVQLVQSGAEVKKPGSSVKVSCKASGYTFTGYAIHWVRQAPGQGLEWIGWISPSSGSTKYAQKFQGRVTITADKSTSTAYMELSSLRSEDTAVYYCARYGYGPVFDYWGQGTLVTVSS

SEQ ID NO: 16 - protein, 109 aa, synthetic construct
  REGION 1..109: note = Synthetic Construct
DIQLTQSPSSLSASVGDRVTITCSASSSVTYIYWYQQKPGKAPKLLIYDASNRATGIPSRFSGSGSGTDFTLTISSLQPEDFATYYCQQYTSSPRGFTFGQGTKVEIKR

SEQ ID NO: 17 - protein, 120 aa, synthetic construct
  REGION 1..120: note = Synthetic Construct
QVQLVQSGAEVKKPGSSVKVSCKASGYSITSGHYWTWIRQAPGQGLEWIGGIIPVFGTPNYAQKFQGRVTITADKSTSTAYMELSSLRSEDTAVYYCASYGYGGAYFDVWGQGTLVTVSS

SEQ ID NO: 18 - protein, 112 aa, synthetic construct
  REGION 1..112: note = Synthetic Construct
DIQLTQSPSSLSASVGDRVTITCRASQSVDFHGISFLHWYQQKPGKAPKLLIYDASNRATGIPSRFSGSGSGTDFTLTISSLQPEDFATYYCVQGIQWPWTFGQGTKVEIKR

SEQ ID NO: 19 - protein, 118 aa, synthetic construct
  REGION 1..118: note = Synthetic Construct
QVQLVQSGAEVKKPGSSVKVSCKASGFTFSRYAIHWVRQAPGQGLEWLGGIIPVFGTANYAQKFQGRVTITADKSTSTAYMELSSLRSEDTAVYYCARQGILGGFAYWGQGTLVTVSS

SEQ ID NO: 20 - protein, 112 aa, synthetic construct
  REGION 1..112: note = Synthetic Construct
DIQLTQSPSSLSASVGDRVTITCRASESVDFHGKSFLAWYQQKPGKAPKLLIYAASSLQSGVPSRFSGSGSGTDFTLTISSLQPEDFATYYCQQYLELPFTFGQGTKVEIKR

SEQ ID NO: 21 - protein, 124 aa, synthetic construct
  REGION 1..124: note = Synthetic Construct
QVQPAQSGAEVKKPGSSVKVSCKASGYSISSGYYWGWIRQAPGQGLEWIGAISYSGSTYYSPSLKSRVTITADKSTSTAYMELSSLRSEDTAVYYCARHYLFAGSTSYDAFDIWGQGTLVTVSS

SEQ ID NO: 22 - protein, 112 aa, synthetic construct
  REGION 1..112: note = Synthetic Construct
DIQLTQSPSSLSASVGDRVTITCRASQSVDFYGFSFLAWYQQKPGKAPKLLIYDASNRATGIPSRFSGSGSGTDFTLTISSLQPEDFATYYCQQSYRTPLTFGQGTKVEIKR

SEQ ID NO: 23 - protein, 118 aa, synthetic construct
  REGION 1..118: note = Synthetic Construct
QVQLVQSGAEVKKPGSSVKVSCKASGFTFSDYGIHWVRQAPGQGLEWIGRIYPSGGSTNYAQKFQGRVTITADKSTSTAYMELSSLRSEDTAVYYCARQRGYGRFAYWGQGTLVTVSS

SEQ ID NO: 24 - protein, 107 aa, synthetic construct
  REGION 1..107: note = Synthetic Construct
DIQLTQSPSSLSASVGDRVTITCSASSSVGYVYWYQQKPGKAPKLLIYDASSLESGVPSRFSGSGSGTDFTLTISSLQPEDFATYFCQQYTYAPFTFGQGTKVEIKR

SEQ ID NO: 25 - protein, 122 aa, synthetic construct
  REGION 1..122: note = Synthetic Construct
QVQLVQSGAEVKKPGSSVKVSCKASGYSISSGHHWNWIRQAPGQGLEWIGAISYSGSTYYSPSLKSRVTITADKSTSTAYMELSSLRSEDTAVYYCARQGYYGGEGYAVDYWGQGTLVTVSS

SEQ ID NO: 26 - protein, 112 aa, synthetic construct
  REGION 1..112: note = Synthetic Construct
DIQLTQSPSSLSASVGDRVTITCRASQSVDFYGISFLHWYQQKPGKAPKLLIYDASNRATGIPSRFSGSGSGTDFTLTISSLQPEDFATYYCVQATQTPFTFGQGTKVEIKR

SEQ ID NO: 27 - protein, 120 aa, synthetic construct
  REGION 1..120: note = Synthetic Construct
EVQLVESGGGLVQPGGSLRLSCAASGFTFTNYAIHWVRQAPGKGLEWVSAISGSGSSTYYADSVKGRFTISRDNSKNTLYLQLNSLRAEDTAVYYCARRGSYGFGAFDYWGQGTLVTVSS

SEQ ID NO: 28 - protein, 108 aa, synthetic construct
  REGION 1..108: note = Synthetic Construct
DIQLTQSPSSLSASVGDRVTITCRASQTIGRYLNWYQQKPGKAPKLLIYDASNRATGIPSRFSGSGSGTDFTLTISSLQPEDFATYYCQQRYPWPYTFGQGTKVEIKR

SEQ ID NO: 29 - protein, 120 aa, synthetic construct
  REGION 1..120: note = Synthetic Construct
EVQLVESGGGLVQPGGSLRLSCAASGFTFSNYAIHWVRQAPGKGLEWVSAISGSGSSTYYADSVKGRFTISRDNSKNTLYLQLNSLRAEDTAVYYCARRGSYGFGAFDYWGQGTLVTVSS

SEQ ID NO: 30 - protein, 108 aa, synthetic construct; identical to SEQ ID NO: 28
  REGION 1..108: note = Synthetic Construct

SEQ ID NO: 31 - protein, 120 aa, synthetic construct
  REGION 1..120: note = Synthetic Construct
EVQLVESGGGLVQPGGSLRLSCAASGFTFSGYAIHWVRQAPGKGLEWVSAISGSGSSTYYADSVKGRFTISRDNSKNTLYLQLNSLRAEDTAVYYCARRGSYGFGAFDYWGQGTLVTVSS

SEQ ID NO: 32 - protein, 108 aa, synthetic construct; identical to SEQ ID NO: 28
  REGION 1..108: note = Synthetic Construct

SEQ ID NO: 33 - protein, 113 aa, synthetic construct
  REGION 1..113: note = Synthetic Construct
EVQLVESGGGLVQPGGSLRLSCAASGFTFSDYWIHWVRQAPGKGLEWVSGISGAGGSTYYADSVKGRFTISRDNSKNTLYLQLNSLRAEDTAVYYCARLGDYWGQGTLVTVSS

SEQ ID NO: 34 - protein, 108 aa, synthetic construct
  REGION 1..108: note = Synthetic Construct
DIQLTQSPSSLSASVGDRVTITCRASQGISSVLAWYQQKPGKAPKLLIYAASTLQSGVPSRFSGSGSGTDFTLTISSLQPEDFATYYCQQYYSIPFTFGQGTKVEIKR

SEQ ID NO: 35 - protein, 5 aa, synthetic construct
  REGION 1..5: note = Synthetic Construct
NYWIH

SEQ ID NO: 36 - protein, 6 aa, synthetic construct
  REGION 1..6: note = Synthetic Construct
SGYYWS

SEQ ID NO: 37 - protein, 6 aa, synthetic construct
  REGION 1..6: note = Synthetic Construct
SGYHWG

SEQ ID NO: 38 - protein, 5 aa, synthetic construct
  REGION 1..5: note = Synthetic Construct
SYGIH

SEQ ID NO: 39 - protein, 5 aa, synthetic construct
  REGION 1..5: note = Synthetic Construct
SYAIH

SEQ ID NO: 40 - protein, 6 aa, synthetic construct
  REGION 1..6: note = Synthetic Construct
SGHYWN

SEQ ID NO: 41 - protein, 6 aa, synthetic construct
  REGION 1..6: note = Synthetic Construct
SGYHWD

SEQ ID NO: 42 - protein, 5 aa, synthetic construct
  REGION 1..5: note = Synthetic Construct
GYAIH

SEQ ID NO: 43 - protein, 6 aa, synthetic construct
  REGION 1..6: note = Synthetic Construct
SGHYWT

SEQ ID NO: 44 - protein, 5 aa, synthetic construct
  REGION 1..5: note = Synthetic Construct
RYAIH

SEQ ID NO: 45 - protein, 6 aa, synthetic construct
  REGION 1..6: note = Synthetic Construct
SGYYWG

SEQ ID NO: 46 - protein, 5 aa, synthetic construct
  REGION 1..5: note = Synthetic Construct
DYGIH

SEQ ID NO: 47 - protein, 6 aa, synthetic construct
  REGION 1..6: note = Synthetic Construct
SGHHWN

SEQ ID NO: 48 - protein, 5 aa, synthetic construct
  REGION 1..5: note = Synthetic Construct
NYAIH

SEQ ID NO: 49 - protein, 5 aa, synthetic construct
  REGION 1..5: note = Synthetic Construct
NYAIH

SEQ ID NO: 50 - protein, 5 aa, synthetic construct
  REGION 1..5: note = Synthetic Construct
GYAIH

SEQ ID NO: 51 - protein, 5 aa, synthetic construct
  REGION 1..5: note = Synthetic Construct
DYWIH

SEQ ID NO: 52 - protein, 17 aa, synthetic construct
  REGION 1..17: note = Synthetic Construct
IIYPSSGSTKYAQKFQG

SEQ ID NO: 53 - protein, 16 aa, synthetic construct
  REGION 1..16: note = Synthetic Construct
EIYHSGNTYYNPSLKS

SEQ ID NO: 54 - protein, 17 aa, synthetic construct
  REGION 1..17: note = Synthetic Construct
RIYPSSGSTKYAQKFQG

SEQ ID NO: 55 - protein, 17 aa, synthetic construct
  REGION 1..17: note = Synthetic Construct
AISGSGGSTYYADSVKG

SEQ ID NO: 56 - protein, 17 aa, synthetic construct
  REGION 1..17: note = Synthetic Construct
IINPNFGSTNYAQKFQG

SEQ ID NO: 57 - protein, 17 aa, synthetic construct
  REGION 1..17: note = Synthetic Construct
IISPSGGGTKYAQKFQG

SEQ ID NO: 58 - protein, 16 aa, synthetic construct
  REGION 1..16: note = Synthetic Construct
RIYWDGDKRYSPSLKS

SEQ ID NO: 59 - protein, 17 aa, synthetic construct
  REGION 1..17: note = Synthetic Construct
WISPSSGSTKYAQKFQG

SEQ ID NO: 60 - protein, 17 aa, synthetic construct
  REGION 1..17: note = Synthetic Construct
GIIPVFGTPNYAQKFQG

SEQ ID NO: 61 - protein, 17 aa, synthetic construct
  REGION 1..17: note = Synthetic Construct
GIIPVFGTANYAQKFQG

SEQ ID NO: 62 - protein, 16 aa, synthetic construct
  REGION 1..16: note = Synthetic Construct
AISYSGSTYYSPSLKS

SEQ ID NO: 63 - protein, 17 aa, synthetic construct
  REGION 1..17: note = Synthetic Construct
RIYPSGGSTNYAQKFQG

SEQ ID NO: 64 - protein, 16 aa, synthetic construct
  REGION 1..16: note = Synthetic Construct
AISYSGSTYYSPSLKS

SEQ ID NO: 65 - protein, 17 aa, synthetic construct
  REGION 1..17: note = Synthetic Construct
AISGSGSSTYYADSVKG

SEQ ID NO: 66 - protein, 17 aa, synthetic construct
  REGION 1..17: note = Synthetic Construct
AISGSGSSTYYADSVKG

SEQ ID NO: 67 - protein, 17 aa, synthetic construct
  REGION 1..17: note = Synthetic Construct
AISGSGSSTYYADSVKG

SEQ ID NO: 68 - protein, 17 aa, synthetic construct
  REGION 1..17: note = Synthetic Construct
GISGAGGSTYYADSVKG

SEQ ID NO: 69 - protein, 15 aa, synthetic construct
  REGION 1..15: note = Synthetic Construct
DRAPGSSGYYDGFDY

SEQ ID NO: 70 - protein, 9 aa, synthetic construct
  REGION 1..9: note = Synthetic Construct
RVPLGAFDY

SEQ ID NO: 71 - protein, 11 aa, synthetic construct
  REGION 1..11: note = Synthetic Construct
GVRGSYGWFDV

SEQ ID NO: 72 - protein, 9 aa, synthetic construct
  REGION 1..9: note = Synthetic Construct
SGHAYDFDY

SEQ ID NO: 73 - protein, 9 aa, synthetic construct
  REGION 1..9: note = Synthetic Construct
YYYGASFDV

SEQ ID NO: 74 - protein, 10 aa, synthetic construct
  REGION 1..10: note = Synthetic Construct
GARGYYALDY

SEQ ID NO: 75 - protein, 7 aa, synthetic construct
  REGION 1..7: note = Synthetic Construct
FSDGFDP

SEQ ID NO: 76 - protein, 9 aa, synthetic construct
  REGION 1..9: note = Synthetic Construct
YGYGPVFDY

SEQ ID NO: 77 - protein, 10 aa, synthetic construct
  REGION 1..10: note = Synthetic Construct
YGYGGAYFDV

SEQ ID NO: 78 - protein, 9 aa, synthetic construct
  REGION 1..9: note = Synthetic Construct
QGILGGFAY

SEQ ID NO: 79 - protein, 15 aa, synthetic construct
  REGION 1..15: note = Synthetic Construct
HYLFAGSTSYDAFDI

SEQ ID NO: 80 - protein, 9 aa, synthetic construct
  REGION 1..9: note = Synthetic Construct
QRGYGRFAY

SEQ ID NO: 81 - protein, 13 aa, synthetic construct
  REGION 1..13: note = Synthetic Construct
QGYYGGEGYAVDY

SEQ ID NO: 82 - protein, 11 aa, synthetic construct
  REGION 1..11: note = Synthetic Construct
RGSYGFGAFDY

SEQ ID NO: 83 - protein, 11 aa, synthetic construct
  REGION 1..11: note = Synthetic Construct
RGSYGFGAFDY

SEQ ID NO: 84 - protein, 11 aa, synthetic construct
  REGION 1..11: note = Synthetic Construct
RGSYGFGAFDY

SEQ ID NO: 85 - protein, 4 aa, synthetic construct
  REGION 1..4: note = Synthetic Construct
LGDY

SEQ ID NO: 86 - protein, 15 aa, synthetic construct
  REGION 1..15: note = Synthetic Construct
RASQSVDFHGISFLH

SEQ ID NO: 87 - protein, 12 aa, synthetic construct
  REGION 1..12: note = Synthetic Construct
RASQTVISRYLA

SEQ ID NO: 88 - protein, 10 aa, synthetic construct
  REGION 1..10: note = Synthetic Construct
SASSRVSYVY

SEQ ID NO: 89 - protein, 15 aa, synthetic construct
  REGION 1..15: note = Synthetic Construct
RASQSVDFYGISFLD

SEQ ID NO: 90 - protein, 16 aa, synthetic construct
  REGION 1..16: note = Synthetic Construct
KSGQSLLHGDVKTYLY

SEQ ID NO: 91 - protein, 12 aa, synthetic construct
  REGION 1..12: note = Synthetic Construct
RASQSVRGRLLA

SEQ ID NO: 92 - protein, 15 aa, synthetic construct
  REGION 1..15: note = Synthetic Construct
RASQSVDFVGISFLH

SEQ ID NO: 93 - protein, 10 aa, synthetic construct
  REGION 1..10: note = Synthetic Construct
SASSSVTYIY

SEQ ID NO: 94 - protein, 15 aa, synthetic construct
  REGION 1..15: note = Synthetic Construct
RASQSVDFHGISFLH

SEQ ID NO: 95 - protein, 15 aa, synthetic construct
  REGION 1..15: note = Synthetic Construct
RASESVDFHGKSFLA

SEQ ID NO: 96 - protein, 15 aa, synthetic construct
  REGION 1..15: note = Synthetic Construct
RASQSVDFYGFSFLA

SEQ ID NO: 97 - protein, 10 aa, synthetic construct
  REGION 1..10: note = Synthetic Construct
SASSSVGYVY

SEQ ID NO: 98 - protein, 15 aa, synthetic construct
  REGION 1..15: note = Synthetic Construct
RASQSVDFYGISFLH

SEQ ID NO: 99 - protein, 11 aa, synthetic construct
  REGION 1..11: note = Synthetic Construct
RASQTIGRYLN

SEQ ID NO: 100 - protein, 11 aa, synthetic construct
  REGION 1..11: note = Synthetic Construct
RASQTIGRYLN

SEQ ID NO: 101 - protein, 11 aa, synthetic construct
  REGION 1..11: note = Synthetic Construct
RASQTIGRYLN

SEQ ID NO: 102 - protein, 11 aa, synthetic construct
  REGION 1..11: note = Synthetic Construct
RASQGISSVLA

SEQ ID NO: 103 - protein, 7 aa, synthetic construct
  REGION 1..7: note = Synthetic Construct
DASSLES

SEQ ID NO: 104 - protein, 7 aa, synthetic construct
  REGION 1..7: note = Synthetic Construct
DASNLET

SEQ ID NO: 105 - protein, 7 aa, synthetic construct
  REGION 1..7: note = Synthetic Construct
AASTLQS

SEQ ID NO: 106 - protein, 7 aa, synthetic construct
  REGION 1..7: note = Synthetic Construct
AASTLQS

SEQ ID NO: 107 - protein, 7 aa, synthetic construct
  REGION 1..7: note = Synthetic Construct
DASNRAT

SEQ ID NO: 108 - protein, 7 aa, synthetic construct
  REGION 1..7: note = Synthetic Construct
AASSLQS

SEQ ID NO: 109 - protein, 7 aa, synthetic construct
  REGION 1..7: note = Synthetic Construct
DASNRAT

SEQ ID NO: 110 - protein, 7 aa, synthetic construct
  REGION 1..7: note = Synthetic Construct
DASNRAT

SEQ ID NO: 111 - protein, 7 aa, synthetic construct
  REGION 1..7: note = Synthetic Construct
DASNRAT

SEQ ID NO: 112 - protein, 7 aa, synthetic construct
  REGION 1..7: note = Synthetic Construct
AASSLQS

SEQ ID NO: 113 - protein, 7 aa, synthetic construct
  REGION 1..7: note = Synthetic Construct
DASNRAT

SEQ ID NO: 114 - protein, 7 aa, synthetic construct
  REGION 1..7: note = Synthetic Construct
DASSLES

SEQ ID NO: 115 - protein, 7 aa, synthetic construct
  REGION 1..7: note = Synthetic Construct
DASNRAT

SEQ ID NO: 116 - protein, 7 aa, synthetic construct
  REGION 1..7: note = Synthetic Construct
DASNRAT

SEQ ID NO: 117 - protein, 7 aa, synthetic construct
  REGION 1..7: note = Synthetic Construct
DASNRAT

SEQ ID NO: 118 - protein, 7 aa, synthetic construct
  REGION 1..7: note = Synthetic Construct
DASNRAT

SEQ ID NO: 119 - protein, 7 aa, synthetic construct
  REGION 1..7: note = Synthetic Construct
AASTLQS

SEQ ID NO: 120 - protein, 9 aa, synthetic construct
  REGION 1..9: note = Synthetic Construct
HQRSTTPLT

SEQ ID NO: 121 - protein, 9 aa, synthetic construct
  REGION 1..9: note = Synthetic Construct
QQYYAWPLT

SEQ ID NO: 122 - protein, 11 aa, synthetic construct
  REGION 1..11: note = Synthetic Construct
QHYVSSPRVYT

SEQ ID NO: 123 - protein, 9 aa, synthetic construct
  REGION 1..9: note = Synthetic Construct
QQGGTSPWT

SEQ ID NO: 124 - protein, 11 aa, synthetic construct
  REGION 1..11: note = Synthetic Construct
QQYASWPPGFT

SEQ ID NO: 125 - protein, 9 aa, synthetic construct
  REGION 1..9: note = Synthetic Construct
AQYLELPYT

SEQ ID NO: 126 - protein, 9 aa, synthetic construct
  REGION 1..9: note = Synthetic Construct
VQGLQWPHT

SEQ ID NO: 127 - protein, 11 aa, synthetic construct
  REGION 1..11: note = Synthetic Construct
QQYTSSPRGFT

SEQ ID NO: 128 - protein, 9 aa, synthetic construct
  REGION 1..9: note = Synthetic Construct
VQGIQWPWT

SEQ ID NO: 129 - protein, 9 aa, synthetic construct
  REGION 1..9: note = Synthetic Construct
QQYLELPFT

SEQ ID NO: 130 - protein, 9 aa, synthetic construct
  REGION 1..9: note = Synthetic Construct
QQSYRTPLT

SEQ ID NO: 131 - protein, 9 aa, synthetic construct
  REGION 1..9: note = Synthetic Construct
QQYTYAPFT

SEQ ID NO: 132 - protein, 9 aa, synthetic construct
  REGION 1..9: note = Synthetic Construct
VQATQTPFT

SEQ ID NO: 133 - protein, 9 aa, synthetic construct
  REGION 1..9: note = Synthetic Construct
QQRYPWPYT

SEQ ID NO: 134 - protein, 9 aa, synthetic construct
  REGION 1..9: note = Synthetic Construct
QQRYPWPYT

SEQ ID NO: 135 - protein, 9 aa, synthetic construct
  REGION 1..9: note = Synthetic Construct
QQRYPWPYT

SEQ ID NO: 136 - protein, 9 aa, synthetic construct
  REGION 1..9: note = Synthetic Construct
QQYYSIPFT

SEQ ID NO: 137 - protein, 16 aa, synthetic construct
  REGION 1..16: note = Synthetic Construct
LTVDYFCDIDPLYCNA

SEQ ID NO: 138 - protein, 13 aa, synthetic construct
  REGION 1..13: note = Synthetic Construct
GGGPLGLAGSGGS

SEQ ID NO: 139 - protein, 34 aa, synthetic construct
  REGION 1..34: note = Synthetic Construct
EVGSYLTVDYFCDIDPLYCNAGGGPLGLAGSGGS

SEQ ID NO: 140 - protein, 214 aa, synthetic construct
  REGION 1..214: note = Synthetic Construct
DIQLTQSPSSLSASVGDRVTITCRASQTIGRYLNWYQQKPGKAPKLLIYDASNRATGIPSRFSGSGSGTDFTLTISSLQPEDFATYYCQQRYPWPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 447 aa, synthetic construct
  REGION 1..447: note = Synthetic Construct
EVQLVESGGGLVQPGGSLRLSCAASGFTFTNYAIHWVRQAPGKGLEWVSAISGSGSSTYYADSVKGRFTISRDNSKNTLYLQLNSLRAEDTAVYYCARRGSYGFGAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 142 - protein, 248 aa, synthetic construct
  REGION 1..248: note = Synthetic Construct
EVGSYLTVDYFCDIDPLYCNAGGGPLGLAGSGGSDIQLTQSPSSLSASVGDRVTITCRASQTIGRYLNWYQQKPGKAPKLLIYDASNRATGIPSRFSGSGSGTDFTLTISSLQPEDFATYYCQQRYPWPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 447 aa, synthetic construct; identical to SEQ ID NO: 141
  REGION 1..447: note = Synthetic Construct

SEQ ID NO: 144 - protein, 214 aa, synthetic construct; identical to SEQ ID NO: 140
  REGION 1..214: note = Synthetic Construct

SEQ ID NO: 145 - protein, 450 aa, synthetic construct
  REGION 1..450: note = Synthetic Construct
EVQLVESGGGLVQPGGSLRLSCAASGFTFTNYAIHWVRQAPGKGLEWVSAISGSGSSTYYADSVKGRFTISRDNSKNTLYLQLNSLRAEDTAVYYCARRGSYGFGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 214 aa, synthetic construct; identical to SEQ ID NO: 140
  REGION 1..214: note = Synthetic Construct

SEQ ID NO: 147 - protein, 450 aa, synthetic construct
  REGION 1..450: note = Synthetic Construct
EVQLVESGGGLVQPGGSLRLSCAASGFTFTNYAIHWVRQAPGKGLEWVSAISGSGSSTYYADSVKGRFTISRDNSKNTLYLQLNSLRAEDTAVYYCARRGSYGFGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 248 aa, synthetic construct; identical to SEQ ID NO: 142
  REGION 1..248: note = Synthetic Construct

SEQ ID NO: 149 - protein, 450 aa, synthetic construct; identical to SEQ ID NO: 145
  REGION 1..450: note = Synthetic Construct

SEQ ID NO: 150 - protein, 248 aa, synthetic construct; identical to SEQ ID NO: 142
  REGION 1..248: note = Synthetic Construct

SEQ ID NO: 151 - protein, 450 aa, synthetic construct; identical to SEQ ID NO: 147
  REGION 1..450: note = Synthetic Construct

SEQ ID NO: 152 - protein, 34 aa, synthetic construct
  REGION 1..34: note = Synthetic Construct
EVGSYNNAAPDCPAADVYCNVGGGPLGLAGSGGS

SEQ ID NO: 153 - protein, 34 aa, synthetic construct
  REGION 1..34: note = Synthetic Construct
EVGSYDDTAFDCDDDFDDCATGGGPLGLAGSGGS

SEQ ID NO: 154 - protein, 34 aa, synthetic construct
  REGION 1..34: note = Synthetic Construct
EVGSYHAYHNDCSDDDDFCAYGGGPLGLAGSGGS

SEQ ID NO: 155 - protein, 34 aa, synthetic construct
  REGION 1..34: note = Synthetic Construct
EVGSYPADDFDCPAADVFCSPGGGPLGLAGSGGS

SEQ ID NO: 156 - protein, 34 aa, synthetic construct
  REGION 1..34: note = Synthetic Construct
EVGSYAPSVDDCPTYDDFCYDGGGPLGLAGSGGS

SEQ ID NO: 157 - protein, 34 aa, synthetic construct; identical to SEQ ID NO: 139
  REGION 1..34: note = Synthetic Construct

SEQ ID NO: 158 - protein, 34 aa, synthetic construct
  REGION 1..34: note = Synthetic Construct
EVGSYNYNDDDCYDDDYACDYGGGPLGLAGSGGS

SEQ ID NO: 159 - protein, 34 aa, synthetic construct
  REGION 1..34: note = Synthetic Construct
EVGSYDTADHDCYDDDDFCYDGGGPLGLAGSGGS

SEQ ID NO: 160 - protein, 34 aa, synthetic construct
  REGION 1..34: note = Synthetic Construct
EVGSYDFLSASCDDDYAPCAFGGGPLGLAGSGGS

SEQ ID NO: 161 - protein, 34 aa, synthetic construct
  REGION 1..34: note = Synthetic Construct
EVGSYYPPDDTCDDDYDPCAIGGGPLGLAGSGGS

SEQ ID NO: 162 - protein, 34 aa, synthetic construct
  REGION 1..34: note = Synthetic Construct
EVGSYAQSSFDCPDHDAFCDPGGGPLGLAGSGGS

SEQ ID NO: 163 - protein, 34 aa, synthetic construct
  REGION 1..34: note = Synthetic Construct
EVGSYAASPATCDDTLFFCPAGGGPLGLAGSGGS

SEQ ID NO: 164 - protein, 32 aa, synthetic construct
  REGION 1..32: note = Synthetic Construct
EVGSYDSCPAEVVGIFCIQGGGPLGLAGSGGS

SEQ ID NO: 165 - protein, 32 aa, synthetic construct
  REGION 1..32: note = Synthetic Construct
EVGSYFYCTGAAVGIFCSAGGGPLGLAGSGGS

SEQ ID NO: 166 - protein, 32 aa, synthetic construct
  REGION 1..32: note = Synthetic Construct
EVGSYVACRIFDDDPFCIVGGGPLGLAGSGGS

SEQ ID NO: 167 - protein, 16 aa, synthetic construct
  REGION 1..16: note = Synthetic Construct
NNAAPDCPAADVYCNV

SEQ ID NO: 168 - protein, 16 aa, synthetic construct
  REGION 1..16: note = Synthetic Construct
DDTAFDCDDDFDDCAT

SEQ ID NO: 169 - protein, 16 aa, synthetic construct
  REGION 1..16: note = Synthetic Construct
HAYHNDCSDDDDFCAY

SEQ ID NO: 170 - protein, 16 aa, synthetic construct
  REGION 1..16: note = Synthetic Construct
PADDFDCPAADVFCSP

SEQ ID NO: 171 - protein, 16 aa, synthetic construct
  REGION 1..16: note = Synthetic Construct
APSVDDCPTYDDFCYD

SEQ ID NO: 172 - protein, 16 aa, synthetic construct
  REGION 1..16: note = Synthetic Construct
LTVDYFCDIDPLYCNA

SEQ ID NO: 173 - protein, 16 aa, synthetic construct
  REGION 1..16: note = Synthetic Construct
NYNDDDCYDDDYACDY

SEQ ID NO: 174 - protein, 16 aa, synthetic construct
  REGION 1..16: note = Synthetic Construct
DTADHDCYDDDDFCYD

SEQ ID NO: 175 - protein, 16 aa, synthetic construct
  REGION 1..16: note = Synthetic Construct
DFLSASCDDDYAPCAF

SEQ ID NO: 176 - protein, 16 aa, synthetic construct
  REGION 1..16: note = Synthetic Construct
YPPDDTCDDDYDPCAI

SEQ ID NO: 177 - protein, 16 aa, synthetic construct
  REGION 1..16: note = Synthetic Construct
AQSSFDCPDHDAFCDP

SEQ ID NO: 178 - protein, 16 aa, synthetic construct
  REGION 1..16: note = Synthetic Construct
AASPATCDDTLFFCPA

SEQ ID NO: 179 - protein, 14 aa, synthetic construct
  REGION 1..14: note = Synthetic Construct
DSCPAEVVGIFCIQ

SEQ ID NO: 180 - protein, 14 aa, synthetic construct
  REGION 1..14: note = Synthetic Construct
FYCTGAAVGIFCSA

SEQ ID NO: 181 - protein, 14 aa, synthetic construct
  REGION 1..14: note = Synthetic Construct
VACRIFDDDPFCIV

SEQ ID NO: 182-183 - skipped (2 entries)

SEQ ID NO: 184 - protein, 16 aa, synthetic construct
  REGION 1..16: note = Synthetic Construct
  VARIANT 1: note = Xaa = Ala, Glu, or Arg
  VARIANT 3: note = Xaa = Ser or Tyr
  VARIANT 4: note = Xaa = His, Trp, or Tyr
  VARIANT 5: note = Xaa = Asp or Ser
  VARIANT 7: note = Xaa = Asp, Asn, or Ser
  VARIANT 8: note = Xaa = Lys or Thr
  VARIANT 9: note = Xaa = Arg or Tyr
  VARIANT 11: note = Xaa = Asn or Ser
XIXXXGXXXYXPSLKS

SEQ ID NO: 185 - protein, 17 aa, synthetic construct
  REGION 1..17: note = Synthetic Construct
  VARIANT 1: note = Xaa = Ala, Gly, Ile, Arg, or Trp
  VARIANT 3: note = Xaa = Ile, Asn, Ser, or Tyr,
  VARIANT 4: note = Xaa = Gly or Pro
  VARIANT 5: note = Xaa = Ala, Asn, Ser, or Val
  VARIANT 6: note = Xaa = Phe, Gly, or Ser
  VARIANT 7: note = Xaa = Gly or Ser
  VARIANT 8: note = Xaa = Gly, Ser, or Thr
  VARIANT 9: note = Xaa = Ala, Pro, or Thr
  VARIANT 10: note = Xaa = Lys, Asn, or Tyr
  VARIANT 13: note = Xaa = Asp or Gln
  VARIANT 14: note = Xaa = Lys or Ser
  VARIANT 15: note = Xaa = Phe or Val
  VARIANT 16: note = Xaa = Lys or Gln
XIXXXXXXXXYAXXXXG

SEQ ID NO: 186-188 - skipped (3 entries)

SEQ ID NO: 189 - protein, 15 aa, synthetic construct
  REGION 1..15: note = Synthetic Construct
  VARIANT 1: note = Xaa = Asp or His
  VARIANT 2: note = Xaa = Arg or Tyr
  VARIANT 3: note = Xaa = Ala or Leu
  VARIANT 4: note = Xaa = Phe or Pro
  VARIANT 5: note = Xaa = Ala or Gly
  VARIANT 6: note = Xaa = Gly or Ser
  VARIANT 8: note = Xaa = Gly or Thr
  VARIANT 9: note = Xaa = Ser or Tyr
  VARIANT 12: note = Xaa = Ala or Gly
  VARIANT 15: note = Xaa = Ile or Tyr
XXXXXXSXXYDXFDX

SEQ ID NO: 190 - protein, 10 aa, synthetic construct
  REGION 1..10: note = Synthetic Construct
  VARIANT 5: note = Xaa = Arg or Ser
  VARIANT 7: note = Xaa = Gly, Ser, or Thr
  VARIANT 9: note = Xaa = Ile or Val
SASSXVXYXY

SEQ ID NO: 191 - protein, 11 aa, synthetic construct
  REGION 1..11: note = Synthetic Construct
  VARIANT 5: note = Xaa = Gly or Thr
  VARIANT 7: note = Xaa = Gly or Ser
  VARIANT 8: note = Xaa = Arg or Ser
  VARIANT 9: note = Xaa = Val or Tyr
  VARIANT 11: note = Xaa = Ala or Asn
RASQXIXXXLX

SEQ ID NO: 192 - protein, 12 aa, synthetic construct
  REGION 1..12: note = Synthetic Construct
  VARIANT 5: note = Xaa = Ser or Thr
  VARIANT 7: note = Xaa = Ile or Arg
  VARIANT 8: note = Xaa = Gly or Ser
  VARIANT 10: note = Xaa = Leu or Tyr
RASQXVXXRXLA

SEQ ID NO: 193 - protein, 15 aa, synthetic construct
  REGION 1..15: note = Synthetic Construct
  VARIANT 4: note = Xaa = Glu or Gln
  VARIANT 9: note = Xaa = His, Val, or Tyr
  VARIANT 11: note = Xaa = Phe, Ile, or Lys
  VARIANT 15: note = Xaa = Ala, Asp, or His
RASXSVDFXGXSFLX

SEQ ID NO: 194-195 - skipped (2 entries)

SEQ ID NO: 196 - protein, 11 aa, synthetic construct
  REGION 1..11: note = Synthetic Construct
  VARIANT 2: note = Xaa = His or Gln
  VARIANT 4: note = Xaa = Ala, Thr, or Val
  VARIANT 6: note = Xaa = Ser or Trp
  VARIANT 8: note = Xaa = Pro or Arg
  VARIANT 9: note = Xaa = Gly or Val
  VARIANT 10: note = Xaa = Phe or Tyr
QXYXSXPXXXT

SEQ ID NO: 197 - protein, 16 aa, synthetic construct
  REGION 1..16: note = Synthetic Construct
  VARIANT 1: note = Xaa = Asp, His, Asn, or Tyr
  VARIANT 2: note = Xaa = Ala, Asp, Phe, Pro, Thr, or Tyr
  VARIANT 3: note = Xaa = Ala, Leu, Asn, Pro, Thr, or Tyr
  VARIANT 4: note = Xaa = Ala, Asp, His, or Ser
  VARIANT 5: note = Xaa = Ala, Asp, Phe, His, or Asn
  VARIANT 6: note = Xaa = Asp, Ser, or Thr
  VARIANT 8: note = Xaa = Asp, Ser, or Tyr
  VARIANT 11: note = Xaa = Asp, Phe, or Tyr
  VARIANT 12: note = Xaa = Ala, Asp, or Tyr
  VARIANT 13: note = Xaa = Ala, Asp, Phe, or Pro
  VARIANT 15: note = Xaa = Ala, Asp, or Tyr
  VARIANT 16: note = Xaa = Asp, Phe, Ile, Thr, or Tyr
XXXXXXCXDDXXXCXX

SEQ ID NO: 198 - protein, 16 aa, synthetic construct
  REGION 1..16: note = Synthetic Construct
  VARIANT 1: note = Xaa = Ala, Asn, or Pro
  VARIANT 2: note = Xaa = Ala, Asn, Pro, or Gln
  VARIANT 3: note = Xaa = Ala, Asp, or Ser
  VARIANT 4: note = Xaa = Ala, Asp, Ser, or Val
  VARIANT 5: note = Xaa = Asp, Phe, or Pro
  VARIANT 9: note = Xaa = Ala, Asp, or Thr
  VARIANT 10: note = Xaa = Ala, His, or Tyr
  VARIANT 12: note = Xaa = Ala, Asp, or Val
  VARIANT 13: note = Xaa = Phe or Tyr
  VARIANT 15: note = Xaa = Asp, Asn, Ser, or Tyr
  VARIANT 16: note = Xaa = Asp, Pro, or Val
XXXXXDCPXXDXXCXX

SEQ ID NO: 199 - protein, 16 aa, synthetic construct
  REGION 1..16: note = Synthetic Construct
  VARIANT 1: note = Xaa = Ala or Leu
  VARIANT 2: note = Xaa = Ala or Thr
  VARIANT 3: note = Xaa = Ser or Val
  VARIANT 4: note = Xaa = Asp or Pro
  VARIANT 5: note = Xaa = Ala or Tyr
  VARIANT 6: note = Xaa = Phe or Thr
  VARIANT 9: note = Xaa = Asp or Ile
  VARIANT 10: note = Xaa = Asp or Thr
  VARIANT 11: note = Xaa = Leu or Pro
  VARIANT 12: note = Xaa = Phe or Leu
  VARIANT 13: note = Xaa = Phe or Tyr
  VARIANT 15: note = Xaa = Asn or Pro
XXXXXXCDXXXXXCXA

SEQ ID NO: 200 - skipped

SEQ ID NO: 201 - protein, 118 aa, synthetic construct
  REGION 1..118: note = Synthetic Construct
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSS

SEQ ID NO: 202 - protein, 113 aa, synthetic construct
  REGION 1..113: note = Synthetic Construct
DIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIK

SEQ ID NO: 203 - protein, 117 aa, synthetic construct
  REGION 1..117: note = Synthetic Construct
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 204 - protein, 112 aa, synthetic construct
  REGION 1..112: note = Synthetic Construct
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

(19) United States
(12) Patent Application Publication
(10) Pub. No.: US 2024/0417463 A1
(43) Pub. Date: Dec. 19, 2024

(54) ANTI-CD47 ANTIBODIES AND METHODS OF USE THEREOF

(71) Applicant: Adagene PTE. LTD., Singapore (SG)

(72) Inventors: Fangyong DU, Suzhou, Jiangsu (CN); Peter Peizhi LUO, Morgan Hill, CA (US); Yan LI, San Jose, CA (US); Bin CAI, Suzhou, Jiangsu (CN); Jianfeng SHI, Suzhou, Jiangsu (CN); Jiangchun XU, San Diego, CA (US); Aaron NGUYEN, San Jose, CA (US); Songmao ZHENG, West Harrison, IN (US); Guizhong LIU, Fort Lee, NJ (US); Zhengxi DAI, Suzhou, Jiangsu (CN)

(73) Assignee: Adagene PTE. LTD., Singapore (SG)

(21) Appl. No.: 18/705,386

(22) PCT Filed: Oct. 27, 2022
(86) PCT No.: PCT/CN2022/127875

§ 371 Date: Apr. 26, 2024

(30) Foreign Application Priority Data
Oct. 27, 2021 (WO) PCT/CN2021/126597

(51) Int. Cl. C07K 16/28; A61K 39/00; A61P 35/00

(52) U.S. Cl. CPC C07K 16/2803; A61P 35/00; A61K 2039/505; C07K 2317/32; C07K 2317/33; C07K 2317/565; C07K 2317/732; C07K 2317/76; C07K 2317/92; C07K 2317/94; C07K 2319/50

(57) ABSTRACT

Provided herein are antibodies, masked antibodies (e.g., activatable antibodies), and antigen-binding fragments that target human CD47, polynucleotides encoding the same, therapeutic compositions thereof, and their use to treat CD47-positive diseases such as cancer. The masked antibodies described herein may comprise an IgG1 Fc region.

### CROSS REFERENCE TO RELATED APPLICATIONS

This application claims the priority benefit of International Application No. PCT/CN2021/126597, filed on Oct. 27, 2021, which is incorporated herein by reference in its entirety.

### REFERENCE TO SEQUENCE LISTING

The content of the following submission on ASCII text file is incorporated herein by reference in its entirety: a computer readable form (CRF) of the Sequence Listing (file name: 695402001641seqlist.xml, date recorded: Oct. 14, 2022, size: 288,462 bytes).

### FIELD

The present disclosure relates to antibodies, masked antibodies (e.g., activatable antibodies), and antigen binding fragments thereof that target human CD47, polynucleotides encoding the same, pharmaceutical compositions thereof, and their use.

### BACKGROUND

The binding of CD47, overexpressed on the surface of many types of cancer cells, to signal-regulatory protein α (SIRPα) on myeloid cells initiates an inhibitory signaling pathway that leads to the “don't eat me” signal and therefore evasion of malignant cells from the host immune system. Therapies targeting the CD47/SIRPα signaling axis have shown success in preclinical models and are now in clinical trials for both solid and hematologic malignancies. The anti-CD47 therapies have demonstrated promising activities in early clinical trials; however, there have also been challenges. For example, many different human cell types, including red blood cells (RBCs), express CD47, presenting a large antigen sink for anti-CD47 antibodies. Blocking CD47 on RBCs, such as by Magrolimab (Hu5F9), has led to transient anemia, requiring step-up dosing in the clinic. It is noteworthy that many anti-CD47 therapies, such as Hu5F9, TJC4, AK117, CC-90002, and IBI188, are antibodies of the IgG4 subclass type. IgG1 antibodies can target ADCC- and ADCP-competent immune cells to cells expressing the antigens targeted by the IgG1 antibodies. As CD47 is also widely expressed on normal tissues, it is commonly believed that the IgG1 subclass type should not be used in a therapeutic anti-CD47 antibody in order to prevent or limit on-target/off-tumor toxicities.

Therefore, there remains a need for novel anti-CD47 antibodies capable of blocking binding of CD47 to SIRPα without exhibiting the undesirable effects of binding to RBCs and other non-cancerous cells.

### BRIEF SUMMARY

To provide anti-CD47 antibodies with powerful anti-tumor activity and reduced off-target effects, the inventors developed a suite of masked anti-CD47 antibodies (including activatable anti-CD47 antibodies), each comprising an N-terminal masking peptide and an IgG1 Fc region. The anti-tumor activity of the antibodies is two-fold: 1) blocking of the CD47-SIRPα interaction to allow recognition and phagocytosis of tumor cells by macrophages, and 2) tumor killing through IgG1 Fc-mediated effector functions such as ADCC and ADCP. The masking peptide is designed to compete with the anti-CD47 antibody moiety for target binding, thereby inhibiting on-target off-tumor activities in healthy tissues and allowing anti-tumor activities in the tumor microenvironment (TME).

A masked antibody is designed to mask its antigen-binding site with a masking moiety in the masking peptide, which prevents the antibody from binding to its target in healthy tissues. Masked antibodies may be activatable, also known as a SAFEBODY™, wherein the masking moiety is designed to be unmasked to activate the antibody in a TME having certain activation conditions, allowing the antigen-binding site to bind to its target. For example, the masking peptide of an activatable antibody may comprise a cleavable moiety that, upon cleavage by a protease, results in removal of the masking moiety from the antibody and activation of the antibody to bind CD47. In normal tissues and in circulation, the masking moiety in the activatable anti-CD47 antibody functions to block binding to CD47, reducing off-target effects associated with anti-CD47 antibodies, such as anemia. However, in the TME where protease activity has been reported to be elevated, the masking moiety is cleaved off, enabling the activated anti-CD47 antibody to bind CD47 on tumor cells. Thus, the activatable anti-CD47 antibodies described herein exhibit enhanced anti-tumor activity, both through blocking of the CD47-SIRPα interaction and ADCC/ADCP effector functions, with reduced toxicity on normal cells as compared to existing anti-CD47 antibodies.

Accordingly, in some aspects, provided herein are antibodies (e.g., isolated antibodies), antigen-binding fragments, and masked antibodies (e.g., activatable antibodies) that bind to CD47 (e.g., human CD47). In some embodiments, the antibodies (e.g., isolated antibodies), antigen-binding fragments, and masked antibodies (e.g., activatable antibodies) have at least one (e.g., at least one, at least two, at least three, at least four, at least five, or all six) of the following functional properties: (a) bind to human CD47 with a Kp of 500 nM or less; (b) are cross-reactive with monkey, rat, or dog CD47; (c) are capable of inhibiting tumor cell growth; (d) have therapeutic effect on a cancer; (e) block binding between CD47 and SIRP proteins (e.g., SIRPα); and (f) induce antibody-dependent cellular cytotoxicity (ADCC) and/or antibody-dependent cellular phagocytosis (ADCP) against CD47-expressing tumor cells.

Accordingly, in some aspects, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM. In some embodiments, 1) the VH comprises a first complementary-determining-region (CDR-H1), a second complementary-determining-region (CDR-H2), and a third complementary-determining-region (CDR-H3), wherein the CDR-H1 comprises the amino acid sequence of SEQ ID NO: 182 or 183; wherein the CDR-H2 comprises the amino acid sequence of SEQ ID NO: 184 or 185; and wherein the CDR-H3 comprises an amino acid sequence selected from the group consisting of SEQ ID NOs: 186-189; and/or 2) the VL comprises a first complementary-determining-region (CDR-L1), a second complementary-determining-region (CDR-L2), and a third complementary-determining-region (CDR-L3), wherein the CDR-L1 comprises an amino acid sequence selected from the group consisting of SEQ ID NOs: 190-193; wherein the CDR-L2 comprises the amino acid sequence of SEQ ID NO: 194; and wherein the CDR-L3 comprises the amino acid sequence of SEQ ID NO: 195 or 196. In certain embodiments, the VH comprises a CDR-H1 comprising the amino acid sequence of SEQ ID NO: 182, a CDR-H2 comprising the amino acid sequence of SEQ ID NO: 185, and a CDR-H3 comprising the amino acid sequence of SEQ ID NO: 188; and b) the VL comprises a CDR-L1 comprising the amino acid sequence of SEQ ID NO: 191, a CDR-L2 comprising the amino acid sequence of SEQ ID NO: 194, and a CDR-L3 comprising the amino acid sequence of SEQ ID NO: 195. In some embodiments, the masked antibody comprises a first polypeptide comprising, from N terminus to C terminus, the masking peptide and the VL, and a second polypeptide comprising the VH. In other embodiments, the masked antibody comprises a first polypeptide comprising, from N terminus to C terminus, the masking peptide and the VH, and a second polypeptide comprising the VL. In additional embodiments, the masked antibody comprises a first polypeptide comprising, from N terminus to C terminus, the masking peptide, the VL, and the VH. In yet additional embodiments, the masked antibody comprises a first polypeptide comprising, from N terminus to C terminus the masking peptide, the VH, and the VL. In some embodiments, the masking moiety (MM) comprises an amino acid sequence selected from the group consisting of SEQ ID NOs: 197-200. In one embodiment, the MM comprises the amino acid sequence of SEQ ID NO: 199. In certain embodiments, the MM comprises an amino acid sequence selected from the group consisting of SEQ ID NOs: 137 and 167-181. In one, the MM comprises the amino acid sequence of SEQ ID NO: 137. In some embodiments, the masking peptide, or a part thereof (e.g., the MM) has a masking efficiency of at least about 50 as determined by a Jurkat NFAT reporter assay. In some embodiments, the masking peptide, or a part thereof (e.g., the MM) has a masking efficiency of at least about 100 as determined by a Jurkat NFAT reporter assay. In some embodiments, the masked antibody comprises a first polypeptide comprising, from N terminus to C terminus the masking peptide and the VL; and a second polypeptide comprising the VH. In certain embodiments, the masked antibody comprises a human IgG4 fragment crystallizable (Fc) region. In certain embodiments, the masked antibody comprises a human IgG1 Fc region. In some embodiments, the human IgG1 Fc region comprises two Fc domains, wherein each of the two Fc domains comprises a S239D substitution and/or an 1332E substitution (e.g., a S239D substitution and an 1332E substitution). In some embodiments, the masked antibody binds to one or more amino acid residues of human CD47 selected from the group consisting of K39, W40, K41, F50, D51, G52, T99, E100, L101, and T102. In some embodiments, the masked antibody does not bind to one or more amino acid residues of human CD47 selected from the group consisting of L2, L3, F4, K6, N27, E29, A30, Q31, T34, E35, V36, Y37, D46, T49, A53, E97, R103, E104, G105, and E106. In certain embodiments, the masked antibody binds to amino acid residues K39, W40, K41, F50, D51, G52, T99, E100, L101, and T102 of human CD47 and/or docs not bind to amino acid residues L2, L3, F4, K6, N27, E29, A30, Q31, T34, E35, V36, Y37, D46, T49, A53, E97, R103, E104, G105, and E106 of human CD47.

In some embodiments, provided herein is a masked antibody comprising: (a) a masking peptide comprising, from N terminus to C terminus, a masking moiety (MM) and a linkage moiety (LM); (b) a target binding moiety (TBM) comprising an antibody heavy chain variable region (VH) and an antibody light chain variable region (VL), wherein the TBM binds to human CD47; and (c) an IgG1 Fc region (e.g., a human IgG1 Fc region), or an IgG Fc region (e.g., a human IgG Fc region) with enhanced antibody-dependent cellular cytotoxicity (ADCC) activity, wherein the masking peptide is linked to the N terminus of the VH or the VL, and wherein the MM competes with human CD47 to bind the TBM. In some embodiments, the masked antibody comprises an IgG1 Fc region. In some embodiments, the masked antibody comprises an IgG Fc region with enhanced ADCC activity. In some embodiments, the human IgG1 Fc region comprises two Fc domains, wherein each of the two Fc domains comprises a S239D substitution and/or an 1332E substitution (e.g., a S239D substitution and an 1332E substitution).

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein (a) the VH comprises a CDR-H1 comprising an amino acid sequence selected from the group consisting of SEQ ID NOs: 35-37 and 39-50, a CDR-H2 comprising an amino acid sequence selected from the group consisting of SEQ ID NOs: 52-54 and 56-67, and a CDR-H3 comprising an amino acid sequence selected from the group consisting of SEQ ID NOs: 69-71 and 73-84; and (b) the VL comprises a CDR-L1 comprising an amino acid sequence selected from the group consisting of SEQ ID NOs: 86-88 and 89-101, a CDR-L2 comprising an amino acid sequence selected from the group consisting of SEQ ID NOs: 103-105 and 106-118, and a CDR-L3 comprising an amino acid sequence selected from the group consisting of SEQ ID NOs: 120-122 and 124-135.

In certain embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises an amino acid sequence selected from the group consisting of SEQ ID NOs: 1, 3, 5, 9, 11, 13, 15, 17, 19, 21, 23, 25, 27, 29, and 31; and/or wherein the VL comprises an amino acid sequence selected from the group consisting of SEQ ID NOs: 2, 4, 6, 10, 12, 14, 16, 18, 20, 22, 24, 26, 28, 30, and 32.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 48, a CDR-H2 comprising the amino acid sequence of SEQ ID NO: 65, and a CDR-H3 comprising the amino acid sequence of SEQ ID NO: 82; and 2) the VL comprises a CDR-L1 comprising the amino acid sequence of SEQ ID NO: 99, a CDR-L2 comprising the amino acid sequence of SEQ ID NO: 116, and a CDR-L3 comprising the amino acid sequence of SEQ ID NO: 133. In certain embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 27, and/or the VL comprises the amino acid sequence of SEQ ID NO: 28. In certain embodiments, the MM comprises the amino acid sequence of SEQ ID NO: 137.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 49, a CDR-H2 comprising the amino acid sequence of SEQ ID NO: 66, and a CDR-H3 comprising the amino acid sequence of SEQ ID NO: 83; and 2) the VL comprises a CDR-L1 comprising the amino acid sequence of SEQ ID NO: 100, a CDR-L2 comprising the amino acid sequence of SEQ ID NO: 117, and a CDR-L3 comprising the amino acid sequence of SEQ ID NO: 134. In certain embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 29, and/or the VL comprises the amino acid sequence of SEQ ID NO: 30. In certain embodiments, the MM comprises the amino acid sequence of SEQ ID NO: 137.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 50, a CDR-H2 comprising the amino acid sequence of SEQ ID NO: 67, and a CDR-H3 comprising the amino acid sequence of SEQ ID NO: 84; and 2) the VL comprises a CDR-L1 comprising the amino acid sequence of SEQ ID NO: 101, a CDR-L2 comprising the amino acid sequence of SEQ ID NO: 118, and a CDR-L3 comprising the amino acid sequence of SEQ ID NO: 135. In certain embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 31, and/or the VL comprises the amino acid sequence of SEQ ID NO: 32. In certain embodiments, the MM comprises the amino acid sequence of SEQ ID NO: 137.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 35, a CDR-H2 comprising the amino acid sequence of SEQ ID NO: 52, and a CDR-H3 comprising the amino acid sequence of SEQ ID NO: 69; and 2) the VL comprises a CDR-L1 comprising the amino acid sequence of SEQ ID NO: 86, a CDR-L2 comprising the amino acid sequence of SEQ ID NO: 103, and a CDR-L3 comprising the amino acid sequence of SEQ ID NO: 120. In certain embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 1, and/or the VL comprises the amino acid sequence of SEQ ID NO: 2. In certain embodiments, the MM comprises the amino acid sequence of SEQ ID NO: 137.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 36, a CDR-H2 comprising the amino acid sequence of SEQ ID NO: 53, and a CDR-H3 comprising the amino acid sequence of SEQ ID NO: 70; and 2) the VL comprises a CDR-L1 comprising the amino acid sequence of SEQ ID NO: 87, a CDR-L2 comprising the amino acid sequence of SEQ ID NO: 104, and a CDR-L3 comprising the amino acid sequence of SEQ ID NO: 121. In certain embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 3, and/or the VL comprises the amino acid sequence of SEQ ID NO: 4. In certain embodiments, the MM comprises the amino acid sequence of SEQ ID NO: 137.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 37, a CDR-H2 comprising the amino acid sequence of SEQ ID NO: 54, and a CDR-H3 comprising the amino acid sequence of SEQ ID NO: 71; and 2) the VL comprises a CDR-L1 comprising the amino acid sequence of SEQ ID NO: 88, a CDR-L2 comprising the amino acid sequence of SEQ ID NO: 105, and a CDR-L3 comprising the amino acid sequence of SEQ ID NO: 122. In certain embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 5, and/or the VL comprises the amino acid sequence of SEQ ID NO: 6. In certain embodiments, the MM comprises the amino acid sequence of SEQ ID NO: 137.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 39, a CDR-H2 comprising the amino acid sequence of SEQ ID NO: 56, and a CDR-H3 comprising the amino acid sequence of SEQ ID NO: 73; and 2) the VL comprises a CDR-L1 comprising the amino acid sequence of SEQ ID NO: 90, a CDR-L2 comprising the amino acid sequence of SEQ ID NO: 107, and a CDR-L3 comprising the amino acid sequence of SEQ ID NO: 124. In certain embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 9, and/or the VL comprises the amino acid sequence of SEQ ID NO: 10. In certain embodiments, the MM comprises the amino acid sequence of SEQ ID NO: 137.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 40, a CDR-H2 comprising the amino acid sequence of SEQ ID NO: 57, and a CDR-H3 comprising the amino acid sequence of SEQ ID NO: 74; and 2) the VL comprises a CDR-L1 comprising the amino acid sequence of SEQ ID NO: 91, a CDR-L2 comprising the amino acid sequence of SEQ ID NO: 108, and a CDR-L3 comprising the amino acid sequence of SEQ ID NO: 125. In certain embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 11, and/or the VL comprises the amino acid sequence of SEQ ID NO: 12. In certain embodiments, the MM comprises the amino acid sequence of SEQ ID NO: 137.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 41, a CDR-H2 comprising the amino acid sequence of SEQ ID NO: 58, and a CDR-H3 comprising the amino acid sequence of SEQ ID NO: 75; and 2) the VL comprises a CDR-L1 comprising the amino acid sequence of SEQ ID NO: 92, a CDR-L2 comprising the amino acid sequence of SEQ ID NO: 109, and a CDR-L3 comprising the amino acid sequence of SEQ ID NO: 126. In certain embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 13, and/or the VL comprises the amino acid sequence of SEQ ID NO: 14. In certain embodiments, the MM comprises the amino acid sequence of SEQ ID NO: 137.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 42, a CDR-H2 comprising the amino acid sequence of SEQ ID NO: 59, and a CDR-H3 comprising the amino acid sequence of SEQ ID NO: 76; and 2) the VL comprises a CDR-L1 comprising the amino acid sequence of SEQ ID NO: 93, a CDR-L2 comprising the amino acid sequence of SEQ ID NO: 110, and a CDR-L3 comprising the amino acid sequence of SEQ ID NO: 127. In certain embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 15, and/or the VL comprises the amino acid sequence of SEQ ID NO: 16. In certain embodiments, the MM comprises the amino acid sequence of SEQ ID NO: 137.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 43, a CDR-H2 comprising the amino acid sequence of SEQ ID NO: 60, and a CDR-H3 comprising the amino acid sequence of SEQ ID NO: 77; and 2) the VL comprises a CDR-L1 comprising the amino acid sequence of SEQ ID NO: 94, a CDR-L2 comprising the amino acid sequence of SEQ ID NO: 111, and a CDR-L3 comprising the amino acid sequence of SEQ ID NO: 128. In certain embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 17, and/or the VL comprises the amino acid sequence of SEQ ID NO: 18. In certain embodiments, the MM comprises the amino acid sequence of SEQ ID NO: 137.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 44, a CDR-H2 comprising the amino acid sequence of SEQ ID NO: 61, and a CDR-H3 comprising the amino acid sequence of SEQ ID NO: 78; and 2) the VL comprises a CDR-L1 comprising the amino acid sequence of SEQ ID NO: 95, a CDR-L2 comprising the amino acid sequence of SEQ ID NO: 112, and a CDR-L3 comprising the amino acid sequence of SEQ ID NO: 129. In certain embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 19, and/or the VL comprises the amino acid sequence of SEQ ID NO: 20. In certain embodiments, the MM comprises the amino acid sequence of SEQ ID NO: 137.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 45, a CDR-H2 comprising the amino acid sequence of SEQ ID NO: 62, and a CDR-H3 comprising the amino acid sequence of SEQ ID NO: 79; and 2) the VL comprises a CDR-L1 comprising the amino acid sequence of SEQ ID NO: 96, a CDR-L2 comprising the amino acid sequence of SEQ ID NO: 113, and a CDR-L3 comprising the amino acid sequence of SEQ ID NO: 130. In certain embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 21, and/or the VL comprises the amino acid sequence of SEQ ID NO: 22. In certain embodiments, the MM comprises the amino acid sequence of SEQ ID NO: 137.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 46, a CDR-H2 comprising the amino acid sequence of SEQ ID NO: 63, and a CDR-H3 comprising the amino acid sequence of SEQ ID NO: 80; and 2) the VL comprises a CDR-L1 comprising the amino acid sequence of SEQ ID NO: 97, a CDR-L2 comprising the amino acid sequence of SEQ ID NO: 114, and a CDR-L3 comprising the amino acid sequence of SEQ ID NO: 131. In certain embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 23, and/or the VL comprises the amino acid sequence of SEQ ID NO: 24. In certain embodiments, the MM comprises the amino acid sequence of SEQ ID NO: 137.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) a) the VH comprises a CDR-H1 comprising the amino acid sequence of SEQ ID NO: 47, a CDR-H2 comprising the amino acid sequence of SEQ ID NO: 64, and a CDR-H3 comprising the amino acid sequence of SEQ ID NO: 81; and 2) the VL comprises a CDR-L1 comprising the amino acid sequence of SEQ ID NO: 98, a CDR-L2 comprising the amino acid sequence of SEQ ID NO: 115, and a CDR-L3 comprising the amino acid sequence of SEQ ID NO: 132. In certain embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 25, and/or the VL comprises the amino acid sequence of SEQ ID NO: 26. In certain embodiments, the MM comprises the amino acid sequence of SEQ ID NO: 137.

In some embodiments according to any one of the masked antibodies described above, the LM does not comprise a cleavable moiety (CM) comprising at least one cleavage site.

In some embodiments according to any one of the masked antibodies described above, the masked antibody is an activatable antibody. Accordingly, in some aspects, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM. In preferred embodiments, the activatable antibody has a higher binding affinity to human CD47 in vitro upon cleavage of the CM than before the cleavage of the CM. In some embodiments, 1) the VH comprises a first complementary-determining-region (CDR-H1), a second complementary-determining-region (CDR-H2), and a third complementary-determining-region (CDR-H3), wherein the CDR-H1 comprises the amino acid sequence of SEQ ID NO: 182 or 183; wherein the CDR-H2 comprises the amino acid sequence of SEQ ID NO: 184 or 185; and wherein the CDR-H3 comprises an amino acid sequence selected from the group consisting of SEQ ID NOs: 186-189; and/or 2) the VL comprises a first complementary-determining-region (CDR-L1), a second complementary-determining-region (CDR-L2), and a third complementary-determining-region (CDR-L3), wherein the CDR-L1 comprises an amino acid sequence selected from the group consisting of SEQ ID NOs: 190-193; wherein the CDR-L2 comprises the amino acid sequence of SEQ ID NO: 194; and wherein the CDR-L3 comprises the amino acid sequence of SEQ ID NO: 195 or 196. In certain embodiments, the VH comprises a CDR-H1 comprising the amino acid sequence of SEQ ID NO: 182, a CDR-H2 comprising the amino acid sequence of SEQ ID NO: 185, and a CDR-H3 comprising the amino acid sequence of SEQ ID NO: 188; and b) the VL comprises a CDR-L1 comprising the amino acid sequence of SEQ ID NO: 191, a CDR-L2 comprising the amino acid sequence of SEQ ID NO: 194, and a CDR-L3 comprising the amino acid sequence of SEQ ID NO: 195. In some embodiments, the activatable antibody comprises a first polypeptide comprising, from N terminus to C terminus, the masking peptide and the VL, and a second polypeptide comprising the VH. In other embodiments, the activatable antibody comprises a first polypeptide comprising, from N terminus to C terminus, the masking peptide and the VH, and a second polypeptide comprising the VL. In additional embodiments, the activatable antibody comprises a first polypeptide comprising, from N terminus to C terminus, the masking peptide, the VL, and the VH. In yet additional embodiments, the activatable antibody comprises a first polypeptide comprising, from N terminus to C terminus the masking peptide, the VH, and the VL. In some embodiments, the cleavage site of the cleavable moiety (CM) is a protease cleavage site for a protease selected from the group consisting of urokinase-type plasminogen activator (uPA), matrix metalloproteinase-1 (MMP-1), MMP-2, MMP-3, MMP-8, MMP-9, MMP-14, Tobacco Etch Virus (TEV) protease, plasmin, Thrombin, Factor X, PSA, PSMA, Cathepsin D, Cathepsin K, Cathepsin S. ADAM10, ADAM12, ADAMTS, Caspase-1, Caspase-2, Caspase-3, Caspase-4, Caspasc-5, Caspase-6, Caspase-7, Caspase-8, Caspase-9, Caspase-10, Caspase-11, Caspase-12, Caspase-13, Caspase-14, and TACE. In certain embodiments, CM comprises an MMP-9 cleavage site that is cleavable by MMP-9. In some embodiments, the CM comprises the amino acid sequence of SEQ ID NO: 138. In some embodiments, the masking peptide comprises an amino acid sequence selected from the group consisting of SEQ ID NOs: 139 and 152-166. In certain embodiments, the masking peptide comprises the amino acid sequence of SEQ ID NO: 139. In some embodiments, the masking peptide, or a part thereof (e.g., the MM) has a masking efficiency of at least about 50 as determined by a Jurkat NFAT reporter assay. In some embodiments, the masking peptide, or a part thereof (e.g., the MM) has a masking efficiency of at least about 100 as determined by a Jurkat NFAT reporter assay. In some embodiments, the activatable antibody comprises a first polypeptide comprising, from N terminus to C terminus the masking peptide and the VL; and a second polypeptide comprising the VH. In certain embodiments, the activatable antibody comprises a human IgG4 fragment crystallizable (Fc) region. In certain embodiments, the activatable antibody comprises a human IgG1 Fc region. In some embodiments, the human IgG1 Fc region comprises two Fc domains, wherein each of the two Fc domains comprises a S239D substitution and/or an 1332E substitution (e.g., a S239D substitution and an 1332E substitution).

In some embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein (a) the VH comprises a CDR-H1 comprising an amino acid sequence selected from the group consisting of SEQ ID NOs: 35-37 and 39-50, a CDR-H2 comprising an amino acid sequence selected from the group consisting of SEQ ID NOs: 52-54 and 56-67, and a CDR-H3 comprising an amino acid sequence selected from the group consisting of SEQ ID NOs: 69-71 and 73-84; and (b) the VL comprises a CDR-L1 comprising an amino acid sequence selected from the group consisting of SEQ ID NOs: 86-88 and 89-101, a CDR-L2 comprising an amino acid sequence selected from the group consisting of SEQ ID NOs: 103-105 and 106-118, and a CDR-L3 comprising an amino acid sequence selected from the group consisting of SEQ ID NOs: 120-122 and 124-135.

In certain embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises an amino acid sequence selected from the group consisting of SEQ ID NOs: 1, 3, 5, 9, 11, 13, 15, 17, 19, 21, 23, 25, 27, 29, and 31; and/or wherein the VL comprises an amino acid sequence selected from the group consisting of SEQ ID NOs: 2, 4, 6, 10, 12, 14, 16, 18, 20, 22, 24, 26, 28, 30, and 32.

In some embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 48, a CDR-H2 comprising the amino acid sequence of SEQ ID NO: 65, and a CDR-H3 comprising the amino acid sequence of SEQ ID NO: 82; and 2) the VL comprises a CDR-L1 comprising the amino acid sequence of SEQ ID NO: 99, a CDR-L2 comprising the amino acid sequence of SEQ ID NO: 116, and a CDR-L3 comprising the amino acid sequence of SEQ ID NO: 133. In certain embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 27, and/or the VL comprises the amino acid sequence of SEQ ID NO: 28. In certain embodiments, the masking peptide comprises the amino acid sequence of SEQ ID NO: 139; the MM comprises the amino acid sequence of SEQ ID NO: 137; and/or the CM comprises the amino acid sequence of SEQ ID NO: 138. In some embodiments, the activatable antibody comprises a first polypeptide comprising, from N terminus to C terminus the masking peptide and the VL; and a second polypeptide comprising the VH. In certain embodiments, the activatable antibody comprises a human IgG4 fragment crystallizable (Fc) region. In one embodiment, the first polypeptide comprises a light chain comprising, from N terminus to C terminus, the masking peptide and the VL, wherein the first polypeptide comprises the amino acid sequence of SEQ ID NO: 142; and wherein the second polypeptide comprises a heavy chain comprising, from N terminus to C terminus, the VH and a human IgG4 Fc domain, wherein the second polypeptide comprises the amino acid sequence of SEQ ID NO: 143. In certain embodiments, the activatable antibody comprises a human IgG1 Fc region. In one embodiment, wherein the first polypeptide comprises a light chain comprising, from N terminus to C terminus, the masking peptide and the VL, wherein the first polypeptide comprises the amino acid sequence of SEQ ID NO: 148; and wherein the second polypeptide comprises a heavy chain comprising, from N terminus to C terminus, the VH and a human IgG1 Fc domain, wherein the second polypeptide comprises the amino acid sequence of SEQ ID NO: 149. In some embodiments, the human IgG1 Fc region comprises two Fc domains, wherein each of the two Fc domains comprises a S239D substitution and/or an 1332E substitution (e.g., a S239D substitution and an 1332E substitution). In one embodiment, the first polypeptide comprises a light chain comprising, from N terminus to C terminus, the masking peptide and the VL, wherein the first polypeptide comprises the amino acid sequence of SEQ ID NO: 150; and wherein the second polypeptide comprises a heavy chain comprising, from N terminus to C terminus, the VH and a human IgG1 Fc domain, wherein the second polypeptide comprises the amino acid sequence of SEQ ID NO: 151.

In some embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 49, a CDR-H2 comprising the amino acid sequence of SEQ ID NO: 66, and a CDR-H3 comprising the amino acid sequence of SEQ ID NO: 83; and 2) the VL comprises a CDR-L1 comprising the amino acid sequence of SEQ ID NO: 100, a CDR-L2 comprising the amino acid sequence of SEQ ID NO: 117, and a CDR-L3 comprising the amino acid sequence of SEQ ID NO: 134. In certain embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 29, and/or the VL comprises the amino acid sequence of SEQ ID NO: 30. In certain embodiments, the masking peptide comprises the amino acid sequence of SEQ ID NO: 139; the MM comprises the amino acid sequence of SEQ ID NO: 137; and/or the CM comprises the amino acid sequence of SEQ ID NO: 138.

In some embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 50, a CDR-H2 comprising the amino acid sequence of SEQ ID NO: 67, and a CDR-H3 comprising the amino acid sequence of SEQ ID NO: 84; and 2) the VL comprises a CDR-L1 comprising the amino acid sequence of SEQ ID NO: 101, a CDR-L2 comprising the amino acid sequence of SEQ ID NO: 118, and a CDR-L3 comprising the amino acid sequence of SEQ ID NO: 135. In certain embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 31, and/or the VL comprises the amino acid sequence of SEQ ID NO: 32. In certain embodiments, the masking peptide comprises the amino acid sequence of SEQ ID NO: 139; the MM comprises the amino acid sequence of SEQ ID NO: 137; and/or the CM comprises the amino acid sequence of SEQ ID NO: 138.

In some embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 35, a CDR-H2 comprising the amino acid sequence of SEQ ID NO: 52, and a CDR-H3 comprising the amino acid sequence of SEQ ID NO: 69; and 2) the VL comprises a CDR-L1 comprising the amino acid sequence of SEQ ID NO: 86, a CDR-L2 comprising the amino acid sequence of SEQ ID NO: 103, and a CDR-L3 comprising the amino acid sequence of SEQ ID NO: 120. In certain embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 1, and/or the VL comprises the amino acid sequence of SEQ ID NO: 2. In certain embodiments, the masking peptide comprises the amino acid sequence of SEQ ID NO: 139; the MM comprises the amino acid sequence of SEQ ID NO: 137; and/or the CM comprises the amino acid sequence of SEQ ID NO: 138.

In some embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 36, a CDR-H2 comprising the amino acid sequence of SEQ ID NO: 53, and a CDR-H3 comprising the amino acid sequence of SEQ ID NO: 70; and 2) the VL comprises a CDR-L1 comprising the amino acid sequence of SEQ ID NO: 87, a CDR-L2 comprising the amino acid sequence of SEQ ID NO: 104, and a CDR-L3 comprising the amino acid sequence of SEQ ID NO: 121. In certain embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 3, and/or the VL comprises the amino acid sequence of SEQ ID NO: 4. In certain embodiments, the masking peptide comprises the amino acid sequence of SEQ ID NO: 139; the MM comprises the amino acid sequence of SEQ ID NO: 137; and/or the CM comprises the amino acid sequence of SEQ ID NO: 138.

In some embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 37, a CDR-H2 comprising the amino acid sequence of SEQ ID NO: 54, and a CDR-H3 comprising the amino acid sequence of SEQ ID NO: 71; and 2) the VL comprises a CDR-L1 comprising the amino acid sequence of SEQ ID NO: 88, a CDR-L2 comprising the amino acid sequence of SEQ ID NO: 105, and a CDR-L3 comprising the amino acid sequence of SEQ ID NO: 122. In certain embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 5, and/or the VL comprises the amino acid sequence of SEQ ID NO: 6. In certain embodiments, the masking peptide comprises the amino acid sequence of SEQ ID NO: 139; the MM comprises the amino acid sequence of SEQ ID NO: 137; and/or the CM comprises the amino acid sequence of SEQ ID NO: 138.

In some embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 39, a CDR-H2 comprising the amino acid sequence of SEQ ID NO: 56, and a CDR-H3 comprising the amino acid sequence of SEQ ID NO: 73; and 2) the VL comprises a CDR-L1 comprising the amino acid sequence of SEQ ID NO: 90, a CDR-L2 comprising the amino acid sequence of SEQ ID NO: 107, and a CDR-L3 comprising the amino acid sequence of SEQ ID NO: 124. In certain embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 9, and/or the VL comprises the amino acid sequence of SEQ ID NO: 10. In certain embodiments, the masking peptide comprises the amino acid sequence of SEQ ID NO: 139; the MM comprises the amino acid sequence of SEQ ID NO: 137; and/or the CM comprises the amino acid sequence of SEQ ID NO: 138.

In some embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 40, a CDR-H2 comprising the amino acid sequence of SEQ ID NO: 57, and a CDR-H3 comprising the amino acid sequence of SEQ ID NO: 74; and 2) the VL comprises a CDR-L1 comprising the amino acid sequence of SEQ ID NO: 91, a CDR-L2 comprising the amino acid sequence of SEQ ID NO: 108, and a CDR-L3 comprising the amino acid sequence of SEQ ID NO: 125. In certain embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 11, and/or the VL comprises the amino acid sequence of SEQ ID NO: 12. In certain embodiments, the masking peptide comprises the amino acid sequence of SEQ ID NO: 139; the MM comprises the amino acid sequence of SEQ ID NO: 137; and/or the CM comprises the amino acid sequence of SEQ ID NO: 138.

In some embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 41, a CDR-H2 comprising the amino acid sequence of SEQ ID NO: 58, and a CDR-H3 comprising the amino acid sequence of SEQ ID NO: 75; and 2) the VL comprises a CDR-L1 comprising the amino acid sequence of SEQ ID NO: 92, a CDR-L2 comprising the amino acid sequence of SEQ ID NO: 109, and a CDR-L3 comprising the amino acid sequence of SEQ ID NO: 126. In certain embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 13, and/or the VL comprises the amino acid sequence of SEQ ID NO: 14. In certain embodiments, the masking peptide comprises the amino acid sequence of SEQ ID NO: 139; the MM comprises the amino acid sequence of SEQ ID NO: 137; and/or the CM comprises the amino acid sequence of SEQ ID NO: 138.

In some embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 42, a CDR-H2 comprising the amino acid sequence of SEQ ID NO: 59, and a CDR-H3 comprising the amino acid sequence of SEQ ID NO: 76; and 2) the VL comprises a CDR-L1 comprising the amino acid sequence of SEQ ID NO: 93, a CDR-L2 comprising the amino acid sequence of SEQ ID NO: 110, and a CDR-L3 comprising the amino acid sequence of SEQ ID NO: 127. In certain embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 15, and/or the VL comprises the amino acid sequence of SEQ ID NO: 16. In certain embodiments, the masking peptide comprises the amino acid sequence of SEQ ID NO: 139; the MM comprises the amino acid sequence of SEQ ID NO: 137; and/or the CM comprises the amino acid sequence of SEQ ID NO: 138.

In some embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 43, a CDR-H2 comprising the amino acid sequence of SEQ ID NO: 60, and a CDR-H3 comprising the amino acid sequence of SEQ ID NO: 77; and 2) the VL comprises a CDR-L1 comprising the amino acid sequence of SEQ ID NO: 94, a CDR-L2 comprising the amino acid sequence of SEQ ID NO: 111, and a CDR-L3 comprising the amino acid sequence of SEQ ID NO: 128. In certain embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 17, and/or the VL comprises the amino acid sequence of SEQ ID NO: 18. In certain embodiments, the masking peptide comprises the amino acid sequence of SEQ ID NO: 139; the MM comprises the amino acid sequence of SEQ ID NO: 137; and/or the CM comprises the amino acid sequence of SEQ ID NO: 138.

In some embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 44, a CDR-H2 comprising the amino acid sequence of SEQ ID NO: 61, and a CDR-H3 comprising the amino acid sequence of SEQ ID NO: 78; and 2) the VL comprises a CDR-L1 comprising the amino acid sequence of SEQ ID NO: 95, a CDR-L2 comprising the amino acid sequence of SEQ ID NO: 112, and a CDR-L3 comprising the amino acid sequence of SEQ ID NO: 129. In certain embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 19, and/or the VL comprises the amino acid sequence of SEQ ID NO: 20. In certain embodiments, the masking peptide comprises the amino acid sequence of SEQ ID NO: 139; the MM comprises the amino acid sequence of SEQ ID NO: 137; and/or the CM comprises the amino acid sequence of SEQ ID NO: 138.

In some embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 45, a CDR-H2 comprising the amino acid sequence of SEQ ID NO: 62, and a CDR-H3 comprising the amino acid sequence of SEQ ID NO: 79; and 2) the VL comprises a CDR-L1 comprising the amino acid sequence of SEQ ID NO: 96, a CDR-L2 comprising the amino acid sequence of SEQ ID NO: 113, and a CDR-L3 comprising the amino acid sequence of SEQ ID NO: 130. In certain embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 21, and/or the VL comprises the amino acid sequence of SEQ ID NO: 22. In certain embodiments, the masking peptide comprises the amino acid sequence of SEQ ID NO: 139; the MM comprises the amino acid sequence of SEQ ID NO: 137; and/or the CM comprises the amino acid sequence of SEQ ID NO: 138.

In some embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) the VH comprises a CDR-H1 comprising the amino acid sequence of SEQ ID NO: 46, a CDR-H2 comprising the amino acid sequence of SEQ ID NO: 63, and a CDR-H3 comprising the amino acid sequence of SEQ ID NO: 80; and 2) the VL comprises a CDR-L1 comprising the amino acid sequence of SEQ ID NO: 97, a CDR-L2 comprising the amino acid sequence of SEQ ID NO: 114, and a CDR-L3 comprising the amino acid sequence of SEQ ID NO: 131. In certain embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 23, and/or the VL comprises the amino acid sequence of SEQ ID NO: 24. In certain embodiments, the masking peptide comprises the amino acid sequence of SEQ ID NO: 139; the MM comprises the amino acid sequence of SEQ ID NO: 137; and/or the CM comprises the amino acid sequence of SEQ ID NO: 138.

In some embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein 1) a) the VH comprises a CDR-H1 comprising the amino acid sequence of SEQ ID NO: 47, a CDR-H2 comprising the amino acid sequence of SEQ ID NO: 64, and a CDR-H3 comprising the amino acid sequence of SEQ ID NO: 81; and 2) the VL comprises a CDR-L1 comprising the amino acid sequence of SEQ ID NO: 98, a CDR-L2 comprising the amino acid sequence of SEQ ID NO: 115, and a CDR-L3 comprising the amino acid sequence of SEQ ID NO: 132. In certain embodiments, provided herein is an activatable antibody comprising: (a) a masking peptide comprising, from N terminus to C terminus, a masking moiety (MM) and a linkage moiety (LM), wherein the LM comprises a cleavable moiety (CM) comprising at least one cleavage site; and (b) a target binding moiety (TBM) comprising an antibody heavy chain variable region (VH) and an antibody light chain variable region (VL), wherein the TBM binds to human CD47; wherein the masking peptide is linked to the N terminus of the VH or the VL; and wherein the MM competes with human CD47 to bind the TBM; wherein the VH comprises the amino acid sequence of SEQ ID NO: 25, and/or the VL comprises the amino acid sequence of SEQ ID NO: 26. In certain embodiments, the masking peptide comprises the amino acid sequence of SEQ ID NO: 139; the MM comprises the amino acid sequence of SEQ ID NO: 137; and/or the CM comprises the amino acid sequence of SEQ ID NO: 138.

In other aspects, provided herein is an isolated antibody, or antigen-binding fragment thereof, that binds to human CD47, comprising a VH and a VL. In some embodiments, 1) the VH comprises a first complementary-determining-region (CDR-H1), a second complementary-determining-region (CDR-H2), and a third complementary-determining-region (CDR-H3), wherein the CDR-H1 comprises the amino acid sequence of SEQ ID NO: 182 or 183; wherein the CDR-H2 comprises the amino acid sequence of SEQ ID NO: 184 or 185; and wherein the CDR-H3 comprises an amino acid sequence selected from the group consisting of SEQ ID NOs: 186-189; and/or 2) the VL comprises a first complementary-determining-region (CDR-L1), a second complementary-determining-region (CDR-L2), and a third complementary-determining-region (CDR-L3), wherein the CDR-L1 comprises an amino acid sequence selected from the group consisting of SEQ ID NOs: 190-193; wherein the CDR-L2 comprises the amino acid sequence of SEQ ID NO: 194; and wherein the CDR-L3 comprises the amino acid sequence of SEQ ID NO: 195 or 196. In certain embodiments, the VH comprises a CDR-H1 comprising the amino acid sequence of SEQ ID NO: 182, a CDR-H2 comprising the amino acid sequence of SEQ ID NO: 185, and a CDR-H3 comprising the amino acid sequence of SEQ ID NO: 188; and b) the VL comprises a CDR-L1 comprising the amino acid sequence of SEQ ID NO: 191, a CDR-L2 comprising the amino acid sequence of SEQ ID NO: 194, and a CDR-L3 comprising the amino acid sequence of SEQ ID NO: 195. In some embodiments, the isolated antibody comprises a human IgG4 Fc region. In other embodiments, the isolated antibody comprises a human IgG1 Fc region. In some embodiments, the human IgG1 Fc region comprises two Fc domains, wherein each of the two Fc domains comprises a S239D substitution and/or an 1332E substitution (e.g., a S239D substitution and an 1332E substitution). In some embodiments, the antigen-binding fragment is selected from the group consisting of a Fab, a Fv, a scFab, and a scFv. In some embodiments, the antibody or antigen-binding fragment binds to one or more amino acid residues of human CD47 selected from the group consisting of K39, W40, K41, F50, D51, G52, T99, E100, L101, and T102. In some embodiments, the antibody or antigen-binding fragment does not bind to one or more amino acid residues of human CD47 selected from the group consisting of L2, L3, F4, K6, N27, E29, A30, Q31, T34, E35, V36, Y37, D46, T49, A53, E97, R103, E104, G105, and E106. In certain embodiments, the antibody or antigen-binding fragment binds to amino acid residues K39, W40, K41, F50, D51, G52, T99, E100, L101, and T102 of human CD47 and/or does not bind to amino acid residues L2, L3, F4, K6, N27, E29, A30, Q31, T34, E35, V36, Y37, D46, T49, A53, E97, R103, E104, G105, and E106 of human CD47.

In some embodiments, provided herein is an isolated antibody, or antigen-binding fragment thereof, that binds to human CD47, comprising a VH and a VL, wherein (a) the VH comprises a CDR-H1 comprising an amino acid sequence selected from the group consisting of SEQ ID NOs: 35-37 and 39-50, a CDR-H2 comprising an amino acid sequence selected from the group consisting of SEQ ID NOs: 52-54 and 56-67, and a CDR-H3 comprising an amino acid sequence selected from the group consisting of SEQ ID NOs: 69-71 and 73-84; and (b) the VL comprises a CDR-L1 comprising an amino acid sequence selected from the group consisting of SEQ ID NOs: 86-88 and 89-101, a CDR-L2 comprising an amino acid sequence selected from the group consisting of SEQ ID NOs: 103-105 and 106-118, and a CDR-L3 comprising an amino acid sequence selected from the group consisting of SEQ ID NOs: 120-122 and 124-135. In certain embodiments, provided herein is an isolated antibody, or antigen-binding fragment thereof, that binds to human CD47, comprising a VH and a VL, wherein the VH comprises an amino acid sequence selected from the group consisting of SEQ ID NOs: 1, 3, 5, 9, 11, 13, 15, 17, 19, 21, 23, 25, 27, 29, and 31; and/or wherein the VL comprises an amino acid sequence selected from the group consisting of SEQ ID NOs: 2, 4, 6, 10, 12, 14, 16, 18, 20, 22, 24, 26, 28, 30, and 32.

In some embodiments, provided herein is an isolated antibody, or antigen-binding fragment thereof, that binds to human CD47, comprising a VH and a VL, wherein 1) the VH comprises a CDR-H1 comprising the amino acid sequence of SEQ ID NO: 48, a CDR-H2 comprising the amino acid sequence of SEQ ID NO: 65, and a CDR-H3 comprising the amino acid sequence of SEQ ID NO: 82; and 2) the VL comprises a CDR-L1 comprising the amino acid sequence of SEQ ID NO: 99, a CDR-L2 comprising the amino acid sequence of SEQ ID NO: 116, and a CDR-L3 comprising the amino acid sequence of SEQ ID NO: 133. In certain embodiments, provided herein is an isolated antibody, or antigen-binding fragment thereof, that binds to human CD47, comprising a VH and a VL, wherein the VH comprises the amino acid sequence of SEQ ID NO: 27, and/or the VL comprises the amino acid sequence of SEQ ID NO: 28. In some embodiments, the isolated antibody comprises a human IgG4 Fc region. In one embodiment, the isolated antibody comprises a first polypeptide comprising a light chain comprising the VL, wherein the first polypeptide comprises the amino acid sequence of SEQ ID NO: 140; and a second polypeptide comprising a heavy chain comprising, from N terminus to C terminus, the VH and a human IgG4 Fc domain, wherein the second polypeptide comprises the amino acid sequence of SEQ ID NO: 141. In other embodiments, the isolated antibody comprises a human IgG1 Fc region. In one embodiment, the isolated antibody comprises a first polypeptide comprising a light chain comprising the VL, wherein the first polypeptide comprises the amino acid sequence of SEQ ID NO: 144; and a second polypeptide comprising a heavy chain comprising, from N terminus to C terminus, the VH and a human IgG1 Fc domain, wherein the second polypeptide comprises the amino acid sequence of SEQ ID NO: 145. In certain embodiments, the human IgG1 Fc region comprises two Fc domains, wherein each of the two Fc domains comprises a S239D substitution and/or an 1332E substitution (e.g., a S239D substitution and an 1332E substitution). In one embodiment, wherein the isolated antibody comprises a first polypeptide comprising a light chain comprising the VL, wherein the first polypeptide comprises the amino acid sequence of SEQ ID NO: 146; and a second polypeptide comprising a heavy chain comprising, from N terminus to C terminus, the VH and a human IgG1 Fc domain, wherein the second polypeptide comprises the amino acid sequence of SEQ ID NO: 147.

In some embodiments, provided herein is an isolated antibody, or antigen-binding fragment thereof, that binds to human CD47, comprising a VH and a VL, wherein 1) the VH comprises a CDR-H1 comprising the amino acid sequence of SEQ ID NO: 49, a CDR-H2 comprising the amino acid sequence of SEQ ID NO: 66, and a CDR-H3 comprising the amino acid sequence of SEQ ID NO: 83; and 2) the VL comprises a CDR-L1 comprising the amino acid sequence of SEQ ID NO: 100, a CDR-L2 comprising the amino acid sequence of SEQ ID NO: 117, and a CDR-L3 comprising the amino acid sequence of SEQ ID NO: 134. In certain embodiments, provided herein is an isolated antibody, or antigen-binding fragment thereof, that binds to human CD47, comprising a VH and a VL, wherein the VH comprises the amino acid sequence of SEQ ID NO: 29, and/or the VL comprises the amino acid sequence of SEQ ID NO: 30. In some embodiments, the isolated antibody comprises a human IgG1 Fc region. In certain embodiments, the human IgG1 Fc region comprises two Fc domains, wherein each of the two Fc domains comprises a S239D substitution and/or an 1332E substitution (e.g., a S239D substitution and an 1332E substitution).

In some embodiments, provided herein is an isolated antibody, or antigen-binding fragment thereof, that binds to human CD47, comprising a VH and a VL, wherein 1) the VH comprises a CDR-H1 comprising the amino acid sequence of SEQ ID NO: 50, a CDR-H2 comprising the amino acid sequence of SEQ ID NO: 67, and a CDR-H3 comprising the amino acid sequence of SEQ ID NO: 84; and 2) the VL comprises a CDR-L1 comprising the amino acid sequence of SEQ ID NO: 101, a CDR-L2 comprising the amino acid sequence of SEQ ID NO: 118, and a CDR-L3 comprising the amino acid sequence of SEQ ID NO: 135. In certain embodiments, provided herein is an isolated antibody, or antigen-binding fragment thereof, that binds to human CD47, comprising a VH and a VL, wherein the VH comprises the amino acid sequence of SEQ ID NO: 31, and/or the VL comprises the amino acid sequence of SEQ ID NO: 32. In some embodiments, the isolated antibody comprises a human IgG1 Fc region. In certain embodiments, the human IgG1 Fc region comprises two Fc domains, wherein each of the two Fc domains comprises a S239D substitution and/or an 1332E substitution (e.g., a S239D substitution and an 1332E substitution).

In some embodiments, provided herein is an isolated antibody, or antigen-binding fragment thereof, that binds to human CD47, comprising a VH and a VL, wherein 1) the VH comprises a CDR-H1 comprising the amino acid sequence of SEQ ID NO: 35, a CDR-H2 comprising the amino acid sequence of SEQ ID NO: 52, and a CDR-H3 comprising the amino acid sequence of SEQ ID NO: 69; and 2) the VL comprises a CDR-L1 comprising the amino acid sequence of SEQ ID NO: 86, a CDR-L2 comprising the amino acid sequence of SEQ ID NO: 103, and a CDR-L3 comprising the amino acid sequence of SEQ ID NO: 120. In certain embodiments, provided herein is an isolated antibody, or antigen-binding fragment thereof, that binds to human CD47, comprising a VH and a VL, wherein the VH comprises the amino acid sequence of SEQ ID NO: 1, and/or the VL comprises the amino acid sequence of SEQ ID NO: 2. In some embodiments, the isolated antibody comprises a human IgG1 Fc region. In certain embodiments, the human IgG1 Fc region comprises two Fc domains, wherein each of the two Fc domains comprises a S239D substitution and/or an 1332E substitution (e.g., a S239D substitution and an 1332E substitution).

In some embodiments, provided herein is an isolated antibody, or antigen-binding fragment thereof, that binds to human CD47, comprising a VH and a VL, wherein 1) the VH comprises a CDR-H1 comprising the amino acid sequence of SEQ ID NO: 36, a CDR-H2 comprising the amino acid sequence of SEQ ID NO: 53, and a CDR-H3 comprising the amino acid sequence of SEQ ID NO: 70; and 2) the VL comprises a CDR-L1 comprising the amino acid sequence of SEQ ID NO: 87, a CDR-L2 comprising the amino acid sequence of SEQ ID NO: 104, and a CDR-L3 comprising the amino acid sequence of SEQ ID NO: 121. In certain embodiments, provided herein is an isolated antibody, or antigen-binding fragment thereof, that binds to human CD47, comprising a VH and a VL, wherein the VH comprises the amino acid sequence of SEQ ID NO: 3, and/or the VL comprises the amino acid sequence of SEQ ID NO: 4. In some embodiments, the isolated antibody comprises a human IgG1 Fc region. In certain embodiments, the human IgG1 Fc region comprises two Fc domains, wherein each of the two Fc domains comprises a S239D substitution and/or an 1332E substitution (e.g., a S239D substitution and an 1332E substitution).

In some embodiments, provided herein is an isolated antibody, or antigen-binding fragment thereof, that binds to human CD47, comprising a VH and a VL, wherein 1) the VH comprises a CDR-H1 comprising the amino acid sequence of SEQ ID NO: 37, a CDR-H2 comprising the amino acid sequence of SEQ ID NO: 54, and a CDR-H3 comprising the amino acid sequence of SEQ ID NO: 71; and 2) the VL comprises a CDR-L1 comprising the amino acid sequence of SEQ ID NO: 88, a CDR-L2 comprising the amino acid sequence of SEQ ID NO: 105, and a CDR-L3 comprising the amino acid sequence of SEQ ID NO: 122. In certain embodiments, provided herein is an isolated antibody, or antigen-binding fragment thereof, that binds to human CD47, comprising a VH and a VL, wherein the VH comprises the amino acid sequence of SEQ ID NO: 5, and/or the VL comprises the amino acid sequence of SEQ ID NO: 6. In some embodiments, the isolated antibody comprises a human IgG1 Fc region. In certain embodiments, the human IgG1 Fc region comprises two Fc domains, wherein each of the two Fc domains comprises a S239D substitution and/or an 1332E substitution (e.g., a S239D substitution and an 1332E substitution).

In some embodiments, provided herein is an isolated antibody, or antigen-binding fragment thereof, that binds to human CD47, comprising a VH and a VL, wherein 1) the VH comprises a CDR-H1 comprising the amino acid sequence of SEQ ID NO: 39, a CDR-H2 comprising the amino acid sequence of SEQ ID NO: 56, and a CDR-H3 comprising the amino acid sequence of SEQ ID NO: 73; and 2) the VL comprises a CDR-L1 comprising the amino acid sequence of SEQ ID NO: 90, a CDR-L2 comprising the amino acid sequence of SEQ ID NO: 107, and a CDR-L3 comprising the amino acid sequence of SEQ ID NO: 124. In certain embodiments, provided herein is an isolated antibody, or antigen-binding fragment thereof, that binds to human CD47, comprising a VH and a VL, wherein the VH comprises the amino acid sequence of SEQ ID NO: 9, and/or the VL comprises the amino acid sequence of SEQ ID NO: 10. In some embodiments, the isolated antibody comprises a human IgG1 Fc region. In certain embodiments, the human IgG1 Fc region comprises two Fc domains, wherein each of the two Fc domains comprises a S239D substitution and/or an 1332E substitution (e.g., a S239D substitution and an 1332E substitution).

In some embodiments, provided herein is an isolated antibody, or antigen-binding fragment thereof, that binds to human CD47, comprising a VH and a VL, wherein 1) the VH comprises a CDR-H1 comprising the amino acid sequence of SEQ ID NO: 40, a CDR-H2 comprising the amino acid sequence of SEQ ID NO: 57, and a CDR-H3 comprising the amino acid sequence of SEQ ID NO: 74; and 2) the VL comprises a CDR-L1 comprising the amino acid sequence of SEQ ID NO: 91, a CDR-L2 comprising the amino acid sequence of SEQ ID NO: 108, and a CDR-L3 comprising the amino acid sequence of SEQ ID NO: 125. In certain embodiments, provided herein is an isolated antibody, or antigen-binding fragment thereof, that binds to human CD47, comprising a VH and a VL, wherein the VH comprises the amino acid sequence of SEQ ID NO: 11, and/or the VL comprises the amino acid sequence of SEQ ID NO: 12. In some embodiments, the isolated antibody comprises a human IgG1 Fc region. In certain embodiments, the human IgG1 Fc region comprises two Fc domains, wherein each of the two Fc domains comprises a S239D substitution and/or an 1332E substitution (e.g., a S239D substitution and an 1332E substitution).

In some embodiments, provided herein is an isolated antibody, or antigen-binding fragment thereof, that binds to human CD47, comprising a VH and a VL, wherein 1) the VH comprises a CDR-H1 comprising the amino acid sequence of SEQ ID NO: 41, a CDR-H2 comprising the amino acid sequence of SEQ ID NO: 58, and a CDR-H3 comprising the amino acid sequence of SEQ ID NO: 75; and 2) the VL comprises a CDR-L1 comprising the amino acid sequence of SEQ ID NO: 92, a CDR-L2 comprising the amino acid sequence of SEQ ID NO: 109, and a CDR-L3 comprising the amino acid sequence of SEQ ID NO: 126. In certain embodiments, provided herein is an isolated antibody, or antigen-binding fragment thereof, that binds to human CD47, comprising a VH and a VL, wherein the VH comprises the amino acid sequence of SEQ ID NO: 13, and/or the VL comprises the amino acid sequence of SEQ ID NO: 14. In some embodiments, the isolated antibody comprises a human IgG1 Fc region. In certain embodiments, the human IgG1 Fc region comprises two Fc domains, wherein each of the two Fc domains comprises a S239D substitution and/or an 1332E substitution (e.g., a S239D substitution and an 1332E substitution).

In some embodiments, provided herein is an isolated antibody, or antigen-binding fragment thereof, that binds to human CD47, comprising a VH and a VL, wherein 1) the VH comprises a CDR-H1 comprising the amino acid sequence of SEQ ID NO: 42, a CDR-H2 comprising the amino acid sequence of SEQ ID NO: 59, and a CDR-H3 comprising the amino acid sequence of SEQ ID NO: 76; and 2) the VL comprises a CDR-L1 comprising the amino acid sequence of SEQ ID NO: 93, a CDR-L2 comprising the amino acid sequence of SEQ ID NO: 110, and a CDR-L3 comprising the amino acid sequence of SEQ ID NO: 127. In certain embodiments, provided herein is an isolated antibody, or antigen-binding fragment thereof, that binds to human CD47, comprising a VH and a VL, wherein the VH comprises the amino acid sequence of SEQ ID NO: 15, and/or the VL comprises the amino acid sequence of SEQ ID NO: 16. In some embodiments, the isolated antibody comprises a human IgG1 Fc region. In certain embodiments, the human IgG1 Fc region comprises two Fc domains, wherein each of the two Fc domains comprises a S239D substitution and/or an 1332E substitution (e.g., a S239D substitution and an 1332E substitution).

In some embodiments, provided herein is an isolated antibody, or antigen-binding fragment thereof, that binds to human CD47, comprising a VH and a VL, wherein 1) the VH comprises a CDR-H1 comprising the amino acid sequence of SEQ ID NO: 43, a CDR-H2 comprising the amino acid sequence of SEQ ID NO: 60, and a CDR-H3 comprising the amino acid sequence of SEQ ID NO: 77; and 2) the VL comprises a CDR-L1 comprising the amino acid sequence of SEQ ID NO: 94, a CDR-L2 comprising the amino acid sequence of SEQ ID NO: 111, and a CDR-L3 comprising the amino acid sequence of SEQ ID NO: 128. In certain embodiments, provided herein is an isolated antibody, or antigen-binding fragment thereof, that binds to human CD47, comprising a VH and a VL, wherein the VH comprises the amino acid sequence of SEQ ID NO: 17, and/or the VL comprises the amino acid sequence of SEQ ID NO: 18. In some embodiments, the isolated antibody comprises a human IgG1 Fc region. In certain embodiments, the human IgG1 Fc region comprises two Fc domains, wherein each of the two Fc domains comprises a S239D substitution and/or an 1332E substitution (e.g., a S239D substitution and an 1332E substitution).

In some embodiments, provided herein is an isolated antibody, or antigen-binding fragment thereof, that binds to human CD47, comprising a VH and a VL, wherein 1) the VH comprises a CDR-H1 comprising the amino acid sequence of SEQ ID NO: 44, a CDR-H2 comprising the amino acid sequence of SEQ ID NO: 61, and a CDR-H3 comprising the amino acid sequence of SEQ ID NO: 78; and 2) the VL comprises a CDR-L1 comprising the amino acid sequence of SEQ ID NO: 95, a CDR-L2 comprising the amino acid sequence of SEQ ID NO: 112, and a CDR-L3 comprising the amino acid sequence of SEQ ID NO: 129. In certain embodiments, provided herein is an isolated antibody, or antigen-binding fragment thereof, that binds to human CD47, comprising a VH and a VL, wherein the VH comprises the amino acid sequence of SEQ ID NO: 19, and/or the VL comprises the amino acid sequence of SEQ ID NO: 20. In some embodiments, the isolated antibody comprises a human IgG1 Fc region. In certain embodiments, the human IgG1 Fc region comprises two Fc domains, wherein each of the two Fc domains comprises a S239D substitution and/or an 1332E substitution (e.g., a S239D substitution and an 1332E substitution).

In some embodiments, provided herein is an isolated antibody, or antigen-binding fragment thereof, that binds to human CD47, comprising a VH and a VL, wherein 1) the VH comprises a CDR-H1 comprising the amino acid sequence of SEQ ID NO: 45, a CDR-H2 comprising the amino acid sequence of SEQ ID NO: 62, and a CDR-H3 comprising the amino acid sequence of SEQ ID NO: 79; and 2) the VL comprises a CDR-L1 comprising the amino acid sequence of SEQ ID NO: 96, a CDR-L2 comprising the amino acid sequence of SEQ ID NO: 113, and a CDR-L3 comprising the amino acid sequence of SEQ ID NO: 130. In certain embodiments, provided herein is an isolated antibody, or antigen-binding fragment thereof, that binds to human CD47, comprising a VH and a VL, wherein the VH comprises the amino acid sequence of SEQ ID NO: 21, and/or the VL comprises the amino acid sequence of SEQ ID NO: 22. In some embodiments, the isolated antibody comprises a human IgG1 Fc region. In certain embodiments, the human IgG1 Fc region comprises two Fc domains, wherein each of the two Fc domains comprises a S239D substitution and/or an 1332E substitution (e.g., a S239D substitution and an 1332E substitution).

In some embodiments, provided herein is an isolated antibody, or antigen-binding fragment thereof, that binds to human CD47, comprising a VH and a VL, wherein 1) the VH comprises a CDR-H1 comprising the amino acid sequence of SEQ ID NO: 46, a CDR-H2 comprising the amino acid sequence of SEQ ID NO: 63, and a CDR-H3 comprising the amino acid sequence of SEQ ID NO: 80; and 2) the VL comprises a CDR-L1 comprising the amino acid sequence of SEQ ID NO: 97, a CDR-L2 comprising the amino acid sequence of SEQ ID NO: 114, and a CDR-L3 comprising the amino acid sequence of SEQ ID NO: 131. In certain embodiments, provided herein is an isolated antibody, or antigen-binding fragment thereof, that binds to human CD47, comprising a VH and a VL, wherein the VH comprises the amino acid sequence of SEQ ID NO: 23, and/or the VL comprises the amino acid sequence of SEQ ID NO: 24. In some embodiments, the isolated antibody comprises a human IgG1 Fc region. In certain embodiments, the human IgG1 Fc region comprises two Fc domains, wherein each of the two Fc domains comprises a S239D substitution and/or an 1332E substitution (e.g., a S239D substitution and an 1332E substitution).

In some embodiments, provided herein is an isolated antibody, or antigen-binding fragment thereof, that binds to human CD47, comprising a VH and a VL, wherein 1) a) the VH comprises a CDR-H1 comprising the amino acid sequence of SEQ ID NO: 47, a CDR-H2 comprising the amino acid sequence of SEQ ID NO: 64, and a CDR-H3 comprising the amino acid sequence of SEQ ID NO: 81; and 2) the VL comprises a CDR-L1 comprising the amino acid sequence of SEQ ID NO: 98, a CDR-L2 comprising the amino acid sequence of SEQ ID NO: 115, and a CDR-L3 comprising the amino acid sequence of SEQ ID NO: 132. In certain embodiments, provided herein is an isolated antibody, or antigen-binding fragment thereof, that binds to human CD47, comprising a VH and a VL, wherein the VH comprises the amino acid sequence of SEQ ID NO: 25, and/or the VL comprises the amino acid sequence of SEQ ID NO: 26. In some embodiments, the isolated antibody comprises a human IgG1 Fc region. In certain embodiments, the human IgG1 Fc region comprises two Fc domains, wherein each of the two Fc domains comprises a S239D substitution and/or an 1332E substitution (e.g., a S239D substitution and an 1332E substitution). In some embodiments, the isolated antibody or antigen-binding fragment binds human CD47 with a KD of about 50 nM or less. In certain embodiments, the isolated antibody or antigen-binding fragment binds human CD47 with a K_{D} of about 10 nM or less. In some embodiments, the isolated antibody or antigen-binding fragment has a half maximal inhibitory concentration (IC50) of about 100 nM or less for blocking binding of human CD47 to human SIRPα in vitro. In certain embodiments, the isolated antibody or antigen-binding fragment has a half maximal inhibitory concentration (IC50) of about 10 nM or less for blocking binding of human CD47 to human SIRPα in vitro. In some embodiments, the isolated antibody or antigen-binding fragment completely blocks binding of human CD47 to human SIRPα in vitro when the isolated antibody or antigen-binding fragment is provided at a concentration of about 1 μM or greater. In some embodiments, the isolated antibody or antigen-binding fragment has a half maximal effective concentration (EC50) of about 10 nM or less for binding to tumor cells in vitro, wherein the tumor cells comprise a B cell lymphoma cell line, a T cell lymphoma cell line, or a combination thereof. In some embodiments, the isolated antibody or antigen-binding fragment has a half maximal effective concentration (EC50) of about 10 nM or less for increasing macrophage phagocytosis of tumor cells in vitro, wherein the tumor cells comprise a B cell lymphoma cell line, a T cell lymphoma cell line, or a combination thereof. In certain embodiments, the isolated antibody or antigen-binding fragment has a half maximal effective concentration (EC50) of about 1 nM or less for increasing macrophage phagocytosis of tumor cells in vitro, wherein the tumor cells comprise a B cell lymphoma cell line, a T cell lymphoma cell line, or a combination thereof.

In some embodiments which may be combined with any of the preceding embodiments, the masked antibody, activatable antibody, isolated antibody, or antigen-binding fragment thereof is cross-reactive with a CD47 polypeptide from at least one non-human species selected from the group consisting of cynomolgus monkey, rat, and dog. In certain embodiments which may be combined with any of the preceding embodiments, the masked antibody, activatable antibody, isolated antibody, or antigen-binding fragment thereof binds to cynomolgus monkey CD47.

In some aspects, provided herein is an isolated polynucleotide encoding one or more polypeptide chains of any masked antibody, activatable antibody, isolated antibody, or antigen-binding fragment described herein.

In other aspects, provided herein is a vector comprising a polynucleotide encoding one or more polypeptide chains of any masked antibody, activatable antibody, isolated antibody, or antigen-binding fragment described herein.

In other aspects, provided herein is a host cell comprising a vector comprising a polynucleotide encoding one or more polypeptide chains of any masked antibody, activatable antibody, isolated antibody, or antigen-binding fragment described herein.

In yet other aspects, provided herein is a method of making a masked antibody, activatable antibody, isolated antibody, or antigen-binding fragment, comprising culturing a host cell under conditions suitable for producing the masked antibody, activatable antibody, isolated antibody, or antigen-binding fragment, wherein the host cell comprises a vector comprising a polynucleotide encoding one or more polypeptide chains of any masked antibody, activatable antibody, isolated antibody, or antigen-binding fragment described herein.

In some aspects, provided herein is a pharmaceutical composition comprising any masked antibody, activatable antibody, isolated antibody, or antigen-binding fragment described herein and a pharmaceutically acceptable carrier.

In other aspects, provided herein is a method for treating a CD47-positive disease or condition in a subject in need thereof, comprising administering to the subject an effective amount of a pharmaceutical composition comprising any masked antibody, activatable antibody, isolated antibody, or antigen-binding fragment described herein, and a pharmaceutically acceptable carrier. In some embodiments, the administering does not cause anemia in the subject. In some embodiments, the disease or condition is cancer. In certain embodiments, the cancer comprises B cell lymphoma, T cell lymphoma, or a combination thereof. In some embodiments, the cancer is selected from the group consisting of lymphoma, leukemia, head and neck cancer, gastric cancer, esophageal cancer, breast cancer, cervical cancer, cholangiocarcinoma, colon cancer, ovarian cancer, thyroid cancer, uterine cancer, endometrial cancer, lung cancer, mesothelioma, and pancreatic cancer. In certain embodiments, the cancer is selected from the group consisting of triple negative breast cancer (TNBC), Her2+ gastric-esophageal junction (GEJ) cancer, small cell lung cancer (SCLC), diffuse large B-cell lymphoma (DLBCL), acute myeloid leukemia (AML), Head and neck squamous cell carcinoma (HNSC), gastric carcinoma (GC), breast invasive carcinoma (BRCA), cervical squamous cell carcinoma and endocervical adenocarcinoma (CESC), cholangiocarcinoma (CHOL), colon adenocarcinoma (COAD), ovarian serous cystadenocarcinoma (OV), thyroid carcinoma (THCA), uterine corpus endometrial carcinoma (UCEC), HER2+ breast cancer, hormone receptor positive breast cancer, lymphoid neoplasm diffuse large B-cell lymphoma (DLBC), lung adenocarcinoma (LUAD), lung squamous cell carcinoma (LUSC), mesothelioma (MESO), and pancreatic adenocarcinoma (PAAD). In some embodiments, the masked antibody, or the antibody or antigen-binding fragment thereof, is administered at a dose of at least about 0.6 mg/kg. In some embodiments, the pharmaceutical compositions is administered at a frequency of at least once every three weeks or at least once every two weeks.

In some embodiments according to any one of the methods for treating a CD47-positive disease or condition described above, the method further comprises administering to the subject an effective amount of one or more additional therapeutic agents. In some embodiments, the one or more therapeutic agents comprise viral gene therapy, an immune checkpoint inhibitor, a target therapy, a radiation therapy, a chemotherapy, or any combination thereof. In certain embodiments, the one or more additional therapeutic agents comprise pomalyst, revlimid, lenalidomide, pomalidomide, thalidomide, a DNA-alkylating platinum-containing derivative, cisplatin, 5-fluorouracil, cyclophosphamide, an anti-CD47 antibody, an anti-CTLA4 antibody, an anti-PD-1 antibody, an anti-PD-L1 antibody, an anti-CD20 antibody, an anti-CD40 antibody, an anti-DR5 antibody, an anti-CD1d antibody, an anti-TIM3 antibody, an anti-SLAMF7 antibody, an anti-KIR receptor antibody, an anti-OX40 antibody, an anti-HER2 antibody, an anti-ErbB-2 antibody, an anti-EGFR antibody, cetuximab, rituximab, trastuzumab, pembrolizumab, radiotherapy, single dose radiation, fractionated radiation, focal radiation, whole organ radiation, IL-12, IFNa, GM-CSF, a chimeric antigen receptor, adoptively transferred T cells, an anti-cancer vaccine, an oncolytic virus, or any combination thereof.

In some aspects, provided herein is a method of treating cancer comprising administering an anti-CD47 activatable antibody, wherein the anti-CD47 activatable antibody comprises a human IgG1 Fc or an IgG1 Fc with enhanced antibody-dependent cellular cytotoxicity (ADCC) activity, wherein the anti-CD47 activatable antibody comprises a masking peptide comprising a cleavable moiety (CM) comprising at least one cleavage site, wherein the activatable antibody has a higher binding affinity to human CD47 when the CM is cleaved at the site of the cancer (e.g., in the tumor microenvironment (TME)) than when the CM is not cleaved.

### DESCRIPTION OF THE FIGURES

The present application can be understood by reference to the following description taken in conjunction with the accompanying figures.

FIG. 1 depicts the ability of the indicated antibodies to induce human RBC hemagglutination.

FIG. 2 depicts the ADCC activity of human NK cells from different donors on Calcein-labeled CEM cells incubated with the indicated antibodies. Percent cell lysis is shown as percent cytotoxicity.

FIG. 3A and FIG. 3B depict the in vivo anti-tumor efficacy of the antibodies, or isotype control, in a B-NDG/Raji-Luc mouse systemic model in two independent experiments. Data points represent group mean; error bars represent standard deviation. FIG. 3A depicts the first independent experiment. FIG. 3B depicts the second independent experiment.

FIG. 4A and FIG. 4B depict the in vivo anti-tumor efficacy of the antibodies, or isotype control, in a B-NDG/Raji mouse subcutaneous tumor model. Data points represent group mean; error bars represent standard deviation. FIG. 4A depicts the first independent experiment. FIG. 4B depicts the second independent experiment.

FIG. 5 depicts the in vivo anti-tumor efficacy of the antibodies, or isotype control, in a SCID/Raji-Luc mouse systemic model. Data points represent group mean; error bars represent standard deviation.

FIGS. 6A-6B show the hematological toxicity of the indicated antibodies on B-hSIRPα/hCD47 humanized mice. FIG. 6A shows the change RBC number in peripheral blood before and after single intraperitoneal injection of the antibodies. FIG. 6B shows the change of hemoglobin level in peripheral blood before and after single intraperitoneal injection of the antibodies.

FIGS. 7A-7F depict hematology toxicity and pharmacokinetics of the indicated antibodies on cynomolgus monkeys after single intravenous injection. FIG. 7A shows the change of RBC percentage after dosing. FIG. 7B shows the change of hemoglobin percentage after dosing. FIG. 7C shows the change of reticulocyte percentage after dosing.

FIG. 7D shows the change of hematocrit after dosing. FIG. 7E shows the change of platelet percentage after dosing. FIG. 7F shows blood concentrations of the indicated antibodies intravenously injected at the indicated doses to cynomolgus monkeys.

FIG. 8 depicts plasma concentrations of the indicated antibodies intravenously injected at indicated doses to CB17-SCID mice.

FIGS. 9A-D depict clustering of CFSE-labeled Raji cells and BMQC-labeled Jurkat cells as detected by flow cytometry. FIG. 9A depicts clustering of CFSE-Raji cells and BMQC-Jurkat cells when treated with a negative isotype control and a negative buffer control, and illustrates the concept of trans binding versus cis binding of antibodies to the cells. FIG. 9B depicts clustering of CFSE-Raji cells and BMQC-Jurkat cells when treated with a positive control known to bind CFSE-Raji cells and BMQC-Jurkat cells in trans. FIG. 9C depicts clustering of CFSE-Raji cells and BMQC-Jurkat cells when treated with anti-CD47 antibody TY21446. FIG. 9D depicts clustering of CFSE-Raji cells and BMQC-Jurkat cells when treated with anti-CD47 benchmark control antibody TAC2204.

FIGS. 10A-C depict the clustering of fluorescent dye-labeled human RBCs as detected by flow cytometry. FIG. 10A depicts the gating strategy for the identification of clustering between CFSE-RBCs and BMQC-RBCs in flow cytometry. FIG. 10B and FIG. 10C depict the effect of test antibodies on human RBC clustering using RBCs from two different donors.

FIGS. 11A-C depict the clustering of human RBCs as detected by flow cytometry. FIG. 11A depicts the identification of clustered RBCs by using FSC-H/FSC-A gating strategy in flow cytometry. FIG. 11B and FIG. 11C depict the effect of test antibodies on human RBC clustering using RBCs from two different donors.

FIGS. 12A-E depict the receptor occupancy (RO) of anti-CD47 antibodies binding in vivo to the extracellular domain (ECD) of human CD47 expressed in hSIRPα/hCD47 knock-in mice that had been injected with Raji tumor cells. FIG. 12A and FIG. 12B show the RO rate of the indicated antibodies binding to red blood cells (RBCs) and blood cells other than RBCs (WBC), respectively, at various doses and times after injection with the indicated antibodies. FIG. 12C shows the RO rate of the indicated antibodies binding to Raji cells in subcutaneous tumor samples collected 72 hours after injection with the indicated antibodies. FIG. 12D shows the RO rate of the indicated antibodies binding to Raji cells in the bone marrow of the mice, harvested 72 hours after injection with the indicated antibodies. FIG. 12E shows the total number of blood cells per liter of in mice 72 hours after injection with the indicated antibodies. A bracket with a double asterisk (**) indicates that the two samples are significantly difference at p<0.01, and a bracket with “ns” indicates that the two samples are not significantly different.

FIGS. 13A-13B depict mapping of the epitope residues of human CD47 that are bound by SIRPα, magrolimab, and TY21446. FIG. 13A depicts the crystal structure of human CD47, with the epitope residues of human CD47 that are bound by SIRPα, magrolimab, and TY21446 shaded dark gray. FIG. 13B depicts the specific residues of human CD47 that are bound by SIRPα, magrolimab, and TY21446, which are shaded gray.

FIGS. 14A-14D depict CD47 receptor occupancy (RO) of TAC2204 and TY26898 and effects thereof on blood cell count and viability. FIG. 14A depicts the CD47 RO of TAC2204 and TY26898 in tissues of mice dosed with each antibody. FIG. 14B depicts the relative CD47 RO of TY26898 vs TAC2204 in tissues of mice dosed with each antibody. FIG. 14C depicts the total blood cell count of blood taken from mice dosed with TAC2204 and TY26898. FIG. 14D depicts the blood cell viability of blood taken from mice dosed with TAC2204 and TY26898.

FIGS. 15A-15D depict the in vivo anti-tumor efficacy of the TY26898, TY26896, TAC2204, or isotype control, in different cancer models. Data points represent group mean; error bars represent standard error of the mean. FIG. 15A depicts in vivo tumor suppression in a OE19 HER2+ GEJ cancer xenograft model in CB17 SCID mice by TY26898 and TAC2204, with Herceptin as a positive control and a vehicle negative control. FIG. 15B depicts in vivo tumor suppression in a MDA-MB-231 triple negative breast cancer (TNBC) xenograft model in CB17 SCID mice by TY26898 and TAC2204, with PBS as a negative control. FIG. 15C depicts in vivo tumor suppression in a SHP-77 small cell lung cancer (SCLC) xenograft model in CB17 SCID mice by TY26898, TY26896, and TAC2204, with PBS as a negative control. FIG. 15D depicts in vivo tumor suppression in a OV90 ovarian cancer xenograft model in CB17 SCID mice by TY26898 and TAC2204, with PBS as a negative control. FIG. 15E depicts in vivo tumor suppression in a OE19 HER2+ GEJ tumor model by TY26898, with PBS as a negative control.

FIGS. 16A-16B depict CD47 receptor occupancy (RO) by TY26898 in different tumor models. FIG. 16A depicts CD47 RO by TY26898 and TAC2204 in OE19 HER2+ GEJ tumors, MDA-MB-231 TNBC tumors and OV90 ovarian tumors,. FIG. 16B depicts CD47 RO in OE19 HER2+ GEJ tumors from mice dose with 3 mg/kg or 10 mg/kg of TY26898.

FIG. 17A-17B depict Western blots and proportions of cleaved and intact TY26898 extracted from tumors of mice that had been treated previously with TY26898. FIG. 17A depicts cleaved and intact TY26898 in a OE19 HER2+ GEJ tumors from mice that had been treated previously with TY26898. FIG. 17B depicts cleaved and intact TY26898 in OV90 ovarian tumors from mice that had been treated previously with TY26898.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

### A. Definitions

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures used in connection with, and techniques of, antibody engineering, immunotherapy, cell and tissue culture, molecular biology, immunology, microbiology, genetics and protein and nucleic acid chemistry described herein are those well-known and commonly used in the art.

As used herein, each of the following terms has the meaning associated with it in this section.

The articles “a” and “an” refer to one or to more than one (i.e., to at least one) of the grammatical object of the article. By way of example, “an element” means one element or more than one element.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. The term “amino acid analogs” refers to compounds that have the same basic chemical structure as a naturally occurring amino acid but the C-terminal carboxy group, the N-terminal amino group, or side chain functional group has been chemically modified to another functional group. The term “amino acid mimetics” refers to chemical compounds that have a structure that is different from the general chemical structure of an amino acid, but that functions similarly to a naturally occurring amino acid. As used herein, the twenty conventional amino acids and their abbreviations follow conventional usage. See Immunology A Synthesis (2nd Edition, E. S. Golub and D. R. Gren, Eds., Sinauer Associates, Sunderland, Mass. (1991)).

The term “amino acid substitution” or “amino acid residue substitution” refers to a change in one of the amino acid residues of an amino acid sequence relative to the referenced sequence. An amino acid sequence may have any number (e.g., 1, 2, 3, 4, 5, or more) of amino acid substitutions relative to the referenced sequence at any residue of the sequence. The term “variant” refers to a polypeptide or amino acid sequence having one or more amino acid substitutions relative to the referenced amino acid sequence.

The term “antibody” is used herein in the broadest sense and specifically covers monoclonal antibodies (including full length monoclonal antibodies), polyclonal antibodies, masked antibodies (e.g., activatable antibodies), multispecific antibodies (e.g., bispecific antibodies), and antibody fragments (e.g., a single-chain variable fragment or scFv) so long as they exhibit the desired biological activity.

The term “antibody” is an art-recognized term and may refer to an antigen-binding protein (i.e., immunoglobulin) having a basic four-polypeptide chain structure consisting of two identical heavy (H) chains and two identical light (L) chains. Each L chain is linked to an H chain by one covalent disulfide bond, while the two H chains are linked to each other by one or more disulfide bonds depending on the H chain isotype. Each heavy chain has, at the N-terminus, a variable region (abbreviated herein as VH) followed by a constant region. The heavy chain constant region is comprised of three domains, CH1, CH2 and CH3. Each light chain has, at the N-terminus, a variable region (abbreviated herein as VL) followed by a constant region at its other end. The light chain constant region is comprised of one domain, CL. The VL is aligned with the VH and the CL is aligned with the first constant domain of the heavy chain (CH1). The pairing of a VH and VL together forms a single antigen-binding site. An IgM antibody consists of 5 of the basic heterotetramer units along with an additional polypeptide called J chain, and therefore contains 10 antigen binding sites, while secreted IgA antibodies can polymerize to form

The term “hypervariable region” or “HVR,” as used herein, refers to each of the regions of an antibody variable domain, which are hypervariable in sequence. HVRs may form structurally defined loops (“hypervariable loops”). Generally, native four-chain antibodies comprise six HVRs; three in the VH (H1, H2, H3), and three in the VL (L1, L2, L3). HVRs are interspersed with regions that are more conserved, termed framework regions (FW). Each VH and VL is composed of three HVRs and four FWs, arranged from amino-terminus to carboxy-terminus in the following order: FW1, HVR1, FW2, HVR2, FW3, HVR3, FW4. HVRs generally comprise amino acid residues from the hypervariable loops and/or from the “complementarity determining regions” (CDRs), CDRs being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3 (Kabat et al., Sequences of Proteins of Immunological Interest, 5th Ed. Public Health Service, National Institutes of Health, Bethesda, MD (1991)). With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3 (Almagro and Fransson, Front. Biosci. 13:1619-1633 (2008)).

Table 1 below provides exemplary CDR definitions according to various algorithms known in the art.

TABLE 1
CDR Definitions.

| | Kabat¹ | Chothia² | MacCallum³ | IMGT⁴ | AHo⁵ |
|---|---|---|---|---|---|
| CDR-H1 | 31-35 | 26-32 | 30-35 | 27-38 | 25-40 |
| CDR-H2 | 50-65 | 53-55 | 47-58 | 56-65 | 58-77 |
| CDR-H3 | 95-102 | 96-101 | 93-101 | 105-117 | 109-137 |
| CDR-L1 | 24-34 | 26-32 | 30-36 | 27-38 | 25-40 |
| CDR-L2 | 50-56 | 50-52 | 46-55 | 56-65 | 58-77 |
| CDR-L3 | 89-97 | 91-96 | 89-96 | 105-117 | 109-137 |

¹Residue numbering follows the nomenclature of Kabat et al., J. Biol. Chem. 252: 6609-6616 (1977); Kabat et al., U.S. Dept. of Health and Human Services, “Sequences of proteins of immunological interest” (1991).
²Residue numbering follows the nomenclature of Chothia et al., J. Mol. Biol. 196: 901-917 (1987); Al-Lazikani B. et al., J. Mol. Biol., 273: 927-948 (1997).
³Residue numbering follows the nomenclature of MacCallum et al., J. Mol. Biol. 262: 732-745 (1996); Abhinandan and Martin, Mol. Immunol., 45: 3832-3839 (2008).
⁴Residue numbering follows the nomenclature of Lefranc M. P. et al., Dev. Comp. Immunol., 27: 55-77 (2003); and Honegger and Pluckthun, J. Mol. Biol., 309: 657-670 (2001).
⁵Residue numbering follows the nomenclature of Honegger and Pluckthun, J. Mol. Biol., 309: 657-670 (2001).

The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. Within light and heavy chains, the variable and constant regions are joined by a “J” region of about 12 or more amino acids, with the heavy chain also including a “D” region of about 10 or more amino acids. See generally, Fundamental Immunology Ch. 7 (Paul, W., ed., 2nd ed. Raven Press, N.Y. (1989)).

The L chain from any vertebrate species can be assigned to one of two clearly distinct types, called kappa and lambda, based on the amino acid sequences of their constant domains. Depending on the amino acid sequence of the constant domain of their heavy chains (CH), antibodies can be assigned to different classes or isotypes. The term “fragment crystallizable (Fc) region” refers to the heavy chain constant domain. There are five classes of antibodies: IgA, IgD, IgE, IgG, and IgM, having heavy chains designated α (alpha), δ (delta), ε (epsilon), γ (gamma), and μ (mu), respectively. The IgG class of antibody can be further classified into four subclasses IgG1, IgG2, IgG3, and IgG4 by the gamma heavy chains, Y1-Y4, respectively.

The term “antigen-binding fragment” or “antigen binding portion” of an antibody refers to one or more portions of an antibody that retain the ability to bind to the antigen that the antibody binds to (e.g., CD47). Examples of “antigen-binding fragment” of an antibody include (i) a Fab fragment, a monovalent fragment consisting of the VL, VH, CL and CH1 domains; (ii) a F(ab′) 2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341:544-546 (1989)), which consists of a VH domain; and (vi) an isolated complementarity determining region (CDR).

The term “masked antibody” refers to an antibody, or an antigen-binding fragment thereof, comprising a peptide that interferes with, obstructs, reduces the ability of, prevents, inhibits, or competes with the target binding moiety (TBM) of the antibody, or antigen-binding fragment thereof, for binding to its target. A masked antibody may be generated by linking a masking peptide to the TBM of an antibody or an antigen-fragment thereof.

The term “activatable antibody” refers to a masked antibody, or an antigen-binding fragment thereof, that exhibits a first binding affinity to a target when in an unactivated (e.g., inhibited, masked, and/or uncleaved) state, and exhibits a second binding affinity to the target in an activated (e.g., uninhibited, unmasked, and/or cleaved) state, where the second binding affinity is greater than the first binding affinity. An activatable antibody may be generated by linking a masking peptide comprising an activatable component (e.g., a cleavable moiety (CM)) to the target-binding moiety (TBM) of an antibody or an antigen-fragment thereof. Activatable antibodies have been described, for example, in U.S. Pat. Pub. No. 2019/0241886 and U.S. Pat. Pub. No. 2021/0207126, the contents of both of which are incorporated herein by reference in their entirety.

A “target binding moiety (TBM)” refers to a structural moiety of an antigen binding portion of an antibody which binds to the target antigen of the antibody. The TBM may comprise a VH and a VL, such as any VH or VL described herein in any combination.

A “masking peptide” refers to a structural moiety of the masked antibody (e.g., activatable antibody) which inhibits binding of the TBM to its target antigen, and typically comprises, from N terminus to C terminus, a masking moiety (MM) and a linkage moiety (LM). The C terminus of the masking peptide is typically linked to the N terminus of the VH or the VL of a masked antibody (e.g., activatable antibody). In some embodiments, the masking peptide, or a portion thereof, interferes with or inhibits binding of the TBM to its target so efficiently that binding of the TBM to its target is extremely low and/or below the limit of detection (e.g., binding cannot be detected in an ELISA or flow cytometry assay). The masked antibodies (e.g., activatable antibodies) described herein may comprise one or more linkers, e.g., within the LM, disposed between MM and LM, LM and VH or VL, or VH and hinge region of an Fc.

The LM of the masking peptide may comprise a cleavable moiety (CM). A CM generally includes an amino acid sequence that is cleavable, for example, serves as the substrate for an enzyme and/or a cysteine-cysteine pair capable of forming a reducible disulfide bond. As such, when the terms “cleavage,” “cleavable,” “cleaved” and the like are used in connection with a CM, the terms encompass enzymatic cleavage, e.g., by a protease, as well as disruption of a disulfide bond between a cysteine-cysteine pair via reduction of the disulfide bond that can result from exposure to a reducing agent. The amino acid sequence of the CM may overlap with or be included within the MM. Activatable antibodies may comprise a CM configured to mediate activation of the antibody. For example, when the CM of an activatable antibody is intact (e.g., uncleaved by a corresponding enzyme, and/or containing an unreduced cysteine-cysteine disulfide bond), the masking peptide, or a portion thereof, may interfere with or inhibit binding of the TBM to its target.

The term “masking efficiency” refers to the efficiency with which the masking peptide inhibits binding of the TBM to the target antigen. Masking efficiency may be measured as the difference in, or the ratio of, a characteristic (e.g., binding affinity for the target antigen) or an activity (e.g., blocking binding of the target antigen to a ligand) of a masked antibody (e.g., activatable antibody) having a TBM and a masking peptide, relative to a corresponding unmasked antibody (“parental antibody”) having the same TBM but lacking the masking peptide. In activatable antibodies, masking efficiency may be measured as the difference in, or the ratio of, a characteristic (e.g., binding affinity for the target antigen) or an activity (e.g., blocking binding of the target antigen to a ligand) of the activatable antibody having a TBM and a masking peptide in its unactivated (e.g., inhibited, masked, and/or uncleaved) state, relative to the activatable antibody in its activated (e.g., uninhibited, unmasked, and/or cleaved) state, or relative to a parental antibody having the same TBM but lacking the masking peptide. For example, the masking efficiency may be measured by dividing the EC50 of an activatable antibody for binding a target antigen in its unactivated (e.g., inhibited, masked, and/or uncleaved) state, relative to the EC50 or KD of the activatable antibody to bind to the target antigen in its activated (e.g., uninhibited, unmasked, and/or cleaved) state, or relative to EC50 or Kp of the parental antigen to bind to the target antigen. The EC50 values may be measured in an ELISA assay, for example, as described in Example 5, or a Jurkat NFAT reporter assay, for example, as described in U.S. Pat. App. Pub. No. US20210207126 A1. The Kp values may be measured by, for example, using surface plasmon resonance using one of the systems described herein.

The term “antibody derivative” or “derivative” of an antibody refers to a molecule that is capable of binding to the same antigen (e.g., CD47) that the antibody binds to and comprises an amino acid sequence of the antibody linked to an additional molecular entity. The amino acid sequence of the antibody that is contained in the antibody derivative may be a full-length heavy chain, a full-length light chain, any portion or portions of a full-length heavy chain, any portion or portions of the full-length light chain of the antibody, any other fragment(s) of an antibody, or the complete antibody. The additional molecular entity may be a chemical or biological molecule. Examples of additional molecular entities include chemical groups, amino acids, peptides, proteins (such as enzymes, antibodies), and chemical compounds. The additional molecular entity may have any utility, such as for use as a detection agent, label, marker, pharmaceutical or therapeutic agent. The amino acid sequence of an antibody may be attached or linked to the additional molecular entity by chemical coupling, genetic fusion, noncovalent association, or otherwise. The term “antibody derivative” also encompasses chimeric antibodies, humanized antibodies, and molecules that are derived from modifications of the amino acid sequences of a CD47 antibody, such as conservation amino acid substitutions, additions, and insertions.

The term “binding molecule” encompasses (1) antibody, (2) antigen-binding fragment of an antibody, (3) masked antibody (e.g., activatable antibody), and (4) derivative of an antibody, each as defined herein.

The term “binding CD47,” “binds CD47,” “binding to CD47,” or “binds to CD47” refers to the binding of a binding molecule (e.g., an antibody, antigen-binding fragment, or masked antibody (e.g., activatable antibody)) to the human CD47 in an in vitro assay, such as a Octet® RED96 assay as described in Example 2, with an affinity (KD) of 100 nM or less.

The terms “CD47” and “CD47 receptor” are used interchangeably in the present application, and include the human CD47 receptor, as well as variants, isoforms, and species homologs thereof. Accordingly, a binding molecule (e.g., an antibody, antigen-binding fragment, or masked antibody (e.g., activatable antibody)) may also bind CD47 from species other than human. In other cases, a binding molecule (e.g., an antibody, antigen-binding fragment, or masked antibody (e.g., activatable antibody)) may be completely specific for the human CD47 and may not exhibit species or other types of cross-reactivity. The term “CD47-ECD” refers to the extracellular domain of CD47.

The term “CD47”, as used in the present application, includes the human CD47 (e.g., UniProt accession number Q08722; NCBI accession number NP_001768), as well as variants, isoforms, and species homologs thereof (e.g., mouse CD47 (e.g., UniProt accession number Q61735; NCBI accession number NP_034711), rat CD47 (e.g., UniProt accession number P97829; NCBI accession number NP_062068), dog CD47 (e.g., UniProt accession number F1P6D7), cynomolgus monkey CD47 (e.g., UniProt accession number G7NZR3; NCBI accession number XP_005548289), rhesus monkey (e.g., NCBI accession number NP_001253446) etc.). Accordingly, a binding molecule (e.g., an antibody, antigen-binding fragment, or masked antibody (e.g., activatable antibody)) may also bind CD47 from species other than human. In other cases, a binding molecule may be completely specific for the human CD47 and may not exhibit species or other types of cross-reactivity.

The term “SIRPα” is used in the present application, and includes the human SIRPα (e.g., UniProt accession number P78324), as well as variants, isoforms, and species homologs thereof (e.g., mouse SIRPα (e.g., UniProt accession number P97797), rat SIRPα (e.g., UniProt accession number P97710), dog SIRPα (e.g., UniProt accession number F1PK00), cynomolgus monkey SIRPα (e.g., NCBI accession number NP_001271679), etc.). Accordingly, a binding molecule (e.g., an antibody, antigen-binding fragment, or masked antibody (e.g., activatable antibody)) may also block binding of CD47, from human or another species, to a SIRPα from species other than human. In other cases, a binding molecule may be completely specific for the interaction of human CD47 to human SIRPα and may not exhibit species or other types of cross-reactivity.

The term “CD47 antibody” or “anti-CD47 antibody” refers to an antibody, as defined herein, capable of binding to human CD47 receptor.

The term “chimeric antibody” refers to an antibody that comprises amino acid sequences derived from different animal species, such as those having a variable region derived from a human antibody and a murine immunoglobulin constant region.

The term “compete for binding” refers to the interaction of two antibodies in their binding to a binding target. A first antibody competes for binding with a second antibody if binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not, be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope, whether to the same, greater, or lesser extent, the antibodies are said to “cross-compete” with each other for binding of their respective epitope(s).

The term “epitope” refers to a part of an antigen to which an antibody (or antigen-binding fragment thereof) binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope can include various numbers of amino acids in a unique spatial conformation. Methods of determining spatial conformation of epitopes include, for example, x-ray crystallography, 2-dimensional nuclear magnetic resonance, deuterium and hydrogen exchange in combination with mass spectrometry, or site-directed mutagenesis, or all methods used in combination with computational modeling of antigen and its complex structure with its binding antibody and its variants. Sec, e.g., Epitope Mapping Protocols in Methods in Molecular Biology, Vol. 66, G. E. Morris, Ed. (1996). Once a desired epitope of an antigen is determined, antibodies to that epitope can be generated, e.g., using the techniques described herein. The generation and characterization of antibodies may also elucidate information about desirable epitopes. From this information, it is then possible to competitively screen antibodies for binding to the same epitope. An approach to achieve this is to conduct cross-competition studies to find antibodies that competitively bind with one another, i.e., the antibodies compete for binding to the antigen. A high throughput process for “binning” antibodies based upon their cross-competition is described in PCT Publication No. WO 03/48731.

The term “glycosylation sites” refers to amino acid residues which are recognized by a eukaryotic cell as locations for the attachment of sugar residues. The amino acids where carbohydrate, such as oligosaccharide, is attached are typically asparagine (N-linkage), serine (O-linkage), and threonine (O-linkage) residues. The specific site of attachment is typically signaled by a sequence of amino acids, referred to herein as a “glycosylation site sequence”. The glycosylation site sequence for N-linked glycosylation is:-Asn-X-Ser- or -Asn-X-Thr-, where X may be any of the conventional amino acids, other than proline. The terms “N-linked” and “O-linked” refer to the chemical group that serves as the attachment site between the sugar molecule and the amino acid residue. N-linked sugars are attached through an amino group; O-linked sugars are attached through a hydroxyl group. The term “glycan occupancy” refers to the existence of a carbohydrate moiety linked to a glycosylation site (i.e., the glycan site is occupied). Where there are at least two potential glycosylation sites on a polypeptide, either none (0-glycan site occupancy), one (1-glycan site occupancy) or both (2-glycan site occupancy) sites can be occupied by a carbohydrate moiety.

The term “host cell” refers to a cellular system which can be engineered to generate proteins, protein fragments, or peptides of interest. Host cells include, without limitation, cultured cells, e.g., mammalian cultured cells derived from rodents (e.g., rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell.”

The term “human antibody” refers to an antibody in which the entire amino acid sequences of the light chains and heavy chains are from the human immunoglobulin genes. A human antibody may contain murine carbohydrate chains if produced in a mouse, in a mouse cell or in a hybridoma derived from a mouse cell. Human antibodies may be prepared in a variety of ways known in the art.

The term “humanized antibody” refers to a chimeric antibody that contains amino acid residues derived from human antibody sequences. A humanized antibody may contain some or all of the CDRs or HVRs from a non-human animal or synthetic antibody while the framework and constant regions of the antibody contain amino acid residues derived from human antibody sequences.

The term “illustrative antibody” or “exemplary antibody” refers to any one of the antibodies, masked antibodies (e.g., activatable antibodies), or antigen-binding fragments described in the disclosure, and designated as those listed in Tables 3A-6. These antibodies may be in any class (e.g., IgA, IgD, IgE, IgG, and IgM). Thus, each antibody identified above encompasses antibodies in all five classes that have the same amino acid sequences for the VL and VH regions. Further, the antibodies in the IgG class may be in any subclass (e.g., IgG1 lgG2, IgG3, and lgG4). Thus, each antibody identified above in the IgG subclass encompasses antibodies in all four subclasses that have the same amino acid sequences for the VL and VH regions. The amino acid sequences of the heavy chain constant regions of human antibodies in the five classes, as well as in the four IgG subclasses, are known in the art. The amino acid sequence of the full length heavy chain and light chain for the IgG4 and IgG1 subclass of each of the illustrative antibodies shown in Table 6 is provided in the disclosure.

The term “isolated antibody” or “isolated binding molecule” refers to an antibody or a binding molecule, as defined herein, that: (1) is not associated with naturally associated components that accompany it in its native state; (2) is free of other proteins from the same species; (3) is expressed by a cell from a different species; or (4) does not occur in nature. Examples of isolated antibodies include a CD47 antibody that has been affinity purified using CD47, a CD47 antibody that has been generated by hybridomas or other cell line in vitro, and a CD47 antibody derived from a transgenic animal.

The terms “isolated nucleic acid” and “isolated polynucleotide” refers to a nucleic acid molecule or polynucleotide of genomic, cDNA, or synthetic origin, or a combination thereof, which is separated from other nucleic acid molecules and polynucleotides present in the natural source of the nucleic acid. For example, with regard to genomic DNA, the term “isolated” includes nucleic acid molecules and polynucleotides which are separated from the chromosome with which the genomic DNA is naturally associated. Preferably, an “isolated” nucleic acid or polynucleotide is free of sequences which naturally flank the nucleic acid or polynucleotide (i.e., sequences located at the 5′ and 3′ ends of the nucleic acid of interest).

The term “ka” refers to the association rate constant of a particular antibody-antigen interaction, whereas the term “ka” refers to the dissociation rate constant of a particular antibody-antigen interaction.

The term “KD” refers to the equilibrium dissociation constant of a particular antibody-antigen interaction. It is obtained from the ratio of ka to ka (i.e., ka/ka) and is expressed as a molar concentration (M). Kpis used as a measure for the affinity of an antibody's binding (i.e., its “binding affinity”) to its binding partner. The smaller the KD, the more tightly bound the antibody is, or the higher the binding affinity between antibody and the antigen. For example, an antibody with a nanomolar (nM) dissociation constant binds more tightly (i.e., has a higher binding affinity) to a particular antigen than an antibody with a micromolar (uM) dissociation constant. Kp values for antibodies can be determined using methods well established in the art. One method for determining the Kp of an antibody is by using surface plasmon resonance, typically using a biosensor system such as a Biacore® system or an Octet® RED96 System. An assay procedure using the Octet® RED96 System is described in the Examples section of this disclosure.

As used herein, the terms “subject”, “patient”, and “individual” are used interchangeably and may refer to a human or a non-human animal. In some examples, “subject”, “patient”, or “individual” refers to a subject, patient, or individual in need of treatment for a disease or disorder. A “non-human animal” may refer to any animal not classified as a human, such as domestic, farm, or zoo animals, sports, pet animals (such as dogs, horses, cats, cows, etc.), as well as animals used in research. Research animals may refer without limitation to nematodes, arthropods, vertebrates, mammals, frogs, rodents (e.g., mice or rats), fish (e.g., zebrafish or pufferfish), birds (e.g., chickens), dogs, cats, and non-human primates (e.g., rhesus monkeys, cynomolgus monkeys, chimpanzees, etc.). In some embodiments, the subject, patient, or individual is a human.

The term “prevent” or “preventing,” with reference to a certain disease condition in a mammal, refers to preventing or delaying the onset of the disease, or preventing the manifestation of clinical or subclinical symptoms thereof.

As used herein, “sequence identity” between two polypeptide sequences indicates the percentage of amino acids that are identical between the sequences. The amino acid sequence identity of polypeptides can be determined conventionally using known computer programs such as Bestfit, FASTA, or BLAST (scc, e.g. Pearson, Methods Enzymol. 183:63-98 (1990); Pearson, Methods Mol. Biol. 132:185-219 (2000); Altschul et al., J. Mol. Biol. 215:403-410 (1990); Altschul et al., Nucelic Acids Res. 25:3389-3402 (1997)). When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, fragments, or variants thereof disclosed herein.

The term “treat”, “treating”, or “treatment”, with reference to a certain disease condition in a mammal, refers causing a desirable or beneficial effect in the mammal having the disease condition. The desirable or beneficial effect may include reduced frequency or severity of one or more symptoms of the disease (e.g., tumor growth and/or metastasis, or other effect mediated by the numbers and/or activity of immune cells, and the like), or arrest or inhibition of further development of the disease, condition, or disorder. In the context of treating cancer in a mammal, the desirable or beneficial effect may include inhibition of further growth or spread of cancer cells, death of cancer cells, inhibition of reoccurrence of cancer, reduction of pain associated with the cancer, or improved survival of the mammal. The effect can be either subjective or objective. For example, if the mammal is human, the human may note improved vigor or vitality or decreased pain as subjective symptoms of improvement or response to therapy. Alternatively, the clinician may notice a decrease in tumor size or tumor burden based on physical exam, laboratory parameters, tumor markers or radiographic findings. Some laboratory signs that the clinician may observe for response to treatment include normalization of tests, such as white blood cell count, red blood cell count, platelet count, erythrocyte sedimentation rate, and various enzyme levels. Additionally, the clinician may observe a decrease in a detectable tumor marker. Alternatively, other tests can be used to evaluate objective improvement, such as sonograms, nuclear magnetic resonance testing and positron emissions testing.

The term “CD47-positive disease” or “CD47-positive condition” refers to a disease or condition that involves one or more cells having an abnormal expression, amount, activity, or function of CD47, and/or can be treated by modulating the binding of CD47 with one or more of its binding partners. For example, CD47-positive diseases include cancers characterized by tumors expressing a high level of CD47 as compared to healthy tissues, and which may be treated by inhibiting the binding of CD47 with one of its binding partners, e.g., SIRPα. Exemplary CD47-positive cancers are provided herein. CD47-positive cancers include, but are not limited to, lymphomas (e.g., diffuse large B-cell lymphoma (DLBCL) and lymphoid neoplasm diffuse large B-cell lymphoma (DLBC)), leukemias (e.g., acute myeloid leukemia (AML)), head and neck cancers (e.g., head and neck squamous cell carcinoma (HNSC)), gastric cancers (e.g., gastric carcinoma (GC) and Her2+ gastric-esophageal junction (GEJ) cancer), breast cancers (e.g., breast invasive carcinoma (BRCA), HER2+ breast cancer, hormone receptor positive breast cancer, and triple negative breast cancer (TNBC)), cervical cancers (e.g., cervical squamous cell carcinoma and endocervical adenocarcinoma (CESC)), cholangiocarcinomas (CHOL), colon cancers (e.g., colon adenocarcinoma (COAD)), ovarian cancers (e.g., ovarian serous cystadenocarcinoma (OV)), thyroid cancers (e.g., thyroid carconima (THCA)), uterine cancers (e.g. uterine corpus endometrial carcinoma (UCEC)), endometrial cancers, lung cancers (e.g. lung adenocarcinoma (LUAD), lung squamous cell carcinoma (LUSC), and small cell lung cancer (SCLC)), mesotheliomas (MESO), and pancreatic cancers (e.g., pancreatic adenocarcinoma (PAAD)).

The term “vector” refers to a nucleic acid molecule capable of transporting a foreign nucleic acid molecule. The foreign nucleic acid molecule is linked to the vector nucleic acid molecule by a recombinant technique, such as ligation or recombination. This allows the foreign nucleic acid molecule to be multiplied, selected, further manipulated or expressed in a host cell or organism. A vector can be a plasmid, phage, transposon, cosmid, chromosome, virus, or virion. One type of vectors can be integrated into the genome of a host cell upon introduction into the host cell, and thereby are replicated along with the host genome (e.g., non-episomal mammalian vectors). Another type of vector is capable of autonomous replication in a host cell into which it is introduced (e.g., bacterial vectors having a bacterial origin of replication and episomal mammalian vectors). Another specific type of vector capable of directing the expression of expressible foreign nucleic acids to which they are operatively linked is commonly referred to as “expression vectors.” Expression vectors generally have control sequences that drive expression of the expressible foreign nucleic acids. Simpler vectors, known as “transcription vectors,” are only capable of being transcribed but not translated: they can be replicated in a target cell but not expressed. The term “vector” encompasses all types of vectors regardless of their function. Vectors capable of directing the expression of expressible nucleic acids to which they are operatively linked are commonly referred to “expression vectors.”

The methods and techniques of the present disclosure are generally performed according to methods well known in the art and as described in various general and more specific references that are cited and discussed throughout the present specification unless otherwise indicated. Such references include, e.g., Sambrook and Russell, Molecular Cloning, A Laboratory Approach, Cold Spring Harbor Press, Cold Spring Harbor, N.Y. (2001), Ausubel et al., Current Protocols in Molecular Biology, John Wiley & Sons, NY (2002), and Harlow and Lane Antibodies: A Laboratory Manual, Cold Spring Harbor Laboratory Press, Cold Spring Harbor, N.Y. (1990). Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

B. Antibodies, Antigen-Binding Fragments, and Masked Antibodies that Bind to Human CD47

The present disclosure provides isolated binding molecules (e.g., an antibody, antigen-binding fragment, or masked antibody (e.g., activatable antibody)) that bind to human CD47, including CD47 antibodies, activatable CD47 antibodies, antigen-binding fragments of the CD47 antibodies, and derivatives of the CD47 antibodies. In some embodiments, the binding molecules are any of the antibodies, masked antibodies (e.g., activatable antibodies), and antigen-binding fragments described herein, including antibodies, masked antibodies (e.g., activatable antibodies), and antigen-binding fragments described with reference to epitope binding and antibodies described with reference to specific amino acid sequences of CDRs, variable regions (VL, VH), and IgG (e.g., IgG1 and IgG4) light and heavy chains. In some embodiments, the present disclosure relates to antibodies, antigen-binding fragments, and masked antibodies (e.g., activatable antibodies) that bind to human CD47, and have at least one (e.g., at least one, at least two, at least three, at least four, at least five, or all six) of the following functional properties: (a) bind to human CD47 with a Kp of 500 nM or less; (b) are cross-reactive with monkey, rat, or dog CD47; (c) are capable of inhibiting tumor cell growth; (d) have therapeutic effect on a cancer; (c) block binding between CD47 and SIRP proteins (e.g., SIRPα); and (f) induce antibody-dependent cellular cytotoxicity (ADCC) and/or antibody-dependent cellular phagocytosis (ADCP) against CD47-expressing tumor cells. In some embodiments, the antibodies, antigen-binding fragments, and masked antibodies (e.g., activatable antibodies) disclosed herein can also block, e.g., completely block, the binding between CD47 and its ligand SIRPα. Also provided herein are one or more anti-CD47 antibodies, antigen-binding fragments, or masked antibodies (e.g., activatable antibodies) that cross-compete for binding to human CD47 with one or more of the antibodies, antigen-binding fragments, or masked antibodies (e.g., activatable antibodies) as described herein.

In some embodiments, the antibodies, antigen-binding fragments, or masked antibodies (e.g., activatable antibodies) alter (e.g., inhibit or enhance) one or more (e.g., one or more, two or more, three or more, etc.) activities of human CD47 when a cell (e.g., a human cell) expressing human CD47 is contacted by the antibody or antigen binding fragment. Various CD47 activities are known in the art and may include, without limitation, inhibition of antibody-dependent cellular phagocytosis (ADCP) by immune cells (e.g., macrophages); inhibition of ADCC by natural killer (NK) cells; and stimulation of cell-cell fusion, T-cell activation, T-cell proliferation, apoptosis, cell proliferation, cell survival, and cell adhesion (Sick et al. “CD47 update: a multifaceted actor in the tumor microenvironment of potential therapeutic interest.” Br J Pharmacol. 2012; 167 (7): 1415-1430). In some embodiments, the one or more CD47 activities is not CD47 binding to its ligand (e.g., SIRPα). Methods of measuring CD47 activity (e.g., inhibition of phagocytosis, inhibition of ADCC, etc.) are known in the art, including, for example, the methods described in Examples 9 and 10 below.

In some embodiments, the antibodies, antigen-binding fragments, and masked antibodies (e.g., activatable antibodies) described herein have enhanced antibody-dependent cellular cytotoxic (ADCC) and/or antibody-dependent cellular phagocytosis (ADCP), function(s). Methods of measuring ADCC of antibodies and antigen binding fragments are well known in the art. Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I. et al., Proc. Nat'l Acad. Sci. USA 82:1499-1502 (1985); U.S. Pat. No. 5,821,337 (see Bruggemann, M. et al, J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96TM non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). The contribution of ADCC to tumor cell killing can be measured, for example, with a specific test that uses NK-92 cells that have been transfected with a high-affinity FcR. Results are compared to wild-type NK-92 cells that do not express the FcR. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). Methods of measuring ADCC of antibodies and antigen binding fragments include, for example, the method described in Example 10 below. Methods of measuring ADCP of antibodies and antigen binding fragments are also well known in the art. For example, to assess ADCP activity of a molecule of interest, an in vitro ADCP assay (see, e.g., Bracher et al., 2007, J. Immunol. Methods 323:160-71) can be performed. Useful phagocytotic cells for such assays include peripheral blood mononuclear cells (PBMC), purified monocytes from PBMC, or U937 cells differentiated to the mononuclear type. Alternatively or additionally, ADCP activity of the molecule of interest may be assessed in vivo, for example, in an animal model (see, e.g., Wallace et al., 2001, J. Immunol. Methods 248:167-82).). Methods of measuring ADCP of antibodies and antigen binding fragments include, for example, the method described in Example 9 below.

In some embodiments, the antibodies, antigen-binding fragments, or masked antibodies (e.g., activatable antibodies) do not cause hemagglutination (e.g., clustering) of red blood cells (RBCs) (e.g., human RBCs) in vitro. In certain embodiments, the antibody, antigen-binding fragment, or masked antibody (e.g., activatable antibody) does not cause hemagglutination of human RBCs in vitro when provided at a concentration of up to about 50 nM, up to about 100 nM, up to about 500 nM, up to about 1000 nM, or up to about 5000 nM.

In some embodiments, the antibodies, antigen-binding fragments, or masked antibodies (e.g., activatable antibodies) have therapeutic effect on a cancer. In some embodiments, the antibodies, antigen-binding fragments, or masked antibodies (e.g., activatable antibodies) reduce one or more signs or symptoms of a cancer. In some embodiments, a subject suffering from a cancer goes into partial or complete remission when administered the antibodies, antigen-binding fragments, or masked antibodies (e.g., activatable antibodies). In some embodiments, the cancer comprises B cell lymphoma, T cell lymphoma, or any combination thereof. In certain embodiments, the cancer is B cell lymphoma. In other embodiments, the cancer is T cell lymphoma. In some embodiments, the cancer is a lymphoma (e.g., diffuse large B-cell lymphoma (DLBCL) and lymphoid neoplasm diffuse large B-cell lymphoma (DLBC)), leukemia (e.g., acute myeloid leukemia (AML)), head and neck cancer (e.g., head and neck squamous cell carcinoma (HNSC)), gastric cancer (e.g., gastric carcinoma (GC) and Her2+ gastric-esophageal junction (GEJ) cancer), breast cancer (e.g., breast invasive carcinoma (BRCA), HER2+ breast cancer, hormone receptor positive breast cancer, and triple negative breast cancer (TNBC)), cervical cancer (e.g., cervical squamous cell carcinoma and endocervical adenocarcinoma (CESC)), cholangiocarcinoma (CHOL), colon cancer (e.g., colon adenocarcinoma (COAD)), ovarian cancer (e.g., ovarian serous cystadenocarcinoma (OV)), thyroid cancer (e.g., thyroid carconima (THCA)), uterine cancer (e.g. uterine corpus endometrial carcinoma (UCEC)), endometrial cancer, lung cancer (e.g. lung adenocarcinoma (LUAD), lung squamous cell carcinoma (LUSC), and small cell lung cancer (SCLC)), mesothelioma (MESO), or pancreatic cancer (e.g., pancreatic adenocarcinoma (PAAD)).

In some embodiments, the antibodies, antigen-binding fragments, or masked antibodies (e.g., activatable antibodies) bind to an epitope of human CD47 that is distinct from other antibodies. In some embodiments, binding of the antibody, antigen-binding fragment, or masked antibody (e.g., activatable antibody) to the distinct epitope of human CD47 causes binding of the antibody, antigen-binding fragment, or masked antibody (e.g., activatable antibody) to human CD47 in a cis configuration, rather than a trans configuration. In some embodiments, binding of the antibody, antigen-binding fragment, or masked antibody (e.g., activatable antibody) to the distinct epitope of human CD47 in a cis configuration causes the antibody to exhibit reduced agglutination of red blood cells than an antibody that binds human CD47 in a trans configuration. In certain embodiments, masked antibody binds to 1, 2, 3, 4, 5, 6, 7, 8, 9, or all 10 amino acid residues of human CD47 selected from the group consisting of K39, W40, K41, F50, D51, G52, T99, E100, L101, and T102. In certain embodiments, which may be combined with the preceding embodiments, the masked antibody does not bind to 1, 2, 3, 4, 5, 6, 7, 8, 9, 10, 11, 12, 13, 14, 15, 16, 17, 18, 19, or all 20 amino acid residues of human CD47 selected from the group consisting of L2, L3, F4, K6, N27, E29, A30, Q31, T34, E35, V36, Y37, D46, T49, A53, E97, R103, E104, G105, and E106. In certain embodiments, the masked antibody binds to amino acid residues K39, W40, K41, F50, D51, G52, T99, E100, L101, and T102 of human CD47 and does not bind to any of amino acid residues L2, L3, F4, K6, N27, E29, A30, Q31, T34, E35, V36, Y37, D46, T49, A53, E97, R103, E104, G105, and E106 of human CD47.

In another aspect, the disclosure provides isolated antibodies that compete or cross-compete for binding to human CD47 with any of the illustrative antibodies, antigen-binding fragments, or masked antibodies (e.g., activatable antibodies) of the disclosure, such as TY25031, TY25034, TY25040, TY21446, TY1447, TY26294 (upon cleavage of the CM), TY26896, TY26897, TY26898 (upon cleavage of the CM), and TY26899 (upon cleavage of the CM). In a particular embodiment, the disclosure provides isolated antibodies that compete or cross-compete for binding to the same epitope on the human CD47 with any of the illustrative antibodies of the disclosure. The ability of an antibody to compete or cross-compete for binding with another antibody can be determined using standard binding assays known in the art, such as BIAcore analysis, ELISA assays, or flow cytometry. For example, one can allow an illustrative antibody, antigen-binding fragment, or masked antibody (e.g., activatable antibody) of the disclosure to bind to human CD47 under saturating conditions and then measure the ability of the test antibody to bind to the CD47. If the test antibody is able to bind to the CD47 at the same time as the illustrative antibody, then the test antibody binds to a different epitope as the illustrative antibody. However, if the test antibody is not able to bind to the CD47 at the same time, then the test antibody binds to the same epitope, an overlapping epitope, or an epitope that is in close proximity to the epitope bound by the illustrative antibody. This experiment can be performed using various methods, such as ELISA, RIA, FACS or surface plasmon resonance.

In one aspect, the present disclosure provides an isolated antibody, an antigen-binding fragment, or a masked antibody (e.g., activatable antibody) having specific complementarity determining regions (CDRs). The isolated antibody, an antigen-binding fragment, or a masked antibody (e.g., activatable antibody) can comprise one or more (e.g., one, two, three, four, five, or six) of any of the CDRs described herein, in any combination.

In some embodiments, the isolated antibody, antigen-binding fragment, or masked antibody (e.g., activatable antibody) comprises an antibody heavy chain variable region (VH), an antibody light chain variable region (VL), or both. In some embodiments, the VH comprises a first complementary-determining-region (CDR-H1), a second complementary-determining-region (CDR-H2), and a third complementary-determining-region (CDR-H3).

In some embodiments, the CDR-H1 comprises an amino acid sequence according to Formula (I): X1YX2IH (SEQ ID NO: 182), wherein X1 is D, G, N, R, or S, and X2 is A, G, or W.

In some embodiments, the CDR-H1 comprises an amino acid sequence according to Formula (II): SGX1X2WX3 (SEQ ID NO: 183), wherein X1 and X2 are each independently H or Y, and X3 is D, G, N, S, or T.

In some embodiments, the CDR-H2 comprises an amino acid sequence according to Formula (III): X1IX2X3X4GX5X6X7YX8PSLKS (SEQ ID NO: 184), wherein X1 is A, E, or R, X2 is S or Y. X3 is H, W, or Y, X4 is D or S, X5 is D, N, or S, X6 is K or T, X7 is R or Y, and X8 is N or S.

In some embodiments, the CDR-H2 comprises an amino acid sequence according to Formula (IV): X1IX2X3X4X5X6X7X8X9YAX10X11X12X13G (SEQ ID NO: 185), wherein X1 is A, G. I. R, or W. X2 is I, N, S, or Y, X3 is G or P, X4 is A, N, S, or V, X5 is F, G, or S, X6 is G or S, X7 is G, S, or T, X8 is A, P, or T, X9 is K, N, or Y, X10 is D or Q, X11 is K or S, X12 is For V, and X13 is K or Q.

In some embodiments, the CDR-H3 comprises an amino acid sequence according to Formula (V): X1X2X3X4X5X6FX7X8 (SEQ ID NO: 186), wherein X1 is Q, R, S, or Y, X2 is G, R, V, or Y, X3 is G, H, I, P, or Y, X4 is A, G, L, or Y, X5 is A, G, P, or Y, X6 is A, D, G, R, S, or V, X7 is A or D, and X8 is V or Y.

In some embodiments, the CDR-H3 comprises an amino acid sequence according to Formula (VI): X1X2X3GX4X5X6X7DX8 (SEQ ID NO: 187), wherein X1 and X4 are each independently G or Y, X2 is A or G, X3 is R or Y, X5 and X6 are each independently A or Y, X7 is For L, and X8 is V or Y.

In some embodiments, the CDR-H3 comprises an amino acid sequence according to Formula (VII): X1X2X3X4X5X6GX7FDX8 (SEQ ID NO: 188), wherein X1 is G or R, X2 is G or V, X3 is R or S, X4 is G or Y, X5 is G or S, X6 is For Y, X7 is A or W, and X8 is V or Y.

In some embodiments, the CDR-H3 comprises an amino acid sequence according to Formula (VIII): X1X2X3X4X5X6SX7X8YDX9FDX10 (SEQ ID NO: 189), wherein X1 is D or H, X2 is R or Y, X3 is A or L, X4 is For P, X5 and X9 are each independently A or G, X6 is G or S, X7 is G or T, X8 is S or Y, and X10 is I or Y.

In some embodiments, the CDR-L1 comprises an amino acid sequence according to Formula (IX): SASSX1VX2YX3Y (SEQ ID NO: 190), wherein X1 is R or S, X2 is G, S, or T, and X3 is I or V.

In some embodiments, the CDR-L1 comprises an amino acid sequence according to Formula (X): RASQX1IX2X3X4LX5 (SEQ ID NO: 191), wherein X1 is G or T, X2 is G or S, X3 is R or S, X4 is V or Y, and X5 is A or N.

In some embodiments, the CDR-L1 comprises an amino acid sequence according to Formula (XI): RASQX1VX2X3RX4LA (SEQ ID NO: 192), wherein X1 is S or T, X2 is I or R, X3 is G or S, and X4 is L or Y.

In some embodiments, the CDR-L1 comprises an amino acid sequence according to Formula (XII): RASX1SVDFX2GX3SFLX4 (SEQ ID NO: 193), wherein X1 is E or Q. X2 is H. V. or Y. X3 is F. I, or K, and X4 is A, D, or H.

In some embodiments, the CDR-L2 comprises an amino acid sequence according to Formula (XIII): X1ASX2X3X4X5 (SEQ ID NO: 194), wherein X1 is A or D, X2 is N, S. or T. X3 is L or R. X4 is A, E, or Q, and X5 is S or T.

In some embodiments, the CDR-L3 comprises an amino acid sequence according to Formula (XIV): X1QX2X3X4X5PX6T (SEQ ID NO: 195), wherein X1 is A, H, Q. or V. X2 is A, G. R. S, or Y, X3 is G, I, L, S, T, or Y, X4 is A, E, P. Q. R. S. T. or Y, X5 is A, I, L, S. T. or W, and X6 is F. H. L. W, or Y.

In some embodiments, the CDR-L3 comprises an amino acid sequence according to Formula (XV): QX1YX2SX3PX4X5X6T (SEQ ID NO: 196), wherein X1 is H or Q, X2 is A. T. or V. X3 is S or W. X4 is Por R, X5 is G or V. and X6 is For Y.

In some embodiments, the isolated antibody, antigen-binding fragment, or masked antibody (e.g., activatable antibody) comprises one or more (e.g., one, two, three, four, five, or six) CDRs comprising an amino acid sequence selected from the group consisting of SEQ ID NO: 182-196. In some embodiments, the isolated antibody, antigen-binding fragment, or masked antibody (e.g., activatable antibody) can comprise two or more (e.g., two, three, four, five, or six) CDRs each comprising an amino acid sequence selected from the group consisting of SEQ ID NO: 182-196.

In some embodiments, the isolated antibody, antigen-binding fragment, or masked antibody (e.g., activatable antibody) comprises a VH and a VL. In some embodiments, the VH comprises a CDR-H1, a CDR-H2, and a CDR-H3. In some embodiments, the CDR-H1 comprises the amino acid sequence of SEQ ID NO: 182 or 183. In some embodiments, CDR-H2 comprises the amino acid sequence of SEQ ID NO: 184 or 185. In some embodiments, the CDR-H3 comprises an amino acid sequence selected from the group consisting of SEQ ID NOs: 186-189. In certain embodiments, the VL comprises a CDR-L1, a CDR-L2, and a CDR-L3. In some embodiments, the CDR-L1 comprises an amino acid sequence selected from the group consisting of SEQ ID NOs: 190-193. In some embodiments, the CDR-L2 comprises the amino acid sequence of SEQ ID NO: 194. In some embodiments, the CDR-L3 comprises the amino acid sequence of SEQ ID NO: 195 or 196.

In some embodiments, the isolated antibody, antigen-binding fragment, or masked antibody (e.g., activatable antibody) can comprise a CDR-H1 having the amino acid sequence of SEQ ID NO: 182 or 183; a CDR-H2 having the amino acid sequence of SEQ ID NO: 184 or 185; a CDR-H3 having an amino acid sequence selected from the group consisting of SEQ ID NOs: 186-189,; a CDR-L1 having an amino acid sequence selected from the group consisting of SEQ ID NOs: 190-193; a CDR-L2 having the amino acid sequence of SEQ ID NO: 194; and/or a CDR-L3 having the amino acid sequence of SEQ ID NO: 195 or 196.

In some embodiments, the isolated antibody, antigen-binding fragment, or masked antibody (e.g., activatable antibody) comprises a VH and a VL, wherein 1) the VH comprises a CDR-H1, a CDR-H2, and a CDR-H3, wherein the CDR-H1 comprises the amino acid sequence of SEQ ID NO: 182 or 183; the CDR-H2 comprises the amino acid sequence of SEQ ID NO: 184 or 185; and/or the CDR-H3 comprises an amino acid sequence selected from the group consisting of SEQ ID NOs: 186-189; and/or 2) the VL comprises a CDR-L1, a CDR-L2, and a CDR-L3, the CDR-L1 comprises an amino acid sequence selected from the group consisting of SEQ ID NOs: 190-193; the CDR-L2 comprises the amino acid sequence of SEQ ID NO: 194; and/or the CDR-L3 comprises the amino acid sequence of SEQ ID NO: 195 or 196.

In certain embodiments, the isolated antibody, antigen-binding fragment, or masked antibody (e.g., activatable antibody) comprises a VH and a VL, wherein 1) the VH comprises a CDR-H1 comprising the amino acid sequence of SEQ ID NO: 182; a CDR-H2 comprising the amino acid sequence of SEQ ID NO: 185; and/or a CDR-H3 comprising the amino acid sequence of SEQ ID NO: 188; and/or b) the VL comprises a CDR-L1 comprising the amino acid sequence of SEQ ID NO: 191; a CDR-L2 comprising the amino acid sequence of SEQ ID NO: 194; and/or a CDR-L3 comprising the amino acid sequence of SEQ ID NO: 195. In one embodiment, the isolated antibody, antigen-binding fragment, or masked antibody (e.g., activatable antibody) comprises a VH and a VL, wherein 1) the VH comprises a CDR-H1 comprising the amino acid sequence of SEQ ID NO: 182, a CDR-H2 comprising the amino acid sequence of SEQ ID NO: 185, and a CDR-H3 comprising the amino acid sequence of SEQ ID NO: 188; and b) the VL comprises a CDR-L1 comprising the amino acid sequence of SEQ ID NO: 191, a CDR-L2 comprising the amino acid sequence of SEQ ID NO: 194, and a CDR-L3 comprising the amino acid sequence of SEQ ID NO: 195.

In some embodiments, the isolated antibody, antigen-binding fragment, or masked antibody (e.g., activatable antibody) comprises a VH and a VL, wherein the VH comprises one or more amino acid sequences selected from the group consisting of SEQ ID NOs: 182-189, and/or the VL comprises one or more amino acid sequences selected from the group consisting of SEQ ID NOs: 190-196. In some embodiments, the isolated antibody, antigen-binding fragment, or masked antibody (e.g., activatable antibody) comprises and a VL, wherein the VH comprises one or more amino acid sequences selected from the group consisting of SEQ ID NOs: 182-189, and the VL comprises one or more amino acid sequences selected from the group consisting of SEQ ID NOs: 190-196.

In certain embodiments, the isolated antibody, antigen-binding fragment, or masked antibody (e.g., activatable antibody) comprises a VH and a VL, wherein 1) the VH comprises one or more amino acid sequences selected from the group consisting of SEQ ID NOs: 182-183, one or more amino acid sequences selected from the group consisting of SEQ ID NOs: 184-185, and/or one or more amino acid sequences selected from the group consisting of SEQ ID NOs: 186-189; and/or 2) the VL comprises one or more amino acid sequences selected from the group consisting of SEQ ID NOs: 190-193, the amino acid sequence of SEQ ID NO: 194, and/or one or more amino acid sequences selected from the group consisting of SEQ ID NOs: 195-196. In certain embodiments, the isolated antibody, antigen-binding fragment, or masked antibody (e.g., activatable antibody) comprises and a VL, wherein 1) the VH comprises one or more amino acid sequences selected from the group consisting of SEQ ID NOs: 182-183, one or more amino acid sequences selected from the group consisting of SEQ ID NOs: 184-185, and one or more amino acid sequence selected from the group consisting of SEQ ID NOs: 186-189; and 2) the VL comprises one or more amino acid sequences selected from the group consisting of SEQ ID NOs: 190-193, the amino acid sequence of SEQ ID NO: 194, and one or more amino acid sequences selected from the group consisting of SEQ ID NOs: 195-196.

In some embodiments, 1) the VH comprises the amino acid sequence of SEQ ID NO: 182; the amino acid sequence of SEQ ID NO: 185; and/or the amino acid sequence of SEQ ID NO: 188; and/or 2) the VL comprises the amino acid sequence of SEQ ID NO: 191; the amino acid sequence of SEQ ID NO: 194; and/or the amino acid sequence of SEQ ID NO: 195. In one embodiment, 1) the VH comprises the amino acid sequence of SEQ ID NO: 182, the amino acid sequence of SEQ ID NO: 185, and the amino acid sequence of SEQ ID NO: 188; and 2) the VL comprises the amino acid sequence of SEQ ID NO: 191, the amino acid sequence of SEQ ID NO: 194, and the amino acid sequence of SEQ ID NO: 195.

In some embodiments, the isolated antibody, antigen-binding fragment, or masked antibody (e.g., activatable antibody) comprises one or more (e.g., one, two, three, four, five, or six) CDRs comprising an amino acid sequence selected from the group consisting of SEQ ID NO: 35-136, or a variant thereof comprising up to 5 (e.g., 1, 2, 3, 4, or 5) amino acid substitutions. The isolated antibody, antigen-binding fragment, or masked antibody (e.g., activatable antibody) can comprise two or more (e.g., two, three, four, five, or six) CDRs each comprising an amino acid sequence selected from the group consisting of SEQ ID NO: 35-136, or a variant thereof comprising up to 5 (e.g., 1, 2, 3, 4, or 5) amino acid substitutions, in any combination. In some embodiments, the isolated antibody, antigen-binding fragment, or masked antibody (e.g., activatable antibody) comprises one or more (e.g., one, two, three, four, five, or six) CDRs of one or more of illustrative antibodies as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises VH and a VL. In some embodiments, the VH comprises a CDR-H1, a CDR-H2, and a CDR-H3. In some embodiments, the CDR-H1 comprises an amino acid sequence selected from the group consisting of SEQ ID NOs: 35-51, or a variant thereof comprising up to 5 (e.g., 1, 2, 3, 4, or 5) amino acid substitutions. In some embodiments, CDR-H2 comprises an amino acid sequence selected from the group consisting of SEQ ID NOs: 52-68, or a variant thereof comprising up to 5 (e.g., 1, 2, 3, 4, or 5) amino acid substitutions. In some embodiments, the CDR-H3 comprises an amino acid sequence selected from the group consisting of SEQ ID NOs: 69-85, or a variant thereof comprising up to 5 (e.g., 1, 2, 3, 4, or 5) amino acid substitutions. In certain embodiments, the VL comprises a CDR-L1, a CDR-L2, and a CDR-L3. In some embodiments, the CDR-L1 comprises an amino acid sequence selected from the group consisting of SEQ ID NOs: 86-102, or a variant thereof comprising up to 5 (e.g., 1, 2, 3, 4, or 5) amino acid substitutions. In some embodiments, the CDR-L2 comprises an amino acid sequence selected from the group consisting of SEQ ID NOs: 103-119, or a variant thereof comprising up to 5 (e.g., 1, 2, 3, 4, or 5) amino acid substitutions. In some embodiments, the CDR-L3 comprises an amino acid sequence selected from the group consisting of SEQ ID NOs: 120-136, or a variant thereof comprising up to 5 (e.g., 1, 2, 3, 4, or 5) amino acid substitutions.

In some embodiments, the antibody can comprise a CDR-H1 having an amino acid sequence selected from the group consisting of SEQ ID NOs: 35-51, or a variant thereof comprising up to 5 (e.g., 1, 2, 3, 4, or 5) amino acid substitutions; a CDR-H2 having an amino acid sequence selected from the group consisting of SEQ ID NOs: 52-68, or a variant thereof comprising up to 5 (e.g., 1, 2, 3, 4, or 5) amino acid substitutions; a CDR-H3 having an amino acid sequence selected from the group consisting of SEQ ID NOs: 69-85, or a variant thereof comprising up to 5 (e.g., 1, 2, 3, 4, or 5) amino acid substitutions; a CDR-L1 having an amino acid sequence selected from the group consisting of SEQ ID NOs: 86-102, or a variant thereof comprising up to 5 (e.g., 1, 2, 3, 4, or 5) amino acid substitutions; a CDR-L2 having an amino acid sequence selected from the group consisting of SEQ ID NOs: 103-119, or a variant thereof comprising up to 5 (e.g., 1, 2, 3, 4, or 5) amino acid substitutions; and/or a CDR-L3 having an amino acid sequence selected from the group consisting of SEQ ID NOs: 120-136, or a variant thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH and a VL, wherein 1) the VH comprises a CDR-H1, a CDR-H2, and a CDR-H3, wherein the CDR-H1 comprises an amino acid sequence selected from the group consisting of SEQ ID NOs: 35-51, or a variant thereof comprising up to 5 (e.g., 1, 2, 3, 4, or 5) amino acid substitutions; the CDR-H2 comprises an amino acid sequence selected from the group consisting of SEQ ID NOs: 52-68, or a variant thereof comprising up to 5 (e.g., 1, 2, 3, 4, or 5) amino acid substitutions; and/or the CDR-H3 comprises an amino acid sequence selected from the group consisting of SEQ ID NOs: 69-85, or a variant thereof comprising up to 5 (e.g., 1, 2, 3, 4, or 5) amino acid substitutions; and/or 2) the VL comprises a CDR-L1, a CDR-L2, and a CDR-L3, the CDR-L1 comprises an amino acid sequence selected from the group consisting of SEQ ID NOs: 86-102, or a variant thereof comprising up to 5 (e.g., 1, 2, 3, 4, or 5) amino acid substitutions; the CDR-L2 comprises an amino acid sequence selected from the group consisting of SEQ ID NOs: 103-119, or a variant thereof comprising up to 5 (e.g., 1, 2, 3, 4, or 5) amino acid substitutions; and/or the CDR-L3 comprises an amino acid sequence selected from the group consisting of SEQ ID NOs: 120-136, or a variant thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH and a VL, wherein the VH comprises one or more amino acid sequences selected from the group consisting of SEQ ID NOs: 35-85, or a variant thereof comprising up to 5 (e.g., 1, 2, 3, 4, or 5) amino acid substitutions; and/or the VL comprises one or more amino acid selected from the group consisting of SEQ ID NOs: 86-136, or a variant thereof comprising up to 5 (e.g., 1, 2, 3, 4, or 5) amino acid substitutions. In certain embodiments, the isolated antibody, antigen-binding fragment, or masked antibody (e.g., activatable antibody) comprises a VH and a VL, wherein 1) the VH comprises one or more amino acid sequences selected from the group consisting of SEQ ID NOs: 35-51, or a variant thereof comprising up to 5 (e.g., 1, 2, 3, 4, or 5) amino acid substitutions; one or more amino acid sequences selected from the group consisting of SEQ ID NOs: 52-68, or a variant thereof comprising up to 5 (e.g., 1, 2, 3, 4, or 5) amino acid substitutions; and/or one or more amino acid sequences selected from the group consisting of SEQ ID NOs: 69-85, or a variant thereof comprising up to 5 (e.g., 1, 2, 3, 4, or 5) amino acid substitutions; and/or 2) the VL comprises one or more amino acid sequences selected from the group consisting of SEQ ID NOs: 86-102, or a variant thereof comprising up to 5 (e.g., 1, 2, 3, 4, or 5) amino acid substitutions; one or more amino acid sequences selected from the group consisting of SEQ ID NOs: 103-119, or a variant thereof comprising up to 5 (e.g., 1, 2, 3, 4, or 5) amino acid substitutions; and/or one or more amino acid sequences selected from the group consisting of SEQ ID NOs: 120-136, or a variant thereof comprising up to 5 (e.g., 1, 2, 3, 4, or 5) amino acid substitutions. In certain embodiments, the isolated antibody, antigen-binding fragment, or masked antibody (e.g., activatable antibody) comprises a VH and a VL, wherein 1) the VH comprises one or more amino acid sequences selected from the group consisting of SEQ ID NOs: 35-51, one or more amino acid sequences selected from the group consisting of SEQ ID NOs: 52-68, and one or more amino acid sequence selected from the group consisting of SEQ ID NOs: 69-85; and 2) the VL comprises one or more amino acid sequences selected from the group consisting of SEQ ID NOs: 86-102, one or more amino acid sequences selected from the group consisting of SEQ ID NOs: 103-119, and one or more amino acid sequences selected from the group consisting of SEQ ID NOs: 120-136. In some embodiments, the isolated antibody, antigen-binding fragment, or masked antibody (e.g., activatable antibody) comprises a VH and a VL, wherein 1) the VH comprises one or more (e.g., one, two, three, four, five, or six) CDRs of an illustrative antibody as shown in Table 3A or variants thereof comprising up to 5 (e.g., 1, 2, 3, 4, or 5) amino acid substitutions; and/or 2) the VL comprises one or one or more (e.g., one, two, three, four, five, or six) CDRs of an illustrative antibodies as shown in Table 3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising an amino acid sequence selected from the group consisting of SEQ ID NOs: 1, 3, 5, 7, 9, 11, 13, 15, 17, 19, 21, 23, 25, 27, 29, 31, and 33, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises a VL comprising an amino acid sequence selected from the group consisting of SEQ ID NOs: 2, 4, 6, 8, 10, 12, 14, 16, 18, 20, 22, 24, 26, 28, 30, 32, and 34, or a variant thereof comprising up to 5 (e.g., 1, 2, 3, 4, or 5) amino acid substitutions;\. In certain embodiments, comprises a VH comprising an amino acid sequence selected from the group consisting of SEQ ID NOs: 1, 3, 5, 7, 9, 11, 13, 15, 17, 19, 21, 23, 25, 27, 29, 31, and 33, or a variant thereof comprising up to 5 (e.g., 1, 2, 3, 4, or 5) amino acid substitutions, and a VL comprising an amino acid sequence selected from the group consisting of SEQ ID NOs: 2, 4, 6, 8, 10, 12, 14, 16, 18, 20, 22, 24, 26, 28, 30, 32, and 34, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises a VH and/or a VL of an illustrative antibody as shown in Table 4A, or one or more amino acid sequences having at least 80% (e.g., at least 85%, 90%, 95%, 98%, or 99%; or 100%) sequence identity with a VH or a VL of an illustrative antibody as shown in Table 4A. The isolated antibody, antigen-binding fragment, or masked antibody (e.g., activatable antibody) can comprise any combination of any VH and any VL described herein.

In certain embodiments, the isolated antibody, antigen-binding fragment, or masked antibody (e.g., activatable antibody) comprises a light chain and/or heavy chain (e.g., those of IgG such as IgG1 or lgG4). In some embodiments, the heavy chain comprises one or more amino acid sequences selected from the group consisting of SEQ ID NOs: 35-85, or a variant thereof comprising up to 5 (e.g., 1, 2, 3, 4, or 5) amino acid substitutions; and/or the light chain comprises one or more amino acid sequences selected from the group consisting of SEQ ID NOs: 86-136, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises a heavy chain and a light chain, wherein 1) the heavy chain comprises one or more amino acid selected from the group consisting of SEQ ID NOs: 35-51, or a variant thereof comprising up to 5 (e.g., 1, 2, 3, 4, or 5) amino acid substitutions; one or more amino acid sequences selected from the group consisting of SEQ ID NOs: 52-68, or a variant thereof comprising up to 5 (e.g., 1, 2, 3, 4, or 5) amino acid substitutions; and/or one or more amino acid sequences selected from the group consisting of SEQ ID NOs: 69-85, or a variant thereof comprising up to 5 (e.g., 1, 2, 3, 4, or 5) amino acid substitutions; and/or 2) the light chain comprises one or more amino acid selected from the group consisting of SEQ ID NOs: 86-102, or a variant thereof comprising up to 5 (e.g., 1, 2, 3, 4, or 5) amino acid substitutions; one or more amino acid sequences selected from the group consisting of SEQ ID NOs: 103-119, or a variant thereof comprising up to 5 (e.g., 1, 2, 3, 4, or 5) amino acid substitutions; and/or one or more amino acid sequences selected from the group consisting of SEQ ID NOs: 120-136, or a variant thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising an CDR-H1 comprising the sequence NYAIH (SEQ ID NO: 48), an CDR-H2 comprising the sequence AISGSGSSTYYADSVKG (SEQ ID NO: 65), and an CDR-H3 comprising the sequence RGSYGFGAFDY (SEQ ID NO: 82), or a variant thereof comprising up to 5 (e.g., 1, 2, 3, 4, or 5) amino acid substitutions of the CDR-H1 sequence, the CDR-H2 sequence, and/or the CDR-H3 sequence; and/or a VL comprising an CDR-L1 comprising the sequence RASQTIGRYLN (SEQ ID NO: 99), an CDR-L2 comprising the sequence DASNRAT (SEQ ID NO: 116), and an CDR-L3 comprising the sequence QQRYPWPYT (SEQ ID NO: 133), or a variant thereof comprising up to 5 (e.g., 1, 2, 3, 4, or 5) amino acid substitutions of the CDR-H1 sequence, the CDR-H2 sequence, and/or the CDR-H3 sequence.

In some embodiments, the isolated antibody, antigen-binding fragment, or masked antibody (e.g., activatable antibody) comprises a VH and a VL, wherein a) the VH comprises a CDR-H1 comprising the amino acid sequence of SEQ ID NO: 48, or a variant thereof comprising up to 5 (e.g., 1, 2, 3, 4, or 5) amino acid substitutions; a CDR-H2 comprising the amino acid sequence of SEQ ID NO: 65, or a variant thereof comprising up to 5 (e.g., 1, 2, 3, 4, or 5) amino acid substitutions; and/or a CDR-H3 comprising the amino acid sequence of SEQ ID NO: 82, or a variant thereof comprising up to 5 (e.g., 1, 2, 3, 4, or 5) amino acid substitutions; and/or b) the VL comprises a CDR-L1 comprising the amino acid sequence of SEQ ID NO: 99, or a variant thereof comprising up to 5 (e.g., 1, 2, 3, 4, or 5) amino acid substitutions; a CDR-L2 comprising the amino acid sequence of SEQ ID NO: 116, or a variant thereof comprising up to 5 (e.g., 1, 2, 3, 4, or 5) amino acid substitutions; and/or a CDR-L3 comprising the amino acid sequence of SEQ ID NO: 133, or a variant thereof comprising up to 5 (e.g., 1, 2, 3, 4, or 5) amino acid substitutions. In certain embodiments, a) the VH comprises a CDR-H1 comprising the amino acid sequence of SEQ ID NO: 48, a CDR-H2 comprising the amino acid sequence of SEQ ID NO: 65, and a CDR-H3 comprising the amino acid sequence of SEQ ID NO: 82; and b) the VL comprises a CDR-L1 comprising the amino acid sequence of SEQ ID NO: 99, a CDR-L2 comprising the amino acid sequence of SEQ ID NO: 116, and a CDR-L3 comprising the amino acid sequence of SEQ ID NO: 133. In some embodiments, the isolated antibody, antigen-binding fragment, or masked antibody (e.g., activatable antibody) comprises a VH and a VL wherein a) the VH comprises the amino acid sequence of SEQ ID NO: 48, or a variant thereof comprising up to 5 (e.g., 1, 2, 3, 4, or 5) amino acid substitutions; the amino acid sequence of SEQ ID NO: 65, or a variant thereof comprising up to 5 (e.g., 1, 2, 3, 4, or 5) amino acid substitutions; and/or the amino acid sequence of SEQ ID NO: 82, or a variant thereof comprising up to 5 (e.g., 1, 2, 3, 4, or 5) amino acid substitutions; and/or b) the VL comprises the amino acid sequence of SEQ ID NO: 99, or a variant thereof comprising up to 5 (e.g., 1, 2, 3, 4, or 5) amino acid substitutions; the amino acid sequence of SEQ ID NO: 116, or a variant thereof comprising up to 5 (e.g., 1, 2, 3, 4, or 5) amino acid substitutions; and/or the amino acid sequence of SEQ ID NO: 133, or a variant thereof comprising up to 5 (e.g., 1, 2, 3, 4, or 5) amino acid substitutions. In certain embodiments, a) the VH comprises the amino acid sequence of SEQ ID NO: 48, the amino acid sequence of SEQ ID NO: 65, and the amino acid sequence of SEQ ID NO: 82; and b) the VL comprises the amino acid sequence of SEQ ID NO: 99, the amino acid sequence of SEQ ID NO: 116, and the amino acid sequence of SEQ ID NO: 133. In some embodiments, the isolated antibody, antigen-binding fragment, or masked antibody (e.g., activatable antibody) comprises one, two, three, four, five, or six CDRs of illustrative antibody TY21446 as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising a CDR-H1, a CDR-H2 and a CDR-H3 of a VH comprising the amino acid sequence of SEQ ID NO: 27, or an amino acid sequence having at least 80% (e.g., at least 85%, 90%, 95%, 98%, or 99%; or 100%) sequence identity with SEQ ID NO: 27; and/or a VL comprising a CDR-L1, a CDR-L2 and a CDR-L3 of a VL comprising the amino acid sequence of SEQ ID NO: 28, or an amino acid sequence having at least 80% (e.g., at least 85%, 90%, 95%, 98%, or 99%; or 100%) sequence identity with SEQ ID NO: 28. In some embodiments the VH comprises the amino acid sequence of SEQ ID NO: 27, or an amino acid sequence having at least 80% (e.g., at least 85%, 90%, 95%, 98%, or 99%; or 100%) sequence identity with SEQ ID NO: 27, and/or the VL comprises the amino acid sequence of SEQ ID NO: 28, or an amino acid sequence having at least 80% (e.g., at least 85%, 90%, 95%, 98%, or 99%; or 100%) sequence identity with SEQ ID NO: 27. In some embodiments, the isolated antibody, antigen-binding fragment, or masked antibody (e.g., activatable antibody) comprises the VH and/or the VL of illustrative antibody TY21446 as shown in Table 4A, or one or more amino acid sequences having at least 80% (e.g., at least 85%, 90%, 95%, 98%, or 99%; or 100%) sequence identity with a VH or a VL of TY21446 as shown in Table 4A.

In some embodiments, the isolated antibody, antigen-binding fragment, or masked antibody (e.g., activatable antibody) comprises a VH and a VL, wherein a) the VH comprises a CDR-H1 comprising the amino acid sequence of SEQ ID NO: 49, or a variant thereof comprising up to 5 (e.g., 1, 2, 3, 4, or 5) amino acid substitutions; a CDR-H2 comprising the amino acid sequence of SEQ ID NO: 66, or a variant thereof comprising up to 5 (e.g., 1, 2, 3, 4, or 5) amino acid substitutions; and/or a CDR-H3 comprising the amino acid sequence of SEQ ID NO: 83, or a variant thereof comprising up to 5 (e.g., 1, 2, 3, 4, or 5) amino acid substitutions; and/or b) the VL comprises a CDR-L1 comprising the amino acid sequence of SEQ ID NO: 100, or a variant thereof comprising up to 5 (e.g., 1, 2, 3, 4, or 5) amino acid substitutions; a CDR-L2 comprising the amino acid sequence of SEQ ID NO: 117, or a variant thereof comprising up to 5 (e.g., 1, 2, 3, 4, or 5) amino acid substitutions; and/or a CDR-L3 comprising the amino acid sequence of SEQ ID NO: 134, or a variant thereof comprising up to 5 (e.g., 1, 2, 3, 4, or 5) amino acid substitutions. In some embodiments, a) the VH comprises the amino acid sequence of SEQ ID NO: 49, or a variant thereof comprising up to 5 (e.g., 1, 2, 3, 4, or 5) amino acid substitutions; the amino acid sequence of SEQ ID NO: 66, or a variant thereof comprising up to 5 (e.g., 1, 2, 3, 4, or 5) amino acid substitutions; and/or the amino acid sequence of SEQ ID NO: 83, or a variant thereof comprising up to 5 (e.g., 1, 2, 3, 4, or 5) amino acid substitutions; and/or b) the VL comprises the amino acid sequence of SEQ ID NO: 100, or a variant thereof comprising up to 5 (e.g., 1, 2, 3, 4, or 5) amino acid substitutions; the amino acid sequence of SEQ ID NO: 117, or a amino acid sequence of SEQ ID NO: 134, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises one, two, three, four, five, or six CDRs of illustrative antibody TY21447 as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising a CDR-H1, a CDR-H2 and a CDR-H3 of a VH comprising the amino acid sequence of SEQ ID NO: 29, or an amino acid sequence having at least 80% (e.g., at least 85%, 90%, 95%, 98%, or 99%; or 100%) sequence identity with SEQ ID NO: 29; and/or a VL comprising a CDR-L1, a CDR-L2 and a CDR-L3 of a VL comprising the amino acid sequence of SEQ ID NO: 30, or an amino acid sequence having at least 80% (e.g., at least 85%, 90%, 95%, 98%, or 99%; or 100%) sequence identity with SEQ ID NO: 30. In certain embodiments, the VH comprises the amino acid sequence of SEQ ID NO: 29, or an amino acid sequence having at least 80% (e.g., at least 85%, 90%, 95%, 98%, or 99%; or 100%) sequence identity with SEQ ID NO: 29; and/or the VL comprises the amino acid sequence of SEQ ID NO: 30, or an amino acid sequence having at least 80% (e.g., at least 85%, 90%, 95%, 98%, or 99%; or 100%) sequence identity with SEQ ID NO: 30. In some embodiments, the isolated antibody, antigen-binding fragment, or masked antibody (e.g., activatable antibody) comprises the VH and/or the VL of illustrative antibody TY21447 as shown in Table 4A, or one or more amino acid sequences having at least 80% (e.g., at least 85%, 90%, 95%, 98%, or 99%; or 100%) sequence identity with a VH or a VL of TY21447 as shown in Table 4A.

In some embodiments, the isolated antibody, antigen-binding fragment, or masked antibody (e.g., activatable antibody) comprises a VH and a VL, wherein a) the VH comprises a CDR-H1 comprising the amino acid sequence of SEQ ID NO: 50, or a variant thereof comprising up to 5 (e.g., 1, 2, 3, 4, or 5) amino acid substitutions; a CDR-H2 comprising the amino acid sequence of SEQ ID NO: 67, or a variant thereof comprising up to 5 (e.g., 1, 2, 3, 4, or 5) amino acid substitutions; and/or a CDR-H3 comprising the amino acid sequence of SEQ ID NO: 84, or a variant thereof comprising up to 5 (e.g., 1, 2, 3, 4, or 5) amino acid substitutions; and/or b) the VL comprises a CDR-L1 comprising the amino acid sequence of SEQ ID NO: 101, or a variant thereof comprising up to 5 (e.g., 1, 2, 3, 4, or 5) amino acid substitutions; a CDR-L2 comprising the amino acid sequence of SEQ ID NO: 118, or a variant thereof comprising up to 5 (e.g., 1, 2, 3, 4, or 5) amino acid substitutions; and/or a CDR-L3 comprising the amino acid sequence of SEQ ID NO: 135, or a variant thereof comprising up to 5 (e.g., 1, 2, 3, 4, or 5) amino acid substitutions. In some embodiments, a) the VH comprises the amino acid sequence of SEQ ID NO: 50, or a variant thereof comprising up to 5 (e.g., 1, 2, 3, 4, or 5) amino acid substitutions; the amino acid sequence of SEQ ID NO: 67, or a variant thereof comprising up to 5 (e.g., 1, 2, 3, 4, or 5) amino acid substitutions; and/or the amino acid sequence of SEQ ID NO: 84, or a variant thereof comprising up to 5 (e.g., 1, 2, 3, 4, or 5) amino acid substitutions; and/or b) the VL comprises the amino acid sequence of SEQ ID NO: 101, or a variant thereof comprising up to 5 (e.g., 1, 2, 3, 4, or 5) amino acid substitutions; the amino acid sequence of SEQ ID NO: 118, or a variant thereof comprising up to 5 (e.g., 1, 2, 3, 4, or 5) amino acid substitutions; and/or the amino acid sequence of SEQ ID NO: 135, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises one, two, three, four, five, or six CDRs of illustrative antibody TY21449 as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising a CDR-H1, a CDR-H2 and a CDR-H3 of a VH comprising the amino acid sequence of SEQ ID NO: 31, or an amino acid sequence having at least 80% (e.g., at least 85%, 90%, 95%, 98%, or 99%; or 100%) sequence identity with SEQ ID NO: 31; and/or a VL comprising a CDR-L1, a CDR-L2 and a CDR-L3 of a VL comprising the amino acid sequence of SEQ ID NO: 32, or an amino acid sequence having at least 80% (e.g., at least 85%, 90%, 95%, 98%, or 99%; or 100%) sequence identity with SEQ ID NO: 32. In certain embodiments, the VH comprises the amino acid sequence of SEQ ID NO: 31, or an amino acid sequence having at least 80% (e.g., at least 85%, 90%, 95%, 98%, or 99%; or 100%) sequence identity with SEQ ID NO: 31; and/or the VL comprises the amino acid sequence of SEQ ID NO: 32, or an amino acid sequence having at least 80% (e.g., at least 85%, 90%, 95%, 98%, or 99%; or 100%) sequence identity with SEQ ID NO: 32. In some embodiments, the isolated antibody, antigen-binding fragment, illustrative antibody TY21449 as shown in Table 4A, or one or more amino acid sequences having at least 80% (e.g., at least 85%, 90%, 95%, 98%, or 99%; or 100%) sequence identity with a VH or a VL of TY21449 as shown in Table 4A.

In some embodiments, the isolated antibody, antigen-binding fragment, or masked antibody (e.g., activatable antibody) comprises a VH and a VL, wherein a) the VH comprises a CDR-H1 comprising the amino acid sequence of SEQ ID NO: 35, or a variant thereof comprising up to 5 (e.g., 1, 2, 3, 4, or 5) amino acid substitutions; a CDR-H2 comprising the amino acid sequence of SEQ ID NO: 52, or a variant thereof comprising up to 5 (e.g., 1, 2, 3, 4, or 5) amino acid substitutions; and/or a CDR-H3 comprising the amino acid sequence of SEQ ID NO: 69, or a variant thereof comprising up to 5 (e.g., 1, 2, 3, 4, or 5) amino acid substitutions; and/or b) the VL comprises a CDR-L1 comprising the amino acid sequence of SEQ ID NO: 86, or a variant thereof comprising up to 5 (e.g., 1, 2, 3, 4, or 5) amino acid substitutions; a CDR-L2 comprising the amino acid sequence of SEQ ID NO: 103, or a variant thereof comprising up to 5 (e.g., 1, 2, 3, 4, or 5) amino acid substitutions; and/or a CDR-L3 comprising the amino acid sequence of SEQ ID NO: 120, or a variant thereof comprising up to 5 (e.g., 1, 2, 3, 4, or 5) amino acid substitutions. In some embodiments, a) the VH comprises the amino acid sequence of SEQ ID NO: 35, or a variant thereof comprising up to 5 (e.g., 1, 2, 3, 4, or 5) amino acid substitutions; the amino acid sequence of SEQ ID NO: 52, or a variant thereof comprising up to 5 (e.g., 1, 2, 3, 4, or 5) amino acid substitutions; and/or the amino acid sequence of SEQ ID NO: 69, or a variant thereof comprising up to 5 (e.g., 1, 2, 3, 4, or 5) amino acid substitutions; and/or b) the VL comprises the amino acid sequence of SEQ ID NO: 86, or a variant thereof comprising up to 5 (e.g., 1, 2, 3, 4, or 5) amino acid substitutions; the amino acid sequence of SEQ ID NO: 103, or a variant thereof comprising up to 5 (e.g., 1, 2, 3, 4, or 5) amino acid substitutions; and/or the amino acid sequence of SEQ ID NO: 120, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises one, two, three, four, five, or six CDRs of illustrative antibody TY25029 as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising a CDR-H1, a CDR-H2 and a CDR-H3 of a VH comprising the amino acid sequence of SEQ ID NO: 1, or an amino acid sequence having at least 80% (e.g., at least 85%, 90%, 95%, 98%, or 99%; or 100%) sequence identity with SEQ ID NO: 1; and/or a VL comprising a CDR-L1, a CDR-L2 and a CDR-L3 of a VL comprising the amino acid sequence of SEQ ID NO: 2, or an amino acid sequence having at least 80% (e.g., at least 85%, 90%, 95%, 98%, or 99%; or 100%) sequence identity with SEQ ID NO: 2. In certain embodiments, the VH comprises the amino acid sequence of SEQ ID NO: 1, or an amino acid sequence having at least 80% (e.g., at least 85%, 90%, 95%, 98%, or 99%; or 100%) sequence identity with SEQ ID NO: 1; and/or the VL comprises the amino acid sequence of SEQ ID NO: 2, or an amino acid sequence having at least 80% (e.g., at least 85%, 90%, 95%, 98%, or 99%; or 100%) sequence identity with SEQ ID NO: 2. In some embodiments, the isolated antibody, antigen-binding fragment, or masked antibody (e.g., activatable antibody) comprises the VH and/or the VL of illustrative antibody TY25029 as shown in Table 4A, or one or more amino acid sequences having at least 80% (e.g., at least 85%, 90%, 95%, 98%, or 99%; or 100%) sequence identity with a VH or a VL of TY25029 as shown in Table 4A.

In some embodiments, the isolated antibody, antigen-binding fragment, or masked antibody (e.g., activatable antibody) comprises a VH and a VL, wherein a) the VH comprises a CDR-H1 comprising the amino acid sequence of SEQ ID NO: 36, or a variant thereof comprising up to 5 (e.g., 1, 2, 3, 4, or 5) amino acid substitutions; a CDR-H2 comprising the amino acid sequence of SEQ ID NO: 53, or a variant thereof comprising up to 5 (e.g., 1, 2, 3, 4, or 5) amino acid substitutions; and/or a CDR-H3 comprising the amino acid sequence of SEQ ID NO: 70, or a variant thereof comprising up to 5 (e.g., 1, 2, 3, 4, or 5) amino acid substitutions; and/or b) the VL comprises a CDR-L1 comprising the amino acid sequence of SEQ ID NO: 87, or a variant thereof comprising up to 5 (e.g., 1, 2, 3, 4, or 5) amino acid substitutions; a CDR-L2 comprising the amino acid sequence of SEQ ID NO: 104, or a variant thereof comprising up to 5 (e.g., 1, 2, 3, 4, or 5) amino acid substitutions; and/or a CDR-L3 comprising the amino acid sequence of SEQ ID NO: 121, or a variant thereof comprising up to 5 (e.g., 1, 2, 3, 4, or 5) amino acid substitutions. In some embodiments, a) the VH comprises the amino acid sequence of SEQ ID NO: 36, or a variant thereof comprising up to 5 (e.g., 1, 2, 3, 4, or 5) amino acid substitutions; the amino acid sequence of SEQ ID NO: 53, or a variant thereof comprising up to 5 (e.g., 1, 2, 3, 4, or 5) amino acid substitutions; and/or the amino acid sequence of SEQ ID NO: 70, or a variant thereof comprising up to 5 (e.g., 1, 2, 3, 4, or 5) amino acid substitutions; and/or b) the VL comprises the amino acid sequence of SEQ ID NO: 87, or a variant thereof comprising up to 5 (e.g., 1, 2, 3, 4, or 5) amino acid substitutions; the amino acid sequence of SEQ ID NO: 104, or a amino acid sequence of SEQ ID NO: 121, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises one, two, three, four, five, or six CDRs of illustrative antibody TY25030 as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising a CDR-H1, a CDR-H2 and a CDR-H3 of a VH comprising the amino acid sequence of SEQ ID NO: 3, or an amino acid sequence having at least 80% (e.g., at least 85%, 90%, 95%, 98%, or 99%; or 100%) sequence identity with SEQ ID NO: 3; and/or a VL comprising a CDR-L1, a CDR-L2 and a CDR-L3 of a VL comprising the amino acid sequence of SEQ ID NO: 4, or an amino acid sequence having at least 80% (e.g., at least 85%, 90%, 95%, 98%, or 99%; or 100%) sequence identity with SEQ ID NO: 4. In certain embodiments, the VH comprises the amino acid sequence of SEQ ID NO: 3, or an amino acid sequence having at least 80% (e.g., at least 85%, 90%, 95%, 98%, or 99%; or 100%) sequence identity with SEQ ID NO: 3; and/or the VL comprises the amino acid sequence of SEQ ID NO: 4, or an amino acid sequence having at least 80% (e.g., at least 85%, 90%, 95%, 98%, or 99%; or 100%) sequence identity with SEQ ID NO: 4. In some embodiments, the isolated antibody, antigen-binding fragment, or masked antibody (e.g., activatable antibody) comprises the VH and/or the VL of illustrative antibody TY25030 as shown in Table 4A, or one or more amino acid sequences having at least 80% (e.g., at least 85%, 90%, 95%, 98%, or 99%; or 100%) sequence identity with a VH or a VL of TY25030 as shown in Table 4A.

In some embodiments, the isolated antibody, antigen-binding fragment, or masked antibody (e.g., activatable antibody) comprises a VH and a VL, wherein a) the VH comprises a CDR-H1 comprising the amino acid sequence of SEQ ID NO: 37, or a variant thereof comprising up to 5 (e.g., 1, 2, 3, 4, or 5) amino acid substitutions; a CDR-H2 comprising the amino acid sequence of SEQ ID NO: 54, or a variant thereof comprising up to 5 (e.g., 1, 2, 3, 4, or 5) amino acid substitutions; and/or a CDR-H3 comprising the amino acid sequence of SEQ ID NO: 71, or a variant thereof comprising up to 5 (e.g., 1, 2, 3, 4, or 5) amino acid substitutions; and/or b) the VL comprises a CDR-L1 comprising the amino acid sequence of SEQ ID NO: 88, or a variant thereof comprising up to 5 (e.g., 1, 2, 3, 4, or 5) amino acid substitutions; a CDR-L2 comprising the amino acid sequence of SEQ ID NO: 105, or a variant thereof comprising up to 5 (e.g., 1, 2, 3, 4, or 5) amino acid substitutions; and/or a CDR-L3 comprising the amino acid sequence of SEQ ID NO: 122, or a variant thereof comprising up to 5 (e.g., 1, 2, 3, 4, or 5) amino acid substitutions. In some embodiments, a) the VH comprises the amino acid sequence of SEQ ID NO: 37, or a variant thereof comprising up to 5 (e.g., 1, 2, 3, 4, or 5) amino acid substitutions; the amino acid sequence of SEQ ID NO: 54, or a variant thereof comprising up to 5 (e.g., 1, 2, 3, 4, or 5) amino acid substitutions; and/or the amino acid sequence of SEQ ID NO: 71, or a variant thereof comprising up to 5 (e.g., 1, 2, 3, 4, or 5) amino acid substitutions; and/or b) the VL comprises the amino acid sequence of SEQ ID NO: 88, or a variant thereof comprising up to 5 (e.g., 1, 2, 3, 4, or 5) amino acid substitutions; the amino acid sequence of SEQ ID NO: 105, or a variant thereof comprising up to 5 (e.g., 1, 2, 3, 4, or 5) amino acid substitutions; and/or the amino acid sequence of SEQ ID NO: 122, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises one, two, three, four, five, or six CDRs of illustrative antibody TY25031 as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising a CDR-H1, a CDR-H2 and a CDR-H3 of a VH comprising the amino acid sequence of SEQ ID NO: 5, or an amino acid sequence having at least 80% (e.g., at least 85%, 90%, 95%, 98%, or 99%; or 100%) sequence identity with SEQ ID NO: 5; and/or a VL comprising a CDR-L1, a CDR-L2 and a CDR-L3 of a VL comprising the amino acid sequence of SEQ ID NO: 6, or an amino acid sequence having at least 80% (e.g., at least 85%, 90%, 95%, 98%, or 99%; or 100%) sequence identity with SEQ ID NO: 6. In certain embodiments, the VH comprises the amino acid sequence of SEQ ID NO: 5, or an amino acid sequence having at least 80% (e.g., at least 85%, 90%, 95%, 98%, or 99%; or 100%) sequence identity with SEQ ID NO: 5; and/or the VL comprises the amino acid sequence of SEQ ID NO: 6, or an amino acid sequence having at least 80% (e.g., at least 85%, 90%, 95%, 98%, or 99%; or 100%) sequence identity with SEQ ID NO: 6. In some embodiments, the isolated antibody, antigen-binding fragment, or masked antibody (e.g., activatable antibody) comprises the VH and/or the VL of illustrative antibody TY25031 as shown in Table 4A, or one or more amino acid sequences having at least 80% (e.g., at least 85%, 90%, 95%, 98%, or 99%; or 100%) sequence identity with a VH or a VL of TY25031 as shown in Table 4A.

In some embodiments, the isolated antibody, antigen-binding fragment, or masked antibody (e.g., activatable antibody) comprises a VH and a VL, wherein a) the VH comprises a CDR-H1 comprising the amino acid sequence of SEQ ID NO: 38, or a variant thereof comprising up to 5 (e.g., 1, 2, 3, 4, or 5) amino acid substitutions; a CDR-H2 comprising the amino acid sequence of SEQ ID NO: 55, or a variant thereof comprising up to 5 (e.g., 1, 2, 3, 4, or 5) amino acid substitutions; and/or a CDR-H3 comprising the amino acid sequence of SEQ ID NO: 72, or a variant thereof comprising up to 5 (e.g., 1, 2, 3, 4, or 5) amino acid substitutions; and/or b) the VL comprises a CDR-L1 comprising the amino acid sequence of SEQ ID NO: 89, or a variant thereof comprising up to 5 (e.g., 1, 2, 3, 4, or 5) amino acid substitutions; a CDR-L2 comprising the amino acid sequence of SEQ ID NO: 106, or a variant thereof comprising up to 5 (e.g., 1, 2, 3, 4, or 5) amino acid substitutions; and/or a CDR-L3 comprising the amino acid sequence of SEQ ID NO: 123, or a variant thereof comprising up to 5 (e.g., 1, 2, 3, 4, or 5) amino acid substitutions. In some embodiments, a) the VH comprises the amino acid sequence of SEQ ID NO: 38, or a variant thereof comprising up to 5 (e.g., 1, 2, 3, 4, or 5) amino acid substitutions; the amino acid sequence of SEQ ID NO: 55, or a variant thereof comprising up to 5 (e.g., 1, 2, 3, 4, or 5) amino acid substitutions; and/or the amino acid sequence of SEQ ID NO: 72, or a variant thereof comprising up to 5 (e.g., 1, 2, 3, 4, or 5) amino acid substitutions; and/or b) the VL comprises the amino acid sequence of SEQ ID NO: 89, or a variant thereof comprising up to 5 (e.g., 1, 2, 3, 4, or 5) amino acid substitutions; the amino acid sequence of SEQ ID NO: 106, or a variant thereof comprising up to 5 (e.g., 1, 2, 3, 4, or 5) amino acid substitutions; and/or the amino acid sequence of SEQ ID NO: 123, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises one, two, three, four, five, or six CDRs of illustrative antibody TY25032 as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising a CDR-H1, a CDR-H2 and a CDR-H3 of a VH comprising the amino acid sequence of SEQ ID NO: 7, or an amino acid sequence having at least 80% (e.g., at least 85%, 90%, 95%, 98%, or 99%; or 100%) sequence identity with SEQ ID NO: 7; and/or a VL comprising a CDR-L1, a CDR-L2 and a CDR-L3 of a VL comprising the amino acid sequence of SEQ ID NO: 8, or an amino acid sequence having at least 80% (e.g., at least 85%, 90%, 95%, 98%, or 99%; or 100%) sequence identity with SEQ ID NO: 8. In certain embodiments, the VH comprises the amino acid sequence of SEQ ID NO: 7, or an amino acid sequence having at least 80% (e.g., at least 85%, 90%, 95%, 98%, or 99%; or 100%) sequence identity with SEQ ID NO: 7; and/or the VL comprises the amino acid sequence of SEQ ID NO: 8, or an amino acid sequence having at least 80% (e.g., at least 85%, 90%, 95%, 98%, or 99%; or 100%) sequence identity with SEQ ID NO: 8. In some embodiments, the isolated antibody, antigen-binding fragment, or masked antibody (e.g., activatable antibody) comprises the VH and/or the VL of illustrative antibody TY25032 as shown in Table 4A, or one or more amino acid sequences having at least 80% (e.g., at least 85%, 90%, 95%, 98%, or 99%; or 100%) sequence identity with a VH or a VL of TY25032 as shown in Table 4A.

In some embodiments, the isolated antibody, antigen-binding fragment, or masked antibody (e.g., activatable antibody) comprises a VH and a VL, wherein a) the VH comprises a CDR-H1 comprising the amino acid sequence of SEQ ID NO: 39, or a variant thereof comprising up to 5 (e.g., 1, 2, 3, 4, or 5) amino acid substitutions; a CDR-H2 comprising the amino acid sequence of SEQ ID NO: 56, or a variant thereof comprising up to 5 (e.g., 1, 2, 3, 4, or 5) amino acid substitutions; and/or a CDR-H3 comprising the amino acid sequence of SEQ ID NO: 73, or a variant thereof comprising up to 5 (e.g., 1, 2, 3, 4, or 5) amino acid substitutions; and/or b) the VL comprises a CDR-L1 comprising the amino acid sequence of SEQ ID NO: 90, or a variant thereof comprising up to 5 (e.g., 1, 2, 3, 4, or 5) amino acid substitutions; a CDR-L2 comprising the amino acid sequence of SEQ ID NO: 107, or a variant thereof comprising up to 5 (e.g., 1, 2, 3, 4, or 5) amino acid substitutions; and/or a CDR-L3 comprising the amino acid sequence of SEQ ID NO: 124, or a variant thereof comprising up to 5 (e.g., 1, 2, 3, 4, or 5) amino acid substitutions. In some embodiments, a) the VH comprises the amino acid sequence of SEQ ID NO: 39, or a variant thereof comprising up to 5 (e.g., 1, 2, 3, 4, or 5) amino acid substitutions; the amino acid sequence of SEQ ID NO: 56, or a variant thereof comprising up to 5 (e.g., 1, 2, 3, 4, or 5) amino acid substitutions; and/or the amino acid sequence of SEQ ID NO: 73, or a variant thereof comprising up to 5 (e.g., 1, 2, 3, 4, or 5) amino acid substitutions; and/or b) the VL comprises the amino acid sequence of SEQ ID NO: 90, or a variant thereof comprising up to 5 (e.g., 1, 2, 3, 4, or 5) amino acid substitutions; the amino acid sequence of SEQ ID NO: 107, or a amino acid sequence of SEQ ID NO: 124, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises one, two, three, four, five, or six CDRs of illustrative antibody TY25033 as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising a CDR-H1, a CDR-H2 and a CDR-H3 of a VH comprising the amino acid sequence of SEQ ID NO: 9, or an amino acid sequence having at least 80% (e.g., at least 85%, 90%, 95%, 98%, or 99%; or 100%) sequence identity with SEQ ID NO: 9; and/or a VL comprising a CDR-L1, a CDR-L2 and a CDR-L3 of a VL comprising the amino acid sequence of SEQ ID NO: 10, or an amino acid sequence having at least 80% (e.g., at least 85%, 90%, 95%, 98%, or 99%; or 100%) sequence identity with SEQ ID NO: 10. In certain embodiments, the VH comprises the amino acid sequence of SEQ ID NO: 9, or an amino acid sequence having at least 80% (e.g., at least 85%, 90%, 95%, 98%, or 99%; or 100%) sequence identity with SEQ ID NO: 9; and/or the VL comprises the amino acid sequence of SEQ ID NO: 10, or an amino acid sequence having at least 80% (e.g., at least 85%, 90%, 95%, 98%, or 99%; or 100%) sequence identity with SEQ ID NO: 10. In some embodiments, the isolated antibody, antigen-binding fragment, or masked antibody (e.g., activatable antibody) comprises the VH and/or the VL of illustrative antibody TY25033 as shown in Table 4A, or one or more amino acid sequences having at least 80% (e.g., at least 85%, 90%, 95%, 98%, or 99%; or 100%) sequence identity with a VH or a VL of TY25033 as shown in Table 4A.

In some embodiments, the isolated antibody, antigen-binding fragment, or masked antibody (e.g., activatable antibody) comprises a VH and a VL, wherein a) the VH comprises a CDR-H1 comprising the amino acid sequence of SEQ ID NO: 40, or a variant thereof comprising up to 5 (e.g., 1, 2, 3, 4, or 5) amino acid substitutions; a CDR-H2 comprising the amino acid sequence of SEQ ID NO: 57, or a variant thereof comprising up to 5 (e.g., 1, 2, 3, 4, or 5) amino acid substitutions; and/or a CDR-H3 comprising the amino acid sequence of SEQ ID NO: 74, or a variant thereof comprising up to 5 (e.g., 1, 2, 3, 4, or 5) amino acid substitutions; and/or b) the VL comprises a CDR-L1 comprising the amino acid sequence of SEQ ID NO: 91, or a variant thereof comprising up to 5 (e.g., 1, 2, 3, 4, or 5) amino acid substitutions; a CDR-L2 comprising the amino acid sequence of SEQ ID NO: 108, or a variant thereof comprising up to 5 (e.g., 1, 2, 3, 4, or 5) amino acid substitutions; and/or a CDR-L3 comprising the amino acid sequence of SEQ ID NO: 125, or a variant thereof comprising up to 5 (e.g., 1, 2, 3, 4, or 5) amino acid substitutions. In some embodiments, a) the VH comprises the amino acid sequence of SEQ ID NO: 40, or a variant thereof comprising up to 5 (e.g., 1, 2, 3, 4, or 5) amino acid substitutions; the amino acid sequence of SEQ ID NO: 57, or a variant thereof comprising up to 5 (e.g., 1, 2, 3, 4, or 5) amino acid substitutions; and/or the amino acid sequence of SEQ ID NO: 74, or a variant thereof comprising up to 5 (e.g., 1, 2, 3, 4, or 5) amino acid substitutions; and/or b) the VL comprises the amino acid sequence of SEQ ID NO: 91, or a variant thereof comprising up to 5 (e.g., 1, 2, 3, 4, or 5) amino acid substitutions; the amino acid sequence of SEQ ID NO: 108, or a variant thereof comprising up to 5 (e.g., 1, 2, 3, 4, or 5) amino acid substitutions; and/or the amino acid sequence of SEQ ID NO: 125, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises one, two, three, four, five, or six CDRs of illustrative antibody TY25034 as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising a CDR-H1, a CDR-H2 and a CDR-H3 of a VH comprising the amino acid sequence of SEQ ID NO: 11, or an amino acid sequence having at least 80% (e.g., at least 85%, 90%, 95%, 98%, or 99%; or 100%) sequence identity with SEQ ID NO: 11; and/or a VL comprising a CDR-L1, a CDR-L2 and a CDR-L3 of a VL comprising the amino acid sequence of SEQ ID NO: 12, or an amino acid sequence having at least 80% (e.g., at least 85%, 90%, 95%, 98%, or 99%; or 100%) sequence identity with SEQ ID NO: 12. In certain embodiments, the VH comprises the amino acid sequence of SEQ ID NO: 11, or an amino acid sequence having at least 80% (e.g., at least 85%, 90%, 95%, 98%, or 99%; or 100%) sequence identity with SEQ ID NO: 11; and/or the VL comprises the amino acid sequence of SEQ ID NO: 12, or an amino acid sequence having at least 80% (e.g., at least 85%, 90%, 95%, 98%, or 99%; or 100%) sequence identity with SEQ ID NO: 12. In some embodiments, the isolated antibody, antigen-binding fragment, illustrative antibody TY25034 as shown in Table 4A, or one or more amino acid sequences having at least 80% (e.g., at least 85%, 90%, 95%, 98%, or 99%; or 100%) sequence identity with a VH or a VL of TY25034 as shown in Table 4A.

In some embodiments, the isolated antibody, antigen-binding fragment, or masked antibody (e.g., activatable antibody) comprises a VH and a VL, wherein a) the VH comprises a CDR-H1 comprising the amino acid sequence of SEQ ID NO: 41, or a variant thereof comprising up to 5 (e.g., 1, 2, 3, 4, or 5) amino acid substitutions; a CDR-H2 comprising the amino acid sequence of SEQ ID NO: 58, or a variant thereof comprising up to 5 (e.g., 1, 2, 3, 4, or 5) amino acid substitutions; and/or a CDR-H3 comprising the amino acid sequence of SEQ ID NO: 75, or a variant thereof comprising up to 5 (e.g., 1, 2, 3, 4, or 5) amino acid substitutions; and/or b) the VL comprises a CDR-L1 comprising the amino acid sequence of SEQ ID NO: 92, or a variant thereof comprising up to 5 (e.g., 1, 2, 3, 4, or 5) amino acid substitutions; a CDR-L2 comprising the amino acid sequence of SEQ ID NO: 109, or a variant thereof comprising up to 5 (e.g., 1, 2, 3, 4, or 5) amino acid substitutions; and/or a CDR-L3 comprising the amino acid sequence of SEQ ID NO: 126, or a variant thereof comprising up to 5 (e.g., 1, 2, 3, 4, or 5) amino acid substitutions. In some embodiments, a) the VH comprises the amino acid sequence of SEQ ID NO: 41, or a variant thereof comprising up to 5 (e.g., 1, 2, 3, 4, or 5) amino acid substitutions; the amino acid sequence of SEQ ID NO: 58, or a variant thereof comprising up to 5 (e.g., 1, 2, 3, 4, or 5) amino acid substitutions; and/or the amino acid sequence of SEQ ID NO: 75, or a variant thereof comprising up to 5 (e.g., 1, 2, 3, 4, or 5) amino acid substitutions; and/or b) the VL comprises the amino acid sequence of SEQ ID NO: 92, or a variant thereof comprising up to 5 (e.g., 1, 2, 3, 4, or 5) amino acid substitutions; the amino acid sequence of SEQ ID NO: 109, or a variant thereof comprising up to 5 (e.g., 1, 2, 3, 4, or 5) amino acid substitutions; and/or the amino acid sequence of SEQ ID NO: 126, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises one, two, three, four, five, or six CDRs of illustrative antibody TY25035 as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising a CDR-H1, a CDR-H2 and a CDR-H3 of a VH comprising the amino acid sequence of SEQ ID NO: 13, or an amino acid sequence having at least 80% (e.g., at least 85%, 90%, 95%, 98%, or 99%; or 100%) sequence identity with SEQ ID NO: 13; and/or a VL comprising a CDR-L1, a CDR-L2 and a CDR-L3 of a VL comprising the amino acid sequence of SEQ ID NO: 14, or an amino acid sequence having at least 80% (e.g., at least 85%, 90%, 95%, 98%, or 99%; or 100%) sequence identity with SEQ ID NO: 14. In certain embodiments, the VH comprises the amino acid sequence of SEQ ID NO: 13, or an amino acid sequence having at least 80% (e.g., at least 85%, 90%, 95%, 98%, or 99%; or 100%) sequence identity with SEQ ID NO: 13; and/or the VL comprises the amino acid sequence of SEQ ID NO: 14, or an amino acid sequence having at least 80% (e.g., at least 85%, 90%, 95%, 98%, or 99%; or 100%) sequence identity with SEQ ID NO: 14. In some embodiments, the isolated antibody, antigen-binding fragment, or masked antibody (e.g., activatable antibody) comprises the VH and/or the VL of illustrative antibody TY25035 as shown in Table 4A, or one or more amino acid sequences having at least 80% (e.g., at least 85%, 90%, 95%, 98%, or 99%; or 100%) sequence identity with a VH or a VL of TY25035 as shown in Table 4A.

In some embodiments, the isolated antibody, antigen-binding fragment, or masked antibody (e.g., activatable antibody) comprises a VH and a VL, wherein a) the VH comprises a CDR-H1 comprising the amino acid sequence of SEQ ID NO: 42, or a variant thereof comprising up to 5 (e.g., 1, 2, 3, 4, or 5) amino acid substitutions; a CDR-H2 comprising the amino acid sequence of SEQ ID NO: 59, or a variant thereof comprising up to 5 (e.g., 1, 2, 3, 4, or 5) amino acid substitutions; and/or a CDR-H3 comprising the amino acid sequence of SEQ ID NO: 76, or a variant thereof comprising up to 5 (e.g., 1, 2, 3, 4, or 5) amino acid substitutions; and/or b) the VL comprises a CDR-L1 comprising the amino acid sequence of SEQ ID NO: 93, or a variant thereof comprising up to 5 (e.g., 1, 2, 3, 4, or 5) amino acid substitutions; a CDR-L2 comprising the amino acid sequence of SEQ ID NO: 110, or a variant thereof comprising up to 5 (e.g., 1, 2, 3, 4, or 5) amino acid substitutions; and/or a CDR-L3 comprising the amino acid sequence of SEQ ID NO: 127, or a variant thereof comprising up to 5 (e.g., 1, 2, 3, 4, or 5) amino acid substitutions. In some embodiments, a) the VH comprises the amino acid sequence of SEQ ID NO: 42, or a variant thereof comprising up to 5 (e.g., 1, 2, 3, 4, or 5) amino acid substitutions; the amino acid sequence of SEQ ID NO: 59, or a variant thereof comprising up to 5 (e.g., 1, 2, 3, 4, or 5) amino acid substitutions; and/or the amino acid sequence of SEQ ID NO: 76, or a variant thereof comprising up to 5 (e.g., 1, 2, 3, 4, or 5) amino acid substitutions; and/or b) the VL comprises the amino acid sequence of SEQ ID NO: 93, or a variant thereof comprising up to 5 (e.g., 1, 2, 3, 4, or 5) amino acid substitutions; the amino acid sequence of SEQ ID NO: 110, or a amino acid sequence of SEQ ID NO: 127, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises one, two, three, four, five, or six CDRs of illustrative antibody TY25036 as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising a CDR-H1, a CDR-H2 and a CDR-H3 of a VH comprising the amino acid sequence of SEQ ID NO: 15, or an amino acid sequence having at least 80% (e.g., at least 85%, 90%, 95%, 98%, or 99%; or 100%) sequence identity with SEQ ID NO: 15; and/or a VL comprising a CDR-L1, a CDR-L2 and a CDR-L3 of a VL comprising the amino acid sequence of SEQ ID NO: 16, or an amino acid sequence having at least 80% (e.g., at least 85%, 90%, 95%, 98%, or 99%; or 100%) sequence identity with SEQ ID NO: 16. In certain embodiments, the VH comprises the amino acid sequence of SEQ ID NO: 15, or an amino acid sequence having at least 80% (e.g., at least 85%, 90%, 95%, 98%, or 99%; or 100%) sequence identity with SEQ ID NO: 15; and/or the VL comprises the amino acid sequence of SEQ ID NO: 16, or an amino acid sequence having at least 80% (e.g., at least 85%, 90%, 95%, 98%, or 99%; or 100%) sequence identity with SEQ ID NO: 16. In some embodiments, the isolated antibody, antigen-binding fragment, or masked antibody (e.g., activatable antibody) comprises the VH and/or the VL of illustrative antibody TY25036 as shown in Table 4A, or one or more amino acid sequences having at least 80% (e.g., at least 85%, 90%, 95%, 98%, or 99%; or 100%) sequence identity with a VH or a VL of TY25036 as shown in Table 4A.

In some embodiments, the isolated antibody, antigen-binding fragment, or masked antibody (e.g., activatable antibody) comprises a VH and a VL, wherein a) the VH comprises a CDR-H1 comprising the amino acid sequence of SEQ ID NO: 43, or a variant thereof comprising up to 5 (e.g., 1, 2, 3, 4, or 5) amino acid substitutions; a CDR-H2 comprising the amino acid sequence of SEQ ID NO: 60, or a variant thereof comprising up to 5 (e.g., 1, 2, 3, 4, or 5) amino acid substitutions; and/or a CDR-H3 comprising the amino acid sequence of SEQ ID NO: 77, or a variant thereof comprising up to 5 (e.g., 1, 2, 3, 4, or 5) amino acid substitutions; and/or b) the VL comprises a CDR-L1 comprising the amino acid sequence of SEQ ID NO: 94, or a variant thereof comprising up to 5 (e.g., 1, 2, 3, 4, or 5) amino acid substitutions; a CDR-L2 comprising the amino acid sequence of SEQ ID NO: 111, or a variant thereof comprising up to 5 (e.g., 1, 2, 3, 4, or 5) amino acid substitutions; and/or a CDR-L3 comprising the amino acid sequence of SEQ ID NO: 128, or a variant thereof comprising up to 5 (e.g., 1, 2, 3, 4, or 5) amino acid substitutions. In some embodiments, a) the VH comprises the amino acid sequence of SEQ ID NO: 43, or a variant thereof comprising up to 5 (e.g., 1, 2, 3, 4, or 5) amino acid substitutions; the amino acid sequence of SEQ ID NO: 60, or a variant thereof comprising up to 5 (e.g., 1, 2, 3, 4, or 5) amino acid substitutions; and/or the amino acid sequence of SEQ ID NO: 77, or a variant thereof comprising up to 5 (e.g., 1, 2, 3, 4, or 5) amino acid substitutions; and/or b) the VL comprises the amino acid sequence of SEQ ID NO: 94, or a variant thereof comprising up to 5 (e.g., 1, 2, 3, 4, or 5) amino acid substitutions; the amino acid sequence of SEQ ID NO: 111, or a variant thereof comprising up to 5 (e.g., 1, 2, 3, 4, or 5) amino acid substitutions; and/or the amino acid sequence of SEQ ID NO: 128, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises one, two, three, four, five, or six CDRs of illustrative antibody TY25037 as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising a CDR-H1, a CDR-H2 and a CDR-H3 of a VH comprising the amino acid sequence of SEQ ID NO: 17, or an amino acid sequence having at least 80% (e.g., at least 85%, 90%, 95%, 98%, or 99%; or 100%) sequence identity with SEQ ID NO: 17; and/or a VL comprising a CDR-L1, a CDR-L2 and a CDR-L3 of a VL comprising the amino acid sequence of SEQ ID NO: 18, or an amino acid sequence having at least 80% (e.g., at least 85%, 90%, 95%, 98%, or 99%; or 100%) sequence identity with SEQ ID NO: 18. In certain embodiments, the VH comprises the amino acid sequence of SEQ ID NO: 17, or an amino acid sequence having at least 80% (e.g., at least 85%, 90%, 95%, 98%, or 99%; or 100%) sequence identity with SEQ ID NO: 17; and/or the VL comprises the amino acid sequence of SEQ ID NO: 18, or an amino acid sequence having at least 80% (e.g., at least 85%, 90%, 95%, 98%, or 99%; or 100%) sequence identity with SEQ ID NO: 18. In some embodiments, the isolated antibody, antigen-binding fragment, illustrative antibody TY25037 as shown in Table 4A, or one or more amino acid sequences having at least 80% (e.g., at least 85%, 90%, 95%, 98%, or 99%; or 100%) sequence identity with a VH or a VL of TY25037 as shown in Table 4A.

In some embodiments, the isolated antibody, antigen-binding fragment, or masked antibody (e.g., activatable antibody) comprises a VH and a VL, wherein a) the VH comprises a CDR-H1 comprising the amino acid sequence of SEQ ID NO: 44, or a variant thereof comprising up to 5 (e.g., 1, 2, 3, 4, or 5) amino acid substitutions; a CDR-H2 comprising the amino acid sequence of SEQ ID NO: 61, or a variant thereof comprising up to 5 (e.g., 1, 2, 3, 4, or 5) amino acid substitutions; and/or a CDR-H3 comprising the amino acid sequence of SEQ ID NO: 78, or a variant thereof comprising up to 5 (e.g., 1, 2, 3, 4, or 5) amino acid substitutions; and/or b) the VL comprises a CDR-L1 comprising the amino acid sequence of SEQ ID NO: 95, or a variant thereof comprising up to 5 (e.g., 1, 2, 3, 4, or 5) amino acid substitutions; a CDR-L2 comprising the amino acid sequence of SEQ ID NO: 112, or a variant thereof comprising up to 5 (e.g., 1, 2, 3, 4, or 5) amino acid substitutions; and/or a CDR-L3 comprising the amino acid sequence of SEQ ID NO: 129, or a variant thereof comprising up to 5 (e.g., 1, 2, 3, 4, or 5) amino acid substitutions. In some embodiments, a) the VH comprises the amino acid sequence of SEQ ID NO: 44, or a variant thereof comprising up to 5 (e.g., 1, 2, 3, 4, or 5) amino acid substitutions; the amino acid sequence of SEQ ID NO: 61, or a variant thereof comprising up to 5 (e.g., 1, 2, 3, 4, or 5) amino acid substitutions; and/or the amino acid sequence of SEQ ID NO: 78, or a variant thereof comprising up to 5 (e.g., 1, 2, 3, 4, or 5) amino acid substitutions; and/or b) the VL comprises the amino acid sequence of SEQ ID NO: 95, or a variant thereof comprising up to 5 (e.g., 1, 2, 3, 4, or 5) amino acid substitutions; the amino acid sequence of SEQ ID NO: 112, or a variant thereof comprising up to 5 (e.g., 1, 2, 3, 4, or 5) amino acid substitutions; and/or the amino acid sequence of SEQ ID NO: 129, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises one, two, three, four, five, or six CDRs of illustrative antibody TY25038 as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising a CDR-H1, a CDR-H2 and a CDR-H3 of a VH comprising the amino acid sequence of SEQ ID NO: 19, or an amino acid sequence having at least 80% (e.g., at least 85%, 90%, 95%, 98%, or 99%; or 100%) sequence identity with SEQ ID NO: 19; and/or a VL comprising a CDR-L1, a CDR-L2 and a CDR-L3 of a VL comprising the amino acid sequence of SEQ ID NO: 20, or an amino acid sequence having at least 80% (e.g., at least 85%, 90%, 95%, 98%, or 99%; or 100%) sequence identity with SEQ ID NO: 20. In certain embodiments, the VH comprises the amino acid sequence of SEQ ID NO: 19, or an amino acid sequence having at least 80% (e.g., at least 85%, 90%, 95%, 98%, or 99%; or 100%) sequence identity with SEQ ID NO: 19; and/or the VL comprises the amino acid sequence of SEQ ID NO: 20, or an amino acid sequence having at least 80% (e.g., at least 85%, 90%, 95%, 98%, or 99%; or 100%) sequence identity with SEQ ID NO: 20. In some embodiments, the isolated antibody, antigen-binding fragment, or masked antibody (e.g., activatable antibody) comprises the VH and/or the VL of illustrative antibody TY25038 as shown in Table 4A, or one or more amino acid sequences having at least 80% (e.g., at least 85%, 90%, 95%, 98%, or 99%; or 100%) sequence identity with a VH or a VL of TY25038 as shown in Table 4A.

In some embodiments, the isolated antibody, antigen-binding fragment, or masked antibody (e.g., activatable antibody) comprises a VH and a VL, wherein a) the VH comprises a CDR-H1 comprising the amino acid sequence of SEQ ID NO: 45, or a variant thereof comprising up to 5 (e.g., 1, 2, 3, 4, or 5) amino acid substitutions; a CDR-H2 comprising the amino acid sequence of SEQ ID NO: 62 or a variant thereof comprising up to 5 (e.g., 1, 2, 3, 4, or 5) amino acid substitutions; and/or a CDR-H3 comprising the amino acid sequence of SEQ ID NO: 79, or a variant thereof comprising up to 5 (e.g., 1, 2, 3, 4, or 5) amino acid substitutions; and/or b) the VL comprises a CDR-L1 comprising the amino acid sequence of SEQ ID NO: 96, or a variant thereof comprising up to 5 (e.g., 1, 2, 3, 4, or 5) amino acid substitutions; a CDR-L2 comprising the amino acid sequence of SEQ ID NO: 113, or a variant thereof comprising up to 5 (e.g., 1, 2, 3, 4, or 5) amino acid substitutions; and/or a CDR-L3 comprising the amino acid sequence of SEQ ID NO: 130, or a variant thereof comprising up to 5 (e.g., 1, 2, 3, 4, or 5) amino acid substitutions. In some embodiments, a) the VH comprises the amino acid sequence of SEQ ID NO: 45, or a variant thereof comprising up to 5 (e.g., 1, 2, 3, 4, or 5) amino acid substitutions; the amino acid sequence of SEQ ID NO: 62, or a variant thereof comprising up to 5 (e.g., 1, 2, 3, 4, or 5) amino acid substitutions; and/or the amino acid sequence of SEQ ID NO: 79, or a variant thereof comprising up to 5 (e.g., 1, 2, 3, 4, or 5) amino acid substitutions; and/or b) the VL comprises the amino acid sequence of SEQ ID NO: 96, or a variant thereof comprising up to 5 (e.g., 1, 2, 3, 4, or 5) amino acid substitutions; the amino acid sequence of SEQ ID NO: 113, or a amino acid sequence of SEQ ID NO: 130, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises one, two, three, four, five, or six CDRs of illustrative antibody TY25039 as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising a CDR-H1, a CDR-H2 and a CDR-H3 of a VH comprising the amino acid sequence of SEQ ID NO: 21, or an amino acid sequence having at least 80% (e.g., at least 85%, 90%, 95%, 98%, or 99%; or 100%) sequence identity with SEQ ID NO: 21; and/or a VL comprising a CDR-L1, a CDR-L2 and a CDR-L3 of a VL comprising the amino acid sequence of SEQ ID NO: 22, or an amino acid sequence having at least 80% (e.g., at least 85%, 90%, 95%, 98%, or 99%; or 100%) sequence identity with SEQ ID NO: 22. In certain embodiments, the VH comprises the amino acid sequence of SEQ ID NO: 21, or an amino acid sequence having at least 80% (e.g., at least 85%, 90%, 95%, 98%, or 99%; or 100%) sequence identity with SEQ ID NO: 21; and/or the VL comprises the amino acid sequence of SEQ ID NO: 22, or an amino acid sequence having at least 80% (e.g., at least 85%, 90%, 95%, 98%, or 99%; or 100%) sequence identity with SEQ ID NO: 22. In some embodiments, the isolated antibody, antigen-binding fragment, or masked antibody (e.g., activatable antibody) comprises the VH and/or the VL of illustrative antibody TY25039 as shown in Table 4A, or one or more amino acid sequences having at least 80% (e.g., at least 85%, 90%, 95%, 98%, or 99%; or 100%) sequence identity with a VH or a VL of TY25039 as shown in Table 4A.

In some embodiments, the isolated antibody, antigen-binding fragment, or masked antibody (e.g., activatable antibody) comprises a VH and a VL, wherein a) the VH comprises a CDR-H1 comprising the amino acid sequence of SEQ ID NO: 46, or a variant thereof comprising up to 5 (e.g., 1, 2, 3, 4, or 5) amino acid substitutions; a CDR-H2 comprising the amino acid sequence of SEQ ID NO: 63, or a variant thereof comprising up to 5 (e.g., 1, 2, 3, 4, or 5) amino acid substitutions; and/or a CDR-H3 comprising the amino acid sequence of SEQ ID NO: 80, or a variant thereof comprising up to 5 (e.g., 1, 2, 3, 4, or 5) amino acid substitutions; and/or b) the VL comprises a CDR-L1 comprising the amino acid sequence of SEQ ID NO: 97, or a variant thereof comprising up to 5 (e.g., 1, 2, 3, 4, or 5) amino acid substitutions; a CDR-L2 comprising the amino acid sequence of SEQ ID NO: 114, or a variant thereof comprising up to 5 (e.g., 1, 2, 3, 4, or 5) amino acid substitutions; and/or a CDR-L3 comprising the amino acid sequence of SEQ ID NO: 131, or a variant thereof comprising up to 5 (e.g., 1, 2, 3, 4, or 5) amino acid substitutions. In some embodiments, the VH comprises the amino acid sequence of SEQ ID NO: 46, or a variant thereof comprising up to 5 (e.g., 1, 2, 3, 4, or 5) amino acid substitutions; the amino acid sequence of SEQ ID NO: 63, or a variant thereof comprising up to 5 (e.g., 1, 2, 3, 4, or 5) amino acid substitutions; and/or the amino acid sequence of SEQ ID NO: 80, or a variant thereof comprising up to 5 (e.g., 1, 2, 3, 4, or 5) amino acid substitutions; and/or b) the VL comprises the amino acid sequence of SEQ ID NO: 97, or a variant thereof comprising up to 5 (e.g., 1, 2, 3, 4, or 5) amino acid substitutions; the amino acid sequence of SEQ ID NO: 114, or a variant thereof comprising up to 5 (e.g., 1, 2, 3, 4, or 5) amino acid substitutions; and/or the amino acid sequence of SEQ ID NO: 131, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises one, two, three, four, five, or six CDRs of illustrative antibody TY25040 as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising a CDR-H1, a CDR-H2 and a CDR-H3 of a VH comprising the amino acid sequence of SEQ ID NO: 23, or an amino acid sequence having at least 80% (e.g., at least 85%, 90%, 95%, 98%, or 99%; or 100%) sequence identity with SEQ ID NO: 23; and/or a VL comprising a CDR-L1, a CDR-L2 and a CDR-L3 of a VL comprising the amino acid sequence of SEQ ID NO: 24, or an amino acid sequence having at least 80% (e.g., at least 85%, 90%, 95%, 98%, or 99%; or 100%) sequence identity with SEQ ID NO: 24. In certain embodiments, the VH comprises the amino acid sequence of SEQ ID NO: 23, or an amino acid sequence having at least 80% (e.g., at least 85%, 90%, 95%, 98%, or 99%; or 100%) sequence identity with SEQ ID NO: 23; and/or the VL comprises the amino acid sequence of SEQ ID NO: 24, or an amino acid sequence having at least 80% (e.g., at least 85%, 90%, 95%, 98%, or 99%; or 100%) sequence identity with SEQ ID NO: 24. In some embodiments, the isolated antibody, antigen-binding fragment, illustrative antibody TY25040 as shown in Table 4A, or one or more amino acid sequences having at least 80% (e.g., at least 85%, 90%, 95%, 98%, or 99%; or 100%) sequence identity with a VH or a VL of TY25040 as shown in Table 4A.

In some embodiments, the isolated antibody, antigen-binding fragment, or masked antibody (e.g., activatable antibody) comprises a VH and a VL, wherein a) the VH comprises a CDR-H1 comprising the amino acid sequence of SEQ ID NO: 47, or a variant thereof comprising up to 5 (e.g., 1, 2, 3, 4, or 5) amino acid substitutions; a CDR-H2 comprising the amino acid sequence of SEQ ID NO: 64, or a variant thereof comprising up to 5 (e.g., 1, 2, 3, 4, or 5) amino acid substitutions; and/or a CDR-H3 comprising the amino acid sequence of SEQ ID NO: 81, or a variant thereof comprising up to 5 (e.g., 1, 2, 3, 4, or 5) amino acid substitutions; and/or b) the VL comprises a CDR-L1 comprising the amino acid sequence of SEQ ID NO: 98, or a variant thereof comprising up to 5 (e.g., 1, 2, 3, 4, or 5) amino acid substitutions; a CDR-L2 comprising the amino acid sequence of SEQ ID NO: 115, or a variant thereof comprising up to 5 (e.g., 1, 2, 3, 4, or 5) amino acid substitutions; and/or a CDR-L3 comprising the amino acid sequence of SEQ ID NO: 132, or a variant thereof comprising up to 5 (e.g., 1, 2, 3, 4, or 5) amino acid substitutions. In some embodiments, a) the VH comprises the amino acid sequence of SEQ ID NO: 47, or a variant thereof comprising up to 5 (e.g., 1, 2, 3, 4, or 5) amino acid substitutions; the amino acid sequence of SEQ ID NO: 64, or a variant thereof comprising up to 5 (e.g., 1, 2, 3, 4, or 5) amino acid substitutions; and/or the amino acid sequence of SEQ ID NO: 81, or a variant thereof comprising up to 5 (e.g., 1, 2, 3, 4, or 5) amino acid substitutions; and/or b) the VL comprises the amino acid sequence of SEQ ID NO: 98, or a variant thereof comprising up to 5 (e.g., 1, 2, 3, 4, or 5) amino acid substitutions; the amino acid sequence of SEQ ID NO: 115, or a variant thereof comprising up to 5 (e.g., 1, 2, 3, 4, or 5) amino acid substitutions; and/or the amino acid sequence of SEQ ID NO: 132, or a variant thereof comprising up to 5 (e.g., 1, 2, 3, 4, or 5) amino acid substitutions. In some embodiments, the isolated antibody, antigen-binding fragment, or masked antibody (e.g., activatable antibody) comprises one, two, three, four, five, or six CDRs of illustrative antibody TY25041 as shown in Tables 3A-3B, or variants thereof comprising up to 5 (e.g., 1, 2, 3, 4, or 5) amino acid substitutions.

In some embodiments, the isolated antibody, antigen-binding fragment, or masked antibody (e.g., activatable antibody) comprises a VH comprising a CDR-H1, a CDR-H2 and a CDR-H3 of a VH comprising the amino acid sequence of SEQ ID NO: 25, or an amino acid sequence having at least 80% (e.g., at least 85%, 90%, 95%, 98%, or 99%; or 100%) sequence identity with SEQ ID NO: 25; and/or a VL comprising a CDR-L1, a CDR-L2 and a CDR-L3 of a VL comprising the amino acid sequence of SEQ ID NO: 26, or an amino acid sequence having at least 80% (e.g., at least 85%, 90%, 95%, 98%, or 99%; or 100%) sequence identity with SEQ ID NO: 26. In certain embodiments, the VH comprises the amino acid sequence of SEQ ID NO: 25, or an amino acid sequence having at least 80% (e.g., at least 85%, 90%, 95%, 98%, or 99%; or 100%) sequence identity with SEQ ID NO: 25; and/or the VL comprises the amino acid sequence of SEQ ID NO: 26, or an amino acid sequence having at least 80% (e.g., at least 85%, 90%, 95%, 98%, or 99%; or 100%) sequence identity with SEQ ID NO: 26. In some embodiments, the isolated antibody, antigen-binding fragment, or masked antibody (e.g., activatable antibody) comprises the VH and/or the VL of illustrative antibody TY25041 as shown in Table 4A, or one or more amino acid sequences having at least 80% (e.g., at least 85%, 90%, 95%, 98%, or 99%; or 100%) sequence identity with a VH or a VL of TY25041 as shown in Table 4A.

### B-1. Isolated Antibodies

In some aspects, the present disclosure provides an isolated antibody that binds to human CD47. In some embodiments, the isolated antibody has an EC50 of about 100 nM or less for binding to human CD47 in vitro. In some embodiments, the isolated antibody has an IC50 of about 100 nM or less for blocking binding of human CD47 to human SIRPα in vitro. In some embodiments, the antibody binds human CD47 with a Kp of 50 nM or less as measured by surface plasmon resonance. In certain embodiments, the antibody can be cross-reactive with at least one non-human species selected from the list consisting of cynomolgus monkey, rat, and dog. The isolated antibodies disclosed herein may be useful for the treatment of a CD47-positive disease or condition (e.g., cancer). The isolated antibody of the present disclosure may comprise any of the CDR, VH, VL, heavy chain, and/or light chain sequences described herein.

The CD47 antibodies described herein can be in any class, such as IgG, IgM, IgE, IgA, or IgD. It is preferred that the CD47 antibodies are in the IgG class, such as IgG1, IgG2, IgG3, or lgG4 subclass. A CD47 antibody can be converted from one class or subclass to another class or subclass using methods known in the art. An exemplary method for producing an antibody in a desired class or subclass comprises the steps of isolating a polynucleotide encoding a heavy chain of an CD47 antibody and a polynucleotide encoding a light chain of a CD47 antibody, isolating the sequence encoding the VH region, ligating the VH sequence to a sequence encoding a heavy chain constant region of the desired class or subclass, expressing the light chain gene and the heavy chain construct in a cell, and collecting the CD47 antibody.

The isolated antibodies described herein may comprise an Fc region of human IgG, IgM, IgE, IgA, or IgD. In a preferred embodiment, the isolated antibody comprises an Fc region of human IgG of the IgG1, IgG2, IgG3, or lgG4 subclass. The Fc region may comprise one or more (e.g., 1, 2, 3, 4, 5, 10, or more) amino acid substitutions relative the known sequence of the Fc region of the human antibody class or subclass.

In some embodiments, the isolated antibody comprises a first polypeptide comprising a light chain comprising a VL and a second polypeptide comprising a heavy chain comprising, from N terminus to C terminus, a VH and a an Fc region. In some embodiments, the Fc region is an IgG4 Fc region. In some embodiments, the Fc region is an IgG1 Fc region. In certain embodiments, the Fc region is an IgG1 Fc region comprising a S239D substitution and/or an 1332E substitution, wherein numbering is according to Kabat. In certain embodiments, the Fc region is an IgG1 Fc region comprising a S239D substitution and an 1332E substitution, wherein numbering is according to Kabat. IgG1 Fc regions comprising a S239D substitution and an 1332E substitution are described in detail in Lazar et al. (Engineered antibody Fc variants with enhanced effector function.” PNAS 103.11 (2006): 4005-4010). The first polypeptide and second polypeptide may comprise any of the CDR, VH, VL, heavy chain, and/or light chain sequences described herein. In some embodiments, a) the VL of the first polypeptide comprises one or more amino acid sequences selected from the group consisting of SEQ ID NOs: 190-196; and/or b) the VH of the second polypeptide comprises one or more amino acid sequences selected from the group consisting of SEQ ID NOs: 182-189. In some variations, a) the VL of the first polypeptide comprises the amino acid sequences of SEQ ID NOs: 191, 194, and/or 195; and/or b) the VH of the second polypeptide comprises the amino acid sequences of SEQ ID NOs: 182, 185, and/or 188. In certain embodiments, a) the VL of the first polypeptide comprises one or more amino acid sequences selected from the group consisting of SEQ ID NOs: 31-85, or a variant thereof comprising up to 5 (e.g., 1, 2, 3, 4, or 5) amino acid substitutions; and/or b) the VH of the second polypeptide comprises one or more amino acid sequences selected from the group consisting of SEQ ID NOs: 86-136 or a variant thereof comprising up to 5 (e.g., 1, 2, 3, 4, or 5) amino acid substitutions. In some variations, a) the VL of the first polypeptide comprises a CDR-L1 comprising an amino acid sequence selected from the group consisting of SEQ ID NOs: 86-102, or a variant thereof comprising up to 5 (e.g., 1, 2, 3, 4, or 5) amino acid substitutions; a CDR-L2 comprising an amino acid sequence selected from the group consisting of SEQ ID NOs: 103-119, or a variant thereof comprising up to 5 (e.g., 1, 2, 3, 4, or 5) amino acid substitutions; and/or a CDR-L3 comprising an amino acid sequence selected from the group consisting of SEQ ID NOs: 120-136 or a variant thereof comprising up to 5 (e.g., 1, 2, 3, 4, or 5) amino acid substitutions; and/or b) the VH of the second polypeptide comprises a CDR-H1 comprising an amino acid sequence selected from the group consisting of SEQ ID NOs: 35-51, or a variant thereof comprising up to 5 (e.g., 1, 2, 3, 4, or 5) amino acid substitutions; a CDR-H2 comprising an amino acid sequence selected from the group consisting of SEQ ID NOs: 52-68, or a variant thereof comprising up to 5 (e.g., 1, 2, 3, 4, or 5) amino acid substitutions; and/or a CDR-H3 comprising an amino acid sequence selected from the group consisting of SEQ ID NOs: 69-85, or a variant thereof comprising up to 5 (e.g., 1, 2, 3, 4, or 5) amino acid substitutions. In certain embodiments, a) the VL of the first polypeptide comprises the amino acid sequence of SEQ ID NO: 99, the amino acid sequence of SEQ ID NO: 116, and/or the amino acid sequence of SEQ ID NO: 133; and/or b) the VH of the second polypeptide comprises the amino acid sequence of SEQ ID NO: 48, the amino acid sequence of SEQ ID NO: 65, and/or the amino acid sequence of SEQ ID NO: 82. In some embodiments, the VL of the first polypeptide comprises one, two, or three CDRs of illustrative antibody TY21446 as shown in Table 3B, or variants thereof comprising up to 5 (e.g., 1, 2, 3, 4, or 5) amino acid substitutions; and/or the VH of the second polypeptide comprises one, two, or three CDRs of illustrative antibody TY21446 as shown in Table 3A, or variants thereof comprising up to 5 (e.g., 1, 2, 3, 4, or 5) amino acid substitutions. In some embodiments, the VL of the first polypeptide comprises the amino acid sequence of SEQ ID NO: 28, or an amino acid sequence having at least 80% (e.g., at least 85%, 90%, 95%, 98%, or 99%; or 100%) sequence identity with the amino acid sequence of SEQ ID NO: 28; and the VH of the second polypeptide comprises the amino acid sequence of SEQ ID NO: 27, or an amino acid sequence having at least 80% (e.g., at least 85%, 90%, 95%, 98%, or 99%; or 100%) sequence identity with the amino acid sequence of SEQ ID NO: 27. In certain embodiments, the first polypeptide comprises the VL of illustrative antibody TY21446 as shown in Table 4A, or an amino acid sequence having at least 80% (e.g., at least 85%, 90%, 95%, 98%, or 99%; or 100%) sequence identity with the VL of illustrative antibody TY21446 as shown in Table 4A; and the second polypeptide comprises the VH of illustrative antibody TY21446 as shown in Table 4A, or an amino acid sequence having at least 80% (e.g., at least 85%, 90%, 95%, 98%, or 99%; or 100%) sequence identity with the VH of illustrative antibody TY21446 as shown in Table 4A.

In some embodiments, provided herein is an isolated anti-CD47 antibody, or antigen-binding fragment thereof, comprising an IgG1 Fc region, wherein the anti-CD47 antibody comprises a) a VH comprising the amino acid sequence of SEQ ID NO: 48, or a variant thereof comprising up to 5 (e.g., 1, 2, 3, 4, or 5) amino acid substitutions; the amino acid sequence of SEQ ID NO: 65, or a variant thereof comprising up to 5 (e.g., 1, 2, 3, 4, or 5) amino acid substitutions; and the amino acid sequence of SEQ ID NO: 82, or a variant thereof comprising up to 5 (e.g., 1, 2, 3, 4, or 5) amino acid substitutions; and b) a VL comprising: the amino acid sequence of SEQ ID NO: 99, or a variant thereof comprising up to 5 (e.g., 1, 2, 3, 4, or 5) amino acid substitutions; the amino acid sequence of SEQ ID NO: 116, or a variant thereof comprising up to 5 (e.g., 1, 2, 3, 4, or 5) amino acid substitutions; and the amino acid sequence of SEQ ID NO: 133, or a variant thereof comprising up to 5 (e.g., 1, 2, 3, 4, or 5) amino acid substitutions. In certain embodiments, provided herein is an isolated anti-CD47 antibody, or antigen-binding fragment thereof, comprising an IgG1 Fc region, wherein the anti-CD47 antibody comprises a VH comprising the amino acid sequence of SEQ ID NO: 48, the amino acid sequence of SEQ ID NO: 65, and the amino acid sequence of SEQ ID NO: 82, or a variant thereof comprising up to 5 (e.g., 1, 2, 3, 4, or 5) amino acid substitutions of each of SEQ ID NO: 48, SEQ ID NO: 65, and/or SEQ ID NO: 82; and b) a VL comprising the amino acid sequence of SEQ ID NO: 99, the amino acid sequence of SEQ ID NO: 116, and the amino acid sequence of SEQ ID NO: 133, or a variant thereof comprising up to 5 (e.g., 1, 2, 3, 4, or 5) amino acid substitutions of each of SEQ ID NO: 99, SEQ ID NO: 116, and/or SEQ ID NO: 133. In some embodiments, provided herein is an isolated anti-CD47 antibody, or antigen-binding fragment thereof, comprising an IgG1 Fc region, wherein the anti-CD47 antibody comprises one, two, three, four, five, or six CDRs of illustrative antibody TY21446 as shown in Tables 3A-3B, or variants thereof comprising up to 5 (e.g., 1, 2, 3, 4, or 5) amino acid substitutions. In certain embodiments, provided herein is an isolated anti-CD47 antibody, or an antigen-binding fragment thereof, comprising an IgG1 Fc region, wherein the anti-CD47 antibody comprises a VH comprising the amino acid sequence of SEQ ID NO: 27, or an amino acid sequence having at least 80% (e.g., at least 85%, 90%, 95%, 98%, or 99%; or 100%) sequence identity with the amino acid sequence of SEQ ID NO: 27; and a VL comprising the amino acid sequence of SEQ ID NO: 28, or an amino acid sequence having at least 80% (e.g., at least 85%, 90%, 95%, 98%, or 99%; or 100%) sequence identity with the amino acid sequence of SEQ ID NO: 28. In some embodiments, provided herein is an isolated anti-CD47 antibody, or an antigen-binding fragment thereof, comprising an IgG1 Fc region, wherein the anti-CD47 antibody comprises the VH and/or the VL of illustrative antibody TY21446 as shown in Table 4A, or one or more amino acid sequences having at least 80% (e.g., at least 85%, 90%, 95%, 98%, or 99%; or 100%) sequence identity with a VH or a VL of TY21446 as shown in Table 4A. In some embodiments, the anti-CD47 antibody comprises a heavy chain comprising the amino acid sequence of SEQ ID NO: 145, or an amino acid sequence having at least 80% (e.g., at least 85%, 90%, 95%, 98%, or 99%; or 100%) sequence identity with the amino acid sequence of SEQ ID NO: 145; and a light chain comprising the amino acid sequence of SEQ ID NO: 144, or an amino acid sequence having at least 80% (e.g., at least 85%, 90%, 95%, 98%, or 99%; or 100%) sequence identity with the amino acid sequence of SEQ ID NO: 144. In some embodiments, the anti-CD47 antibody comprises a heavy chain and/or a light chain of illustrative antibody TY26896 as shown in Table 6, or one or more amino acid sequences having at least 80% (e.g., at least 85%, 90%, 95%, 98%, or 99%; or 100%) sequence identity with a heavy chain or a light chain of illustrative antibody TY26896 as shown in Table 6. In some embodiments, the IgG1 Fc region comprises a S239D substitution and/or an 1332E substitution. In certain embodiments, the IgG1 Fc region comprises a S239D substitution and an 1332E substitution. In certain embodiments, the human IgG1 Fc region comprises two Fc domains, wherein each of the two Fc domains comprises a S239D substitution and/or an 1332E substitution (e.g., a S239D substitution and an 1332E substitution). In some embodiments, the anti-CD47 antibody comprises a heavy chain comprising the amino acid sequence of SEQ ID NO: 147, or an amino acid sequence having at least 80% (e.g., at least 85%, 90%, 95%, 98%, or 99%; or 100%) sequence identity with the amino acid sequence of SEQ ID NO: 147; and a light chain comprising the amino acid sequence of SEQ ID NO: 146, or an amino acid sequence having at least 80% (e.g., at least 85%, 90%, 95%, 98%, or 99%; or 100%) sequence identity with the amino acid sequence of SEQ ID NO: 146. In some embodiments, the anti-CD47 antibody comprises a heavy chain and/or a light chain of illustrative antibody TY26897 as shown in Table 6, or one or more amino acid sequences having at least 80% (e.g., at least 85%, 90%, 95%, 98%, or 99%; or 100%) sequence identity with a heavy chain or a light chain of illustrative antibody TY26897 as shown in Table 6. In some embodiments, the IgG1 Fc region has enhanced antibody-dependent cellular cytotoxic (ADCC) and/or antibody-dependent cellular phagocytosis (ADCP) function(s).

In some embodiments, provided herein is an isolated anti-CD47 antibody, or antigen-binding fragment thereof, comprising an IgG1 Fc region, wherein the anti-CD47 antibody comprises a) a VH comprising the amino acid sequence of SEQ ID NO: 49, or a variant thereof comprising up to 5 (e.g., 1, 2, 3, 4, or 5) amino acid substitutions; the amino acid sequence of SEQ ID NO: 66, or a variant thereof comprising up to 5 (e.g., 1, 2, 3, 4, or 5) amino acid substitutions; and the amino acid sequence of SEQ ID NO: 83, or a variant thereof comprising up to 5 (e.g., 1, 2, 3, 4, or 5) amino acid substitutions; and b) a VL comprising: the amino acid sequence of SEQ ID NO: 100, or a variant thereof comprising up to 5 (e.g., 1, 2, 3, 4, or 5) amino acid substitutions; the amino acid sequence of SEQ ID NO: 117, or a variant thereof comprising up to 5 (e.g., 1, 2, 3, 4, or 5) amino acid substitutions; and the amino acid sequence of SEQ ID NO: 134, or a variant thereof comprising up to 5 (e.g., 1, 2, 3, 4, or 5) amino acid substitutions. In certain embodiments, provided herein is an isolated anti-CD47 antibody, or antigen-binding fragment thereof, comprising an IgG1 Fc region, wherein the anti-CD47 antibody comprises a VH comprising the amino acid sequence of SEQ ID NO: 49, the amino acid sequence of SEQ ID NO: 66, and the amino acid sequence of SEQ ID NO: 83, or a variant thereof comprising up to 5 (e.g., 1, 2, 3, 4, or 5) amino acid substitutions of each of SEQ ID NO: 49, SEQ ID NO: 66, and/or SEQ ID NO: 83; and b) a VL comprising: the amino acid sequence of SEQ ID NO: 100, the amino acid sequence of SEQ ID NO: 117, and the amino acid sequence of SEQ ID NO: 134, or a variant thereof comprising up to 5 (e.g., 1, 2, 3, 4, or 5) amino acid substitutions of each of SEQ ID NO: 100, SEQ ID NO: 117, and/or SEQ ID NO: 134. In some embodiments, provided herein is an isolated anti-CD47 antibody, or antigen-binding fragment thereof, comprising an IgG1 Fc region, wherein the anti-CD47 antibody comprises one, two, three, four, five, or six CDRs of illustrative antibody TY21447 as shown in Tables 3A-3B, or variants thereof comprising up to 5 (e.g., 1, 2, 3, 4, or 5) amino acid substitutions. In certain embodiments, provided herein is an isolated anti-CD47 antibody, or an antigen-binding fragment thereof, comprising an IgG1 Fc region, wherein the anti-CD47 antibody comprises a VH comprising the amino acid sequence of SEQ ID NO: 29, or an amino acid sequence having at least 80% (e.g., at least 85%, 90%, 95%, 98%, or 99%; or 100%) sequence identity with the amino acid sequence of SEQ ID NO: 29; and a VL comprising the amino acid sequence of SEQ ID NO: 30, or an amino acid sequence having at least 80% (e.g., at least 85%, 90%, 95%, 98%, or 99%; or 100%) sequence identity with the amino acid sequence of SEQ ID NO: 30. In some embodiments, provided herein is an isolated anti-CD47 antibody, or an antigen-binding fragment thereof, comprising an IgG1 Fc region, wherein the anti-CD47 antibody comprises the VH and/or the VL of illustrative antibody TY21447 as shown in Table 4A, or one or more amino acid sequences having at least 80% (e.g., at least 85%, 90%, 95%, 98%, or 99%; or 100%) sequence identity with a VH or a VL of TY21447 as shown in Table 4A. In some embodiments, the IgG1 Fc region comprises a S239D substitution and/or an 1332E substitution. In certain embodiments, the IgG1 Fc region comprises a S239D substitution and an 1332E substitution. In certain embodiments, the human IgG1 Fc region comprises two Fc domains, wherein each of the two Fc domains comprises a S239D substitution and/or an 1332E substitution (e.g., a S239D substitution and an 1332E substitution). In some embodiments, the IgG1 Fc region has enhanced antibody-dependent cellular cytotoxic (ADCC) and/or antibody-dependent cellular phagocytosis (ADCP) function(s).

In some embodiments, provided herein is an isolated anti-CD47 antibody, or antigen-binding fragment thereof, comprising an IgG1 Fc region, wherein the anti-CD47 antibody comprises a) a VH comprising the amino acid sequence of SEQ ID NO: 50, or a variant thereof comprising up to 5 (e.g., 1, 2, 3, 4, or 5) amino acid substitutions; the amino acid sequence of SEQ ID NO: 67, or a variant thereof comprising up to 5 (e.g., 1, 2, 3, 4, or 5) amino acid substitutions; and the amino acid sequence of SEQ ID NO: 84, or a variant thereof comprising up to 5 (e.g., 1, 2, 3, 4, or 5) amino acid substitutions; and b) a VL comprising: the amino acid sequence of SEQ ID NO: 101, or a variant thereof comprising up to 5 (e.g., 1, 2, 3, 4, or 5) amino acid substitutions; the amino acid sequence of SEQ ID NO: 118, or a variant thereof comprising up to 5 (e.g., 1, 2, 3, 4, or 5) amino acid substitutions; and the amino acid sequence of SEQ ID NO: 135, or a variant thereof comprising up to 5 (e.g., 1, 2, 3, 4, or 5) amino acid substitutions. In certain embodiments, provided herein is an isolated anti-CD47 antibody, or antigen-binding fragment thereof, comprising an IgG1 Fc region, wherein the anti-CD47 antibody comprises a VH comprising the amino acid sequence of SEQ ID NO: 50, the amino acid sequence of SEQ ID NO: 67, and the amino acid sequence of SEQ ID NO: 84, or a variant thereof comprising up to 5 (e.g., 1, 2, 3, 4, or 5) amino acid substitutions of each of SEQ ID NO: 50, SEQ ID NO: 67, and/or SEQ ID NO: 84; and b) a VL comprising: the amino acid sequence of SEQ ID NO: 101, the amino acid sequence of SEQ ID NO: 118, and the amino acid sequence of SEQ ID NO: 135, or a variant thereof comprising up to 5 (e.g., 1, 2, 3, 4, or 5) amino acid substitutions of each of SEQ ID NO: 101, SEQ ID NO: 118, and/or SEQ ID NO: 135. In some embodiments, provided herein is an isolated anti-CD47 antibody, or antigen-binding fragment thereof, comprising an IgG1 Fc region, wherein the anti-CD47 antibody comprises one, two, three, four, five, or six CDRs of illustrative antibody TY21449 as shown in Tables 3A-3B, or variants thereof comprising up to 5 (e.g., 1, 2, 3, 4, or 5) amino acid substitutions. In certain embodiments, provided herein is an isolated anti-CD47 antibody, or an antigen-binding fragment thereof, comprising an IgG1 Fc region, wherein the anti-CD47 antibody comprises a VH comprising the amino acid sequence of SEQ ID NO: 31, or an amino acid sequence having at least 80% (e.g., at least 85%, 90%, 95%, 98%, or 99%; or 100%) sequence identity with the amino acid sequence of SEQ ID NO: 31; and a VL comprising the amino acid sequence of SEQ ID NO: 32, or an amino acid sequence having at least 80% (e.g., at least 85%, 90%, 95%, 98%, or 99%; or 100%) sequence identity with the amino acid sequence of SEQ ID NO: 32. In some embodiments, provided herein is an isolated anti-CD47 antibody, or an antigen-binding fragment thereof, comprising an IgG1 Fc region, wherein the anti-CD47 antibody comprises the VH and/or the VL of illustrative antibody TY21449 as shown in Table 4A, or one or more amino acid sequences having at least 80% (e.g., at least 85%, 90%, 95%, 98%, or 99%; or 100%) sequence identity with a VH or a VL of TY21449 as shown in Table 4A. In some embodiments, the IgG1 Fc region comprises a S239D substitution and/or an 1332E substitution. In certain embodiments, the IgG1 Fc region comprises a S239D substitution and an 1332E substitution. In certain embodiments, the human IgG1 Fc region comprises two Fc domains, wherein each of the two Fc domains comprises a S239D substitution and/or an 1332E substitution (e.g., a S239D substitution and an 1332E substitution). In some embodiments, the IgG1 Fc region has enhanced antibody-dependent cellular cytotoxic (ADCC) and/or antibody-dependent cellular phagocytosis (ADCP) function(s).

In some embodiments, the isolated antibody comprises a human IgG4 Fc region. An isolated antibody of the present disclosure may comprise an IgG4 Fc region and any combination of the CDR, VH, VL, and/or light chain sequences described herein. In some embodiments, the isolated antibody comprises a first polypeptide comprising a light chain comprising a VL and a second polypeptide comprising a heavy chain comprising, from N terminus to C terminus, a VH and a human IgG4 Fc region. In certain embodiments, a) the VL of the first polypeptide comprises the amino acid sequence of SEQ ID NO: 99, the amino acid sequence of SEQ ID NO: 116, and/or the amino acid sequence of SEQ ID NO: 133; and/or b) the VH of the second polypeptide comprises the amino acid sequence of SEQ ID NO: 48, the amino acid sequence of SEQ ID NO: 65, and/or the amino acid sequence of SEQ ID NO: 82. In some embodiments, the VL of the first polypeptide comprises one, two, or three CDRs of illustrative antibody TY21446 as shown in Table 3B, or variants thereof comprising up to 5 (e.g., 1, 2, 3, 4, or 5) amino acid substitutions; and/or the VH of the second polypeptide comprises one, two, or three CDRs of illustrative antibody TY21446 as shown in Table 3A, or variants thereof comprising up to 5 (e.g., 1, 2, 3, 4, or 5) amino acid substitutions. In some embodiments, the VL of the first polypeptide comprises the amino acid sequence of SEQ ID NO: 28, and/or the VH of the second polypeptide comprises the amino acid sequence of SEQ ID NO: 27. In certain embodiments, the first polypeptide comprises the VL of illustrative antibody TY21446 as shown in Table 4A, or an amino acid sequence having at least 80% (e.g., at least 85%, 90%, 95%, 98%, or 99%; or 100%) sequence identity with the VL of illustrative antibody TY21446 as shown in Table 4A; and the second polypeptide comprises the VH of illustrative antibody TY21446 as shown in Table 4A, or an amino acid sequence having at least 80% (e.g., at least 85%, 90%, 95%, 98%, or 99%; or 100%) sequence identity with the VH of illustrative antibody TY21446 as shown in Table 4A. In certain embodiments, the first polypeptide comprises the amino acid sequence of SEQ ID NO: 140, or an amino acid sequence having at least 80% (e.g., at least 85%, 90%, 95%, 98%, or 99%; or 100%) sequence identity with the amino acid sequence of SEQ ID NO: 140; and/or the second polypeptide comprises the amino acid sequence of SEQ ID NO: 141, or an amino acid sequence having at least 80% (e.g., at least 85%, 90%, 95%, 98%, or 99%; or 100%) sequence identity with the amino acid sequence of SEQ ID NO: 141. In some embodiments, the first polypeptide comprises the light chain of antibody TY21446 as shown in Table 6, or an amino acid sequence having at least 80% (e.g., at least 85%, 90%, 95%, 98%, or 99%; or 100%) sequence identity with the amino acid sequence of the light chain of illustrative antibody TY21446 as shown in Table 6; and/or the second polypeptide comprises the heavy chain of TY21446 as shown in Table 6, or an amino acid sequence having at least 80% (e.g., at least 85%, 90%, 95%, 98%, or 99%; or 100%) sequence identity with the amino acid sequence of the heavy chain of illustrative antibody TY21446 as shown in Table 6.

In some embodiments, the isolated antibody comprises a human IgG1 Fc region. An isolated antibody of the present disclosure may comprise an IgG1 Fc region and any combination of the CDR, VH, VL, and/or light chain sequences described herein. In some embodiments, the isolated antibody comprises a first polypeptide comprising a light chain comprising a VL and a second polypeptide comprising a heavy chain comprising, from N terminus to C terminus, a VH and a human IgG1 Fc region. In certain embodiments, a) the VL of the first polypeptide comprises the amino acid sequence of SEQ ID NO: 99, the amino acid sequence of SEQ ID NO: 116, and/or the amino acid sequence of SEQ ID NO: 133; and/or b) the VH of the second polypeptide comprises the amino acid sequence of SEQ ID NO: 48, the amino acid sequence of SEQ ID NO: 65, and/or the amino acid sequence of SEQ ID NO: 82. In some embodiments, the VL of the first polypeptide comprises the amino acid sequence of SEQ ID NO: 28, and/or the VH of the second polypeptide comprises the amino acid sequence of SEQ ID NO: 27. In certain embodiments, the first polypeptide comprises the amino acid sequence of SEQ ID NO: 144, or an amino acid sequence having at least 80% (e.g., at least 85%, 90%, 95%, 98%, or 99%; or 100%) sequence identity with the amino acid sequence of SEQ ID NO: 144; and/or the second polypeptide comprises the amino acid sequence of SEQ ID NO: 145, or an amino acid sequence having at least 80% (e.g., at least 85%, 90%, 95%, 98%, or 99%; or 100%) sequence identity with the amino acid sequence of SEQ ID NO: 145. In some embodiments, the first polypeptide comprises the light chain of antibody TY26896 as shown in Table 6, or an amino acid sequence having at least 80% (e.g., at least 85%, 90%, 95%, 98%, or 99%; or 100%) sequence identity with the amino acid sequence of the light chain of illustrative antibody TY26896 as shown in Table 6; and/or the second polypeptide comprises the heavy chain of TY26896 as shown in Table 6, or an amino acid sequence having at least 80% (e.g., at least 85%, 90%, 95%, 98%, or 99%; or 100%) sequence identity with the amino acid sequence of the heavy chain of illustrative antibody TY26896 as shown in Table 6.

In some embodiments, the isolated antibody comprises a human IgG1 Fc region having one or more amino acid substitutions. The IgG1 Fc region can comprise any amino acid substitution known in the art to confer a desired property to the antibody having the IgG1 Fc region. In some embodiments, the IgG1 Fc region has enhanced antibody-dependent cellular cytotoxic (ADCC) and/or antibody-dependent cellular phagocytosis (ADCP) function(s). In certain embodiments, the human IgG1 Fc region comprises a S239D substitution and/or an 1332E substitution. In certain embodiments, the human IgG1 Fc region comprises two Fc domains, wherein each of the two Fc domains comprises a S239D substitution and/or an 1332E substitution (e.g., a S239D substitution and an 1332E substitution). An isolated antibody of the present disclosure may comprise an IgG1 Fc region comprising a S239D substitution and/or an 1332E substitution and any combination of the CDR, VH, VL, and/or light chain sequences described herein. In some embodiments, the isolated antibody comprises a first polypeptide comprising a light chain comprising a VL and a second polypeptide comprising a heavy chain comprising, from N terminus to C terminus, a VH and a human IgG1 Fc region comprising a S239D substitution and/or an 1332E substitution. In certain embodiments, the isolated antibody has enhanced ADCC activity. In some embodiments, the isolated antibody comprises a first polypeptide comprising a light chain comprising a VL and a second polypeptide comprising a heavy chain comprising, from N terminus to C terminus, a VH and a human IgG1 Fc region. In certain embodiments, a) the VL of the first polypeptide comprises the amino acid sequence of SEQ ID NO: 99, the amino acid sequence of SEQ ID NO: 116, and/or the amino acid sequence of SEQ ID NO: 133; and/or b) the VH of the second polypeptide comprises the amino acid sequence of SEQ ID NO: 48, the amino acid sequence of SEQ ID NO: 65, and/or the amino acid sequence of SEQ ID NO: 82. In some embodiments, the VL of the first polypeptide comprises the amino acid sequence of SEQ ID NO: 28, and/or the VH of the second polypeptide comprises the amino acid sequence of SEQ ID NO: 27. In certain embodiments, the first polypeptide comprises the amino acid sequence of SEQ ID NO: 146, or an amino acid sequence having at least 80% (e.g., at least 85%, 90%, 95%, 98%, or 99%; or 100%) sequence identity with the amino acid sequence of SEQ ID NO: 146; and/or the second polypeptide comprises the amino acid sequence of SEQ ID NO: 147, or an amino acid sequence having at least 80% (e.g., at least 85%, 90%, 95%, 98%, or 99%; or 100%) sequence identity with the amino acid sequence of SEQ ID NO: 147. In some embodiments, the first polypeptide comprises the light chain of antibody TY26897 as shown in Table 6, or an amino acid sequence having at least 80% (e.g., at least 85%, 90%, 95%, 98%, or 99%; or 100%) sequence identity with the amino acid sequence of the light chain of illustrative antibody TY26897 as shown in Table 6; and/or the second polypeptide comprises the heavy chain of TY26897 as shown in Table 6, or an amino acid sequence having at least 80% (e.g., at least 85%, 90%, 95%, 98%, or 99%; or 100%) sequence identity with the amino acid sequence of the heavy chain of illustrative antibody TY26897 as shown in Table 6.

Further, the antibodies provided by the present disclosure can be monoclonal or polyclonal. In a preferred embodiment, the isolated antibody is monoclonal.

Examples of specific isolated antibodies provided by the present disclosure include those listed in Tables 3A-6. The amino acid sequences of the heavy chain variable region, full length heavy chain for the IgG1 and IgG4 subclass, light chain variable region, and full length light chain of these antibodies are also provided.

Also provided are anti-CD47 antibodies that competitively bind to the same epitope as any one of the anti-CD47 antibodies as described herein, including antibodies listed in Tables 3A-6.

In some embodiments, the isolated antibody, or antigen binding fragment thereof, binds to human CD47 with a Kp of about 500 nM or less (e.g., about 500 nM or less, about 400 nM or less, about 300 nM or less, about 200 nM or less, about 150 nM or less, about 100 nM or less, about 90 nM or less, about 80 nM or less, about 75 nM or less, about 70 nM or less, about 60 nM or less, about 50 nM or less, about 40 nM or less, about 30 nM or less, about 25 nM or less, about 20 nM or less, about 10 nM or less, about 1 nM or less, about 0.1 nM or less, etc.). In some embodiments, the isolated antibody, or antigen binding fragment thereof, binds to human CD47 with a Kp of about 100 nM or less. In some embodiments, the isolated antibody, or antigen binding fragment thereof, binds to human CD47 with a Kp of about 50 nM or less. In some embodiments, the isolated antibody, or antigen binding fragment thereof, bind to human CD47 with a Kp of about 10 nM or less. Methods of measuring the Kp of an isolated antibody, or antigen binding fragment thereof, may be carried out using any method known in the art, including for example, by surface plasmon resonance, an ELISA, isothermal titration calorimetry, a filter binding assay, an EMSA, etc. In some embodiments, the Kp is measured by surface plasmon resonance (See e.g., Example 2 below).

In some embodiments, the antibody, or antigen binding fragment thereof, has a half maximal effective concentration (EC50) of about 500 nM or less (e.g., about 500 nM or less, about 400 nM or less, about 300 nM or less, about 200 nM or less, about 150 nM or less, about 100 nM or less, about 90 nM or less, about 80 nM or less, about 75 nM or less, about 70 nM or less, about 60 nM or less, about 50 nM or less, about 40 nM or less, about 30 nM or less, about 25 nM or less, about 20 nM or less, about 10 nM or less, about 1 nM or less, about 0.1 nM or less, etc.) for binding to human CD47 in vitro. In some embodiments, the antibody, or antigen binding fragment thereof, has an EC50 of about 100 nM or less for binding to human CD47 in vitro. In some embodiments, the antibody, or antigen binding fragment thereof, has an EC50 of about 10 nM or less for binding to human CD47 in vitro. In some embodiments, the antibody, or antigen binding fragment thereof, has an EC50 of about 1 nM or less for binding to human CD47 in vitro. Methods of measuring the EC50 of an antibody, or antigen binding fragment thereof, to bind a target (e.g., CD47) may be carried out using any method known in the art, including for example, an ELISA, a filter binding assay, an EMSA, etc. In some embodiments, the EC50 is measured by ELISA (Sec e.g., Example 2 below).

In some embodiments, the antibody, or antigen binding fragment thereof, has a half maximal inhibitory concentration (IC50) of about 500 nM or less (e.g., about 500 nM or less, about 400 nM or less, about 300 nM or less, about 200 nM or less, about 150 nM or less, about 100 nM or less, about 90 nM or less, about 80 nM or less, about 75 nM or less, about 70 nM or less, about 60 nM or less, about 50 nM or less, about 40 nM or less, about 30 nM or less, about 25 nM or less, about 20 nM or less, about 10 nM or less, about 1 nM or less, about 0.1 nM or less, etc.) for blocking binding of human CD47 to human SIRPα in vitro. In some embodiments, the antibody, or antigen binding fragment thereof, has an IC50 of about 100 nM or less for blocking binding of human CD47 to human SIRPα in vitro. In some embodiments, the antibody, or antigen binding fragment thereof, has an IC50 of about 25 nM or less for blocking binding of human CD47 to human SIRPα in vitro. In some embodiments, the antibody, or antigen binding fragment thereof, has an IC50 of about 10 nM or less for blocking binding of human CD47 to human SIRPα in vitro. In some embodiments, the antibody, or antigen binding fragment thereof, has an IC50 of about 5 nM or less for blocking binding of human CD47 to human SIRPα in vitro. Methods of measuring the IC50 of an antibody, or antigen binding fragment thereof, to bind a target (e.g., CD47) may be carried out using any method known in the art, including for example, an ELISA, a filter binding assay, an EMSA, etc. In some embodiments, the EC50 is measured by ELISA (Scc e.g., Example 2 below).

In certain embodiments, the antibody, or antigen binding fragment thereof, completely blocks binding of human CD47 to human SIRPα in vitro when the antibody, or antigen binding fragment thereof, is provided at a concentration of about 1 nM or greater (e.g., about 1 nM or greater, about 5 nM or greater, about 10 nM or greater, about 20 nM or greater, about 40 nM or greater, about 60 nM or greater, about 80 nM or greater, about 100 nM or greater, about 200 nM or greater, about 400 nM or greater, about 600 nM or greater, about 800 nM or greater, about 1 μM or greater, about 2 μM or greater, about 4 μM or greater, about 6 μM or greater, about 8 μM or greater, about 10 UM or greater, about 20 μM or greater, about 40 μM or greater, about 60 μM or greater, about 80 μM or greater, about 100 μM or greater, etc.). In some embodiments, the antibody, or antigen binding fragment thereof, completely blocks binding of human CD47 to human SIRPα in vitro when the isolated antibody, or antigen binding fragment thereof, is provided at a concentration of about 1 μM or greater. In some embodiments, the antibody, or antigen binding fragment thereof, completely blocks binding of human CD47 to human SIRPα in vitro when the antibody, or antigen binding fragment thereof, is provided at a concentration of about 100 nM or greater. In some embodiments, the or antigen-binding fragment completely blocks binding of human CD47 to human SIRPα in vitro when the antibody, or antigen binding fragment thereof, is provided at a concentration of about 100 nM or greater. In some embodiments, the antibody, or antigen binding fragment thereof, completely blocks binding of human CD47 to human SIRPα in vitro when the antibody, or antigen binding fragment thereof, is provided at a concentration of about 10 nM or greater. As used herein, the term “complete blocking” or “completely blocks” refers to the antibody, antigen binding fragment's ability to reduce binding between a first protein and a second protein by at least about 80% (e.g., at least about 80%, at least about 85%, at least about 90%, at least about 95%, at least about 99%, etc.). Methods of measuring the ability of an antibody, or antigen binding fragment thereof, to block binding of a first protein (e.g., a CD47) and a second protein (e.g., SIRPα) are known in the art, including, without limitation, via BIAcore analysis, ELISA assays, and flow cytometry (See e.g., Example 2 below).

In some embodiments, the antibody, or antigen binding fragment thereof, has a half maximal effective concentration (EC50) of about 500 nM or less (e.g., about 500 nM or less, about 400 nM or less, about 300 nM or less, about 200 nM or less, about 150 nM or less, about 100 nM or less, about 90 nM or less, about 80 nM or less, about 75 nM or less, about 70 nM or less, about 60 nM or less, about 50 nM or less, about 40 nM or less, about 30 nM or less, about 25 nM or less, about 20 nM or less, about 10 nM or less, about 1 nM or less, about 0.1 nM or less, etc.) for binding to tumor cells in vitro. In some embodiments, the antibody, or antigen binding fragment thereof, has an EC50 of about 50 nM or less for binding to tumor cells in vitro. In some embodiments, the antibody, or antigen binding fragment thereof, has an EC50 of about 10 nM or less for binding to tumor cells in vitro. In some embodiments, the antibody, or antigen binding fragment thereof, has an EC50 of about 5 nM or less for binding to tumor cells in vitro. In some embodiments, the antibody, or antigen binding fragment thereof, has an EC50 of about 1 nM or less for binding to tumor cells in vitro. In certain embodiments, the tumor cells comprise a B cell lymphoma cell line (e.g., Raji cell line). In further embodiments, the tumor cells comprise a T cell lymphoma cell line (e.g., CEM cell line). Methods of measuring the EC50 of an antibody, or antigen binding fragment thereof, to bind to tumor cells may be carried out using any method known in the art, including for example, flow cytometry (Scc e.g., Example 6 below).

In some embodiments, the antibody, or antigen binding fragment thereof, has a half maximal effective concentration (EC50) of about 100 nM or less (e.g., about 100 nM or less, about 90 nM or less, about 80 nM or less, about 75 nM or less, about 70 nM or less, about 60 nM or less, about 50 nM or less, about 40 nM or less, about 30 nM or less, about 25 nM or less, about 20 nM or less, about 10 nM or less, about 5 nM or less, about 2 nM or less, about 1 nM or less, about 0.5 nM or less, about 0.4 nM or less, about 0.3 nM or less, about 0.2 nM or less, about 0.1 nM or less, etc.) for increasing macrophage phagocytosis of tumor cells in vitro. In some embodiments, the antibody, or antigen binding fragment thereof, has an EC50 of 10 nM or less for increasing macrophage phagocytosis of tumor cells in vitro. In some embodiments, the antibody, or antigen binding fragment thereof, has an EC50 of 5 nM or less for increasing macrophage phagocytosis of tumor cells in vitro. In some embodiments, the antibody, or antigen binding fragment thereof, has an EC50 of 1 nM or less for increasing macrophage phagocytosis of tumor cells in vitro. In some embodiments, the antibody, or antigen binding fragment thereof, results in a maximum macrophage phagocytosis of tumor cells in vitro of about 20% or greater (e.g., about 20% or greater, about 30% or greater, about 40% or greater, about 50% or greater, about 60% or greater, about 70% or greater, about 80% or greater, about 90% or greater, about 95% or greater, etc.) when provided at a concentration of 1 μM or greater. In certain embodiments, the antibody, or antigen binding fragment thereof, results in an increase in maximum macrophage phagocytosis of tumor cells in vitro of about 50% or greater (e.g., about 50% or greater, about 2 fold or greater, about 3 fold or greater, about 4 fold or greater, about 5 fold or greater, about 10 fold or greater, etc.) when provided at a concentration of 1 μM or greater, as compared to an antibody, or antigen binding fragment thereof, that does not bind CD47 when provided at the same concentration. In certain embodiments, the tumor cells comprise a B cell lymphoma cell line (e.g., Raji cell line). In further embodiments, the tumor cells comprise a T cell lymphoma cell line (e.g., CEM cell line). Methods of measuring the EC50 of an antibody, or antigen binding fragment thereof, to increase macrophage phagocytosis of tumor cells in vitro may be carried out using any method known in the art, including, for example, flow cytometry (See e.g., Example 9 below).

In some embodiments, the antibody, or antigen binding fragment thereof, has antibody-dependent cellular cytotoxicity (ADCC) activity. ADCC is mediated by immune cells (e.g., natural killer (NK) cells) and plays an important role in the humoral immune response (e.g., to infection, to tumor cells, etc.). In some embodiments, the antibody, or antigen binding fragment thereof, has ADCC activity resulting in tumor cell lysis of about 5% or greater (e.g., about 5% or greater, about 10% or greater, about 15% or greater, about 20% or greater, about 25% or greater, about 30% or greater, about 40% or greater, about 50% or greater, etc.) when provided at a concentration of 0.01 nM or greater. In some embodiments, the antibody, or antigen binding fragment thereof, has ADCC activity resulting in tumor cell lysis of about 10% or greater (e.g., about 10% or greater, about 15% or greater, about 20% or greater, about 25% or greater, about 30% or greater, about 40% or greater, about 50% or greater, etc.) when provided at a concentration of 0.1 nM or greater. In some embodiments, the antibody, or antigen binding fragment thereof, has ADCC activity resulting in tumor cell lysis of about 20% or greater (e.g., about 20% or greater, about 25% or greater, about 30% or greater, about 40% or greater, about 50% or greater, etc.) when provided at a concentration of 1 nM or greater. In certain embodiments, the tumor cells comprise a B cell lymphoma cell line (e.g., Raji cell line). In further embodiments, the tumor cells comprise a T cell lymphoma cell line (e.g., CEM cell line). Methods of measuring ADCC of antibodies and antigen binding fragments are known in the art and include, for example, the method described in Example 10 below.

In some embodiments, the antibodies, antigen-binding fragments, or activatable antibodies are cross-reactive with monkey (e.g., cynomolgus monkey), rat, and/or dog CD47. In some embodiments, the antibodies, antigen-binding fragments, or activatable antibodies are cross-reactive with monkey CD47. In some embodiments, the antibodies, antigen-binding fragments, or activatable antibodies are cross-reactive with rat CD47. In some embodiments, the antibodies, antigen-binding fragments, or activatable antibodies are cross-reactive with dog CD47. In some embodiments, antibodies, antigen-binding fragments, or activatable antibodies are cross reactive with monkey and rat CD47; monkey and dog CD47; rat and dog CD47; or monkey, rat, and dog CD47. In some embodiments, the antibodies, antigen-binding fragments, or activatable antibodies are cross-reactive at about 100 nM or less (e.g., at about 1 nM, at about 10 nM, at about 25 nM, at about 50 nM, at about 75 nM, at about 100 nM) with monkey (e.g., cynomolgus monkey), rat, and/or dog CD47. Methods of measuring cross-reactivity of an antibody, or antigen binding fragment thereof, are known in the art, including, without limitation, surface plasmon resonance, an ELISA, isothermal titration calorimetry, a filter binding assay, an EMSA, etc. In some embodiments, the cross-reactivity is measured by ELISA (Scc e.g., Example 5 below).

In certain embodiments, the antibodies, antigen-binding fragments, or activatable antibodies are cross-reactive with cynomolgus monkey CD47. In some embodiments, the antibody, or antigen binding fragment thereof, has a half maximal effective concentration (EC50) of about 100 nM or less (e.g., about 100 nM or less, about 90 nM or less, about 80 nM or less, about 75 nM or less, about 70 nM or less, about 60 nM or less, about 50 nM or less, about 40 nM or less, about 30 nM or less, about 25 nM or less, about 20 nM or less, about 10 nM or less, about 5 nM or less, about 2 nM or less, about 1 nM or less, about 0.5 nM or less, about 0.4 nM or less, about 0.3 nM or less, about 0.2 nM or less, about 0.1 nM or less, etc.) for binding to cynomolgus monkey CD47 in vitro. In some embodiments, the antibody, or antigen binding fragment thereof, has an EC50 of about 10 nM or less for binding to cynomolgus monkey CD47 in vitro. In some embodiments, the antibody, or antigen binding fragment thereof, has an EC50 of about 5 nM or less for binding to cynomolgus monkey CD47 in vitro. In some embodiments, the antibody, or antigen binding fragment thereof, has an EC50 of about 1 nM or less for binding to cynomolgus monkey CD47 in vitro.

Antibodies of the present disclosure can be produced by techniques known in the art, including conventional monoclonal antibody methodology e.g., the standard somatic cell hybridization technique (See e.g., Kohler and Milstein, Nature 256:495 (1975), viral or oncogenic transformation of B lymphocytes, or recombinant antibody technologies as described in detail herein below.

Hybridoma production is a very well-established procedure. The common animal system for preparing hybridomas is the murine system. Immunization protocols and techniques for isolation of immunized splenocytes for fusion are known in the art. Fusion partners (e.g., murine myeloma cells) and fusion procedures are also known. One well-known method that may be used for making human CD47 antibodies provided by the present disclosure involves the use of a XenoMouse™ animal system. XenoMouse™ mice are engineered mouse strains that comprise large fragments of human immunoglobulin heavy chain and light chain loci and are deficient in mouse antibody production. See, e.g., Green et al., Nature Genetics 7:13-21 (1994) and WO2003/040170. The animal is immunized with a CD47 antigen. The CD47 antigen is isolated and/or purified CD47, preferably CD47. It may be a fragment of CD47, such as the extracellular domain (ECD) of CD47, particularly a CD47 ECD fragment comprising amino acid resides 34-108 or 34-93 of SEQ ID NO: 1. Immunization of animals can be carried out by any method known in the art. Sec, e.g., Harlow and Lane, Antibodies: A Laboratory Manual, New York: Cold Spring Harbor Press, 1990. Methods for immunizing non-human animals such as mice, rats, sheep, goats, pigs, cattle and horses are well known in the art. See, e.g., Harlow and Lane, supra, and U.S. Pat. No. 5,994,619. The CD47 antigen may be administered with an adjuvant to stimulate the immune response. Exemplary adjuvants include complete or incomplete Freund's adjuvant, RIBI (muramyl dipeptides) or ISCOM (immunostimulating complexes). After immunization of an animal with a CD47 antigen, antibody-producing immortalized cell lines are prepared from cells isolated from the immunized animal. After immunization, the animal is sacrificed and lymph node and/or splenic B cells are immortalized. Methods of immortalizing cells include, but are not limited to, transferring them with oncogenes, inflecting them with the oncogenic virus cultivating them under conditions that select for immortalized cells, subjecting them to carcinogenic or mutating compounds, fusing them with an immortalized cell, e.g., a myeloma cell, and inactivating a tumor suppressor gene. Sec, e.g., Harlow and Lane, supra. If fusion with myeloma cells is used, the myeloma cells preferably do not secrete immunoglobulin polypeptides (a non-secretory cell line). Immortalized cells are screened using CD47, a portion thereof, or a cell expressing CD47. CD47 antibody-producing cells, e.g., hybridomas, are selected, cloned and further screened for desirable characteristics, including robust growth, high antibody production and desirable antibody characteristics, as discussed further below. Hybridomas can be expanded in vivo in syngeneic animals, in animals that lack an immune system, e.g., nude mice, or in cell culture in vitro. Methods of selecting, cloning and expanding hybridomas are well known to those of ordinary skill in the art.

Antibodies of the disclosure can also be prepared using phage display or yeast display methods. Such display methods for isolating human antibodies are established in the art, such as Achim Knappik, et al., “Fully Synthetic Human Combinatorial Antibody Libraries (HuCAL) Based on Modular Consensus Frameworks and CDRs Randomized with Trinucleotides.” J. Mol. Biol. (2000) 296, 57-86; and Michael J. Feldhaus, et al, “Flow-cytometric isolation of human antibodies from a non-immune Saccharomyces cerevisiae surface display library” Nat Biotechnol (2003) 21:163-170.

### B-2. Masked Antibodies

In some aspects, provided herein are masked antibodies that bind to CD47. In some embodiments, the masked antibody comprises (a) a masking peptide comprising, from N terminus to C terminus, a masking moiety (MM) and a linkage moiety (LM); and (b) a target binding moiety (TBM) comprising an antibody heavy chain variable region (VH) and an antibody light chain variable region (VL), wherein the TBM binds to human CD47. In certain embodiments, the masking peptide is linked to the N terminus of the VH or the VL. In preferred embodiments, the MM competes with human CD47 to bind the TBM. The TBM may comprise one or more sequences of the anti-CD47 antibodies or antigen-binding fragments described herein, including antibodies or antigen-binding fragments described with reference to specific amino acid sequences of CDRs, variable regions (VL, VH), and/or light and heavy chains (e.g., IgG1, IgG2, IgG4). In some embodiments, TBM comprises a full length antibody light chain and/or a full length antibody heavy chain of one or more of the anti-CD47 antibodies described herein.

In some embodiments, the present disclosure relates to masked antibodies that bind to human CD47, and have at least one (e.g., at least one, at least two, at least three, at least four, at least five, at least six, at least seven, at least eight, at least nine, at least 10, at least 11, at least 12, or all 13) of the following functional properties: (a) has a greater Kp for binding to human CD47 as compared to a parental antibody having the same TBM but lacking the masking peptide; (b) has a greater half maximal effective concentration (EC50) for binding to human CD47 as compared to a parental antibody having the same TBM but lacking the masking peptide; (c) has a higher half maximal inhibitory concentration (IC50) for blocking binding of human CD47 to human SIRPα in vitro as compared to a parental antibody having the same TBM but lacking the masking peptide; (d) completely blocks binding of human CD47 to human SIRPα in vitro when the masked antibody is provided at a concentration of about 1 nM or greater; (e) has a greater EC50 for binding to tumor cells in vitro as compared to a parental antibody having the same TBM but lacking the masking peptide; (f) has a reduced binding to red blood cells (RBCs) in vitro as compared to a parental antibody having the same TBM but lacking the masking peptide; (g) has a greater EC50 for increasing macrophage phagocytosis of tumor cells in vitro as compared to a parental antibody having the same TBM but lacking the masking peptide; (h) binds to human CD47 with a Kp of 500 nM or less; (i) is cross-reactive with monkey, rat, or dog CD47; (j) is capable of inhibiting tumor cell growth; (k) has one or more therapeutic effects on a cancer; (1) blocks binding between CD47 and SIRP proteins (e.g., SIRPα); and (m) exhibits antibody-dependent cellular cytotoxicity (ADCC) against CD47-expressing tumor cells.

In some embodiments, the masked antibody selectively binds to tissues and cells with high CD47 expression levels. Without wishing to be bound by theory, because the MM competes with human CD47 to bind the TBM, the masked antibody selectively binds to CD47 in cells and tissues with adequately high CD47 expression levels and, thus, adequately high local concentrations of CD47 on the cell surface. This phenomenon of binding to the target (e.g., CD47) selectively in tissues and on cells having high expression levels of the target (e.g., CD47) may be referred to as selective target engagement. In certain embodiments, selective target engagement of an anti-CD47 masked antibody described herein results in a reduction of on-target, off-tumor effects (e.g., antigen sink effects, anemia) as compared to an anti-CD47 antibody having the same TBM but lacking the masking peptide comprising the masking moiety.

In some embodiments, the masked antibody comprises a target-binding moiety (TBM). In some embodiments, the TBM comprises an antibody light chain variable region and/or an antibody heavy chain variable region. In some embodiments, the TBM comprises an antibody light chain variable region. In some embodiments, the TBM comprises an antibody heavy chain variable region. In some embodiments, the TBM comprises an antibody light chain variable region and an antibody heavy chain variable region. In some embodiments, the antibody heavy chain variable region is C-terminal to the antibody light chain variable region. In some embodiments, the antibody light chain variable region is C-terminal to the antibody heavy chain variable region. In some embodiments, a TBM of the present disclosure comprises an antibody light chain variable region and/or an antibody heavy chain variable region with specificity for CD47. In some embodiments, the TBM comprises a full length antibody light chain and/or a full length antibody heavy chain. The antibody light chain may be a kappa or lambda light chain. The antibody heavy chain may be in any class, such as IgG, IgM, IgE, IgA, or IgD. In some embodiments, the antibody heavy chain is in the IgG class, such as IgG1, IgG2, IgG3, or lgG4 subclass. An antibody heavy chain described herein may be converted from one class or subclass to another class or subclass using methods known in the art. Any one or more of the TBMs described herein may incorporate any of the CDR sequences (e.g., one, two, or three of the heavy chain variable region CDR sequences, and/or one, two, or three of the light chain variable region CDR sequences), heavy chain variable region sequences, and/or light chain variable region sequences of any of the anti-CD47 antibodies described herein.

In some embodiments, the masked antibody, or an antigen-binding fragment thereof, comprises a first polypeptide comprising, from N terminus to C terminus, the masking peptide and the VL, and a second polypeptide comprising the VH (e.g., a Fab fragment). In other embodiments, the masked antibody or an antigen-binding fragment thereof, comprises a first polypeptide comprising, from N terminus to C terminus, the masking peptide and the VH, and a second polypeptide comprising the VL (e.g., a Fab fragment). In other embodiments, the masked antibody, or an antigen-binding fragment thereof, comprises a first polypeptide comprising, from N terminus to C terminus, the masking peptide, the VL, and the VH (e.g., an scFv). In yet additional embodiments, the masked antibody, or an antigen-binding fragment thereof, comprises a first polypeptide comprising, from N terminus to C terminus the masking peptide, the VH, and the VL (e.g., an scFv).

In some embodiments, the masked antibody, or an antigen-binding fragment thereof, comprises a first polypeptide comprising, from N terminus to C terminus, the masking peptide and the VL, a second polypeptide comprising the VH, a third polypeptide comprising the masking peptide and the VL, and a fourth polypeptide comprising the VH. In certain embodiments, the masked antibody comprises a first polypeptide comprising, from N terminus to C terminus, the masking peptide and the VL, a second polypeptide comprising the VH and a first Fc domain, a third polypeptide comprising the masking peptide and the VL, and a fourth polypeptide comprising the VH and a second Fc domain. In certain embodiments, the first and second Fc domains are the same. In other embodiments, the first and second Fc domains are different. In some embodiments, the first and second Fc domains are both IgG4 Fc domains (e.g., human IgG4 Fc domains). The first and second IgG4 Fc domains may be the same IgG4 Fc domain or different IgG4 Fc domains. In other embodiments, the first and second Fc domains are both IgG1 Fc domains (e.g., human IgG1 Fc domains). The first and second IgG1 Fc domains may be the same IgG1 Fc domain or different IgG1 Fc domains. In some embodiments, the first and second IgG1 Fc domains each comprise a S239D substitution and/or an 1332E substitution. In certain embodiments, the first and second IgG1 Fc domains each comprise a S239D substitution and an 1332E substitution.

In some embodiments, the masked antibody comprises a masking peptide comprising, from N terminus to C terminus, and MM and a LM. In some embodiments, the MM comprises an amino acid sequence according to Formula (XVI): X1X2X3X4X5X6CX7DDX8X9X10CX11X12 (SEQ ID NO: 197), wherein X1 is D, H, N, or Y, X2 is A, D, F. P. T. or Y, X3 is A, L. N. P. T, or Y, X4 is A, D. H, or S, X5 is A, D, F. H, or N, X6 is D, S, or T. X7 is D, S, or Y, X8 is D, F, or Y, X9 and X11 are each independently A, D, or Y, X10 is A, D, F, or P, and X12 is D, F. I. T, or Y. In certain embodiments, the MM comprises an amino acid sequence according to Formula (XVII): X1X2X3X4X5DCPX6X7DX8X9CX10X11 (SEQ ID NO: 198), wherein X1 is A, N, or P, X2 is A, N. P, or Q, X3 is A, D, or S, X4 is A, D, S, or V, X5 is D, F, or P, X6 is A, D, or T, X7 is A, H, or Y, X8 is A, D, or V, X9 is For Y, X10 is D, N, S, or Y, and X11 is D, P, or V. In other embodiments, the MM comprises an amino acid sequence according to Formula (XVIII): X1X2X3X4X5X6CDX7X8X9X10X11CX12A (SEQ ID NO: 199), wherein X1 is A or L, X2 is A or T, X3 is S or V. X4 is D or P. X5 is A or Y, X6 is For T, X7 is D or I, X8 is D or T, X9 is L or P, X10 is For L, X11 is For Y, and X12 is N or P. In yet other embodiments, the MM comprises an amino acid sequence according to Formula (XIX): X1X2CX3X4X5X6X7X8X9FCX10X11 (SEQ ID NO: 200), wherein X1 is D, F, or V, X2 is A. S, or Y, X3 is P. R. or T, X4 is A, G, or I, X5 is A, E, or F. X6 is A, D, or V, X7 is D or V. X8 is D or G, X9 is I or P. X10 is I or S, and X11 is A, Q, or V. In some embodiments, the MM comprises an amino acid sequence selected from the group consisting of SEQ ID NOS: 137 and 167-181, or a variant thereof comprising up to 5 (e.g., 1, 2, 3, 4, or 5) amino acid substitutions. In certain embodiments, the MM comprises the amino acid sequence of SEQ ID NO: 137, or a variant thereof comprising up to 5 (e.g., 1, 2, 3, 4, or 5) amino acid substitutions. In some embodiments, the MM comprises the amino acid sequence of the MM of an illustrative antibody as described in Table 5A, or a variant thereof comprising up to 5 (e.g., 1, 2, 3, 4, or 5) amino acid substitutions. In certain embodiments, the MM comprises the amino acid sequence of the MM of illustrative antibody TY26294 as described in Table 5A, or a variant thereof comprising up to 5 (e.g., 1, 2, 3, 4, or 5) amino acid substitutions.

In some embodiments, the masking peptide further comprises one or more (e.g., one, two, three, or more) linkers. In certain embodiments, the MM further comprises one or more (e.g., one, two, three, or more) linkers. Any suitable linker (e.g., a flexible linker) known in the art may be used, including, for example: glycine polymers (G) n, where n is an integer of at least 1 (e.g., at least one, at least 2, at least 3, at least 4, at least 5, at least 6, at least 7, at least 8, at least 9, at least 10, etc.); glycine-serine polymers (GS) n, where n is an integer of at least 1 (e.g., at least one, at least 2, at least 3, at least 4, at least 5, at least 6, at least 7, at least 8, at least 9, at least 10, etc.); glycine-alanine polymers; alanine-serine polymers; and the like. Linker sequences may be of any length, such as from about 1 amino acid (e.g., glycine or serine) to about 20 amino acids (e.g., 20 amino acid glycine polymers or glycine-serine polymers), about 1 amino acid to about 15 amino acids, about 3 amino acids to about 12 amino acids, about 4 amino acids to about 10 amino acids, about 5 amino acids to about 9 amino acids, about 6 amino acids to about 8 amino acids, etc. In some embodiments, the linker is any of about 1, 2, 3, 4, 5, 6, 7, 8, 9, 10, 11, 12, 13, 14, 15, 16, 17, 18, 19, or 20 amino acids in length.

In some embodiments, any of the masking peptides described herein may further comprise one or more additional amino acid sequences (e.g., one or more polypeptide tags). Examples of suitable additional amino acid sequence may include, without limitation, purification tags (such as his-tags, flag-tags, maltose binding protein and glutathione-S-transferase tags), detection tags (such as tags that may be detected photometrically (e.g., red or green fluorescent protein, etc.)), tags that have a detectable enzymatic activity (e.g., alkaline phosphatase, etc.), tags containing secretory sequences, leader sequences, and/or stabilizing sequences, protease cleavage sites (e.g., furin cleavage sites, TEV cleavage sites, Thrombin cleavage sites), and the like. In some embodiments, the one or more additional amino acid sequences are at the N-terminus of the masking peptide.

In some embodiments, the masking peptide does not comprise a cleavable moiety (CM). In other embodiments, the masking peptide comprises a cleavable moiety (CM). In certain embodiments, the CM comprises at least one cleavage site. The cleavage site may be any cleavage site known in the art or described herein.

In some embodiments, the linkage moiety (LM) does not comprise a cleavable moiety (CM). In other embodiments, the linkage moiety (LM) comprises a cleavable moiety (CM). In certain embodiments, the CM comprises at least one cleavage site. The cleavage site may be any cleavage site known in the art or described herein.

In certain embodiments, the masked antibody comprises a first polypeptide comprising, from N terminus to C terminus, the masking peptide and the VL; and a second polypeptide comprising the VH. In some embodiments, the second polypeptide further comprises an Fc region. In some embodiments, the Fc region is an IgG4 Fc region. In some embodiments, the Fc region is an IgG1 Fc region. In certain embodiments, the Fc region is an IgG1 Fc region comprising a S239D substitution and/or an 1332E substitution, wherein numbering is according to Kabat. In certain embodiments, the Fc region is an IgG1 Fc region comprising a S239D substitution and an 1332E substitution, wherein numbering is according to Kabat. IgG1 Fc regions comprising a S239D substitution and an 1332E substitution are described in detail in Lazar et al. (Engineered antibody Fc variants with enhanced effector function.” PNAS 103.11 (2006): 4005-4010). In some embodiments, a) the VL of the first polypeptide comprises one or more amino acid sequences selected from the group consisting of SEQ ID NOs: 190-196; and/or b) the VH of the second polypeptide comprises one or more amino acid sequences selected from the group consisting of SEQ ID NOs: 182-189. In some variations, a) the VL of the first polypeptide comprises the amino acid sequences of SEQ ID NOs: 191, 194, and/or 195; and/or b) the VH of the second polypeptide comprises the amino acid sequences of SEQ ID NOs: 182, 185, and/or 188. In certain embodiments, a) the VL of the first polypeptide comprises one or more amino acid sequences selected from the group consisting of SEQ ID NOs: 31-85, or a variant thereof comprising up to 5 (e.g., 1, 2, 3, 4, or 5) amino acid substitutions; and/or b) the VH of the second polypeptide comprises one or more amino acid sequences selected from the group consisting of SEQ ID NOs: 86-136, or a variant thereof comprising up to 5 (e.g., 1, 2, 3, 4, or 5) amino acid substitutions. In some variations, a) the VL of the first polypeptide comprises a CDR-L1 comprising an amino acid sequence selected from the group consisting of SEQ ID NOs: 86-102, or a variant thereof comprising up to 5 (e.g., 1, 2, 3, 4, or 5) amino acid substitutions; a CDR-L2 comprising an amino acid sequence selected from the group consisting of SEQ ID NOs: 103-119, or a variant thereof comprising up to 5 (e.g., 1, 2, 3, 4, or 5) amino acid substitutions; and/or a CDR-L3 comprising an amino acid sequence selected from the group consisting of SEQ ID NOs: 120-136, or a variant thereof comprising up to 5 (e.g., 1, 2, 3, 4, or 5) amino acid substitutions; and/or b) the VH of the second polypeptide comprises a CDR-H1 comprising an amino acid sequence selected from the group consisting of SEQ ID NOs: 35-51, or a variant thereof comprising up to 5 (e.g., 1, 2, 3, 4, or 5) amino acid substitutions; a CDR-H2 comprising an amino acid sequence selected from the group consisting of SEQ ID NOs: 52-68, or a variant thereof comprising up to 5 (e.g., 1, 2, 3, 4, or 5) amino acid substitutions; and/or a CDR-H3 comprising an amino acid sequence selected from the group consisting of SEQ ID NOs: 69-85, or a variant thereof comprising up to 5 (e.g., 1, 2, 3, 4, or 5) amino acid substitutions. In certain embodiments, a) the VL of the first polypeptide comprises the amino acid sequence of SEQ ID NO: 99, or a variant thereof comprising up to 5 (e.g., 1, 2, 3, 4, or 5) amino acid substitutions; the amino acid sequence of SEQ ID NO: 116, or a variant thereof comprising up to 5 (e.g., 1, 2, 3, 4, or 5) amino acid substitutions; and/or the amino acid sequence of SEQ ID NO: 133 or a variant thereof comprising up to 5 (e.g., 1, 2, 3, 4, or 5) amino acid substitutions; and/or b) the VH of the second polypeptide comprises the amino acid sequence of SEQ ID NO: 48, or a variant thereof comprising up to 5 (e.g., 1, 2, 3, 4, or 5) amino acid substitutions; the amino acid sequence of SEQ ID NO: 65, or a variant thereof comprising up to 5 (e.g., 1, 2, 3, 4, or 5) amino acid substitutions; and/or the amino acid sequence of SEQ ID NO: 82, or a variant thereof comprising up to 5 (e.g., 1, 2, 3, 4, or 5) amino acid substitutions. In some embodiments, the VL of the first polypeptide comprises one, two, or three CDRs of illustrative antibody TY21446 as shown in Table 3B, or variants thereof comprising up to 5 (e.g., 1, 2, 3, 4, or 5) amino acid substitutions; and/or the VH of the second polypeptide comprises one, two, or three CDRs of illustrative antibody TY21446 as shown in Table 3A, or variants thereof comprising up to 5 (e.g., 1, 2, 3, 4, or 5) amino acid substitutions. In some embodiments, the VL of the first polypeptide comprises the amino acid sequence of SEQ ID NO: 28, or an amino acid sequence having at least 80% (e.g., at least 85%, 90%, 95%, 98%, or 99%; or 100%) sequence identity with the amino acid sequence of SEQ ID NO: 28; and the VH of the second polypeptide comprises the amino acid sequence of SEQ ID NO: 27, or an amino acid sequence having at least 80% (e.g., at least 85%, 90%, 95%, 98%, or 99%; or 100%) sequence identity with the amino acid sequence of SEQ ID NO: 27. In certain embodiments, the first polypeptide comprises the VL of illustrative antibody TY21446 as shown in Table 4A, or an amino acid sequence having at least 80% (e.g., at least 85%, 90%, 95%, 98%, or 99%; or 100%) sequence identity with the VL of illustrative antibody TY21446 as shown in Table 4A; and the second polypeptide comprises the VH of illustrative antibody TY21446 as shown in Table 4A, or an amino acid sequence having at least 80% (e.g., at least 85%, 90%, 95%, 98%, or 99%; or 100%) sequence identity with the VH of illustrative antibody TY21446 as shown in Table 4A.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VL; and wherein the MM competes with human CD47 to bind the TBM; wherein: 1) the VH comprises a CDR-H1 comprising the amino acid sequence of SEQ ID NO: 48, or a variant thereof comprising up to 5 (e.g., 1, 2, 3, 4, or 5) amino acid substitutions; a CDR-H2 comprising the amino acid sequence of SEQ ID NO: 65, or a variant thereof comprising up to 5 (e.g., 1, 2, 3, 4, or 5) amino acid substitutions; and a CDR-H3 comprising the amino acid sequence of SEQ ID NO: 82, or a variant thereof comprising up to 5 (e.g., 1, 2, 3, 4, or 5) amino acid substitutions; and 2) the VL comprises a CDR-L1 comprising the amino acid sequence of SEQ ID NO: 99, or a variant thereof comprising up to 5 (e.g., 1, 2, 3, 4, or 5) amino acid substitutions; a CDR-L2 comprising the amino acid sequence of SEQ ID NO: 116, or a variant thereof comprising up to 5 (e.g., 1, 2, 3, 4, or 5) amino acid substitutions; and a CDR-L3 comprising the amino acid sequence of SEQ ID NO: 133, or a variant thereof comprising up to 5 (e.g., 1, 2, 3, 4, or 5) amino acid substitutions. In some embodiments, the masked antibody comprises (a) a masking peptide comprising, from N terminus to C terminus, an MM and a LM, and (b) a TBM comprising a VH and a VL, wherein the TBM binds to human CD47; wherein the masking peptide is linked to the N terminus of the VL; and wherein the MM competes with human CD47 to bind the TBM; wherein: 1) the VH comprises a CDR-H1 comprising the amino acid sequence of SEQ ID NO: 48; a CDR-H2 comprising the amino acid sequence of SEQ ID NO: 65; and a CDR-H3 comprising the amino acid sequence of SEQ ID NO: 82; and 2) the VL comprises a CDR-L1 comprising the amino acid sequence of SEQ ID NO: 99; a CDR-L2 comprising the amino acid sequence of SEQ ID NO: 116; and a CDR-L3 comprising the amino acid sequence of SEQ ID NO: 133. In some embodiments, the masked antibody comprises (a) a masking peptide comprising, from N terminus to C terminus, an MM and a LM, and (b) a TBM comprising a VH and a VL, wherein the TBM binds to human CD47; wherein the masking peptide is linked to the N terminus of the VL; and wherein the MM competes with human CD47 to bind the TBM; wherein: 1) the VH comprises the amino acid sequence of SEQ ID NO: 48, or a variant thereof comprising up to 5 (e.g., 1, 2, 3, 4, or 5) amino acid substitutions; the amino acid sequence of SEQ ID NO: 65, or a variant thereof comprising up to 5 (e.g., 1, 2, 3, 4, or 5) amino acid substitutions; and the amino acid sequence of SEQ ID NO: 82, or a variant thereof comprising up to 5 (e.g., 1, 2, 3, 4, or 5) amino acid substitutions; and 2) the VL comprises the amino acid sequence of SEQ ID NO: 99, or a variant thereof comprising up to 5 (e.g., 1, 2, 3, 4, or 5) amino acid substitutions; the amino acid sequence of SEQ ID NO: 116, or a variant thereof comprising up to 5 (e.g., 1, 2, 3, 4, or 5) amino acid substitutions; and the amino acid sequence of SEQ ID NO: 133, or a variant thereof comprising up to 5 (e.g., 1, 2, 3, 4, or 5) amino acid substitutions. In some embodiments, the masked antibody comprises one, two, three, four, five, or six CDRs of illustrative antibody TY21446 as shown in Tables 3A-3B, or variants thereof comprising up to 5 (e.g., 1, 2, 3, 4, or 5) amino acid substitutions. In some embodiments, the MM comprises the amino acid sequence of SEQ ID NO: 137, or a variant thereof comprising up to 5 (e.g., 1, 2, 3, 4, or 5) amino acid substitutions. In some embodiments, the MM comprises the amino acid sequence of the MM of illustrative antibody TY26294 as described in Table 5A, or a variant thereof comprising up to 5 (e.g., 1, 2, 3, 4, or 5) amino acid substitutions. In some embodiments, the masked antibody comprises (a) a masking peptide comprising, from N terminus to C terminus, an MM and a LM, and (b) a TBM comprising a VH and a VL, wherein the TBM binds to human CD47; wherein the masking peptide is linked to the N terminus of the VL; and wherein the MM competes with human CD47 to bind the TBM; wherein: 1) the VH comprises the amino acid sequence of SEQ ID NO: 27, or an amino acid sequence having at least 80% (e.g., at least 85%, 90%, 95%, 98%, or 99%; or 100%) sequence identity with the amino acid sequence of SEQ ID NO: 27; and 2) the VL comprises the amino acid sequence of SEQ ID NO: 28, or an amino acid sequence having at least 80% (e.g., at least 85%, 90%, 95%, 98%, or 99%; or 100%) sequence identity with the amino acid sequence of SEQ ID NO: 28. In some embodiments, the masked antibody comprises the VH and/or the VL of illustrative antibody TY21446 as shown in Table 4A, or one or more amino acid sequences having at least 80% (e.g., at least 85%, 90%, 95%, 98%, or 99%; or 100%) sequence identity with a VH or a VL of TY21446 as shown in Table 4A. In some embodiments, the masked antibody comprises an IgG1 Fc region. In certain embodiments, the masked antibody comprises a human IgG1 Fc region. In some embodiments, the IgG1 Fc region comprises a S239D substitution and/or an 1332E substitution. In some embodiments, the IgG1 Fc region comprises a S239D substitution and an 1332E substitution. In certain embodiments, the human IgG1 Fc region comprises a S239D substitution and an 1332E substitution. In certain embodiments, the human IgG1 Fc region comprises two Fc domains, wherein each of the two Fc domains comprises a S239D substitution and/or an 1332E substitution (e.g., a S239D substitution and an 1332E substitution). In some embodiments, the IgG1 Fc region has enhanced antibody-dependent cellular cytotoxic (ADCC) and/or antibody-dependent cellular phagocytosis (ADCP), function(s). In some embodiments, the masked antibody comprises an IgG4 Fc region. In certain embodiments, the masked antibody comprises a human IgG4 Fc region.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VL; and wherein the MM competes with human CD47 to bind the TBM; wherein: 1) the VH comprises a CDR-H1 comprising the amino acid sequence of SEQ ID NO: 49, or a variant thereof comprising up to 5 (e.g., 1, 2, 3, 4, or 5) amino acid substitutions; a CDR-H2 comprising the amino acid sequence of SEQ ID NO: 66, or a variant thereof comprising up to 5 (e.g., 1, 2, 3, 4, or 5) amino acid substitutions; and a CDR-H3 comprising the amino acid sequence of SEQ ID NO: 83, or a variant thereof comprising up to 5 (e.g., 1, 2, 3, 4, or 5) amino acid substitutions; and 2) the VL comprises a CDR-L1 comprising the amino acid sequence of SEQ ID NO: 100, or a variant thereof comprising up to 5 (e.g., 1, 2, 3, 4, or 5) amino acid substitutions; a CDR-L2 comprising the amino acid sequence of SEQ ID NO: 117, or a variant thereof comprising up to 5 (e.g., 1, 2, 3, 4, or 5) amino acid substitutions; and a CDR-L3 comprising the amino acid sequence of SEQ ID NO: 134, or a variant thereof comprising up to 5 (e.g., 1, 2, 3, 4, or 5) amino acid substitutions. In some embodiments, the masked antibody comprises (a) a masking peptide comprising, from N terminus to C terminus, an MM and a LM, and (b) a TBM comprising a VH and a VL, wherein the TBM binds to human CD47; wherein the masking peptide is linked to the N terminus of the VL; and wherein the MM competes with human CD47 to bind the TBM; wherein: 1) the VH comprises a CDR-H1 comprising the amino acid sequence of SEQ ID NO: 49; a CDR-H2 comprising the amino acid sequence of SEQ ID NO: 66; and a CDR-H3 comprising the amino acid sequence of SEQ ID NO: 83; and 2) the VL comprises a CDR-L1 comprising the amino acid sequence of SEQ ID NO: 100; a CDR-L2 comprising the amino acid sequence of SEQ ID NO: 117; and a CDR-L3 comprising the amino acid sequence of SEQ ID NO: 134. In some embodiments, the masked antibody comprises (a) a masking peptide comprising, from N terminus to C terminus, an MM and a LM, and (b) a TBM comprising a VH and a VL, wherein the TBM binds to human CD47; wherein the masking peptide is linked to the N terminus of the VL; and wherein the MM competes with human CD47 to bind the TBM; wherein: 1) the VH comprises the amino acid sequence of SEQ ID NO: 49, or a variant thereof comprising up to 5 (e.g., 1, 2, 3, 4, or 5) amino acid substitutions; the amino acid sequence of SEQ ID NO: 66, or a variant thereof comprising up to 5 (e.g., 1, 2, 3, 4, or 5) amino acid substitutions; and the amino acid sequence of SEQ ID NO: 83, or a variant thereof comprising up to 5 (e.g., 1, 2, 3, 4, or 5) amino acid substitutions; and 2) the VL comprises the amino acid sequence of SEQ ID NO: 100, or a variant thereof comprising up to 5 (e.g., 1, 2, 3, 4, or 5) amino acid substitutions; the amino acid sequence of SEQ ID NO: 117, or a variant thereof comprising up to 5 (e.g., 1, 2, 3, 4, or 5) amino acid substitutions; and the amino acid sequence of SEQ ID NO: 134, or a variant thereof comprising up to 5 (e.g., 1, 2, 3, 4, or 5) amino acid substitutions. In some embodiments, the masked antibody comprises one, two, three, four, five, or six CDRs of illustrative antibody TY21447 as shown in Tables 3A-3B, or variants thereof comprising up to 5 (e.g., 1, 2, 3, 4, or 5) amino acid substitutions. In some embodiments, the MM comprises the amino acid sequence of SEQ ID NO: 137, or a variant thereof comprising up to 5 (e.g., 1, 2, 3, 4, or 5) amino acid substitutions. In some embodiments, the MM comprises the amino acid sequence of the MM of illustrative antibody TY26294 as described in Table 5A, or a variant thereof comprising up to 5 (e.g., 1, 2, 3, 4, or 5) amino acid substitutions. In some embodiments, the masked antibody comprises (a) a masking peptide comprising, from N terminus to C terminus, an MM and a LM, and (b) a TBM comprising a VH and a VL, wherein the TBM binds to human CD47; wherein the masking peptide is linked to the N terminus of the VL; and wherein the MM competes with human CD47 to bind the TBM; wherein: 1) the VH comprises the amino acid sequence of SEQ ID NO: 29, or an amino acid sequence having at least 80% (e.g., at least 85%, 90%, 95%, 98%, or 99%; or 100%) sequence identity with the amino acid sequence of SEQ ID NO: 27; and 2) the VL comprises the amino acid sequence of SEQ ID NO: 28, or an amino acid sequence having at least 80% (e.g., at least 85%, 90%, 95%, 98%, or 99%; or 100%) sequence identity with the amino acid sequence of SEQ ID NO: 30. In some embodiments, the masked antibody comprises the VH and/or the VL of illustrative antibody TY21447 as shown in Table 4A, or one or more amino acid sequences having at least 80% (e.g., at least 85%, 90%, 95%, 98%, or 99%; or 100%) sequence identity with a VH or a VL of TY21447 as shown in Table 4A. In some embodiments, the masked antibody comprises an IgG1 Fc region. In certain embodiments, the IgG1 Fc region comprises a S239D substitution and/or an 1332E substitution. In some embodiments, the masked antibody comprises a human IgG1 Fc region. In certain embodiments, the human IgG1 Fc region comprises a S239D substitution and/or an 1332E substitution. In certain embodiments, the human IgG1 Fc region comprises two Fc domains, wherein each of the two Fc domains comprises a S239D substitution and/or an 1332E substitution (e.g., a S239D substitution and an 1332E substitution). In some embodiments, the IgG1 Fc region has enhanced antibody-dependent cellular cytotoxic (ADCC) and/or antibody-dependent cellular phagocytosis (ADCP), function(s). In some embodiments, the masked antibody comprises an IgG4 Fc region. In certain embodiments, the masked antibody comprises a human IgG4 Fc region.

In some embodiments, provided herein is a masked antibody comprising: (a) a masking peptide comprising, from N terminus to C terminus, a masking moiety (MM) and a linkage moiety (LM), and (b) a target binding moiety (TBM) comprising an antibody heavy chain variable region (VH) and an antibody light chain variable region (VL), wherein the TBM binds to human CD47; wherein the masking peptide is linked to the N terminus of the VL; and wherein the MM competes with human CD47 to bind the TBM; wherein: 1) the VH comprises a CDR-H1 comprising the amino acid sequence of SEQ ID NO: 50, or a variant thereof comprising up to 5 (e.g., 1, 2, 3, 4, or 5) amino acid substitutions; a CDR-H2 comprising the amino acid sequence of SEQ ID NO: 67, or a variant thereof comprising up to 5 (e.g., 1, 2, 3, 4, or 5) amino acid substitutions; and a CDR-H3 comprising the amino acid sequence of SEQ ID NO: 84, or a variant thereof comprising up to 5 (e.g., 1, 2, 3, 4, or 5) amino acid substitutions; and 2) the VL comprises a CDR-L1 comprising the amino acid sequence of SEQ ID NO: 101, or a variant thereof comprising up to 5 (e.g., 1, 2, 3, 4, or 5) amino acid substitutions; a CDR-L2 comprising the amino acid sequence of SEQ ID NO: 118, or a variant thereof comprising up to 5 (e.g., 1, 2, 3, 4, or 5) amino acid substitutions; and a CDR-L3 comprising the amino acid sequence of SEQ ID NO: 135, or a variant thereof comprising up to 5 (e.g., 1, 2, 3, 4, or 5) amino acid substitutions. In some embodiments, the masked antibody comprises (a) a masking peptide comprising, from N terminus to C terminus, an MM and a LM, and (b) a TBM comprising a VH and a VL, wherein the TBM binds to human CD47; wherein the masking peptide is linked to the N terminus of the VL; and wherein the MM competes with human CD47 to bind the TBM; wherein: 1) the VH comprises a CDR-H1 comprising the amino acid sequence of SEQ ID NO: 50; a CDR-H2 comprising the amino acid sequence of SEQ ID NO: 67; and a CDR-H3 comprising the amino acid sequence of SEQ ID NO: 84; and 2) the VL comprises a CDR-L1 comprising the amino acid sequence of SEQ ID NO: 101; a CDR-L2 comprising the amino acid sequence of SEQ ID NO: 118; and a CDR-L3 comprising the amino acid sequence of SEQ ID NO: 135. In some embodiments, the masked antibody comprises (a) a masking peptide comprising, from N terminus to C terminus, an MM and a LM, and (b) a TBM comprising a VH and a VL, wherein the TBM binds to human CD47; wherein the masking peptide is linked to the N terminus of the VL; and wherein the MM competes with human CD47 to bind the TBM; wherein: 1) the VH comprises the amino acid sequence of SEQ ID NO: 50, or a variant thereof comprising up to 5 (e.g., 1, 2, 3, 4, or 5) amino acid substitutions; the amino acid sequence of SEQ ID NO: 67, or a variant thereof comprising up to 5 (e.g., 1, 2, 3, 4, or 5) amino acid substitutions; and the amino acid sequence of SEQ ID NO: 84, or a variant thereof comprising up to 5 (e.g., 1, 2, 3, 4, or 5) amino acid substitutions; and 2) the VL comprises the amino acid sequence of SEQ ID NO: 101, or a variant thereof comprising up to 5 (e.g., 1, 2, 3, 4, or 5) amino acid substitutions; the amino acid sequence of SEQ ID NO: 118, or a variant thereof comprising up to 5 (e.g., 1, 2, 3, 4, or 5) amino acid substitutions; and the amino acid sequence of SEQ ID NO: 135, or a variant thereof comprising up to 5 (e.g., 1, 2, 3, 4, or 5) amino acid substitutions. In some embodiments, the masked antibody comprises one, two, three, four, five, or six CDRs of illustrative antibody TY21449 as shown in Tables 3A-3B, or variants thereof comprising up to 5 (e.g., 1, 2, 3, 4, or 5) amino acid substitutions. In some embodiments, the MM comprises the amino acid sequence of SEQ ID NO: 137, or a variant thereof comprising up to 5 (e.g., 1, 2, 3, 4, or 5) amino acid substitutions. In some embodiments, the MM comprises the amino acid sequence of the MM of illustrative antibody TY26294 as described in Table 5A, or a variant thereof comprising up to 5 (e.g., 1, 2, 3, 4, or 5) amino acid substitutions. In some embodiments, the masked antibody comprises (a) a masking peptide comprising, from N terminus to C terminus, an MM and a LM, and (b) a TBM comprising a VH and a VL, wherein the TBM binds to human CD47; wherein the masking peptide is linked to the N terminus of the VL; and wherein the MM competes with human CD47 to bind the TBM; wherein: 1) the VH comprises the amino acid sequence of SEQ ID NO: 27, or an amino acid sequence having at least 80% (e.g., at least 85%, 90%, 95%, 98%, or 99%; or 100%) sequence identity with the amino acid sequence of SEQ ID NO: 31; and 2) the VL comprises the amino acid sequence of SEQ ID NO: 28, or an amino acid sequence having at least 80% (e.g., at least 85%, 90%, 95%, 98%, or 99%; or 100%) sequence identity with the amino acid sequence of SEQ ID NO: 32. In some embodiments, the masked antibody comprises the VH and/or the VL of illustrative antibody TY21449 as shown in Table 4A, or one or more amino acid sequences having at least 80% (e.g., at least 85%, 90%, 95%, 98%, or 99%; or 100%) sequence identity with a VH or a VL of TY21449 as shown in Table 4A. In some embodiments, the masked antibody comprises an IgG1 Fc region. In certain embodiments, the IgG1 Fc region comprises a S239D substitution and/or an 1332E substitution. In some embodiments, the masked antibody comprises a human IgG1 Fc region. In certain embodiments, the human IgG1 Fc region comprises a S239D substitution and/or an 1332E substitution. In certain embodiments, the human IgG1 Fc region comprises two Fc domains, wherein each of the two Fc domains comprises a S239D substitution and/or an 1332E substitution (e.g., a S239D substitution and an 1332E substitution). In some embodiments, the IgG1 Fc region has enhanced antibody-dependent cellular cytotoxic (ADCC) and/or antibody-dependent cellular phagocytosis (ADCP), function(s). In some embodiments, the masked antibody comprises an IgG4 Fc region. In certain embodiments, the masked antibody comprises a human IgG4 Fc region.

In some embodiments, the masked antibody comprises a human IgG4 Fc region. A masked antibody of the present disclosure may comprise an IgG4 Fc region and any combination of the CDR, VH, VL, and/or light chain sequences described herein. In some embodiments, the masked antibody comprises a first polypeptide comprising a light chain comprising a VL and a second polypeptide comprising a heavy chain comprising, from N terminus to C terminus, a VH and a human IgG4 Fc region. In certain embodiments, a) the VL of the first polypeptide comprises the amino acid sequence of SEQ ID NO: 99, or a variant thereof comprising up to 5 (e.g., 1, 2, 3, 4, or 5) amino acid substitutions; the amino acid sequence of SEQ ID NO: 116, or a variant thereof comprising up to 5 (e.g., 1, 2, 3, 4, or 5) amino acid substitutions; and/or the amino acid sequence of SEQ ID NO: 133 or a variant thereof comprising up to 5 (e.g., 1, 2, 3, 4, or 5) amino acid substitutions; and/or b) the VH of the second polypeptide comprises the amino acid sequence of SEQ ID NO: 48, or a variant thereof comprising up to 5 (e.g., 1, 2, 3, 4, or 5) amino acid substitutions; the amino acid sequence of SEQ ID NO: 65, or a variant thereof comprising up to 5 (e.g., 1, 2, 3, 4, or 5) amino acid substitutions; and/or the amino acid sequence of SEQ ID NO: 82, or a variant thereof comprising up to 5 (e.g., 1, 2, 3, 4, or 5) amino acid substitutions. In some embodiments, the VL of the first polypeptide comprises one, two, or three CDRs of illustrative antibody TY21446 as shown in Table 3B, or variants thereof comprising up to 5 (e.g., 1, 2, 3, 4, or 5) amino acid substitutions; and/or the VH of the second polypeptide comprises one, two, or three CDRs of illustrative antibody TY21446 as shown in Table 3A, or variants thereof comprising up to 5 (e.g., 1, 2, 3, 4, or 5) amino acid substitutions. In some embodiments, the VL of the first polypeptide comprises the amino acid sequence of SEQ ID NO: 28, or an amino acid sequence having at least 80% (e.g., at least 85%, 90%, 95%, 98%, or 99%; or 100%) sequence identity with the amino acid sequence of SEQ ID NO: 28; and/or the VH of the second polypeptide comprises the amino acid sequence of SEQ ID NO: 27, or an amino acid sequence having at least 80% (e.g., at least 85%, 90%, 95%, 98%, or 99%; or 100%) sequence identity with the amino acid sequence of SEQ ID NO: 27. In certain embodiments, the first polypeptide comprises the VL of illustrative antibody TY21446 as shown in Table 4A, or an amino acid sequence having at least 80% (e.g., at least 85%, 90%, 95%, 98%, or 99%; or 100%) sequence identity with the VL of illustrative antibody TY21446 as shown in Table 4A; and the second polypeptide comprises the VH of illustrative antibody TY21446 as shown in Table 4A, or an amino acid sequence having at least 80% (e.g., at least 85%, 90%, 95%, 98%, or 99%; or 100%) sequence identity with the VH of illustrative antibody TY21446 as shown in Table 4A. In some embodiments, the first polypeptide comprises a light chain comprising, from N terminus to C terminus, the masking peptide and the VL, and/or the second polypeptide comprises a heavy chain comprising, from N terminus to C terminus, the VH and a human IgG4 Fc domain.

In some embodiments, the masked antibody comprises a human IgG1 Fc region. A masked antibody of the present disclosure may comprise an IgG1 Fc region and any combination of the CDR. VH, VL, and/or light chain sequences described herein. In some embodiments, the masked antibody comprises a first polypeptide comprising a light chain comprising a VL and a second polypeptide comprising a heavy chain comprising, from N terminus to C terminus, a VH and a human IgG1 Fc region. In certain embodiments, a) the VL of the first polypeptide comprises the amino acid sequence of SEQ ID NO: 99, or a variant thereof comprising up to 5 (e.g., 1, 2, 3, 4, or 5) amino acid substitutions; the amino acid sequence of SEQ ID NO: 116, or a variant thereof comprising up to 5 (e.g., 1, 2, 3, 4, or 5) amino acid substitutions; and/or the amino acid sequence of SEQ ID NO: 133, or a variant thereof comprising up to 5 (e.g., 1, 2, 3, 4, or 5) amino acid substitutions; and/or b) the VH of the second polypeptide comprises the amino acid sequence of SEQ ID NO: 48, or a variant thereof comprising up to 5 (e.g., 1, 2, 3, 4, or 5) amino acid substitutions; the amino acid sequence of SEQ ID NO: 65, or a variant thereof comprising up to 5 (e.g., 1, 2, 3, 4, or 5) amino acid substitutions; and/or the amino acid sequence of SEQ ID NO: 82, or a variant thereof comprising up to 5 (e.g., 1, 2, 3, 4, or 5) amino acid substitutions. In some embodiments, the VL of the first polypeptide comprises one, two, or three CDRs of illustrative antibody TY21446 as shown in Table 3B, or variants thereof comprising up to 5 (e.g., 1, 2, 3, 4, or 5) amino acid substitutions; and/or the VH of the second polypeptide comprises one, two, or three CDRs of illustrative antibody TY21446 as shown in Table 3A, or variants thereof comprising up to 5 (e.g., 1, 2, 3, 4, or 5) amino acid substitutions. In some embodiments, the VL of the first polypeptide comprises the amino acid sequence of SEQ ID NO: 28, or an amino acid sequence having at least 80% (e.g., at least 85%, 90%, 95%, 98%, or 99%; or 100%) sequence identity with the amino acid sequence of SEQ ID NO: 28; and/or the VH of the second polypeptide comprises the amino acid sequence of SEQ ID NO: 27, or an amino acid sequence having at least 80% (e.g., at least 85%, 90%, 95%, 98%, or 99%; or 100%) sequence identity with the amino acid sequence of SEQ ID NO: 27. In certain embodiments, the first polypeptide comprises the VL of illustrative antibody TY21446 as shown in Table 4A, or an amino acid sequence having at least 80% (e.g., at least 85%, 90%, 95%, 98%, or 99%; or 100%) sequence identity with the VL of illustrative antibody TY21446 as shown in Table 4A; and the second polypeptide comprises the VH of illustrative antibody TY21446 as shown in Table 4A, or an amino acid sequence having at least 80% (e.g., at least 85%, 90%, 95%, 98%, or 99%; or 100%) sequence identity with the VH of illustrative antibody TY21446 as shown in Table 4A. In some embodiments, the first polypeptide comprises a light chain comprising, from N terminus to C terminus, the masking peptide and the VL, and/or the second polypeptide comprises a heavy chain comprising, from N terminus to C terminus, the VH and a human IgG1 Fc domain.

In some embodiments, the masked antibody comprises a human IgG1 Fc region having one or more amino acid substitutions. The IgG1 Fc region can comprise any amino acid substitution known in the art to confer a desired property to the antibody having the IgG1 Fc region. In certain embodiments, the human IgG1 Fc region comprises a S239D substitution and/or an 1332E substitution. In certain embodiments, the human IgG1 Fc region comprises two Fc domains, wherein each of the two Fc domains comprises a S239D substitution and/or an 1332E substitution (e.g., a S239D substitution and an 1332E substitution). A masked antibody of the present disclosure may comprise an IgG1 Fc region comprising a S239D substitution and/or an 1332E substitution and any combination of the CDR, VH, VL, and/or light chain sequences described herein. In some embodiments, the masked antibody comprises a first polypeptide comprising a light chain comprising a VL and a second polypeptide comprising a heavy chain comprising, from N terminus to C terminus, a VH and a human IgG1 Fc region comprising a S239D substitution and/or an 1332E substitution. In some embodiments, the masked antibody comprises a first polypeptide comprising a light chain comprising a VL and a second polypeptide comprising a heavy chain comprising, from N terminus to C terminus, a VH and a human IgG1 Fc region. In certain embodiments, a) the VL of the first polypeptide comprises the amino acid sequence of SEQ ID NO: 99, or a variant thereof comprising up to 5 (e.g., 1, 2, 3, 4, or 5) amino acid substitutions; the amino acid sequence of SEQ ID NO: 116, or a variant thereof comprising up to 5 (e.g., 1, 2, 3, 4, or 5) amino acid substitutions; and/or the amino acid sequence of SEQ ID NO: 133, or a variant thereof comprising up to 5 (e.g., 1, 2, 3, 4, or 5) amino acid substitutions; and/or b) the VH of the second polypeptide comprises the amino acid sequence of SEQ ID NO: 48, or a variant thereof comprising up to 5 (e.g., 1, 2, 3, 4, or 5) amino acid substitutions; the amino acid sequence of SEQ ID NO: 65, or a variant thereof comprising up to 5 (e.g., 1, 2, 3, 4, or 5) amino acid substitutions; and/or the amino acid sequence of SEQ ID NO: 82, or a variant thereof comprising up to 5 (e.g., 1, 2, 3, 4, or 5) amino acid substitutions. In some embodiments, the VL of the first polypeptide comprises one, two, or three CDRs of illustrative antibody TY21446 as shown in Table 3B, or variants thereof comprising up to 5 (e.g., 1, 2, 3, 4, or 5) amino acid substitutions; and/or the VH of the second polypeptide comprises one, two, or three CDRs of illustrative antibody TY21446 as shown in Table 3A, or variants thereof comprising up to 5 (e.g., 1, 2, 3, 4, or 5) amino acid substitutions. In some embodiments, the VL of the first polypeptide comprises the amino acid sequence of SEQ ID NO: 28, or an amino acid sequence having at least 80% (e.g., at least 85%, 90%, 95%, 98%, or 99%; or 100%) sequence identity with the amino acid sequence of SEQ ID NO: 28; and/or the VH of the second polypeptide comprises the amino acid sequence of SEQ ID NO: 27, or an amino acid sequence having at least 80% (e.g., at least 85%, 90%, 95%, 98%, or 99%; or 100%) sequence identity with the amino acid sequence of SEQ ID NO: 27. In certain embodiments, the first polypeptide comprises the VL of illustrative antibody TY21446 as shown in Table 4A, or an amino acid sequence having at least 80% (e.g., at least 85%, 90%, 95%, 98%, or 99%; or 100%) sequence identity with the VL of illustrative antibody TY21446 as shown in Table 4A; and the second polypeptide comprises the VH of illustrative antibody TY21446 as shown in Table 4A, or an amino acid sequence having at least 80% (e.g., at least 85%, 90%, 95%, 98%, or 99%; or 100%) sequence identity with the VH of illustrative antibody TY21446 as shown in Table 4A. In some embodiments, the first polypeptide comprises a light chain comprising, from N terminus to C terminus, the masking peptide and the VL, and/or the second polypeptide comprises a heavy chain comprising, from N terminus to C terminus, the VH and a human IgG1 Fc domain comprising a S239D substitution and/or an 1332E substitution.

Further, the masked antibodies provided by the present disclosure can be monoclonal or polyclonal. In a preferred embodiment, the masked antibody is monoclonal.

In some embodiments, the masked peptide, or a portion thereof (e.g., the masking moiety (MM)) interferes with, obstructs, reduces the ability of, prevents, inhibits, or competes with the target binding moiety (TBM) for binding to its target (e.g., CD47). In some embodiments, the masking peptide, or a portion thereof (e.g., the MM), has a dissociation constant for binding to the TBM that is greater (e.g., at least about 1.5-fold greater, at least about 2-fold greater, at least about 2.5-fold greater, at least about 3-fold greater, at least about 3.5-fold greater, at least about 4-fold greater, at least about 4.5-fold greater, at least about 5-fold greater, at least about 10-fold greater, at least about 100-fold greater, at least about 500-fold greater, etc.) than the dissociation constant of the masked antibody, or a part thereof (e.g., the TBM), for its target (e.g., CD47).

In some embodiments, the masking peptide, or a portion thereof (e.g., the masking moiety (MM), has a measurable masking efficiency. In some embodiments, masking efficiency is measured as the difference in affinity of a masked antibody comprising a masking peptide for binding its target relative to the affinity of a polypeptide lacking the masking peptide, for binding its target (e.g., the difference in affinity for a target antigen (such as CD47) of a masked antibody comprising a masking peptide relative to a parental antibody lacking the masking peptide). In some embodiments, the masking efficiency is measured by dividing the EC50 for binding of a masked antibody comprising a masking peptide by the EC50 of the parental antibody lacking the masking peptide (e.g., by measuring EC50 by ELISA; see e.g., the methods of Example 5). In some embodiments, the masking peptide, or a portion thereof (e.g., the MM) binds to the target binding moiety (TBM), and prevents the masked antibody from binding to its target (e.g., CD47). In some embodiments, the masking peptide, or a portion thereof (e.g., the MM) has a dissociation constant for binding to the TBM that is greater than the dissociation constant of the TBM for its target (e.g., CD47). Dissociation constants can be measured, e.g., by techniques such as ELISA, surface plasmon resonance or Bio-Layer Interferometry (BLI), or flow cytometry.

In some embodiments, the masking peptide, or a portion thereof (e.g., the MM), has a masking efficiency of at least about 2.0 (e.g., at least about 2.0, at least about 3.0, at least about 4.0, at least about 5.0, at least about 6.0, at least about 7.0, at least about 8.0, at least about 9.0, at least about 10, at least about 25, at least about 50, at least about 75, at least about 100, at least about 150, at least about 200, at least about 300, at least about 400, at least about 500, etc.) as determined by a Jurkat NFAT reporter assay for example, as described in U.S. patent application Ser. No. 16/966,848. In some embodiments, the masking peptide, or a portion thereof (e.g., the MM), has a masking efficiency of at least about 50 as determined by a Jurkat NFAT reporter assay. In certain embodiments, the masking peptide, or a portion thereof (e.g., the MM), has a masking efficiency of at least about 100 as determined by a Jurkat NFAT reporter assay.

In some embodiments, the masked antibody comprising the masking peptide and target-binding moiety (TBM) has a reduced binding to red blood cells (RBCs) in vitro as compared to a parental antibody having the same TBM but lacking the masking peptide. In some embodiments, the masked antibody having a masking peptide, a VH, and a VL has an EC50 of greater than 100 nM (e.g., about 110 nM, about 120 nM, about 130 nM, about 140 nM, about 150 nM, about 200 nM, about 300 nM, about 400 nM, about 500 nM, about 1 μM, or greater) for binding to RBCs in vitro, and an antibody having the same VH and VL and lacking the masking peptide has an EC50 of less than 10 nM (e.g., about 9 nM, about 8 nM, about 7 nM, about 7 nM, about 5 nM, about 4 nM, about 3 nM, about 2 nM, about 1 nM, about 0.5 nM, about 0.4 nM, about 0.3 nM, about 0.2 nM, about 0.1 nM, or less) for binding to RBCs in vitro under the same assay conditions. In certain embodiments, the masked antibody having a masking peptide, a VH, and a VL has an EC50 for binding to RBCs in vitro of about 10 fold, about 50 fold, about 100 fold, about 200 fold, about 300 fold, about 400 fold, about 500 fold, about 600 fold, about 700 fold, about 800 fold, about 900 fold, about 1000 fold, about 1500 fold, about 2000 fold, or about 2500 fold greater than that of an antibody having the same VH and VL and lacking the masking peptide.

In some embodiments, the masked antibody is not an activatable antibody. In other embodiments, the masked antibody is an activatable antibody. In some embodiments, the masked antibody is an activatable antibody and the linkage moiety (LM) comprises a cleavable moiety (CM). In certain embodiments, the masked antibody is an activatable antibody, the LM comprises a CM, and the activatable antibody has a higher binding affinity to human CD47 in vitro upon cleavage of the CM than before the cleavage of the CM.

### B-3. Activatable Antibodies

In some aspects, provided herein are activatable antibodies that bind to CD47. In some embodiments, the activatable antibody comprises (a) a masking peptide comprising, from N terminus to C terminus, a masking moiety (MM) and a cleavable moiety (CM), wherein the CM comprises at least one cleavage site; and (b) a target binding moiety (TBM) comprising an antibody heavy chain variable region (VH) and an antibody light chain variable region (VL). In certain embodiments, the masking peptide is linked to the N terminus of the VH or the VL. In preferred embodiments, the activatable antibody has a higher binding affinity to human CD47 in vitro upon cleavage of the CM than before the cleavage of the CM. The TBM may comprise one or more sequences of the anti-CD47 antibodies or antigen-binding fragments described herein, including antibodies or antigen-binding fragments described with reference to specific amino acid sequences of CDRs, variable regions (VL, VH), and/or light and heavy chains (e.g., IgG1, IgG2, IgG4). In some embodiments, TBM comprises a full length antibody light chain and/or a full length antibody heavy chain of one or more of the anti-CD47 antibodies described herein.

In some embodiments, the present disclosure relates to activatable antibodies that bind to human CD47, and have at least one (e.g., at least one, at least two, at least three, at least four, at least five, at least six, at least seven, at least eight, at least nine, at least 10, at least 11, at least 12, or all 13) of the following functional properties: (a) has a greater Kp for binding to human CD47 when the CM is not cleaved than when the CM is cleaved; (b) has a greater half maximal effective concentration (EC50) for binding to human CD47 when the CM is not cleaved than when the CM is cleaved; (c) has a higher half maximal inhibitory concentration (IC50) for blocking binding of human CD47 to human SIRPα in vitro when the CM is not cleaved than when the CM is cleaved; (d) completely blocks binding of human CD47 to human SIRPα in vitro when the activatable antibody is provided at a concentration of about 1 nM or greater when the CM is cleaved; (e) has a greater EC50 for binding to tumor cells in vitro when the CM is not cleaved than when the CM is cleaved; (f) has a reduced binding to red blood cells (RBCs) in vitro when the CM is not cleaved as compared to a parental antibody having the same TBM but lacking the masking peptide, or as compared to the activatable antibody when the CM is cleaved; (g) has a greater EC50 for increasing macrophage phagocytosis of tumor cells in vitro when the CM is not cleaved than when the CM is cleaved; (h) binds to human CD47 with a Kp of 500 nM or less when the CM is cleaved; (i) is cross-reactive with monkey, rat, or dog CD47 when the CM is cleaved; (j) is capable of inhibiting tumor cell growth when the CM is cleaved; (k) has one or more therapeutic effects on a cancer; (1) blocks binding between CD47 and SIRP proteins (e.g., SIRPα) when the CM is cleaved; and (m) exhibits antibody-dependent cellular cytotoxicity (ADCC) against CD47-expressing tumor cells when the CM is cleaved.

In some embodiments, the activatable antibody comprises a target-binding moiety (TBM). In some embodiments, the TBM comprises an antibody light chain variable region and/or an antibody heavy chain variable region. In some embodiments, the TBM comprises an antibody light chain variable region. In some embodiments, the TBM comprises an antibody heavy chain variable region. In some embodiments, the TBM comprises an antibody light chain variable region and an antibody heavy chain variable region. In some embodiments, the antibody heavy chain variable region is C-terminal to the antibody light chain variable region. In some embodiments, the antibody light chain variable region is C-terminal to the antibody heavy chain variable region. In some embodiments, a TBM of the present disclosure comprises an antibody light chain variable region and/or an antibody heavy chain variable region with specificity for CD47. In some embodiments, the TBM comprises a full length antibody light chain and/or a full length antibody heavy chain. The antibody light chain may be a kappa or lambda light chain. The antibody heavy chain may be in any class, such as IgG, IgM, IgE, IgA, or IgD. In some embodiments, the antibody heavy chain is in the IgG class, such as IgG1, IgG2, IgG3, or lgG4 subclass. An antibody heavy chain described herein may be converted from one class or subclass to another class or subclass using methods known in the art. Any one or more of the TBMs described herein may incorporate any of the CDR sequences (e.g., one, two, or three of the heavy chain variable region CDR sequences, and/or one, two, or three of the light chain variable region CDR sequences), heavy chain variable region sequences, and/or light chain variable region sequences of any of the anti-CD47 antibodies described herein.

In some embodiments, the activatable antibody, or an antigen-binding fragment thereof, comprises a first polypeptide comprising, from N terminus to C terminus, the masking peptide and the VL, and a second polypeptide comprising the VH (e.g., a Fab fragment). In other embodiments, the activatable antibody or an antigen-binding fragment thereof, comprises a first polypeptide comprising, from N terminus to C terminus, the masking peptide and the VH, and a second polypeptide comprising the VL (e.g., a Fab fragment). In other embodiments, the activatable antibody, or an antigen-binding fragment thereof, comprises a first polypeptide comprising, from N terminus to C terminus, the masking peptide, the VL, and the VH (e.g., an scFv). In yet additional embodiments, the activatable antibody, or an antigen-binding fragment thereof, comprises a first polypeptide comprising, from N terminus to C terminus the masking peptide, the VH, and the VL (e.g., an scFv).

In some embodiments, the activatable antibody comprises a masking peptide comprising, from N terminus to C terminus, and MM and a CM. In some embodiments, the MM comprises an amino acid sequence according to Formula (XVI): X1X2X3X4X5X6CX7DDX8X9X10CX11X12 (SEQ ID NO: 197), wherein X1 is D, H, N. or Y, X2 is A, D, F. P. T, or Y, X3 is A, L, N, P. T, or Y, X4 is A, D, H, or S, X5 is A, D, F. H, or N, X6 is D, S, or T. X7 is D. S, or Y, X8 is D, F, or Y, X9 and X11 are each independently A, D, or Y, X10 is A, D, F, or P, and X12 is D, F. I. T, or Y. In certain embodiments, the MM comprises an amino acid sequence according to Formula (XVII): X1X2X3X4X5DCPX6X7DX8X9CX10X11 (SEQ ID NO: 198), wherein X1 is A, N, or P, X2 is A. N. P. or Q, X3 is A, D, or S, X4 is A, D, S, or V. X5 is D, F, or P. X6 is A, D, or T, X7 is A, H, or Y, X8 is A, D, or V, X9 is For Y, X10 is D. N. S, or Y, and X11 is D, P, or V. In other embodiments, the MM comprises an amino acid sequence according to Formula (XVIII): X1X2X3X4X5X6CDX7X8X9X10X11CX12A (SEQ ID NO: 199), wherein X1 is A or L, X2 is A or T, X3 is S or V. X4 is D or P. X5 is A or Y, X6 is F or T, X7 is D or I, X8 is D or T. X9 is L or P, X10 is For L. X11 is For Y, and X12 is N or P. In yet other embodiments, the MM comprises an amino acid sequence according to Formula (XIX): X1X2CX3X4X5X6X7X8X9FCX10X11 (SEQ ID NO: 200), wherein X1 is D, F, or V, X2 is A. S, or Y, X3 is P. R. or T, X4 is A, G, or I, X5 is A, E, or F. X6 is A, D, or V, X7 is D or V. X8 is D or G, X9 is I or P. X10 is I or S, and X11 is A, Q, or V. In some embodiments, the MM comprises an amino acid sequence selected from the group consisting of SEQ ID NOS: 137 and 167-181, or a variant thereof comprising up to 5 (e.g., 1, 2, 3, 4, or 5) amino acid substitutions. In certain embodiments, the MM comprises the amino acid sequence of SEQ ID NO: 137, or a variant thereof comprising up to 5 (e.g., 1, 2, 3, 4, or 5) amino acid substitutions. In some embodiments, the MM comprises the amino acid sequence of the MM of an illustrative antibody as described in Table 5A, or a variant thereof comprising up to 5 (e.g., 1, 2, 3, 4, or 5) amino acid substitutions. In certain embodiments, the MM comprises the amino acid sequence of the MM of illustrative antibody TY26294 as described in Table 5A, or a variant thereof comprising up to 5 (e.g., 1, 2, 3, 4, or 5) amino acid substitutions.

In some embodiments, the activatable antibody comprises a masking peptide comprising, from N terminus to C terminus, and MM and a CM, wherein the CM comprises at least one (e.g., one, two, three, or more) cleavage site. In some embodiments, the cleavage site is a protease cleavage site. Any suitable protease cleavage site recognized and/or cleaved by any protease (e.g., a protease that is known to be co-localized with a target of a polypeptide comprising the CM) known in the art may be used, including, for example, a protease cleavage site recognized and/or cleaved by urokinase-type plasminogen activator (uPA); matrix metalloproteinases (e.g., MMP-1, MMP-2, MMP-3, MMP-7, MMP-8, MMP-9, MMP-10, MMP-11, MMP-12, MMP-13, MMP-14, MMP-15, MMP-16, MMP-17, MMP-19, MMP-20, MMP-23, MMP-24, MMP-26, and/or MMP-27); Tobacco Etch Virus (TEV) protease; plasmin; Thrombin; PSA; PSMA; ADAMS/ADAMTS (e.g., ADAM 8, ADAM 9, ADAM10, ADAM12, ADAM15, ADAM17/TACE, ADAMDECI, ADAMTS1, ADAMTS4, and/or ADAMTS5); caspases (e.g., Caspase-1, Caspase-2, Caspase-3, Caspase-4, Caspasc-5, Caspase-6, Caspase-7, Caspase-8, Caspase-9, Caspase-10, Caspase-11, Caspase-12, Caspase-13, and/or Caspase-14); aspartate proteases (e.g., RACE and/or Renin); aspartic cathepsins (e.g., Cathepsin D and/or Cathepsin E); cysteine cathepsins (e.g., Cathepsin B, Cathepsin C. Cathepsin K, Cathepsin L, Cathepsin S, Cathepsin V/L2, and/or Cathepsin X/Z/P); cysteine protcinases (e.g., Cruzipain, Legumain, and/or Otubain-2); KLKs (e.g., KLK4, KLK5, KLK6, KLK7, KLK8, KLK10, KLK11, KLK13, and/or KLK14); metallo proteainases (e.g., Meprin, Neprilysin, PSMA, and/or BMP-1); serine proteases (e.g., activated protein C, Cathepsin A. Cathepsin G, Chymase, and/or coagulation factor proteases (such as FVIIa, FIXa, FXa, FXIa, FXIIa)); clastase; granzyme B; guanidinobenzoatase; HtrA1; human neutrophil elastase; lactoferrin; marapsin; NS3/4A; PACE4; tPA; tryptase; type II transmembrane serine proteases (TTSPs) (e.g., DESC1, DPP-4, FAP, Hepsin, Matriptase-2, MT-SP1/Matriptase, TMPRSS2, TMPRSS3 and/or TMPRSS4); etc., In certain embodiments, the one or more protease cleavage sites are substrates for one or more proteases selected from the group consisting of urokinase-type plasminogen activator (uPA), matrix metalloproteinase-1 (MMP-1), MMP-2, MMP-3, MMP-8, MMP-9, MMP-14, Tobacco Etch Virus (TEV) protease, plasmin, Thrombin, Factor X, PSA, PSMA, Cathepsin D, Cathepsin K, Cathepsin S, ADAM10, ADAM12, ADAMTS, Caspase-1, Caspase-2, Caspasc-3, Caspase-4, Caspase-5, Caspase-6, Caspase-7, Caspase-8, Caspase-9, Caspase-10, Caspase-11, Caspase-12, Caspasc-13, Caspase-14, and TACE. In some embodiments, CM comprises an MMP-9 cleavage site that is cleavable by MMP-9. In certain embodiments, the CM comprises the amino acid sequence of SEQ ID NO: 138, or a variant thereof comprising up to 5 (e.g., 1, 2, 3, 4, or 5) amino acid substitutions. In certain embodiments, the CM comprises the amino acid sequence of the CM of illustrative antibody TY26294 as described in Table 5A, or a variant thereof comprising up to 5 (e.g., 1, 2, 3, 4, or 5) amino acid substitutions.

In some embodiments, the masking peptide further comprises one or more (e.g., one, two, three, or more) linkers. In some embodiments, the CM further comprises one or more (e.g., one, two, three, or more) linkers, which may be C-terminal to the cleavage site or N-terminal to the cleavage site. In certain embodiments, the MM further comprises one or more (e.g., one, two, three, or more) linkers. Any suitable linker (e.g., a flexible linker) known in the art may be used, including, for example: glycine polymers (G) n, where n is an integer of at least 1 (e.g., at least one, at least 2, at least 3, at least 4, at least 5, at least 6, at least 7, at least 8, at least 9, at least 10, etc.); glycine-serine polymers (GS) n, where n is an integer of at least 1 (e.g., at least one, at least 2, at least 3, at least 4, at least 5, at least 6, at least 7, at least 8, at least 9, at least 10, etc.); glycine-alanine polymers; alanine-serine polymers; and the like. Linker sequences may be of any length, such as from about 1 amino acid (e.g., glycine or serine) to about 20 amino acids (e.g., 20 amino acid glycine polymers or glycine-serine polymers), about 1 amino acid to about 15 amino acids, about 3 amino acids to about 12 amino acids, about 4 amino acids to about 10 amino acids, about 5 amino acids to about 9 amino acids, about 6 amino acids to about 8 amino acids, etc. In some embodiments, the linker is any of about 1, 2, 3, 4, 5, 6, 7, 8, 9, 10, 11, 12, 13, 14, 15, 16, 17, 18, 19, or 20 amino acids in length.

In some embodiments, the activatable antibody comprises a masking peptide comprising an amino acid sequence selected from the group consisting of SEQ ID NOs: 139 and 152-166, or a variant thereof comprising up to 5 (e.g., 1, 2, 3, 4, or 5) amino acid substitutions. In certain embodiments, the activatable antibody comprises a masking peptide comprising the amino acid sequence of SEQ ID NO: 139, or a variant thereof comprising up to 5 (e.g., 1, 2, 3, 4, or 5) amino acid substitutions. In some embodiments, the masking peptide comprises the amino acid sequence of the masking peptide of an illustrative antibody as described in Table 5B, or a variant thereof comprising up to 5 (e.g., 1, 2, 3, 4, or 5) amino acid substitutions. In certain embodiments, the masking peptide comprises the amino acid sequence of the masking peptide of illustrative antibody TY26294 as described in Table 5B, or a variant thereof comprising up to 5 (e.g., 1, 2, 3, 4, or 5) amino acid substitutions.

In some embodiments, any of the masking peptides described herein may further comprise one or more additional amino acid sequences (e.g., one or more polypeptide tags). Examples of suitable additional amino acid sequence may include, without limitation, purification tags (such as his-tags, flag-tags, maltose binding protein and glutathione-S-transferase tags), detection tags (such as tags that may be detected photometrically (e.g., red or green fluorescent protein, etc.)), tags that have a detectable enzymatic activity (e.g., alkaline phosphatase, etc.), tags containing secretory sequences, leader sequences, and/or stabilizing sequences, protease cleavage sites (e.g., furin cleavage sites, TEV cleavage sites, Thrombin cleavage sites), and the like. In some embodiments, the one or more additional amino acid sequences are at the N-terminus of the masking peptide.

In certain embodiments, the activatable antibody comprises a first polypeptide comprising, from N terminus to C terminus, the masking peptide and the VL; and a second polypeptide comprising the VH. In some embodiments, the second polypeptide further comprises an Fc region. In some embodiments, the Fc region is an IgG4 Fc region. In some embodiments, the Fc region is an IgG1 Fc region. In certain embodiments, the Fc region is an IgG1 Fc region comprising a S239D substitution and/or an 1332E substitution, wherein numbering is according to Kabat. In certain embodiments, the Fc region is an IgG1 Fc region comprising a S239D substitution and an 1332E substitution, wherein numbering is according to Kabat. IgG1 Fc regions comprising a S239D substitution and an 1332E substitution are described in detail in Lazar et al. (Engineered antibody Fc variants with enhanced effector function.” PNAS 103.11 (2006): 4005-4010). In some embodiments, a) the VL of the first polypeptide comprises one or more amino acid sequences selected from the group consisting of SEQ ID NOs: 190-196; and/or b) the VH of the second polypeptide comprises one or more amino acid sequences selected from the group consisting of SEQ ID NOs: 182-189. In some variations, a) the VL of the first polypeptide comprises the amino acid sequences of SEQ ID NOs: 191, 194, and/or 195; and/or b) the VH of the second polypeptide comprises the amino acid sequences of SEQ ID NOs: 182, 185, and/or 188. In certain embodiments, a) the VL of the first polypeptide comprises one or more amino acid sequences selected from the group consisting of SEQ ID NOs: 31-85, or a variant thereof comprising up to 5 (e.g., 1, 2, 3, 4, or 5) amino acid substitutions; and/or b) the VH of the second polypeptide comprises one or more amino acid sequences selected from the group consisting of SEQ ID NOs: 86-136, or a variant thereof comprising up to 5 (e.g., 1, 2, 3, 4, or 5) amino acid substitutions. In some variations, a) the VL of the first polypeptide comprises a CDR-L1 comprising an amino acid sequence selected from the group consisting of SEQ ID NOs: 86-102, or a variant thereof comprising up to 5 (e.g., 1, 2, 3, 4, or 5) amino acid substitutions; a CDR-L2 comprising an amino acid sequence selected from the group consisting of SEQ ID NOs: 103-119, or a variant thereof comprising up to 5 (e.g., 1, 2, 3, 4, or 5) amino acid substitutions; and/or a CDR-L3 comprising an amino acid sequence selected from the group consisting of SEQ ID NOs: 120-136, or a variant thereof comprising up to 5 (e.g., 1, 2, 3, 4, or 5) amino acid substitutions; and/or b) the VH of the second polypeptide comprises a CDR-H1 comprising an amino acid sequence selected from the group consisting of SEQ ID NOs: 35-51, or a variant thereof comprising up to 5 (e.g., 1, 2, 3, 4, or 5) amino acid substitutions; a CDR-H2 comprising an amino acid sequence selected from the group consisting of SEQ ID NOs: 52-68, or a variant thereof comprising up to 5 (e.g., 1, 2, 3, 4, or 5) amino acid substitutions; and/or a CDR-H3 comprising an amino acid sequence selected from the group consisting of SEQ ID NOs: 69-85, or a variant thereof comprising up to 5 (e.g., 1, 2, 3, 4, or 5) amino acid substitutions. In certain embodiments, a) the VL of the first polypeptide comprises the amino acid sequence of SEQ ID NO: 99, or a variant thereof comprising up to 5 (e.g., 1, 2, 3, 4, or 5) amino acid substitutions; the amino acid sequence of SEQ ID NO: 116, or a variant thereof comprising up to 5 (e.g., 1, 2, 3, 4, or 5) amino acid substitutions; and/or the amino acid sequence of SEQ ID NO: 133 or a variant thereof comprising up to 5 (e.g., 1, 2, 3, 4, or 5) amino acid substitutions; and/or b) the VH of the second polypeptide comprises the amino acid sequence of SEQ ID NO: 48, or a variant thereof comprising up to 5 (e.g., 1, 2, 3, 4, or 5) amino acid substitutions; the amino acid sequence of SEQ ID NO: 65, or a variant thereof comprising up to 5 (e.g., 1, 2, 3, 4, or 5) amino acid substitutions; and/or the amino acid sequence of SEQ ID NO: 82, or a variant thereof comprising up to 5 (e.g., 1, 2, 3, 4, or 5) amino acid substitutions. In some embodiments, the VL of the first polypeptide comprises one, two, or three CDRs of illustrative antibody TY21446 as shown in Table 3B, or variants thereof comprising up to 5 (e.g., 1, 2, 3, 4, or 5) amino acid substitutions; and/or the VH of the second polypeptide comprises one, two, or three CDRs of illustrative antibody TY21446 as shown in Table 3A, or variants thereof comprising up to 5 (e.g., 1, 2, 3, 4, or 5) amino acid substitutions. In some embodiments, the VL of the first polypeptide comprises the amino acid sequence of SEQ ID NO: 28, or an amino acid sequence having at least 80% (e.g., at least 85%, 90%, 95%, 98%, or 99%; or 100%) sequence identity with the amino acid sequence of SEQ ID NO: 28; and the VH of the second polypeptide comprises the amino acid sequence of SEQ ID NO: 27, or an amino acid sequence having at least 80% (e.g., at least 85%, 90%, 95%, 98%, or 99%; or 100%) sequence identity with the amino acid sequence of SEQ ID NO: 27. In certain embodiments, the first polypeptide comprises the VL of illustrative antibody TY21446 as shown in Table 4A, or an amino acid sequence having at least 80% (e.g., at least 85%, 90%, 95%, 98%, or 99%; or 100%) sequence identity with the VL of illustrative antibody TY21446 as shown in Table 4A; and the second polypeptide comprises the VH of illustrative antibody TY21446 as shown in Table 4A, or an amino acid sequence having at least 80% (e.g., at least 85%, 90%, 95%, 98%, or 99%; or 100%) sequence identity with the VH of illustrative antibody TY21446 as shown in Table 4A.

In some embodiments, provided herein is an activatable antibody comprising: (a) a masking peptide comprising, from N terminus to C terminus, a masking moiety (MM) and a cleavable moiety (CM), wherein the CM comprises at least one cleavage site, and (b) a target binding moiety (TBM) comprising an antibody heavy chain variable region (VH) and an antibody light chain variable region (VL); wherein the masking peptide is linked to the N terminus of the VL; and wherein the activatable antibody has a higher binding affinity to human CD47 in vitro upon cleavage of the CM than before the cleavage of the CM; wherein: 1) the VH comprises a CDR-H1 comprising the amino acid sequence of SEQ ID NO: 48, or a variant thereof comprising up to 5 (e.g., 1, 2, 3, 4, or 5) amino acid substitutions; a CDR-H2 comprising the amino acid sequence of SEQ ID NO: 65, or a variant thereof comprising up to 5 (e.g., 1, 2, 3, 4, or 5) amino acid substitutions; and a CDR-H3 comprising the amino acid sequence of SEQ ID NO: 82, or a variant thereof comprising up to 5 (e.g., 1, 2, 3, 4, or 5) amino acid substitutions; and 2) the VL comprises a CDR-L1 comprising the amino acid sequence of SEQ ID NO: 99, or a variant thereof comprising up to 5 (e.g., 1, 2, 3, 4, or 5) amino acid substitutions; a CDR-L2 comprising the amino acid sequence of SEQ ID NO: 116, or a variant thereof comprising up to 5 (e.g., 1, 2, 3, 4, or 5) amino acid substitutions; and a CDR-L3 comprising the amino acid sequence of SEQ ID NO: 133, or a variant thereof comprising up to 5 (e.g., 1, 2, 3, 4, or 5) amino acid substitutions. In some embodiments, the activatable antibody comprises (a) a masking peptide comprising, from N terminus to C terminus, an MM and a CM, wherein the CM comprises at least one cleavage site, and (b) a TBM comprising a VH and a VL; wherein the masking peptide is linked to the N terminus of the VL; and wherein the activatable antibody has a higher binding affinity to human CD47 in vitro upon cleavage of the CM than before the cleavage of the CM; wherein: 1) the VH comprises a CDR-H1 comprising the amino acid sequence of SEQ ID NO: 48; a CDR-H2 comprising the amino acid sequence of SEQ ID NO: 65; and a CDR-H3 comprising the amino acid sequence of SEQ ID NO: 82; and 2) the VL comprises a CDR-L1 comprising the amino acid sequence of SEQ ID NO: 99; a CDR-L2 comprising the amino acid sequence of SEQ ID NO: 116; and a CDR-L3 comprising the amino acid sequence of SEQ ID NO: 133. In some embodiments, the activatable antibody comprises (a) a masking peptide comprising, from N terminus to C terminus, an MM and a CM, wherein the CM comprises at least one cleavage site, and (b) a TBM comprising a VH and a VL; wherein the masking peptide is linked to the N terminus of the VL; and wherein the activatable antibody has a higher binding affinity to human CD47 in vitro upon cleavage of the CM than before the cleavage of the CM; wherein: 1) the VH comprises the amino acid sequence of SEQ ID NO: 48, or a variant thereof comprising up to 5 (e.g., 1, 2, 3, 4, or 5) amino acid substitutions; the amino acid sequence of SEQ ID NO: 65, or a variant thereof comprising up to 5 (e.g., 1, 2, 3, 4, or 5) amino acid substitutions; and the amino acid sequence of SEQ ID NO: 82, or a variant thereof comprising up to 5 (e.g., 1, 2, 3, 4, or 5) amino acid substitutions; and 2) the VL comprises the amino acid sequence of SEQ ID NO: 99, or a variant thereof comprising up to 5 (e.g., 1, 2, 3, 4, or 5) amino acid substitutions; the amino acid sequence of SEQ ID NO: 116, or a variant thereof comprising up to 5 (e.g., 1, 2, 3, 4, or 5) amino acid substitutions; and the amino acid sequence of SEQ ID NO: 133, or a variant thereof comprising up to 5 (e.g., 1, 2, 3, 4, or 5) amino acid substitutions. In some embodiments, the activatable antibody comprises one, two, three, four, five, or six CDRs of illustrative antibody TY21446 as shown in Tables 3A-3B, or variants thereof comprising up to 5 (e.g., 1, 2, 3, 4, or 5) amino acid substitutions. In some embodiments, the MM comprises the amino acid sequence of SEQ ID NO: 137, or a variant thereof comprising up to 5 (e.g., 1, 2, 3, 4, or 5) amino acid substitutions. In some embodiments, the MM comprises the amino acid sequence of the MM of illustrative antibody TY26294 as described in Table 5A, or a variant thereof comprising up to 5 (e.g., 1, 2, 3, 4, or 5) amino acid substitutions. In some embodiments, the CM comprises the amino acid sequence of SEQ ID NO: 138, or a variant thereof comprising up to 5 (e.g., 1, 2, 3, 4, or 5) amino acid substitutions. In some embodiments, the CM comprises the amino acid sequence of the CM of illustrative antibody TY26294 as described in Table 5A, or a variant thereof comprising up to 5 (e.g., 1, 2, 3, 4, or 5) amino acid substitutions. In some embodiments, the masking peptide comprises the amino acid sequence of SEQ ID NO: 139, or a variant thereof comprising up to 5 (e.g., 1, 2, 3, 4, or 5) amino acid substitutions. In some embodiments, the masking peptide comprises the amino acid sequence of the masking peptide of illustrative antibody TY26294 as described in Table 5B, or a variant thereof comprising up to 5 (e.g., 1, 2, 3, 4, or 5) amino acid substitutions. In some embodiments, the activatable antibody comprises (a) a masking peptide comprising, from N terminus to C terminus, an MM and a CM, wherein the CM comprises at least one cleavage site, and (b) a TBM comprising a VH and a VL; wherein the masking peptide is linked to the N terminus of the VL; and wherein the activatable antibody has a higher binding affinity to human CD47 in vitro upon cleavage of the CM than before the cleavage of the CM; wherein: 1) the VH comprises the amino acid sequence of SEQ ID NO: 27, or an amino acid sequence having at least 80% (e.g., at least 85%, 90%, 95%, 98%, or 99%; or 100%) sequence identity with the amino acid sequence of SEQ ID NO: 27; and 2) the VL comprises the amino acid sequence of SEQ ID NO: 28, or an amino acid sequence having at least 80% (e.g., at least 85%, 90%, 95%, 98%, or 99%; or 100%) sequence identity with the amino acid sequence of SEQ ID NO: 28. In some embodiments, the activatable antibody comprises the VH and/or the VL of illustrative antibody TY21446 as shown in Table 4A, or one or more an amino acid sequences having at least 80% (e.g., at least 85%, 90%, 95%, 98%, or 99%; or 100%) sequence identity with a VH or a VL of TY21446 as shown in Table 4A. In some embodiments, the activatable antibody comprises an IgG1 Fc region. In certain embodiments, the activatable antibody comprises a human IgG1 Fc region. In some embodiments, the activatable antibody comprises a first polypeptide and a second polypeptide, wherein the first polypeptide comprises a light chain comprising, from N terminus to C terminus, the masking peptide and the VL, wherein the first polypeptide comprises the amino acid sequence of SEQ ID NO: 148, or an amino acid sequence having at least 80% (e.g., at least 85%, 90%, 95%, 98%, or 99%; or 100%) sequence identity with the amino acid sequence of SEQ ID NO: 148; and wherein the second polypeptide comprises a heavy chain comprising, from N terminus to C terminus, the VH and a human IgG1 Fc domain, wherein the second polypeptide comprises the amino acid sequence of SEQ ID NO: 149, or an amino acid sequence having at least 80% (e.g., at least 85%, 90%, 95%, 98%, or 99%; or 100%) sequence identity with the amino acid sequence of SEQ ID NO: 149. In some embodiments, the activatable antibody comprises a heavy chain and/or a light chain of illustrative antibody TY26898 as shown in Table 6, or one or more amino acid sequences having at least 80% (e.g., at least 85%, 90%, 95%, 98%, or 99%; or 100%) sequence identity with a heavy chain or a light chain of illustrative antibody TY26898 as shown in Table 6. In some embodiments, the IgG1 Fc region comprises a S239D substitution and/or an 1332E substitution. In some embodiments, the IgG1 Fc region comprises a S239D substitution and an 1332E substitution. In certain embodiments, the human IgG1 Fc region comprises a S239D substitution and an 1332E substitution. In certain embodiments, the human IgG1 Fc region comprises two Fc domains, wherein each of the two Fc domains comprises a S239D substitution and/or an 1332E substitution (e.g., a S239D substitution and an 1332E substitution). In some embodiments, the activatable antibody comprises a first polypeptide and a second polypeptide, wherein the first polypeptide comprises a light chain comprising, from N terminus to C terminus, the masking peptide and the VL, wherein the first polypeptide comprises the amino acid sequence of SEQ ID NO: 150, or an amino acid sequence having at least 80% (e.g., at least 85%, 90%, 95%, 98%, or 99%; or 100%) sequence identity with the amino acid sequence of SEQ ID NO: 150; and wherein the second polypeptide comprises a heavy chain comprising, from N terminus to C terminus, the VH and a human IgG1 Fc domain, wherein the second polypeptide comprises the amino acid sequence of SEQ ID NO: 151, or an amino acid sequence having at least 80% (e.g., at least 85%, 90%, 95%, 98%, or 99%; or 100%) sequence identity with the amino acid sequence of SEQ ID NO: 151. In some embodiments, the activatable antibody comprises a heavy chain and/or a light chain of illustrative antibody TY26899 as shown in Table 6, or one or more amino acid sequences having at least 80% (e.g., at least 85%, 90%, 95%, 98%, or 99%; or 100%) sequence identity with a heavy chain or a light chain of illustrative antibody TY26899 as shown in Table 6. In some embodiments, the IgG1 Fc region has enhanced antibody-dependent cellular cytotoxic (ADCC) and/or antibody-dependent cellular phagocytosis (ADCP), function(s). In some embodiments, the activatable antibody comprises an IgG4 Fc region. In certain embodiments, the activatable antibody comprises a human IgG4 Fc region. In some embodiments, the activatable antibody comprises a first polypeptide and a second polypeptide, wherein the first polypeptide comprises a light chain comprising, from N terminus to C terminus, the masking peptide and the VL, wherein the first polypeptide comprises the amino acid sequence of SEQ ID NO: 142, or an amino acid sequence having at least 80% (e.g., at least 85%, 90%, 95%, 98%, or 99%; or 100%) sequence identity with the amino acid sequence of SEQ ID NO: 142; and wherein the second polypeptide comprises a heavy chain comprising, from N terminus to C terminus, the VH and a human IgG4 Fc domain, wherein the second polypeptide comprises the amino acid sequence of SEQ ID NO: 143, or an amino acid sequence having at least 80% (e.g., at least 85%, 90%, 95%, 98%, or 99%; or 100%) sequence identity with the amino acid sequence of SEQ ID NO: 143. In some embodiments, the activatable antibody comprises a heavy chain and/or a light chain of illustrative antibody TY26294 as shown in Table 6, or one or more amino acid sequences having at least 80% (e.g., at least 85%, 90%, 95%, 98%, or 99%; or 100%) sequence identity with a heavy chain or a light chain of illustrative antibody TY26294 as shown in Table 6.

In some embodiments, provided herein is an activatable antibody comprising: (a) a masking peptide comprising, from N terminus to C terminus, a masking moiety (MM) and a cleavable moiety (CM), wherein the CM comprises at least one cleavage site, and (b) a target binding moiety (TBM) comprising an antibody heavy chain variable region (VH) and an antibody light chain variable region (VL); wherein the masking peptide is linked to the N terminus of the VL; and wherein the activatable antibody has a higher binding affinity to human CD47 in vitro upon cleavage of the CM than before the cleavage of the CM; wherein: 1) the VH comprises a CDR-H1 comprising the amino acid sequence of SEQ ID NO: 49, or a variant thereof comprising up to 5 (e.g., 1, 2, 3, 4, or 5) amino acid substitutions; a CDR-H2 comprising the amino acid sequence of SEQ ID NO: 66, or a variant thereof comprising up to 5 (e.g., 1, 2, 3, 4, or 5) amino acid substitutions; and a CDR-H3 comprising the amino acid sequence of SEQ ID NO: 83, or a variant thereof comprising up to 5 (e.g., 1, 2, 3, 4, or 5) amino acid substitutions; and 2) the VL comprises a CDR-L1 comprising the amino acid sequence of SEQ ID NO: 100, or a variant thereof comprising up to 5 (e.g., 1, 2, 3, 4, or 5) amino acid substitutions; a CDR-L2 comprising the amino acid sequence of SEQ ID NO: 117, or a variant thereof comprising up to 5 (e.g., 1, 2, 3, 4, or 5) amino acid substitutions; and a CDR-L3 comprising the amino acid sequence of SEQ ID NO: 134, or a variant thereof comprising up to 5 (e.g., 1, 2, 3, 4, or 5) amino acid substitutions. In some embodiments, the activatable antibody comprises (a) a masking peptide comprising, from N terminus to C terminus, an MM and a CM, wherein the CM comprises at least one cleavage site, and (b) a TBM comprising a VH and a VL; wherein the masking peptide is linked to the N terminus of the VL; and wherein the activatable antibody has a higher binding affinity to human CD47 in vitro upon cleavage of the CM than before the cleavage of the CM; wherein: 1) the VH comprises a CDR-H1 comprising the amino acid sequence of SEQ ID NO: 49; a CDR-H2 comprising the amino acid sequence of SEQ ID NO: 66; and a CDR-H3 comprising the amino acid sequence of SEQ ID NO: 83; and 2) the VL comprises a CDR-L1 comprising the amino acid sequence of SEQ ID NO: 100; a CDR-L2 comprising the amino acid sequence of SEQ ID NO: 117; and a CDR-L3 comprising the amino acid sequence of SEQ ID NO: 134. In some embodiments, the activatable antibody comprises (a) a masking peptide comprising, from N terminus to C terminus, an MM and a CM, wherein the CM comprises at least one cleavage site, and (b) a TBM comprising a VH and a VL; wherein the masking peptide is linked to the N terminus of the VL; and wherein the activatable antibody has a higher binding affinity to human CD47 in vitro upon cleavage of the CM than before the cleavage of the CM; wherein: 1) the VH comprises the amino acid sequence of SEQ ID NO: 49, or a variant thereof comprising up to 5 (e.g., 1, 2, 3, 4, or 5) amino acid substitutions; the amino acid sequence of SEQ ID NO: 66, or a variant thereof comprising up to 5 (e.g., 1, 2, 3, 4, or 5) amino acid substitutions; and the amino acid sequence of SEQ ID NO: 83, or a variant thereof comprising up to 5 (e.g., 1, 2, 3, 4, or 5) amino acid substitutions; and 2) the VL comprises the amino acid sequence of SEQ ID NO: 100, or a variant thereof comprising up to 5 (e.g., 1, 2, 3, 4, or 5) amino acid substitutions; the amino acid sequence of SEQ ID NO: 117, or a variant thereof comprising up to 5 (e.g., 1, 2, 3, 4, or 5) amino acid substitutions; and the amino acid sequence of SEQ ID NO: 134, or a variant thereof comprising up to 5 (e.g., 1, 2, 3, 4, or 5) amino acid substitutions. In some embodiments, the activatable antibody comprises one, two, three, four, five, or six CDRs of illustrative antibody TY21447 as shown in Tables 3A-3B, or variants thereof comprising up to 5 (e.g., 1, 2, 3, 4, or 5) amino acid substitutions. In some embodiments, the MM comprises the amino acid sequence of SEQ ID NO: 137, or a variant thereof comprising up to 5 (e.g., 1, 2, 3, 4, or 5) amino acid substitutions. In some embodiments, the MM comprises the amino acid sequence of the MM of illustrative antibody TY26294 as described in Table 5A, or a variant thereof comprising up to 5 (e.g., 1, 2, 3, 4, or 5) amino acid substitutions. In some embodiments, the CM comprises the amino acid sequence of SEQ ID NO: 138, or a variant thereof comprising up to 5 (e.g., 1, 2, 3, 4, or 5) amino acid substitutions. In some embodiments, the CM comprises the amino acid sequence of the CM of illustrative antibody TY26294 as described in Table 5A, or a variant thereof comprising up to 5 (e.g., 1, 2, 3, 4, or 5) amino acid substitutions. In some embodiments, the masking peptide comprises the amino acid sequence of SEQ ID NO: 139, or a variant thereof comprising up to 5 (e.g., 1, 2, 3, 4, or 5) amino acid substitutions. In some embodiments, the masking peptide comprises the amino acid sequence of the masking peptide of illustrative antibody TY26294 as described in Table 5B, or a variant thereof comprising up to 5 (e.g., 1, 2, 3, 4, or 5) amino acid substitutions. In some embodiments, the activatable antibody comprises (a) a masking peptide comprising, from N terminus to C terminus, an MM and a CM, wherein the CM comprises at least one cleavage site, and (b) a TBM comprising a VH and a VL; wherein the masking peptide is linked to the N terminus of the VL; and wherein the activatable antibody has a higher binding affinity to human CD47 in vitro upon cleavage of the CM than before the cleavage of the CM; wherein: 1) the VH comprises the amino acid sequence of SEQ ID NO: 29, or an amino acid sequence having at least 80% (e.g., at least 85%, 90%, 95%, 98%, or 99%; or 100%) sequence identity with the amino acid sequence of SEQ ID NO: 27; and 2) the VL comprises the amino acid sequence of SEQ ID NO: 28, or an amino acid sequence having at least 80% (e.g., at least 85%, 90%, 95%, 98%, or 99%; or 100%) sequence identity with the amino acid sequence of SEQ ID NO: 30. In some embodiments, the activatable antibody comprises the VH and/or the VL of illustrative antibody TY21446 as shown in Table 4A, or one or more amino acid sequences having at least 80% (e.g., at least 85%, 90%, 95%, 98%, or 99%; or 100%) sequence identity with a VH or a VL of TY21447 as shown in Table 4A. In some embodiments, the activatable antibody comprises an IgG1 Fc region. In certain embodiments, the IgG1 Fc region comprises a S239D substitution and/or an 1332E substitution. In some embodiments, the activatable antibody comprises a human IgG1 Fc region. In certain embodiments, the human IgG1 Fc region comprises a S239D substitution and/or an 1332E substitution. In certain embodiments, the human IgG1 Fc region comprises two Fc domains, wherein each of the two Fc domains comprises a S239D substitution and/or an 1332E substitution (e.g., a S239D substitution and an 1332E substitution). In some embodiments, the IgG1 Fc region has enhanced antibody-dependent cellular cytotoxic (ADCC) and/or antibody-dependent cellular phagocytosis (ADCP), function(s). In some embodiments, the activatable antibody comprises an IgG4 Fc region. In certain embodiments, the activatable antibody comprises a human IgG4 Fc region.

In some embodiments, provided herein is an activatable antibody comprising: (a) a masking peptide comprising, from N terminus to C terminus, a masking moiety (MM) and a cleavable moiety (CM), wherein the CM comprises at least one cleavage site, and (b) a target binding moiety (TBM) comprising an antibody heavy chain variable region (VH) and an antibody light chain variable region (VL); wherein the masking peptide is linked to the N terminus of the VL; and wherein the activatable antibody has a higher binding affinity to human CD47 in vitro upon cleavage of the CM than before the cleavage of the CM; wherein: 1) the VH comprises a CDR-H1 comprising the amino acid sequence of SEQ ID NO: 50, or a variant thereof comprising up to 5 (e.g., 1, 2, 3, 4, or 5) amino acid substitutions; a CDR-H2 comprising the amino acid sequence of SEQ ID NO: 67, or a variant thereof comprising up to 5 (e.g., 1, 2, 3, 4, or 5) amino acid substitutions; and a CDR-H3 comprising the amino acid sequence of SEQ ID NO: 84, or a variant thereof comprising up to 5 (e.g., 1, 2, 3, 4, or 5) amino acid substitutions; and 2) the VL comprises a CDR-L1 comprising the amino acid sequence of SEQ ID NO: 101, or a variant thereof comprising up to 5 (e.g., 1, 2, 3, 4, or 5) amino acid substitutions; a CDR-L2 comprising the amino acid sequence of SEQ ID NO: 118, or a variant thereof comprising up to 5 (e.g., 1, 2, 3, 4, or 5) amino acid substitutions; and a CDR-L3 comprising the amino acid sequence of SEQ ID NO: 135, or a variant thereof comprising up to 5 (e.g., 1, 2, 3, 4, or 5) amino acid substitutions. In some embodiments, the activatable antibody comprises (a) a masking peptide comprising, from N terminus to C terminus, an MM and a CM, wherein the CM comprises at least one cleavage site, and (b) a TBM comprising a VH and a VL; wherein the masking peptide is linked to the N terminus of the VL; and wherein the activatable antibody has a higher binding affinity to human CD47 in vitro upon cleavage of the CM than before the cleavage of the CM; wherein: 1) the VH comprises a CDR-H1 comprising the amino acid sequence of SEQ ID NO: 50; a CDR-H2 comprising the amino acid sequence of SEQ ID NO: 67; and a CDR-H3 comprising the amino acid sequence of SEQ ID NO: 84; and 2) the VL comprises a CDR-L1 comprising the amino acid sequence of SEQ ID NO: 101; a CDR-L2 comprising the amino acid sequence of SEQ ID NO: 118; and a CDR-L3 comprising the amino acid sequence of SEQ ID NO: 135. In some embodiments, the activatable antibody comprises (a) a masking peptide comprising, from N terminus to C terminus, an MM and a CM, wherein the CM comprises at least one cleavage site, and (b) a TBM comprising a VH and a VL; wherein the masking peptide is linked to the N terminus of the VL; and wherein the activatable antibody has a higher binding affinity to human CD47 in vitro upon cleavage of the CM than before the cleavage of the CM; wherein: 1) the VH comprises the amino acid sequence of SEQ ID NO: 50, or a variant thereof comprising up to 5 (e.g., 1, 2, 3, 4, or 5) amino acid substitutions; the amino acid sequence of SEQ ID NO: 67, or a variant thereof comprising up to 5 (e.g., 1, 2, 3, 4, or 5) amino acid substitutions; and the amino acid sequence of SEQ ID NO: 84, or a variant thereof comprising up to 5 (e.g., 1, 2, 3, 4, or 5) amino acid substitutions; and 2) the VL comprises the amino acid sequence of SEQ ID NO: 101, or a variant thereof comprising up to 5 (e.g., 1, 2, 3, 4, or 5) amino acid substitutions; the amino acid sequence of SEQ ID NO: 118, or a variant thereof comprising up to 5 (e.g., 1, 2, 3, 4, or 5) amino acid substitutions; and the amino acid sequence of SEQ ID NO: 135, or a variant thereof comprising up to 5 (e.g., 1, 2, 3, 4, or 5) amino acid substitutions. In some embodiments, the activatable antibody comprises one, two, three, four, five, or six CDRs of illustrative antibody TY21449 as shown in Tables 3A-3B, or variants thereof comprising up to 5 (e.g., 1, 2, 3, 4, or 5) amino acid substitutions. In some embodiments, the MM comprises the amino acid sequence of SEQ ID NO: 137, or a variant thereof comprising up to 5 (e.g., 1, 2, 3, 4, or 5) amino acid substitutions. In some embodiments, the MM comprises the amino acid sequence of the MM of illustrative antibody TY26294 as described in Table 5A, or a variant thereof comprising up to 5 (e.g., 1, 2, 3, 4, or 5) amino acid substitutions. In some embodiments, the CM comprises the amino acid sequence of SEQ ID NO: 138, or a variant thereof comprising up to 5 (e.g., 1, 2, 3, 4, or 5) amino acid substitutions. In some embodiments, the CM comprises the amino acid sequence of the CM of illustrative antibody TY26294 as described in Table 5A, or a variant thereof comprising up to 5 (e.g., 1, 2, 3, 4, or 5) amino acid substitutions. In some embodiments, the masking peptide comprises the amino acid sequence of SEQ ID NO: 139, or a variant thereof comprising up to 5 (e.g., 1, 2, 3, 4, or 5) amino acid substitutions. In some embodiments, the masking peptide comprises the amino acid sequence of the masking peptide of illustrative antibody TY26294 as described in Table 5B, or a variant thereof comprising up to 5 (e.g., 1, 2, 3, 4, or 5) amino acid substitutions. In some embodiments, the activatable antibody comprises (a) a masking peptide comprising, from N terminus to C terminus, an MM and a CM, wherein the CM comprises at least one cleavage site, and (b) a TBM comprising a VH and a VL; wherein the masking peptide is linked to the N terminus of the VL; and wherein the activatable antibody has a higher binding affinity to human CD47 in vitro upon cleavage of the CM than before the cleavage of the CM; wherein: 1) the VH comprises the amino acid sequence of SEQ ID NO: 27, or an amino acid sequence having at least 80% (e.g., at least 85%, 90%, 95%, 98%, or 99%; or 100%) sequence identity with the amino acid sequence of SEQ ID NO: 31; and 2) the VL comprises the amino acid sequence of SEQ ID NO: 28, or an amino acid sequence having at least 80% (e.g., at least 85%, 90%, 95%, 98%, or 99%; or 100%) sequence identity with the amino acid sequence of SEQ ID NO: 32. In some embodiments, the activatable antibody comprises the VH and/or the VL of illustrative antibody TY21446 as shown in Table 4A, or one or more amino acid sequences having at least 80% (e.g., at least 85%, 90%, 95%, 98%, or 99%; or 100%) sequence identity with a VH or a VL of TY21449 as shown in Table 4A. In some embodiments, the activatable antibody comprises an IgG1 Fc region. In certain embodiments, the IgG1 Fc region comprises a S239D substitution and/or an 1332E substitution. In some embodiments, the activatable antibody comprises a human IgG1 Fc region. In certain embodiments, the human IgG1 Fc region comprises a S239D substitution and/or an 1332E substitution. In certain embodiments, the human IgG1 Fc region comprises two Fc domains, wherein each of the two Fc domains comprises a S239D substitution and/or an 1332E substitution (e.g., a S239D substitution and an 1332E substitution). In some embodiments, the IgG1 Fc region has enhanced antibody-dependent cellular cytotoxic (ADCC) and/or antibody-dependent cellular phagocytosis (ADCP), function(s). In some embodiments, the activatable antibody comprises an IgG4 Fc region. In certain embodiments, the activatable antibody comprises a human IgG4 Fc region.

In some embodiments, the activatable antibody comprises a human IgG4 Fc region. An activatable antibody of the present disclosure may comprise an IgG4 Fc region and any combination of the CDR, VH, VL, and/or light chain sequences described herein. In some embodiments, the activatable antibody comprises a first polypeptide comprising a light chain comprising a VL and a second polypeptide comprising a heavy chain comprising, from N terminus to C terminus, a VH and a human IgG4 Fc region. In certain embodiments, a) the VL of the first polypeptide comprises the amino acid sequence of SEQ ID NO: 99, or a variant thereof comprising up to 5 (e.g., 1, 2, 3, 4, or 5) amino acid substitutions; the amino acid sequence of SEQ ID NO: 116, or a variant thereof comprising up to 5 (e.g., 1, 2, 3, 4, or 5) amino acid substitutions; and/or the amino acid sequence of SEQ ID NO: 133 or a variant thereof comprising up to 5 (e.g., 1, 2, 3, 4, or 5) amino acid substitutions; and/or b) the VH of the second polypeptide comprises the amino acid sequence of SEQ ID NO: 48, or a variant thereof comprising up to 5 (e.g., 1, 2, 3, 4, or 5) amino acid substitutions; the amino acid sequence of SEQ ID NO: 65, or a variant thereof comprising up to 5 (e.g., 1, 2, 3, 4, or 5) amino acid substitutions; and/or the amino acid sequence of SEQ ID NO: 82, or a variant thereof comprising up to 5 (e.g., 1, 2, 3, 4, or 5) amino acid substitutions. In some embodiments, the VL of the first polypeptide comprises one, two, or three CDRs of illustrative antibody TY21446 as shown in Table 3B, or variants thereof comprising up to 5 (e.g., 1, 2, 3, 4, or 5) amino acid substitutions; and/or the VH of the second polypeptide comprises one, two, or three CDRs of illustrative antibody TY21446 as shown in Table 3A, or variants thereof comprising up to 5 (e.g., 1, 2, 3, 4, or 5) amino acid substitutions. In some embodiments, the VL of the first polypeptide comprises the amino acid sequence of SEQ ID NO: 28, or an amino acid sequence having at least 80% (e.g., at least 85%, 90%, 95%, 98%, or 99%; or 100%) sequence identity with the amino acid sequence of SEQ ID NO: 28; and/or the VH of the second polypeptide comprises the amino acid sequence of SEQ ID NO: 27, or an amino acid sequence having at least 80% (e.g., at least 85%, 90%, 95%, 98%, or 99%; or 100%) sequence identity with the amino acid sequence of SEQ ID NO: 27. In certain embodiments, the first polypeptide comprises the VL of illustrative antibody TY21446 as shown in Table 4A, or an amino acid sequence having at least 80% (e.g., at least 85%, 90%, 95%, 98%, or 99%; or 100%) sequence identity with the VL of illustrative antibody TY21446 as shown in Table 4A; and the second polypeptide comprises the VH of illustrative antibody TY21446 as shown in Table 4A, or an amino acid sequence having at least 80% (e.g., at least 85%, 90%, 95%, 98%, or 99%; or 100%) sequence identity with the VH of illustrative antibody TY21446 as shown in Table 4A. In some embodiments, the first polypeptide comprises a light chain comprising, from N terminus to C terminus, the masking peptide and the VL, and/or the second polypeptide comprises a heavy chain comprising, from N terminus to C terminus, the VH and a human IgG4 Fc domain. In certain embodiments, the first polypeptide comprises the amino acid sequence of SEQ ID NO: 142, or an amino acid sequence having at least 80% (e.g., at least 85%, 90%, 95%, 98%, or 99%; or 100%) sequence identity with the amino acid sequence of SEQ ID NO: 142; and/or the second polypeptide comprises the amino acid sequence of SEQ ID NO: 143, or an amino acid sequence having at least 80% (e.g., at least 85%, 90%, 95%, 98%, or 99%; or 100%) sequence identity with the amino acid sequence of SEQ ID NO: 143. In some embodiments, the activatable antibody comprises a heavy chain and/or a light chain of illustrative antibody TY26294 as shown in Table 6, or one or more amino acid sequences having at least 80% (e.g., at least 85%, 90%, 95%, 98%, or 99%; or 100%) sequence identity with a heavy chain or a light chain of illustrative antibody TY26294 as shown in Table 6.

In some embodiments, the activatable antibody comprises a human IgG1 Fc region. An activatable antibody of the present disclosure may comprise an IgG1 Fc region and any combination of the CDR, VH, VL, and/or light chain sequences described herein. In some embodiments, the activatable antibody comprises a first polypeptide comprising a light chain comprising a VL and a second polypeptide comprising a heavy chain comprising, from N terminus to C terminus, a VH and a human IgG1 Fc region. In certain embodiments, a) the VL of the first polypeptide comprises the amino acid sequence of SEQ ID NO: 99, or a variant thereof comprising up to 5 (e.g., 1, 2, 3, 4, or 5) amino acid substitutions; the amino acid sequence of SEQ ID NO: 116, or a variant thereof comprising up to 5 (e.g., 1, 2, 3, 4, or 5) amino acid substitutions; and/or the amino acid sequence of SEQ ID NO: 133, or a variant thereof comprising up to 5 (e.g., 1, 2, 3, 4, or 5) amino acid substitutions; and/or b) the VH of the second polypeptide comprises the amino acid sequence of SEQ ID NO: 48, or a variant thereof comprising up to 5 (e.g., 1, 2, 3, 4, or 5) amino acid substitutions; the amino acid sequence of SEQ ID NO: 65, or a variant thereof comprising up to 5 (e.g., 1, 2, 3, 4, or 5) amino acid substitutions; and/or the amino acid sequence of SEQ ID NO: 82, or a variant thereof comprising up to 5 (e.g., 1, 2, 3, 4, or 5) amino acid substitutions. In some embodiments, the VL of the first polypeptide comprises one, two, or three CDRs of illustrative antibody TY21446 as shown in Table 3B, or variants thereof comprising up to 5 (e.g., 1, 2, 3, 4, or 5) amino acid substitutions; and/or the VH of the second polypeptide comprises one, two, or three CDRs of illustrative antibody TY21446 as shown in Table 3A, or variants thereof comprising up to 5 (e.g., 1, 2, 3, 4, or 5) amino acid substitutions. In some embodiments, the VL of the first polypeptide comprises the amino acid sequence of SEQ ID NO: 28, or an amino acid sequence having at least 80% (e.g., at least 85%, 90%, 95%, 98%, or 99%; or 100%) sequence identity with the amino acid sequence of SEQ ID NO: 28; and/or the VH of the second polypeptide comprises the amino acid sequence of SEQ ID NO: 27, or an amino acid sequence having at least 80% (e.g., at least 85%, 90%, 95%, 98%, or 99%; or 100%) sequence identity with the amino acid sequence of SEQ ID NO: 27. In certain embodiments, the first polypeptide comprises the VL of illustrative antibody TY21446 as shown in Table 4A, or an amino acid sequence having at least 80% (e.g., at least 85%, 90%, 95%, 98%, or 99%; or 100%) sequence identity with the VL of illustrative antibody TY21446 as shown in Table 4A; and the second polypeptide comprises the VH of illustrative antibody TY21446 as shown in Table 4A, or an amino acid sequence having at least 80% (e.g., at least 85%, 90%, 95%, 98%, or 99%; or 100%) sequence identity with the VH of illustrative antibody TY21446 as shown in Table 4A. In some embodiments, the first polypeptide comprises a light chain comprising, from N terminus to C terminus, the masking peptide and the VL, and/or the second polypeptide comprises a heavy chain comprising, from N terminus to C terminus, the VH and a human IgG1 Fc domain. In certain embodiments, the first polypeptide comprises the amino acid sequence of SEQ ID NO: 148, or an amino acid sequence having at least 80% (e.g., at least 85%, 90%, 95%, 98%, or 99%; or 100%) sequence identity with the amino acid sequence of SEQ ID NO: 148; and/or the second polypeptide comprises the amino acid sequence of SEQ ID NO: 149, or an amino acid sequence having at least 80% (e.g., at least 85%, 90%, 95%, 98%, or 99%; or 100%) sequence identity with the amino acid sequence of SEQ ID NO: 149. In some embodiments, the activatable antibody comprises a heavy chain and/or a light chain of illustrative antibody TY26898 as shown in Table 6, or one or more amino acid sequences having at least 80% (e.g., at least 85%, 90%, 95%, 98%, or 99%; or 100%) sequence identity with a heavy chain or a light chain of illustrative antibody TY26898 as shown in Table 6.

In some embodiments, the activatable antibody comprises a human IgG1 Fc region having one or more amino acid substitutions. The IgG1 Fc region can comprise any amino acid substitution known in the art to confer a desired property to the antibody having the IgG1 Fc region. In certain embodiments, the human IgG1 Fc region comprises a S239D substitution and/or an 1332E substitution. In certain embodiments, the human IgG1 Fc region comprises two Fc domains, wherein each of the two Fc domains comprises a S239D substitution and/or an 1332E substitution (e.g., a S239D substitution and an 1332E substitution). An activatable antibody of the present disclosure may comprise an IgG1 Fc region comprising a S239D substitution and/or an 1332E substitution and any combination of the CDR, VH, VL, and/or light chain sequences described herein. In some embodiments, the activatable antibody comprises a first polypeptide comprising a light chain comprising a VL and a second polypeptide comprising a heavy chain comprising, from N terminus to C terminus, a VH and a human IgG1 Fc region comprising a S239D substitution and/or an 1332E substitution. In some embodiments, the activatable antibody comprises a first polypeptide comprising a light chain comprising a VL and a second polypeptide comprising a heavy chain comprising, from N terminus to C terminus, a VH and a human IgG1 Fc region. In certain embodiments, a) the VL of the first polypeptide comprises the amino acid sequence of SEQ ID NO: 99, or a variant thereof comprising up to 5 (e.g., 1, 2, 3, 4, or 5) amino acid substitutions; the amino acid sequence of SEQ ID NO: 116, or a variant thereof comprising up to 5 (e.g., 1, 2, 3, 4, or 5) amino acid substitutions; and/or the amino acid sequence of SEQ ID NO: 133, or a variant thereof comprising up to 5 (e.g., 1, 2, 3, 4, or 5) amino acid substitutions; and/or b) the VH of the second polypeptide comprises the amino acid sequence of SEQ ID NO: 48, or a variant thereof comprising up to 5 (e.g., 1, 2, 3, 4, or 5) amino acid substitutions; the amino acid sequence of SEQ ID NO: 65, or a variant thereof comprising up to 5 (e.g., 1, 2, 3, 4, or 5) amino acid substitutions; and/or the amino acid sequence of SEQ ID NO: 82, or a variant thereof comprising up to 5 (e.g., 1, 2, 3, 4, or 5) amino acid substitutions. In some embodiments, the VL of the first polypeptide comprises one, two, or three CDRs of illustrative antibody TY21446 as shown in Table 3B, or variants thereof comprising up to 5 (e.g., 1, 2, 3, 4, or 5) amino acid substitutions; and/or the VH of the second polypeptide comprises one, two, or three CDRs of illustrative antibody TY21446 as shown in Table 3A, or variants thereof comprising up to 5 (e.g., 1, 2, 3, 4, or 5) amino acid substitutions. In some embodiments, the VL of the first polypeptide comprises the amino acid sequence of SEQ ID NO: 28, or an amino acid sequence having at least 80% (e.g., at least 85%, 90%, 95%, 98%, or 99%; or 100%) sequence identity with the amino acid sequence of SEQ ID NO: 28; and/or the VH of the second polypeptide comprises the amino acid sequence of SEQ ID NO: 27, or an amino acid sequence having at least 80% (e.g., at least 85%, 90%, 95%, 98%, or 99%; or 100%) sequence identity with the amino acid sequence of SEQ ID NO: 27. In certain embodiments, the first polypeptide comprises the VL of illustrative antibody TY21446 as shown in Table 4A, or an amino acid sequence having at least 80% (e.g., at least 85%, 90%, 95%, 98%, or 99%; or 100%) sequence identity with the VL of illustrative antibody TY21446 as shown in Table 4A; and the second polypeptide comprises the VH of illustrative antibody TY21446 as shown in Table 4A, or an amino acid sequence having at least 80% (e.g., at least 85%, 90%, 95%, 98%, or 99%; or 100%) sequence identity with the VH of illustrative antibody TY21446 as shown in Table 4A. In some embodiments, the first polypeptide comprises a light chain comprising, from N terminus to C terminus, the masking peptide and the VL, and/or the second polypeptide comprises a heavy chain comprising, from N terminus to C terminus, the VH and a human IgG1 Fc domain comprising a S239D substitution and/or an 1332E substitution. In certain embodiments, the first polypeptide comprises the amino acid sequence of SEQ ID NO: 150, or an amino acid sequence having at least 80% (e.g., at least 85%, 90%, 95%, 98%, or 99%; or 100%) sequence identity with the amino acid sequence of SEQ ID NO: 150; and/or the second polypeptide comprises the amino acid sequence of SEQ ID NO: 151, or an amino acid sequence having at least 80% (e.g., at least 85%, 90%, 95%, 98%, or 99%; or 100%) sequence identity with the amino acid sequence of SEQ ID NO: 151. In some embodiments, the activatable antibody comprises a heavy chain and/or a light chain of illustrative antibody TY26899 as shown in Table 6, or one or more amino acid sequences having at least 80% (e.g., at least 85%, 90%, 95%, 98%, or 99%; or 100%) sequence identity with a heavy chain or a light chain of illustrative antibody TY26899 as shown in Table 6.

Further, the activatable antibodies provided by the present disclosure can be monoclonal or polyclonal. In a preferred embodiment, the activatable antibody is monoclonal.

In some embodiments, the masking peptide, or a portion thereof (e.g., the masking moiety (MM)) interferes with, obstructs, reduces the ability of, prevents, inhibits, or competes with the target binding moiety (TBM) for binding to its target (e.g., CD47). In some embodiments, the masking peptide, or a portion thereof (e.g., the MM) interferes with, obstructs, reduces, prevents, inhibits, or competes with the TBM for binding to its target (e.g., CD47) only when the polypeptide has not been activated (e.g., activated by a change in pH (increased or decreased), activated by a temperature shift (increased or decreased), activated after being contacted with a second molecule (such as a small molecule or a protein ligand), etc.). In some embodiments, the masking peptide, or a portion thereof (e.g., the MM), does not interfere with, obstruct, reduce the ability of, prevent, inhibit, or compete with the target binding moiety (TBM) for binding to its target (e.g., CD47) after the polypeptide has been activated (e.g., activated by treatment with one or more proteases that cleave within the cleavable moiety (CM), activated by a change in pH (increased or decreased), activated by a temperature shift (increased or decreased), activated after being contacted with a second molecule (such as an enzyme), etc.). In some embodiments, the masking peptide, or a portion thereof (e.g., the MM), does not interfere with, obstruct, reduce the ability of, prevent, inhibit, or compete with the TBM for binding to its target after the CM has been cleaved by one or more proteases that cleave within the CM. In some embodiments, the masking peptide, or a portion thereof (e.g., MM), binds to the TBM and inhibits the TBM from binding to its target (e.g., CD47) before activation (e.g., before treatment with one or more proteases that cleave within the cleavable moiety (CM), before undergoing a (local) change in pH (increased or decreased), before a temperature shift (increased or decreased), before being contacted with a second molecule (such as a small molecule or a protein ligand), etc.), but does not bind to the TBM and/or inhibit the polypeptide from binding to its target (e.g., CD47) after activation (e.g., after treatment with one or more proteases that cleave within the CM, after undergoing a (local) change in pH (increased or decreased), after a temperature shift (increased or decreased), after being contacted with a second molecule (such as a small molecule or a protein ligand), etc.). In some embodiments, activation induces cleavage of the polypeptide within the CM. In some embodiments, activation induces conformation changes in the polypeptide (e.g., displacement of the MM or masking peptide), leading to the masking peptide no longer preventing the activatable antibody from binding to its target (e.g., CD47). In some embodiments, the masking peptide, or a portion thereof (e.g., the MM) interferes with, obstructs, reduces the ability of, prevents, inhibits, or competes with the TBM for binding to its target (e.g., CD47) only when the CM has not been cleaved by one or more proteases that cleave within CM. In some embodiments, the masking peptide, or a portion thereof (e.g., the MM), inhibits binding of the activatable antibody to its target (e.g., CD47) when the CM is not cleaved, but does not inhibit binding of the activatable antibody to its target (e.g., CD47) when the CM is cleaved.

In some embodiments, the masking peptide, or a portion thereof (e.g., the MM), has a dissociation constant for binding to the TBM that is greater (e.g., at least about 1.5-fold greater, at least about 2-fold greater, at least about 2.5-fold greater, at least about 3-fold greater, at least about 3.5-fold greater, at least about 4-fold greater, at least about 4.5-fold greater, at least about 5-fold greater, at least about 10-fold greater, at least about 100-fold greater, at least about 500-fold greater, etc.) than the dissociation constant of the activatable antibody, or a part thereof (e.g., the TBM), for its target (e.g., CD47).

In some embodiments, the masking peptide, or a portion thereof (e.g., the masking moiety (MM), has a measurable masking efficiency. In some embodiments, masking efficiency is measured as the difference in affinity of an activatable antibody comprising a masking peptide for binding its target (before activation) relative to the affinity of a polypeptide lacking the masking peptide, for binding its target (e.g., the difference in affinity for a target antigen (such as CD47) of an activatable antibody comprising a masking peptide (before activation) relative to a parental antibody lacking the masking peptide, or the difference in affinity for a target antigen (such as CD47) of an activatable antibody comprising a masking peptide (before activation) relative to the affinity for the target antigen of the activatable antibody after activation). In some embodiments, the masking efficiency is measured by dividing the EC50 for binding of an activatable antibody comprising a masking peptide (before activation) by the EC50 of the parental antibody lacking the masking peptide (e.g., by measuring EC50 by ELISA; see e.g., the methods of Example 5). In some embodiments, masking efficiency is measured as the difference in affinity of an activatable antibody comprising the masking peptide for binding its target before activation relative to the affinity of the activatable antibody comprising the masking peptide, or a portion thereof (e.g., the MM) for binding its target after activation (e.g., the difference in affinity for a target antigen (such as CD47) of an activatable antibody before activation relative to the activatable antibody after activation). In some embodiments, the masking peptide, or a portion thereof (e.g., the MM) binds to the target binding moiety (TBM), and prevents the activatable antibody from binding to its target (e.g., CD47). In some embodiments, the masking peptide, or a portion thereof (e.g., the MM) has a dissociation constant for binding to the TBM that is greater than the dissociation constant of the TBM for its target (e.g., CD47). Dissociation constants can be measured, e.g., by techniques such as ELISA, surface plasmon resonance or Bio-Layer Interferometry (BLI), or flow cytometry.

In some embodiments, the masking peptide, or a portion thereof (e.g., the MM), has a masking efficiency of at least about 2.0 (e.g., at least about 2.0, at least about 3.0, at least about 4.0, at least about 5.0, at least about 6.0, at least about 7.0, at least about 8.0, at least about 9.0, at least about 10, at least about 25, at least about 50, at least about 75, at least about 100, at least about 150, at least about 200, at least about 300, at least about 400, at least about 500, etc.) as determined by a Jurkat NFAT reporter assay for example, as described in U.S. patent application Ser. No. 16/966,848. In some embodiments, the masking peptide, or a portion thereof (e.g., the MM), has a masking efficiency of at least about 50 as determined by a Jurkat NFAT reporter assay. In certain embodiments, the masking peptide, or a portion thereof (e.g., the MM), has a masking efficiency of at least about 100 as determined by a Jurkat NFAT reporter assay. In some embodiments, the masking peptide, or a portion thereof (e.g., the MM), has a masking efficiency of at least about 2.0 (e.g., at least about 2.0, at least about 3.0, at least about 4.0, at least about 5.0, at least about 6.0, at least about 7.0, at least about 8.0, at least about 9.0, at least about 10, at least about 25, at least about 50, at least about 75, at least about 100, at least about 150, at least about 200, at least about 300, at least about 400, at least about 500, etc.) prior to activation. In some embodiments, the masking peptide, or a portion thereof (e.g., the MM), has a masking efficiency of at most about 1.75 (e.g., at most about 1.75, at most about 1.5, at most about 1.4, at most about 1.3, at most about 1.2, at most about 1.1, at most about 1.0, at most about 0.9, at most about 0.8, at most about 0.7, at most about 0.6, or at most about 0.5, etc.) after to activation (e.g., the relative affinity of the activatable antibody after activation as compared to the affinity of a parental antibody).

In some embodiments, the activatable antibody has a greater Kp for binding to CD47 (e.g., human CD47) when the CM is not cleaved than when the CM is cleaved. In some embodiments, the activatable antibody binds human CD47 with a Kp of greater than 50 nM (e.g., about 60 nM, about 70 nM, about 80 nM, about 90 nM, about 100 nM, about 150 nM, about 200 nM, about 300 nM, about 400 nM, about 500 nM, about 1 μM, or greater) when the CM is not cleaved, and/or binds human CD47 with a Kp of less than 50 nM (e.g., about 40 nM, about 30 nM, about 20 nM, about 10 nM, about 5 nM, about 4 nM, about 3 nM, about 2 nM, about 1 nM, about 0.5 nM, about 0.4 nM, about 0.3 nM, about 0.2 nM, about 0.1 nM, or less) when the CM is cleaved. In some embodiments, the activatable antibody binds human CD47 with a Kp of greater than 50 nM (e.g., about 60 nM, about 70 nM, about 80 nM, about 90 nM, about 100 nM, about 150 nM, about 200 nM, about 300 nM, about 400 nM, about 500 nM, about 1 μM, or greater) when the CM is not cleaved, and/or binds human CD47 with a Kp of less than 10 nM (e.g., about 9 nM, about 8 nM, about 7 nM, about 7 nM, about 5 nM, about 4 nM, about 3 nM, about 2 nM, about 1 nM, about 0.5 nM, about 0.4 nM, about 0.3 nM, about 0.2 nM, about 0.1 nM, or less) when the CM is cleaved. In some embodiments, the activatable antibody binds human CD47 with a Kp of greater than 100 nM (e.g., about 110 nM, about 120 nM, about 130 nM, about 140 nM, about 150 nM, about 200 nM, about 300 nM, about 400 nM, about 500 nM, about 1 μM, or greater) when the CM is not cleaved, and/or binds human CD47 with a Kp of less than 10 nM (e.g., about 9 nM, about 8 nM, about 7 nM, about 7 nM, about 5 nM, about 4 nM, about 3 nM, about 2 nM, about 1 nM, about 0.5 nM, about 0.4 nM, about 0.3 nM, about 0.2 nM, about 0.1 nM, or less) when the CM is cleaved.

In some embodiments, the activatable antibody has a greater half maximal effective concentration (EC50) for binding to CD47 (e.g., human CD47) when the CM is not cleaved than when the CM is cleaved. In some embodiments, the activatable antibody has an EC50 of greater than 50 nM (e.g., about 60 nM, about 70 nM, about 80 nM, about 90 nM, about 100 nM, about 150 nM, about 200 nM, about 300 nM, about 400 nM, about 500 nM, about 1 μM, or greater) for binding human CD47 in vitro when the CM is not cleaved, and/or has an EC50 of less than 50 nM (e.g., about 40 nM, about 30 nM, about 20 nM, about 10 nM, about 5 nM, about 4 nM, about 3 nM, about 2 nM, about 1 nM, about 0.5 nM, about 0.4 nM, about 0.3 nM, about 0.2 nM, about 0.1 nM, or less) for binding human CD47 in vitro when the CM is cleaved. In some embodiments, the activatable antibody has an EC50 of greater than 50 nM (e.g., about 60 nM, about 70 nM, about 80 nM, about 90 nM, about 100 nM, about 150 nM, about 200 nM, about 300 nM, about 400 nM, about 500 nM, about 1 μM, or greater) for binding human CD47 in vitro when the CM is not cleaved, and/or has an EC50 of less than 10 nM (e.g., about 9 nM, about 8 nM, about 7 nM, about 7 nM, about 5 nM, about 4 nM, about 3 nM, about 2 nM, about 1 nM, about 0.5 nM, about 0.4 nM, about 0.3 nM, about 0.2 nM, about 0.1 nM, or less) for binding human CD47 in vitro when the CM is cleaved. In some embodiments, the activatable antibody has an EC50 of greater than 100 nM (e.g., about 110 nM, about 120 nM, about 130 nM, about 140 nM, about 150 nM, about 200 nM, about 300 nM, about 400 nM, about 500 nM, about 1 μM, or greater) for binding human CD47 in vitro when the CM is not cleaved, and/or has an EC50 of less than 10 nM (e.g., about 9 nM, about 8 nM, about 7 nM, about 7 nM, about 5 nM, about 4 nM, about 3 nM, about 2 nM, about 1 nM, about 0.5 nM, about 0.4 nM, about 0.3 nM, about 0.2 nM, about 0.1 nM, or less) for binding human CD47 in vitro when the CM is cleaved.

In some embodiments, the activatable antibody has a higher half maximal inhibitory concentration (IC50) for blocking binding of CD47 (e.g., human CD47) to SIRPα (e.g., human SIRPα) in vitro when the CM is not cleaved than when the CM is cleaved. In some embodiments, the activatable antibody has an IC50 of greater than 50 nM (e.g., about 60 nM, about 70 nM, about 80 nM, about 90 nM, about 100 nM, about 150 nM, about 200 nM, about 300 nM, about 400 nM, about 500 nM, about 1 μM, or greater) for blocking binding of human CD47 to human SIRPα in vitro when the CM is not cleaved, and/or has an IC50 of less than 50 nM (e.g., about 40 nM, about 30 nM, about 20 nM, about 10 nM, about 5 nM, about 4 nM, about 3 nM, about 2 nM, about 1 nM, about 0.5 nM, about 0.4 nM, about 0.3 nM, about 0.2 nM, about 0.1 nM, or less) for blocking binding of human CD47 to human SIRPα in vitro when the CM is cleaved. In some embodiments, the activatable antibody has an IC50 of greater than 50 nM (e.g., about 60 nM, about 70 nM, about 80 nM, about 90 nM, about 100 nM, about 150 nM, about 200 nM, about 300 nM, about 400 nM, about 500 nM, about 1 μM, or greater) for blocking binding of human CD47 to human SIRPα in vitro when the CM is not cleaved, and/or has an IC50 of less than 10 nM (e.g., about 9 nM, about 8 nM, about 7 nM, about 7 nM, about 5 nM, about 4 nM, about 3 nM, about 2 nM, about 1 nM, about 0.5 nM, about 0.4 nM, about 0.3 nM, about 0.2 nM, about 0.1 nM, or less) for blocking binding of human CD47 to human SIRPα in vitro when the CM is cleaved. In some embodiments, the activatable antibody has an IC50 of greater than 100 nM (e.g., about 110 nM, about 120 nM, about 130 nM, about 140 nM, about 150 nM, about 200 nM, about 300 nM, about 400 nM, about 500 nM, about 1 μM, or greater) for blocking binding of human CD47 to human SIRPα in vitro when the CM is not cleaved, and/or has an IC50 of less than 10 nM (e.g., about 9 nM, about 8 nM, about 7 nM, about 7 nM, about 5 nM, about 4 nM, about 3 nM, about 2 nM, about 1 nM, about 0.5 nM, about 0.4 nM, about 0.3 nM, about 0.2 nM, about 0.1 nM, or less) for blocking binding of human CD47 to human SIRPα in vitro when the CM is cleaved.

In certain embodiments, the activatable antibody completely blocks binding of human CD47 to human SIRPα in vitro when the activatable antibody is provided at a concentration of about 1 nM or greater (e.g., about 1 nM or greater, about 5 nM or greater, about 10 nM or greater, about 20 nM or greater, about 40 nM or greater, about 60 nM or greater, about 80 nM or greater, about 100 nM or greater, about 200 nM or greater, about 400 nM or greater, about 600 nM or greater, about 800 nM or greater, about 1 μM or greater, about 2 μM or greater, about 4 μM or greater, about 6 μM or greater, about 8 UM or greater, about 10 UM or greater, about 20 μM or greater, about 40 μM or greater, about 60 μM or greater, about 80 UM or greater, about 100 UM or greater, etc.) and the CM is cleaved. In some embodiments, activatable antibody completely blocks binding of human CD47 to human SIRPα in vitro when the activatable antibody is provided at a concentration of about 1 UM or greater and the CM is cleaved.

In some embodiments, the activatable antibody has a greater half maximal effective concentration (EC50) for binding to tumor cells in vitro when the CM is not cleaved than when the CM is cleaved. In some embodiments, the activatable antibody has an EC50 of greater than 10 nM (e.g., about 15 nM, about 20 nM, about 30 nM, about 40 nM, about 50 nM, about 100 nM, about 200 nM, about 300 nM, about 400 nM, about 500 nM, about 1 μM, or greater) for binding to tumor cells in vitro when the CM is not cleaved, and/or has an EC50 of less than 10 nM (e.g., about 9 nM, about 8 nM, about 7 nM, about 7 nM, about 5 nM, about 4 nM, about 3 nM, about 2 nM, about 1 nM, about 0.5 nM, about 0.4 nM, about 0.3 nM, about 0.2 nM, about 0.1 nM, or less) for binding to tumor cells in vitro when the CM is cleaved. In some embodiments, the activatable antibody has an EC50 of greater than 10 nM (e.g., about 15 nM, about 20 nM, about 30 nM, about 40 nM, about 50 nM, about 100 nM, about 200 nM, about 300 nM, about 400 nM, about 500 nM, about 1 μM, or greater) for binding to tumor cells in vitro when the CM is not cleaved, and/or has an EC50 of less than 1 nM (e.g., about 0.9 nM, about 0.8 nM, about 0.7 nM, about 0.6 nM, about 0.5 nM, about 0.4 nM, about 0.3 nM, about 0.2 nM, about 0.1 nM, or less) for binding to tumor cells in vitro when the CM is cleaved. In some embodiments, the activatable antibody has an EC50 of greater than 50 nM (e.g., about 60 nM, about 70 nM, about 80 nM, about 90 nM, about 100 nM, about 150 nM, about 200 nM, about 300 nM, about 400 nM, about 500 nM, about 1 μM, or greater) for binding to tumor cells in vitro when the CM is not cleaved, and/or has an EC50 of less than 1 nM (e.g., about 0.9 nM, about 0.8 nM, about 0.7 nM, about 0.6 nM, about 0.5 nM, about 0.4 nM, about 0.3 nM, about 0.2 nM, about 0.1 nM, or less) for binding to tumor cells in vitro when the CM is cleaved. In certain embodiments, the tumor cells comprise a B cell lymphoma cell line (e.g., Raji cell line). In further embodiments, the tumor cells comprise a T cell lymphoma cell line (e.g., CEM cell line). In some embodiments, the activatable antibody has an EC50 of greater than 10 nM for binding to tumor cells comprising a B cell lymphoma cell line (e.g., Raji cell line) in vitro when the CM is not cleaved, and/or has an EC50 of less than 1 nM for binding to tumor cells comprising the B cell lymphoma cell line (e.g., Raji cell line) in vitro when the CM is cleaved. In some embodiments, the activatable antibody has an EC50 of greater than 50 nM for binding to tumor cells comprising a T cell lymphoma cell line (e.g., CEM cell line) in vitro when the CM is not cleaved, and/or has an EC50 of less than 1 nM for binding to tumor cells comprising the T cell lymphoma cell line (e.g., CEM cell line) in vitro when the CM is cleaved.

In some embodiments, the activatable antibody comprising the masking peptide and target-binding moiety (TBM) has a reduced binding to red blood cells (RBCs) in vitro as compared to a parental antibody having the same TBM but lacking the masking peptide. In some embodiments, the activatable antibody has a greater half maximal effective concentration (EC50) for binding to RBCs in vitro when the CM is not cleaved than an antibody having the same TBM (e.g., the same VH and VL) and lacking the masking peptide. In some embodiments, the activatable antibody having a masking peptide, a VH, and a VL has an EC50 of greater than 100 nM (e.g., about 110 nM, about 120 nM, about 130 nM, about 140 nM, about 150 nM, about 200 nM, about 300 nM, about 400 nM, about 500 nM, about 1 μM, or greater) for binding to RBCs in vitro when the CM is not cleaved, and an antibody having the same VH and VL and lacking the masking peptide has an EC50 of less than 10 nM (e.g., about 9 nM, about 8 nM, about 7 nM, about 7 nM, about 5 nM, about 4 nM, about 3 nM, about 2 nM, about 1 nM, about 0.5 nM, about 0.4 nM, about 0.3 nM, about 0.2 nM, about 0.1 nM, or less) for binding to RBCs in vitro under the same assay conditions. In certain embodiments, the activatable antibody having a masking peptide, a VH, and a VL has an EC50 for binding to RBCs in vitro when the CM is not cleaved of about 10 fold, about 50 fold, about 100 fold, about 200 fold, about 300 fold, about 400 fold, about 500 fold, about 600 fold, about 700 fold, about 800 fold, about 900 fold, about 1000 fold, about 1500 fold, about 2000 fold, or about 2500 fold greater than that of an antibody having the same VH and VL and lacking the masking peptide.

In certain embodiments, the activatable antibody has reduced binding to RBCs in vitro when the cleavable moiety (CM) is not cleaved as compared to the same activatable antibody when the CM is cleaved. In some embodiments, the activatable antibody has a greater half maximal effective concentration (EC50) for binding to RBCs in vitro when the CM is not cleaved than when the CM is cleaved. In some embodiments, the activatable antibody has an EC50 of greater than 100 nM (e.g., about 110 nM, about 120 nM, about 130 nM, about 140 nM, about 150 nM, about 200 nM, about 300 nM, about 400 nM, about 500 nM, about 1 μM, or greater) for binding to RBCs in vitro when the CM is not cleaved, and/or has an EC50 of greater less 50 nM (e.g., about 40 nM, about 30 nM, about 20 nM, about 10 nM, about 5 nM, about 4 nM, about 3 nM, about 2 nM, about 1 nM, about 0.5 nM, about 0.4 nM, about 0.3 nM, about 0.2 nM, about 0.1 nM, or less) for binding to RBCs in vitro when the CM is cleaved. In some embodiments, the activatable antibody does not induce hemagglutination of RBCs in vitro when provided at a concentration of about 1 mM or less (e.g., about 1 mM or less, about 0.5 mM or less, about 0.1 mM or less, about 50 μM or less, about 10 UM or less, about 5 μM or less, about 4 μM or less, about 3 μM or less, about 2 μM or less, about 1 μM or less, about 500 μM or less, about 100 μM or less, or about 50 μM or less).

In some embodiments, the activatable antibody has a greater half maximal effective concentration (EC50) for increasing macrophage phagocytosis of tumor cells in vitro when the CM is not cleaved than when the CM is cleaved. In certain embodiments, the activatable antibody has an EC50 of greater than 20 nM (e.g., about 20 mM, about 30 nM, about 40 nM, about 50 nM, about 60 nM, about 70 nM, about 80 nM, about 90 nM, about 100 nM, about 150 nM, about 200 nM, about 300 nM, about 400 nM, about 500 nM, about 1 μM, or greater) for increasing macrophage phagocytosis of tumor cells in vitro when the CM is not cleaved, and/or has an IC50 of less than 20 nM (e.g., about 15 nM, about 10 nM, about 5 nM, about 4 nM, about 3 nM, about 2 nM, about 1 nM, about 0.5 nM, about 0.4 nM, about 0.3 nM, about 0.2 nM, about 0.1 nM, or less) for increasing macrophage phagocytosis of tumor cells in vitro when the CM is cleaved. In certain embodiments, the activatable antibody has an EC50 of greater than 20 nM (e.g., about 20 mM, about 30 nM, about 40 nM, about 50 nM, about 60 nM, about 70 nM, about 80 nM, about 90 nM, about 100 nM, about 150 nM, about 200 nM, about 300 nM, about 400 nM, about 500 nM, about 1 μM, or greater) for increasing macrophage phagocytosis of tumor cells in vitro when the CM is not cleaved, and/or has an IC50 of less than 10 nM (e.g., about 9 nM, about 8 nM, about 7 nM, about 7 nM, about 5 nM, about 4 nM, about 3 nM, about 2 nM, about 1 nM, about 0.5 nM, about 0.4 nM, about 0.3 nM, about 0.2 nM, about 0.1 nM, or less) for increasing macrophage phagocytosis of tumor cells in vitro when the CM is cleaved. In certain embodiments, the activatable antibody has an EC50 of about 20 nM or greater for increasing macrophage phagocytosis of tumor cells in vitro when the CM is not cleaved; and/or has an EC50 of about 1 nM or less for increasing macrophage phagocytosis of tumor cells in vitro when the CM is cleaved. In some embodiments, providing the activatable antibody at a concentration of about 1 μM or greater results in a maximum macrophage phagocytosis of tumor cells in vitro of about 20% or less (e.g., about 20%, about 19%, about 18%, about 17%, about 16%, about 15%, about 10%, about 5%, or less) when the CM is not cleaved, and/or results in a maximum macrophage phagocytosis of tumor cells in vitro of about 50% or greater (e.g., about 50%, about 60%, about 70%, about 80%, about 90%, about 95%, or greater) when the CM is cleaved.

### B-4. Antigen-Binding Fragments

In some other aspects, the present disclosure provides antigen-binding fragments of any of the CD47 antibodies or masked antibodies (e.g., activatable antibodies) provided by the present disclosure.

The antigen-binding fragment may comprise any sequences of the antibody or masked antibody (e.g., activatable antibody). In some embodiments, the antigen-binding fragment comprises the amino acid sequence of: (1) a light chain of a CD47 antibody or masked antibody (e.g., activatable antibody); (2) a heavy chain of a CD47 antibody or masked antibody (e.g., activatable antibody); (3) a variable region from the light chain of a CD47 antibody or masked antibody (e.g., activatable antibody); (4) a variable region from the heavy chain of a CD47 antibody or masked antibody (e.g., activatable antibody); (5) one or more CDRs (two, three, four, five, or six CDRs) of a CD47 antibody or masked antibody (e.g., activatable antibody); or (6) three CDRs from the light chain and three CDRs from the heavy chain of a CD47 antibody or masked antibody (e.g., activatable antibody); or (7) a target binding moiety (TBM) of a CD47 antibody or masked antibody (e.g., activatable antibody).

In some particular embodiments, the disclosure provides an antigen-binding fragment of an antibody or masked antibody (e.g., activatable antibody) selected from those listed in Tables 3A-6.

In some other particular embodiments, the antigen-binding fragments of an CD47 antibody or masked antibody (e.g., activatable antibody) include: (i) a Fab fragment, which is a monovalent fragment consisting of the VL, VH, CL and CH1 domains; (ii) a F(ab′) 2 fragment, which is a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody or masked antibody (e.g., activatable antibody); (v) a dAb fragment (Ward et al., (1989) Nature 341:544-546), which consists of a VH domain; (vi) an isolated CDR, and (vii) single chain antibody (scFv), which is a polypeptide comprising a VL region of an antibody or masked antibody (e.g., activatable antibody) linked to a VH region of an antibody or masked antibody (e.g., activatable antibody). Bird et al., (1988) Science 242:423-426 and Huston et al., (1988) Proc. Natl. Acad. Sci. USA 85:5879-5883.

### B-5. Illustrative Anti-CD47 Antibodies, Masked Antibodies, and Antigen-Binding Fragments

Provided herein are illustrative antibodies, masked antibodies (including, e.g., activatable antibodies), and antigen-binding fragments thereof that target CD47 (e.g., human CD47), as described in Tables 3A-6.

TABLE 3A
Heavy chain variable region complementarity-determining region (VH-
CDRs) sequences of illustrative anti-CD47 antibodies, masked antibodies (e.g.,
activatable antibodies), and antigen-binding fragments.

| Illustrative | | | |
|---|---|---|---|
| Antibody | VH-CDR1 | VH-CDR2 | VH-CDR3 |
| TY25029 | NYWIH | IIYPSSGSTKYAQKFQG | DRAPGSSGYYDGFDY |
| | (SEQ ID NO: 35) | (SEQ ID NO: 52) | (SEQ ID NO: 69) |
| TY25030 | SGYYWS | EIYHSGNTYYNPSLKS | RVPLGAFDY |
| | (SEQ ID NO: 36) | (SEQ ID NO: 53) | (SEQ ID NO: 70) |
| TY25031 | SGYHWG | RIYPSSGSTKYAQKFQG | GVRGSYGWFDV |
| | (SEQ ID NO: 37) | (SEQ ID NO: 54) | (SEQ ID NO: 71) |
| TY25032 | SYGIH | AISGSGGSTYYADSVKG | SGHAYDFDY |
| | (SEQ ID NO: 38) | (SEQ ID NO: 55) | (SEQ ID NO: 72) |
| TY25033 | SYAIH | IINPNFGSTNYAQKFQG | YYYGASFDV |
| | (SEQ ID NO: 39) | (SEQ ID NO: 56) | (SEQ ID NO: 73) |
| TY25034 | SGHYWN | IISPSGGGTKYAQKFQG | GARGYYALDY |
| | (SEQ ID NO: 40) | (SEQ ID NO: 57) | (SEQ ID NO: 74) |
| TY25035 | SGYHWD | RIYWDGDKRYSPSLKS | FSDGFDP |
| | (SEQ ID NO: 41) | (SEQ ID NO: 58) | (SEQ ID NO: 75) |
| TY25036 | GYAIH | WISPSSGSTKYAQKFQG | YGYGPVFDY |
| | (SEQ ID NO: 42) | (SEQ ID NO: 59) | (SEQ ID NO: 76) |
| TY25037 | SGHYWT | GIIPVFGTPNYAQKFQG | YGYGGAYFDV |
| | (SEQ ID NO: 43) | (SEQ ID NO: 60) | (SEQ ID NO: 77) |
| TY25038 | RYAIH | GIIPVFGTANYAQKFQG | QGILGGFAY |
| | (SEQ ID NO: 44) | (SEQ ID NO: 61) | (SEQ ID NO: 78) |
| TY25039 | SGYYWG | AISYSGSTYYSPSLKS | HYLFAGSTSYDAFDI |
| | (SEQ ID NO: 45) | (SEQ ID NO: 62) | (SEQ ID NO: 79) |
| TY25040 | DYGIH | RIYPSGGSTNYAQKFQG | QRGYGRFAY |
| | (SEQ ID NO: 46) | (SEQ ID NO: 63) | (SEQ ID NO: 80) |
| TY25041 | SGHHWN | AISYSGSTYYSPSLKS | QGYYGGEGYAVDY |
| | (SEQ ID NO: 47) | (SEQ ID NO: 64) | (SEQ ID NO: 81) |
| TY21446 | NYAIH | AISGSGSSTYYADSVKG | RGSYGFGAFDY |
| | (SEQ ID NO: 48) | (SEQ ID NO: 65) | (SEQ ID NO: 82) |
| TY21447 | NYAIH | AISGSGSSTYYADSVKG | RGSYGFGAFDY |
| | (SEQ ID NO: 49) | (SEQ ID NO: 66) | (SEQ ID NO: 83) |
| TY21449 | GYAIH | AISGSGSSTYYADSVKG | RGSYGFGAFDY |
| | (SEQ ID NO: 50) | (SEQ ID NO: 67) | (SEQ ID NO: 84) |
| TY21451 | DYWIH | GISGAGGSTYYADSVKG | LGDY |
| | (SEQ ID NO: 51) | (SEQ ID NO: 68) | (SEQ ID NO: 85) |

TABLE 3B
Light chain variable region complementarity-determining region (VL-CDRs)
sequences of illustrative anti-CD47 antibodies, masked antibodies
(e.g., activatable antibodies), and antigen-binding fragments.

| Illustrative | | | |
|---|---|---|---|
| Antibody | VL-CDR1 | VL-CDR2 | VL-CDR3 |
| TY25029 | RASQSVDFHGISFLH | DASSLES | HQRSTTPLT |
| | (SEQ ID NO: 86) | (SEQ ID NO: 103) | (SEQ ID NO: 120) |
| TY25030 | RASQTVISRYLA | DASNLET | QQYYAWPLT |
| | (SEQ ID NO: 87) | (SEQ ID NO: 104) | (SEQ ID NO: 121) |
| TY25031 | SASSRVSYVY | AASTLQS | QHYVSSPRVYT |
| | (SEQ ID NO: 88) | (SEQ ID NO: 105) | (SEQ ID NO: 122) |
| TY25032 | RASQSVDFYGISFLD | AASTLQS | QQGGTSPWT |
| | (SEQ ID NO: 89) | (SEQ ID NO: 106) | (SEQ ID NO: 123) |
| TY25033 | KSGQSLLHGDVKTYLY | DASNRAT | QQYASWPPGFT |
| | (SEQ ID NO: 90) | (SEQ ID NO: 107) | (SEQ ID NO: 124) |
| TY25034 | RASQSVRGRLLA | AASSLQS | AQYLELPYT |
| | (SEQ ID NO: 91) | (SEQ ID NO: 108) | (SEQ ID NO: 125) |
| TY25035 | RASQSVDFVGISFLH | DASNRAT | VQGLQWPHT |
| | (SEQ ID NO: 92) | (SEQ ID NO: 109) | (SEQ ID NO: 126) |
| TY25036 | SASSSVTYIY | DASNRAT | QQYTSSPRGFT |
| | (SEQ ID NO: 93) | (SEQ ID NO: 110) | (SEQ ID NO: 127) |
| TY25037 | RASQSVDFHGISFLH | DASNRAT | VQGIQWPWT |
| | (SEQ ID NO: 94) | (SEQ ID NO: 111) | (SEQ ID NO: 128) |
| TY25038 | RASESVDFHGKSFLA | AASSLQS | QQYLELPFT |
| | (SEQ ID NO: 95) | (SEQ ID NO: 112) | (SEQ ID NO: 129) |
| TY25039 | RASQSVDFYGFSFLA | DASNRAT | QQSYRTPLT |
| | (SEQ ID NO: 96) | (SEQ ID NO: 113) | (SEQ ID NO: 130) |
| TY25040 | SASSSVGYVY | DASSLES | QQYTYAPFT |
| | (SEQ ID NO: 97) | (SEQ ID NO: 114) | (SEQ ID NO: 131) |
| TY25041 | RASQSVDFYGISFLH | DASNRAT | VQATQTPFT |
| | (SEQ ID NO: 98) | (SEQ ID NO: 115) | (SEQ ID NO: 132) |
| TY21446 | RASQTIGRYLN | DASNRAT | QQRYPWPYT |
| | (SEQ ID NO: 99) | (SEQ ID NO: 116) | (SEQ ID NO: 133) |
| TY21447 | RASQTIGRYLN | DASNRAT | QQRYPWPYT |
| | (SEQ ID NO: 100) | (SEQ ID NO: 117) | (SEQ ID NO: 134) |
| TY21449 | RASQTIGRYLN | DASNRAT | QQRYPWPYT |
| | (SEQ ID NO: 101) | (SEQ ID NO: 118) | (SEQ ID NO: 135) |
| TY21451 | RASQGISSVLA | AASTLQS | QQYYSIPFT |
| | (SEQ ID NO: 102) | (SEQ ID NO: 119) | (SEQ ID NO: 136) |

TABLE 4A
Heavy chain variable region (VH) and light chain variable region (VL)
sequences of illustrative anti-CD47 antibodies, masked antibodies
(e.g., activatable antibodies), and antigen-binding fragments.

| | Illustrative | |
|---|---|---|
| | Antibody | |
| SEQ ID NO | and Region | Amino Acid Sequence |
| SEQ ID | TY25029- | EVQLVESGGGLVQPGGSLRLSCAASGYTFSNYWIHWVRQAP |
| NO: 1 | VH | GKGLEWIGIIYPSSGSTKYAQKFQGRVTISRDNSKNTLYLQLN |
| | | SLRAEDTAVYYCARDRAPGSSGYYDGFDYWGQGTLVTVSS |
| SEQ ID | TY25029- | DIQLTQSPSSLSASVGDRVTITCRASQSVDFHGISFLHWYQQK |
| NO: 2 | VL | PGKAPKLLIYDASSLESGVPSRFSGSGSGTDFTLTISSLQPEDFA |
| | | TYYCHQRSTTPLTFGQGTKVEIKR |
| SEQ ID | TY25030- | EVQLVESGGGLVQPGGSLRLSCAASGYSITSGYYWSWIRQAP |
| NO: 3 | VH | GKGLEWIGEIYHSGNTYYNPSLKSRVTISRDNSKNTLYLQLNS |
| | | LRAEDTAVYYCARRVPLGAFDYWGQGTLVTVSS |
| SEQ ID | TY25030- | DIQLTQSPSSLSASVGDRVTITCRASQTVISRYLAWYQQKPGK |
| NO: 4 | VL | APKLLIYDASNLETGVPSRFSGSGSGTDFTLTISSLQPEDFATY |
| | | YCQQYYAWPLTFGQGTKVEIKR |
| SEQ ID | TY25031- | EVQLVESGGGLVQPGGSLRLSCAASGYSITSGYHWGWIRQAP |
| NO: 5 | VH | GKGLEWIGRIYPSSGSTKYAQKFQGRVTISRDNSKNTLYLQLN |
| | | SLRAEDTAVYYCARGVRGSYGWFDVWGQGTLVTVSS |
| SEQ ID | TY25031- | DIQLTQSPSSLSASVGDRVTITCSASSRVSYVYWYQQKPGKAP |
| NO: 6 | VL | KLLIYAASTLQSGVPSRFSGSGSGTDFTLTISSLQPEDFATYYC |
| | | QHYVSSPRVYTFGQGTKVEIKR |
| SEQ ID | TY25032- | EVQLVESGGGLVQPGGSLRLSCAASGFTFTSYGIHWVRQAPG |
| NO: 7 | VH | KGLEWVSAISGSGGSTYYADSVKGRFTISRDNSKNTLYLQLN |
| | | SLRAEDTAVYYCARSGHAYDFDYWGQGTLVTVSS |
| SEQ ID | TY25032- | DIQLTQSPSSLSASVGDRVTITCRASQSVDFYGISFLDWYQQK |
| NO: 8 | VL | PGKAPKLLIYAASTLQSGVPSRFSGSGSGTDFTLTISSLQPEDF |
| | | ATYYCQQGGTSPWTFAQGTKVEIKR |
| SEQ ID | TY25033- | QVQLVQSGAEVKKPGSSVKVSCKASGFTFTSYAIHWVRQAPG |
| NO: 9 | VH | QGLEWIGIINPNFGSTNYAQKFQGRVTITADKSTSTAYMELSS |
| | | LRSEDTAVYYCARYYYGASFDVWGQGTLVTVSS |
| SEQ ID | TY25033- | DIQLTQSPSSLSASVGDRVTITCKSGQSLLHGDVKTYLYWYQ |
| NO: 10 | VL | QKPGKAPKLLIYDASNRATGIPSRESGSGSGTDFTLTISSLQPE |
| | | DFATYYCQQYASWPPGFTFGQGTKVEIKR |
| SEQ ID | TY25034- | EVQLVESGGGLVQPGGSLRLSCAASGYSITSGHYWNWIRQAP |
| NO: 11 | VH | GKGLEWIGIISPSGGGTKYAQKFQGRVTISRDNSKNTLYLQLN |
| | | SLRAEDTAVYYCARGARGYYALDYWGQGTLVTVSS |
| SEQ ID | TY25034- | EIVLTQSPATLSLSPGERATLSCRASQSVRGRLLAWYQQKPGQ |
| NO: 12 | VL | APRLLIYAASSLQSGVPARFSGSGSGTDFTLTISSLEPEDFAVY |
| | | YCAQYLELPYTFGQGTKVEIKR |
| SEQ ID | TY25035- | EVQLVESGGGLVQPGGSLRLSCAASGYSISSGYHWDWIRQAP |
| NO: 13 | VH | GKGLEWLARIYWDGDKRYSPSLKSRLTISRDNSKNTLYLQLN |
| | | SLRAEDTAVYYCARFSDGFDPWGQGTLVTVSS |
| SEQ ID | TY25035- | EIVLTQSPATLSLSPGERATLSCRASQSVDFVGISFLHWYQQKP |
| NO: 14 | VL | GQAPRLLIYDASNRATGIPARFSGSGSGTDFTLTISSLEPEDFA |
| | | VYYCVQGLQWPHTFGQGTKVEIKR |
| SEQ ID | TY25036- | QVQLVQSGAEVKKPGSSVKVSCKASGYTFTGYAIHWVRQAP |
| NO: 15 | VH | GQGLEWIGWISPSSGSTKYAQKFQGRVTITADKSTSTAYMELS |
| | | SLRSEDTAVYYCARYGYGPVFDYWGQGTLVTVSS |
| SEQ ID | TY25036- | DIQLTQSPSSLSASVGDRVTITCSASSSVTYIYWYQQKPGKAP |
| NO: 16 | VL | KLLIYDASNRATGIPSRFSGSGSGTDFTLTISSLQPEDFATYYC |
| | | QQYTSSPRGFTFGQGTKVEIKR |
| SEQ ID | TY25037- | QVQLVQSGAEVKKPGSSVKVSCKASGYSITSGHYWTWIRQAP |
| NO: 17 | VH | GQGLEWIGGIIPVFGTPNYAQKFQGRVTITADKSTSTAYMELS |
| | | SLRSEDTAVYYCASYGYGGAYFDVWGQGTLVTVSS |
| SEQ ID | TY25037- | DIQLTQSPSSLSASVGDRVTITCRASQSVDFHGISFLHWYQQK |
| NO: 18 | VL | PGKAPKLLIYDASNRATGIPSRFSGSGSGTDFTLTISSLQPEDFA |
| | | TYYCVQGIQWPWTFGQGTKVEIKR |
| SEQ ID | TY25038- | QVQLVQSGAEVKKPGSSVKVSCKASGFTFSRYAIHWVRQAP |
| NO: 19 | VH | GQGLEWLGGIIPVFGTANYAQKFQGRVTITADKSTSTAYMEL |
| | | SSLRSEDTAVYYCARQGILGGFAYWGQGTLVTVSS |
| SEQ ID | TY25038- | DIQLTQSPSSLSASVGDRVTITCRASESVDFHGKSFLAWYQQK |
| NO: 20 | VL | PGKAPKLLIYAASSLQSGVPSRFSGSGSGTDFTLTISSLQPEDF |
| | | ATYYCQQYLELPFTFGQGTKVEIKR |
| SEQ ID | TY25039- | QVQPAQSGAEVKKPGSSVKVSCKASGYSISSGYYWGWIRQAP |
| NO: 21 | VH | GQGLEWIGAISYSGSTYYSPSLKSRVTITADKSTSTAYMELSSL |
| | | RSEDTAVYYCARHYLFAGSTSYDAFDIWGQGTLVTVSS |
| SEQ ID | TY25039- | DIQLTQSPSSLSASVGDRVTITCRASQSVDFYGFSFLAWYQQK |
| NO:: 22 | VL | PGKAPKLLIYDASNRATGIPSRESGSGSGTDFTLTISSLQPEDFA |
| | | TYYCQQSYRTPLTFGQGTKVEIKR |
| SEQ ID | TY25040- | QVQLVQSGAEVKKPGSSVKVSCKASGFTFSDYGIHWVRQAP |
| NO: 23 | VH | GQGLEWIGRIYPSGGSTNYAQKFQGRVTITADKSTSTAYMEL |
| | | SSLRSEDTAVYYCARQRGYGRFAYWGQGTLVTVSS |
| SEQ ID | TY25040- | DIQLTQSPSSLSASVGDRVTITCSASSSVGYVYWYQQKPGKAP |
| NO: 24 | VL | KLLIYDASSLESGVPSRFSGSGSGTDFTLTISSLQPEDFATYFCQ |
| | | QYTYAPFTFGQGTKVEIKR |
| SEQ ID | TY25041- | QVQLVQSGAEVKKPGSSVKVSCKASGYSISSGHHWNWIRQA |
| NO: 25 | VH | PGQGLEWIGAISYSGSTYYSPSLKSRVTITADKSTSTAYMELSS |
| | | LRSEDTAVYYCARQGYYGGEGYAVDYWGQGTLVTVSS |
| SEQ ID | TY25041- | DIQLTQSPSSLSASVGDRVTITCRASQSVDFYGISFLHWYQQK |
| NO: 26 | VL | PGKAPKLLIYDASNRATGIPSRFSGSGSGTDFTLTISSLQPEDFA |
| | | TYYCVQATQTPFTFGQGTKVEIKR |
| SEQ ID | TY21446- | EVQLVESGGGLVQPGGSLRLSCAASGFTFTNYAIHWVRQAPG |
| NO: 27 | VH | KGLEWVSAISGSGSSTYYADSVKGRFTISRDNSKNTLYLQLNS |
| | | LRAEDTAVYYCARRGSYGFGAFDYWGQGTLVTVSS |
| SEQ ID | TY21446- | DIQLTQSPSSLSASVGDRVTITCRASQTIGRYLNWYQQKPGKA |
| NO: 28 | VL | PKLLIYDASNRATGIPSRFSGSGSGTDFTLTISSLQPEDFATYYC |
| | | QQRYPWPYTFGQGTKVEIKR |
| SEQ ID | TY21447- | EVQLVESGGGLVQPGGSLRLSCAASGFTFSNYAIHWVRQAPG |
| NO: 29 | VH | KGLEWVSAISGSGSSTYYADSVKGRFTISRDNSKNTLYLQLNS |
| | | LRAEDTAVYYCARRGSYGFGAFDYWGQGTLVTVSS |
| SEQ ID | TY21447- | DIQLTQSPSSLSASVGDRVTITCRASQTIGRYLNWYQQKPGKA |
| NO: 30 | VL | PKLLIYDASNRATGIPSRFSGSGSGTDFTLTISSLQPEDFATYYC |
| | | QQRYPWPYTFGQGTKVEIKR |
| SEQ ID | TY21449- | EVQLVESGGGLVQPGGSLRLSCAASGFTFSGYAIHWVRQAPG |
| NO: 31 | VH | KGLEWVSAISGSGSSTYYADSVKGRFTISRDNSKNTLYLQLNS |
| | | LRAEDTAVYYCARRGSYGFGAFDYWGQGTLVTVSS |
| SEQ ID | TY21449- | DIQLTQSPSSLSASVGDRVTITCRASQTIGRYLNWYQQKPGKA |
| NO: 32 | VL | PKLLIYDASNRATGIPSRESGSGSGTDFTLTISSLQPEDFATYYC |
| | | QQRYPWPYTFGQGTKVEIKR |
| SEQ ID | TY21451- | EVQLVESGGGLVQPGGSLRLSCAASGFTFSDYWIHWVRQAPG |
| NO: 33 | VH | KGLEWVSGISGAGGSTYYADSVKGRFTISRDNSKNTLYLQLN |
| | | SLRAEDTAVYYCARLGDYWGQGTLVTVSS |
| SEQ ID | TY21451- | DIQLTQSPSSLSASVGDRVTITCRASQGISSVLAWYQQKPGKA |
| NO: 34 | VL | PKLLIYAASTLQSGVPSRFSGSGSGTDFTLTISSLQPEDFATYY |
| | | CQQYYSIPFTFGQGTKVEIKR |

TABLE 4B
Heavy chain variable region (VH) and light chain variable
region (VL) sequences of benchmark anti-CD47 antibodies.

| | Benchmark | |
|---|---|---|
| | Antibody | |
| SEQ ID NO | and Region | Amino Acid Sequence |
| SEQ ID NO: 201 | TAC2407_VH | EVQLVESGGGLVKPGGSLRLSCAASGLTFERAWMNWVRQ |
| | | APGKGLEWVGRIKRKTDGETTDYAAPVKGRFSISRDDSKNT |
| | | LYLQMNSLKTEDTAVYYCAGSNRAFDIWGQGTMVTVSS |
| SEQ ID NO: 202 | TAC2407_VL | DIVMTQSPDSLAVSLGERATINCKSSQSVLYAGNNRNYLAW |
| | | YQQKPGQPPKLLINQASTRASGVPDRFSGSGSGTEFTLIISSL |
| | | QAEDVAIYYCQQYYTPPLAFGGGTKLEIK |
| SEQ ID NO: 203 | TAC2204_VH | QVQLVQSGAEVKKPGASVKVSCKASGYTFTNYNMHWVRQ |
| | | APGQRLEWMGTIYPGNDDTSYNQKFKDRVTITADTSASTAY |
| | | MELSSLRSEDTAVYYCARGGYRAMDYWGQGTLVTVSS |
| SEQ ID NO: 204 | TAC2204_VL | DIVMTQSPLSLPVTPGEPASISCRSSQSIVYSNGNTYLGWYL |
| | | QKPGQSPQLLIYKVSNRFSGVPDRFSGSGSGTDFTLKISRVE |
| | | AEDVGVYYCFQGSHVPYTFGQGTKLEIK |

TABLE 5A
Masking moiety (MM) and cleavable moiety
(CM) sequences of illustrative
anti-CD47 activatable antibodies and
antigen-binding fragments thereof.

| | Illustrative | |
|---|---|---|
| | Antibody | |
| SEQ ID NO | and Region | Amino Acid Sequence |
| SEQ ID NO: 137 | TY26294_MM | LTVDYFCDIDPLYCNA |
| SEQ ID NO: 138 | TY26294_CM | GGGPLGLAGSGGS |
| SEQ ID NO: 167 | TY26137_MM | NNAAPDCPAADVYCNV |
| SEQ ID NO: 168 | TY26138_MM | DDTAFDCDDDFDDCAT |
| SEQ ID NO: 169 | TY26139_MM | HAYHNDCSDDDDFCAY |
| SEQ ID NO: 170 | TY26292_MM | PADDFDCPAADVFCSP |
| SEQ ID NO: 171 | TY26293_MM | APSVDDCPTYDDFCYD |
| SEQ ID NO: 172 | TY26294_MM | LTVDYFCDIDPLYCNA |
| SEQ ID NO: 173 | TY26295_MM | NYNDDDCYDDDYACDY |
| SEQ ID NO: 174 | TY26296_MM | DTADHDCYDDDDFCYD |
| SEQ ID NO: 175 | TY26297_MM | DFLSASCDDDYAPCAF |
| SEQ ID NO: 176 | TY26298_MM | YPPDDTCDDDYDPCAI |
| SEQ ID NO: 177 | TY26299_MM | AQSSFDCPDHDAFCDP |
| SEQ ID NO: 178 | TY26300_MM | AASPATCDDTLFFCPA |
| SEQ ID NO: 179 | TY26140_MM | DSCPAEVVGIFCIQ |
| SEQ ID NO: 180 | TY26141_MM | FYCTGAAVGIFCSA |
| SEQ ID NO: 181 | TY26301_MM | VACRIFDDDPFCIV |

TABLE 5B
Masking peptide sequences of illustrative
anti-CD47 activatable antibodies
and antigen-binding fragments thereof.

| | Illustrative | |
|---|---|---|
| SEQ ID NO | Antibody | Amino Acid Sequence |
| SEQ ID NO: 139 | TY26294 | EVGSYLTVDYFCDIDPLYCNA |
| | | GGGPLGLAGSGGS |
| SEQ ID NO: 152 | TY26137 | EVGSYNNAAPDCPAADVYCNV |
| | | GGGPLGLAGSGGS |
| SEQ ID NO: 153 | TY26138 | EVGSYDDTAFDCDDDFDDCAT |
| | | GGGPLGLAGSGGS |
| SEQ ID NO: 154 | TY26139 | EVGSYHAYHNDCSDDDDFCAY |
| | | GGGPLGLAGSGGS |
| SEQ ID NO: 155 | TY26292 | EVGSYPADDEDCPAADVFCSP |
| | | GGGPLGLAGSGGS |
| SEQ ID NO: 156 | TY26293 | EVGSYAPSVDDCPTYDDFCYD |
| | | GGGPLGLAGSGGS |
| SEQ ID NO: 157 | TY26294 | EVGSYLTVDYFCDIDPLYCNA |
| | | GGGPLGLAGSGGS |
| SEQ ID NO: 158 | TY26295 | EVGSYNYNDDDCYDDDYACDY |
| | | GGGPLGLAGSGGS |
| SEQ ID NO: 159 | TY26296 | EVGSYDTADHDCYDDDDFCYD |
| | | GGGPLGLAGSGGS |
| SEQ ID NO: 160 | TY26297 | EVGSYDFLSASCDDDYAPCAF |
| | | GGGPLGLAGSGGS |
| SEQ_ID_NO: 161 | TY26298 | EVGSYYPPDDTCDDDYDPCAI |
| | | GGGPLGLAGSGGS |
| SEQ ID NO: 162 | TY26299 | EVGSYAQSSFDCPDHDAFCDP |
| | | GGGPLGLAGSGGS |
| SEQ ID NO: 163 | TY26300 | EVGSYAASPATCDDTLFFCPA |
| | | GGGPLGLAGSGGS |
| SEQ ID NO: 164 | TY26140 | EVGSYDSCPAEVVGIFCIQGG |
| | | GPLGLAGSGGS |
| SEQ ID NO: 165 | TY26141 | EVGSYFYCTGAAVGIFCSAGG |
| | | GPLGLAGSGGS |
| SEQ ID NO: 166 | TY26301 | EVGSYVACRIFDDDPFCIVGG |
| | | GPLGLAGSGGS |

TABLE 6A
Light chain (LC) and heavy chain (HC) sequences of illustrative anti-CD47
antibodies, masked antibodies (e.g., activatable antibodies),
and antigen-binding fragments.

| | Illustrative | | |
|---|---|---|---|
| | Antibody | | |
| SEQ ID NO | and Chain | Antibody Type | Amino Acid Sequence |
| SEQ ID | TY21446_LC | Non- | DIQLTQSPSSLSASVGDRVTITCRASQTIGRY |
| NO: 140 | | activatable | LNWYQQKPGKAPKLLIYDASNRATGIPSRFS |
| | | | GSGSGTDFTLTISSLQPEDFATYYCQQRYPW |
| | | | PYTFGQGTKVEIKRTVAAPSVFIFPPSDEQLK |
| | | | SGTASVVCLLNNFYPREAKVQWKVDNALQS |
| | | | GNSQESVTEQDSKDSTYSLSSTLTLSKADYE |
| | | | KHKVYACEVTHQGLSSPVTKSFNRGEC |
| SEQ ID | TY21446_HC | Non- | EVQLVESGGGLVQPGGSLRLSCAASGFTFTN |
| NO: 141 | | activatable | YAIHWVRQAPGKGLEWVSAISGSGSSTYYA |
| | | | DSVKGRFTISRDNSKNTLYLQLNSLRAEDTA |
| | | | VYYCARRGSYGFGAFDYWGQGTLVTVSSAS |
| | | | TKGPSVFPLAPCSRSTSESTAALGCLVKDYFP |
| | | | EPVTVSWNSGALTSGVHTFPAVLQSSGLYSL |
| | | | SSVVTVPSSSLGTKTYTCNVDHKPSNTKVDK |
| | | | RVESKYGPPCPPCPAPEFLGGPSVFLFPPKPK |
| | | | DTLMISRTPEVTCVVVDVSQEDPEVQFNWY |
| | | | VDGVEVHNAKTKPREEQFNSTYRVVSVLTV |
| | | | LHQDWLNGKEYKCKVSNKGLPSSIEKTISKA |
| | | | KGQPREPQVYTLPPSQEEMTKNQVSLTCLVK |
| | | | GFYPSDIAVEWESNGQPENNYKTTPPVLDSD |
| | | | GSFFLYSRLTVDKSRWQEGNVFSCSVMHEA |
| | | | LHNHYTQKSLSLSLGK |
| SEQ ID | TY26294_LC | Activatable | EVGSYLTVDYFCDIDPLYCNAGGGPLGLAGS |
| NO: 142 | | | GGSDIQLTQSPSSLSASVGDRVTITCRASQTI |
| | | | GRYLNWYQQKPGKAPKLLIYDASNRATGIPS |
| | | | RFSGSGSGTDFTLTISSLQPEDFATYYCQQRY |
| | | | PWPYTFGQGTKVEIKRTVAAPSVFIFPPSDEQ |
| | | | LKSGTASVVCLLNNFYPREAKVQWKVDNAL |
| | | | QSGNSQESVTEQDSKDSTYSLSSTLTLSKAD |
| | | | YEKHKVYACEVTHQGLSSPVTKSENRGEC |
| SEQ ID | TY26294_HC | Activatable | EVQLVESGGGLVQPGGSLRLSCAASGFTFTN |
| NO: 143 | | | YAIHWVRQAPGKGLEWVSAISGSGSSTYYA |
| | | | DSVKGRFTISRDNSKNTLYLQLNSLRAEDTA |
| | | | VYYCARRGSYGFGAFDYWGQGTLVTVSSAS |
| | | | TKGPSVFPLAPCSRSTSESTAALGCLVKDYFP |
| | | | EPVTVSWNSGALTSGVHTFPAVLQSSGLYSL |
| | | | SSVVTVPSSSLGTKTYTCNVDHKPSNTKVDK |
| | | | RVESKYGPPCPPCPAPEFLGGPSVFLFPPKPK |
| | | | DTLMISRTPEVTCVVVDVSQEDPEVQFNWY |
| | | | VDGVEVHNAKTKPREEQFNSTYRVVSVLTV |
| | | | LHQDWLNGKEYKCKVSNKGLPSSIEKTISKA |
| | | | KGQPREPQVYTLPPSQEEMTKNQVSLTCLVK |
| | | | GFYPSDIAVEWESNGQPENNYKTTPPVLDSD |
| | | | GSFFLYSRLTVDKSRWQEGNVFSCSVMHEA |
| | | | LHNHYTQKSLSLSLGK |
| SEQ ID | TY26896_LC | Non- | DIQLTQSPSSLSASVGDRVTITCRASQTIGRY |
| NO: 144 | | activatable | LNWYQQKPGKAPKLLIYDASNRATGIPSRFS |
| | | | GSGSGTDFTLTISSLQPEDFATYYCQQRYPW |
| | | | PYTFGQGTKVEIKRTVAAPSVFIFPPSDEQLK |
| | | | SGTASVVCLLNNFYPREAKVQWKVDNALQS |
| | | | GNSQESVTEQDSKDSTYSLSSTLTLSKADYE |
| | | | KHKVYACEVTHQGLSSPVTKSFNRGEC |
| SEQ ID | TY26896_HC | Non- | EVQLVESGGGLVQPGGSLRLSCAASGFTFTN |
| NO: 145 | | activatable | YAIHWVRQAPGKGLEWVSAISGSGSSTYYA |
| | | | DSVKGRFTISRDNSKNTLYLQLNSLRAEDTA |
| | | | VYYCARRGSYGFGAFDYWGQGTLVTVSSAS |
| | | | TKGPSVFPLAPSSKSTSGGTAALGCLVKDYF |
| | | | PEPVTVSWNSGALTSGVHTFPAVLQSSGLYS |
| | | | LSSVVTVPSSSLGTQTYICNVNHKPSNTKVD |
| | | | KKVEPKSCDKTHTCPPCPAPELLGGPSVFLFP |
| | | | PKPKDTLMISRTPEVTCVVVDVSHEDPEVKF |
| | | | NWYVDGVEVHNAKTKPREEQYNSTYRVVS |
| | | | VLTVLHQDWLNGKEYKCKVSNKALPAPIEK |
| | | | TISKAKGQPREPQVYTLPPSRDELTKNQVSLT |
| | | | CLVKGFYPSDIAVEWESNGQPENNYKTTPPV |
| | | | LDSDGSFFLYSKLTVDKSRWQQGNVFSCSV |
| | | | MHEALHNHYTQKSLSLSPGK |
| SEQ ID | TY26897_LC | Non- | DIQLTQSPSSLSASVGDRVTITCRASQTIGRY |
| NO: 146 | | activatable | LNWYQQKPGKAPKLLIYDASNRATGIPSRES |
| | | | GSGSGTDFTLTISSLQPEDFATYYCQQRYPW |
| | | | PYTFGQGTKVEIKRTVAAPSVFIFPPSDEQLK |
| | | | SGTASVVCLLNNFYPREAKVQWKVDNALQS |
| | | | GNSQESVTEQDSKDSTYSLSSTLTLSKADYE |
| | | | KHKVYACEVTHQGLSSPVTKSFNRGEC |
| SEQ ID | TY26897_HC | Non- | EVQLVESGGGLVQPGGSLRLSCAASGFTFTN |
| NO: 147 | | activatable | YAIHWVRQAPGKGLEWVSAISGSGSSTYYA |
| | | | DSVKGRFTISRDNSKNTLYLQLNSLRAEDTA |
| | | | VYYCARRGSYGFGAFDYWGQGTLVTVSSAS |
| | | | TKGPSVFPLAPSSKSTSGGTAALGCLVKDYF |
| | | | PEPVTVSWNSGALTSGVHTFPAVLQSSGLYS |
| | | | LSSVVTVPSSSLGTQTYICNVNHKPSNTKVD |
| | | | KKVEPKSCDKTHTCPPCPAPELLGGPDVFLF |
| | | | PPKPKDTLMISRTPEVTCVVVDVSHEDPEVK |
| | | | FNWYVDGVEVHNAKTKPREEQYNSTYRVV |
| | | | SVLTVLHQDWLNGKEYKCKVSNKALPAPEE |
| | | | KTISKAKGQPREPQVYTLPPSRDELTKNQVS |
| | | | LTCLVKGFYPSDIAVEWESNGQPENNYKTTP |
| | | | PVLDSDGSFFLYSKLTVDKSRWQQGNVFSCS |
| | | | VMHEALHNHYTQKSLSLSPGK |
| SEQ ID | TY26898_LC | Activatable | EVGSYLTVDYFCDIDPLYCNAGGGPLGLAGS |
| NO: 148 | | | GGSDIQLTQSPSSLSASVGDRVTITCRASQTI |
| | | | GRYLNWYQQKPGKAPKLLIYDASNRATGIPS |
| | | | RFSGSGSGTDFTLTISSLQPEDFATYYCQQRY |
| | | | PWPYTFGQGTKVEIKRTVAAPSVFIFPPSDEQ |
| | | | LKSGTASVVCLLNNFYPREAKVQWKVDNAL |
| | | | QSGNSQESVTEQDSKDSTYSLSSTLTLSKAD |
| | | | YEKHKVYACEVTHQGLSSPVTKSFNRGEC |
| SEQ ID | TY26898_HC | Activatable | EVQLVESGGGLVQPGGSLRLSCAASGFTFTN |
| NO: 149 | | | YAIHWVRQAPGKGLEWVSAISGSGSSTYYA |
| | | | DSVKGRFTISRDNSKNTLYLQLNSLRAEDTA |
| | | | VYYCARRGSYGFGAFDYWGQGTLVTVSSAS |
| | | | TKGPSVFPLAPSSKSTSGGTAALGCLVKDYF |
| | | | PEPVTVSWNSGALTSGVHTFPAVLQSSGLYS |
| | | | LSSVVTVPSSSLGTQTYICNVNHKPSNTKVD |
| | | | KKVEPKSCDKTHTCPPCPAPELLGGPSVFLFP |
| | | | PKPKDTLMISRTPEVTCVVVDVSHEDPEVKF |
| | | | NWYVDGVEVHNAKTKPREEQYNSTYRVVS |
| | | | VLTVLHQDWLNGKEYKCKVSNKALPAPIEK |
| | | | TISKAKGQPREPQVYTLPPSRDELTKNQVSLT |
| | | | CLVKGFYPSDIAVEWESNGQPENNYKTTPPV |
| | | | LDSDGSFFLYSKLTVDKSRWQQGNVFSCSV |
| | | | MHEALHNHYTQKSLSLSPGK |
| SEQ ID | TY26899_LC | Activatable | EVGSYLTVDYFCDIDPLYCNAGGGPLGLAGS |
| NO: 150 | | | GGSDIQLTQSPSSLSASVGDRVTITCRASQTI |
| | | | GRYLNWYQQKPGKAPKLLIYDASNRATGIPS |
| | | | RFSGSGSGTDFTLTISSLQPEDFATYYCQQRY |
| | | | PWPYTFGQGTKVEIKRTVAAPSVFIFPPSDEQ |
| | | | LKSGTASVVCLLNNFYPREAKVQWKVDNAL |
| | | | QSGNSQESVTEQDSKDSTYSLSSTLTLSKAD |
| | | | YEKHKVYACEVTHQGLSSPVTKSFNRGEC |
| SEQ ID | TY26899_HC | Activatable | EVQLVESGGGLVQPGGSLRLSCAASGFTFTN |
| NO: 151 | | | YAIHWVRQAPGKGLEWVSAISGSGSSTYYA |
| | | | DSVKGRFTISRDNSKNTLYLQLNSLRAEDTA |
| | | | VYYCARRGSYGFGAFDYWGQGTLVTVSSAS |
| | | | TKGPSVFPLAPSSKSTSGGTAALGCLVKDYF |
| | | | PEPVTVSWNSGALTSGVHTFPAVLQSSGLYS |
| | | | LSSVVTVPSSSLGTQTYICNVNHKPSNTKVD |
| | | | KKVEPKSCDKTHTCPPCPAPELLGGPDVFLF |
| | | | PPKPKDTLMISRTPEVTCVVVDVSHEDPEVK |
| | | | FNWYVDGVEVHNAKTKPREEQYNSTYRVV |
| | | | SVLTVLHQDWLNGKEYKCKVSNKALPAPEE |
| | | | KTISKAKGQPREPQVYTLPPSRDELTKNQVS |
| | | | LTCLVKGFYPSDIAVEWESNGQPENNYKTTP |
| | | | PVLDSDGSFFLYSKLTVDKSRWQQGNVFSCS |
| | | | VMHEALHNHYTQKSLSLSPGK |

TABLE 6B
Sequences of illustrative anti-CD47 antibodies and activatable antibodies.

| | | | | SEQ ID NOs | | | | | | | | | | | | |
|---|---|---|---|---|---|---|---|---|---|---|---|---|---|---|---|---|
| | | Unmasked | | CDRs | | | | | | | | Heavy | Light | | | Masking |
| Ab | Masked? | Parent | Fc | H1 | H2 | H3 | L1 | L2 | L3 | VH | VL | chain | chain | MM | CM | peptide |
| TY21446 | No | NA* | hIgG4 | 48 | 65 | 82 | 99 | 116 | 133 | 27 | 28 | 141 | 140 | NA | NA | NA |
| TY26294 | Yes | TY21446 | hIgG4 | 48 | 65 | 82 | 99 | 116 | 133 | 27 | 28 | 143 | 142 | 137 | 138 | 139 |
| TY26896 | No | NA | hIgG1 | 48 | 65 | 82 | 99 | 116 | 133 | 27 | 28 | 145 | 144 | NA | NA | NA |
| TY26898 | Yes | TY26896 | hIgG1 | 48 | 65 | 82 | 99 | 116 | 133 | 27 | 28 | 149 | 148 | 137 | 138 | 139 |
| TY26897 | No | NA | hIgG1 | 48 | 65 | 82 | 99 | 116 | 133 | 27 | 28 | 147 | 146 | NA | NA | NA |
| | | | S239D | | | | | | | | | | | | | |
| | | | and I332E | | | | | | | | | | | | | |
| TY26899 | Yes | TY26897 | hIgG1 | 48 | 65 | 82 | 99 | 116 | 133 | 27 | 28 | 151 | 150 | 137 | 138 | 139 |
| | | | S239D | | | | | | | | | | | | | |
| | | | and I332E | | | | | | | | | | | | | |

*NA = not applicable

In some embodiments, provided herein is an isolated anti-CD47 antibody, or antigen-binding fragment thereof, wherein the anti-CD47 antibody comprises one, two, three, four, five, or six CDRs of illustrative antibody TY21446, TY26294, TY26896, TY26897, TY26898, or TY26899 as shown in Table 6B, or variants thereof comprising up to 5 (e.g., 1, 2, 3, 4, or 5) amino acid substitutions. In certain embodiments, provided herein is an isolated anti-CD47 antibody, or an antigen-binding fragment thereof, wherein the anti-CD47 antibody comprises a VH of illustrative antibody TY21446, TY26294, TY26896, TY26897, TY26898, or TY26899 as shown in Table 6B, or an amino acid sequence having at least 80% (e.g., at least 85%, 90%, 95%, 98%, or 99%; or 100%) sequence identity thereto; and a VL of illustrative antibody TY21446, TY26294, TY26896, TY26897, TY26898, or TY26899 as shown in Table 6B, or an amino acid sequence having at least 80% (e.g., at least 85%, 90%, 95%, 98%, or 99%; or 100%) sequence identity thereto. In certain embodiments, provided herein is an isolated anti-CD47 antibody, or an antigen-binding fragment thereof, wherein the anti-CD47 antibody comprises a heavy chain of illustrative antibody TY21446, TY26896, or TY26897 as shown in Table 6B, or an amino acid sequence having at least 80% (e.g., at least 85%, 90%, 95%, 98%, or 99%; or 100%) sequence identity thereto; and a light chain of illustrative antibody TY21446, TY26896, or TY26897 as shown in Table 6B, or an amino acid sequence having at least 80% (e.g., at least 85%, 90%, 95%, 98%, or 99%; or 100%) sequence identity thereto.

In some embodiments, provided herein is a multispecific antibody comprising comprises one, two, three, four, five, or six CDRs of illustrative antibody TY21446, TY26294, TY26896, TY26897. TY26898, or TY26899 as shown in Table 6B, or variants thereof comprising up to 5 (e.g., 1, 2, 3, 4, or 5) amino acid substitutions. In certain embodiments, provided herein is a multispecific antibody comprising a VH of illustrative antibody TY21446, TY26294, TY26896, TY26897, TY26898, or TY26899 as shown in Table 6B, or an amino acid sequence having at least 80% (e.g., at least 85%, 90%, 95%, 98%, or 99%; or 100%) sequence identity thereto; and a VL of illustrative antibody TY21446, TY26294, TY26896, TY26897, TY26898, or TY26899 as shown in Table 6B, or an amino acid sequence having at least 80% (e.g., at least 85%, 90%, 95%, 98%, or 99%; or 100%) sequence identity thereto. In some embodiments, provided herein is a bispecific antibody comprising comprises one, two, three, four, five, or six CDRs of illustrative antibody TY21446, TY26294, TY26896, TY26897, TY26898, or TY26899 as shown in Table 6B, or variants thereof comprising up to 5 (e.g., 1, 2, 3, 4, or 5) amino acid substitutions. In certain embodiments, provided herein is a bispecific antibody comprising a VH of illustrative antibody TY21446, TY26294, TY26896, TY26897, TY26898, or TY26899 as shown in Table 6B, or an amino acid sequence having at least 80% (e.g., at least 85%, 90%, 95%, 98%, or 99%; or 100%) sequence identity thereto; and a VL of illustrative antibody TY21446, TY26294, TY26896, TY26897, TY26898, or TY26899 as shown in Table 6B, or an amino acid sequence having at least 80% (e.g., at least 85%, 90%, 95%, 98%, or 99%; or 100%) sequence identity thereto.

In some embodiments, provided herein is an isolated anti-CD47 antibody, or an antigen-binding fragment thereof, comprising an IgG4 Fc region, wherein the anti-CD47 antibody comprises one, two, three, four, five, or six CDRs of illustrative antibody TY21446 as shown in Table 6B, or variants thereof comprising up to 5 (e.g., 1, 2, 3, 4, or 5) amino acid substitutions. In some embodiments, provided herein is an isolated anti-CD47 antibody, or an antigen-binding fragment thereof, comprising an IgG4 Fc region, wherein the anti-CD47 antibody comprises the antibody heavy chain variable region (VH) of illustrative antibody TY21446 as shown in Table 6B, or an amino acid sequence having at least 80% (e.g., at least 85%, 90%, 95%, 98%, or 99%; or 100%) sequence identity thereto, and the antibody light chain variable region (VL) of illustrative antibody TY21446 as shown in Table 6B, or an amino acid sequence having at least 80% (e.g., at least 85%, 90%, 95%, 98%, or 99%; or 100%) sequence identity thereto. In certain embodiments, provided herein is an isolated anti-CD47 antibody, or an antigen-binding fragment thereof, comprising an IgG4 Fc region, wherein the anti-CD47 antibody comprises the heavy chain of illustrative antibody TY21446 as shown in Table 6B, or an amino acid sequence having at least 80% (e.g., at least 85%, 90%, 95%, 98%, or 99%; or 100%) sequence identity thereto; and the light chain of illustrative antibody TY21446 as shown in Table 6B, or an amino acid sequence having at least 80% (e.g., at least 85%, 90%, 95%, 98%, or 99%; or 100%) sequence identity thereto. In certain embodiments, the isolated anti-CD47 antibody is a multispecific (e.g., bispecific) antibody.

In some embodiments, provided herein is an isolated anti-CD47 antibody, or an antigen-binding fragment thereof, comprising an IgG4 Fc region, wherein the anti-CD47 antibody comprises one, two, three, four, five, or six CDRs of illustrative antibody TY26896 as shown in Table 6B, or variants thereof comprising up to 5 (e.g., 1, 2, 3, 4, or 5) amino acid substitutions. In some embodiments, provided herein is an isolated anti-CD47 antibody, or an antigen-binding fragment thereof, comprising an IgG4 Fc region, wherein the anti-CD47 antibody comprises the antibody heavy chain variable region (VH) of illustrative antibody TY26896 as shown in Table 6B, or an amino acid sequence having at least 80% (e.g., at least 85%, 90%, 95%, 98%, or 99%; or 100%) sequence identity thereto, and the antibody light chain variable region (VL) of illustrative antibody TY26896 as shown in Table 6B, or an amino acid sequence having at least 80% (e.g., at least 85%, 90%, 95%, 98%, or 99%; or 100%) sequence identity thereto. In certain embodiments, provided herein is an isolated anti-CD47 antibody, or an antigen-binding fragment thereof, comprising an IgG1 Fc region, wherein the anti-CD47 antibody comprises the heavy chain of illustrative antibody TY26896 as shown in Table 6B, or an amino acid sequence having at least 80% (e.g., at least 85%, 90%, 95%, 98%, or 99%; or 100%) sequence identity thereto; and the light chain of illustrative antibody TY26896 as shown in Table 6B, or an amino acid sequence having at least 80% (e.g., at least 85%, 90%, 95%, 98%, or 99%; or 100%) sequence identity thereto. In certain embodiments, the isolated anti-CD47 antibody is a multispecific (e.g., bispecific) antibody.

In some embodiments, provided herein is an isolated anti-CD47 antibody, or an antigen-binding fragment thereof, comprising an IgG4 Fc region, wherein the anti-CD47 antibody comprises one, two, three, four, five, or six CDRs of illustrative antibody TY26897 as shown in Table 6B, or variants thereof comprising up to 5 (e.g., 1, 2, 3, 4, or 5) amino acid substitutions. In some embodiments, provided herein is an isolated anti-CD47 antibody, or an antigen-binding fragment thereof, comprising an IgG4 Fc region, wherein the anti-CD47 antibody comprises the antibody heavy chain variable region (VH) of illustrative antibody TY26897 as shown in Table 6B, or an amino acid sequence having at least 80% (e.g., at least 85%, 90%, 95%, 98%, or 99%; or 100%) sequence identity thereto, and the antibody light chain variable region (VL) of illustrative antibody TY26897 as shown in Table 6B, or an amino acid sequence having at least 80% (e.g., at least 85%, 90%, 95%, 98%, or 99%; or 100%) sequence identity thereto. In certain embodiments, provided herein is an isolated anti-CD47 antibody, or an antigen-binding fragment thereof, comprising an IgG1 Fc region comprising a S239D substitution and an 1332E substitution, wherein the anti-CD47 antibody comprises the heavy chain of illustrative antibody TY26897 as shown in Table 6B, or an amino acid sequence having at least 80% (e.g., at least 85%, 90%, 95%, 98%, or 99%; or 100%) sequence identity thereto; and the light chain of illustrative antibody TY26897 as shown in Table 6B, or an amino acid sequence having at least 80% (e.g., at least 85%, 90%, 95%, 98%, or 99%; or 100%) sequence identity thereto. In certain embodiments, the isolated anti-CD47 antibody is a multispecific (e.g., bispecific) antibody. In certain embodiments, the human IgG1 Fc region comprises two Fc domains, wherein each of the two Fc domains comprises a S239D substitution and an 1332E substitution. In some embodiments, the IgG1 Fc region has enhanced antibody-dependent cellular cytotoxic (ADCC) and/or antibody-dependent cellular phagocytosis (ADCP) function(s).

In some embodiments, provided herein is an activatable antibody comprising: (a) a masking peptide comprising, from N terminus to C terminus, a masking moiety (MM) and a cleavable moiety (CM), wherein the CM comprises at least one cleavage site, and (b) a target binding moiety (TBM) comprising an antibody heavy chain variable region (VH) and an antibody light chain variable region (VL); wherein the masking peptide is linked to the N terminus of the VL; and wherein the activatable antibody has a higher binding affinity to human CD47 in vitro upon cleavage of the CM than before the cleavage of the CM; wherein the activatable antibody comprises one, two, three, four, five, or six CDRs of illustrative antibody TY21446, TY26294, TY26896, TY26897, TY26898, or TY26899 as shown in Table 6B, or variants thereof comprising up to 5 (e.g., 1, 2, 3, 4, or 5) amino acid substitutions. In some embodiments, provided herein is an activatable antibody comprising: (a) a masking peptide comprising, from N terminus to C terminus, a masking moiety (MM) and a cleavable moiety (CM), wherein the CM comprises at least one cleavage site, and (b) a target binding moiety (TBM) comprising an antibody heavy chain variable region (VH) of illustrative antibody TY21446, TY26294, TY26896, TY26897, TY26898, or TY26899 as shown in Table 6B, or an amino acid sequence having at least 80% (e.g., at least 85%, 90%, 95%, 98%, or 99%; or 100%), and an antibody light chain variable region (VL) of illustrative antibody TY21446, TY26294, TY26896, TY26897, TY26898, or TY26899 as shown in Table 6B, or an amino acid sequence having at least 80% (e.g., at least 85%, 90%, 95%, 98%, or 99%; or 100%) sequence identity thereto; wherein the masking peptide is linked to the N terminus of the VL; and wherein the activatable antibody has a higher binding affinity to human CD47 in vitro upon cleavage of the CM than before the cleavage of the CM.

In some embodiments, provided herein is an activatable antibody comprising: (a) a masking peptide comprising, from N terminus to C terminus, a masking moiety (MM) of illustrative antibody TY26294, TY26898, or TY26899 as shown in Table 6B and a cleavable moiety (CM) of illustrative antibody TY26294, TY26898, or TY26899 as shown in Table 6B, and (b) a target binding moiety (TBM) comprising an antibody heavy chain variable region (VH) and an antibody light chain variable region (VL); wherein the masking peptide is linked to the N terminus of the VL; and wherein the activatable antibody has a higher binding affinity to human CD47 in vitro upon cleavage of the CM than before the cleavage of the CM; wherein the activatable antibody comprises one, two, three, four, five, or six CDRs of illustrative antibody TY26294, TY26898, or TY26899 as shown in Table 6B, or variants thereof comprising up to 5 (e.g., 1, 2, 3, 4, or 5) amino acid substitutions. In some embodiments, provided herein is an activatable antibody comprising: (a) a masking peptide comprising, from N terminus to C terminus, a masking moiety (MM) of illustrative antibody TY26294, TY26898, or TY26899 as shown in Table 6B and a cleavable moiety (CM) of illustrative antibody TY26294, TY26898, or TY26899 as shown in Table 6B, and (b) a target binding moiety (TBM) comprising an antibody heavy chain variable region (VH) of illustrative antibody TY21446, TY26294, TY26896, TY26897, TY26898, or TY26899 as shown in Table 6B, or an amino acid sequence having at least 80% (e.g., at least 85%, 90%, 95%, 98%, or 99%; or 100%) sequence identity thereto, and an antibody light chain variable region (VL) of illustrative antibody TY26294, TY26898, or TY26899 as shown in Table 6B, or an amino acid sequence having at least 80% (e.g., at least 85%, 90%, 95%, 98%, or 99%; or 100%) sequence identity thereto; wherein the masking peptide is linked to the N terminus of the VL; and wherein the activatable antibody has a higher binding affinity to human CD47 in vitro upon cleavage of the CM than before the cleavage of the CM.

In some embodiments, provided herein is an activatable antibody comprising: (a) a masking peptide of illustrative antibody TY26294, TY26898, or TY26899 as shown in Table 6B, and (b) a target binding moiety (TBM) comprising an antibody heavy chain variable region (VH) and an antibody light chain variable region (VL); wherein the masking peptide is linked to the N terminus of the VL; and wherein the activatable antibody comprises one, two, three, four, five, or six CDRs of illustrative antibody TY26294, TY26898, or TY26899 as shown in Table 6B, or variants thereof comprising up to 5 (e.g., 1, 2, 3, 4, or 5) amino acid substitutions. In some embodiments, provided herein is an activatable antibody comprising: (a) a masking peptide of illustrative antibody TY26294, TY26898, or TY26899 as shown in Table 6B, and (b) a target binding moiety (TBM) comprising an antibody heavy chain variable region (VH) of illustrative antibody TY21446, TY26294, TY26896, TY26897, TY26898, or TY26899 as shown in Table 6B, or an amino acid sequence having at least 80% (e.g., at least 85%, 90%, 95%, 98%, or 99%; or 100%) sequence identity thereto, and an antibody light chain variable region (VL) of illustrative antibody TY26294, TY26898, or TY26899 as shown in Table 6B, or an amino acid sequence having at least 80% (e.g., at least 85%, 90%, 95%, 98%, or 99%; or 100%) sequence identity thereto; wherein the masking peptide is linked to the N terminus of the VL.

In some embodiments, provided herein is an activatable antibody comprising: (a) a masking peptide comprising, from N terminus to C terminus, the masking moiety (MM) of illustrative antibody TY26294 as shown in Table 6B, or a variant thereof comprising up to 5 (e.g., 1, 2, 3, 4, or 5) amino acid substitutions, and the cleavable moiety (CM) of illustrative antibody TY26294 as shown in Table 6B, or a variant thereof comprising up to 5 (e.g., 1, 2, 3, 4, or 5) amino acid substitutions, and (b) a target binding moiety (TBM) comprising an antibody heavy chain variable region (VH) and an antibody light chain variable region (VL); wherein the masking peptide is linked to the N terminus of the VL; and wherein the activatable antibody has a higher binding affinity to human CD47 in vitro upon cleavage of the CM than before the cleavage of the CM; wherein the activatable antibody comprises one, two, three, four, five, or six CDRs of illustrative antibody TY26294 as shown in Table 6B, or variants thereof comprising up to 5 (e.g., 1, 2, 3, 4, or 5) amino acid substitutions. In some embodiments, provided herein is an activatable antibody comprising: (a) a masking peptide comprising, from N terminus to C terminus, the masking moiety (MM) of illustrative antibody TY26294 as shown in Table 6B, or a variant thereof comprising up to 5 (e.g., 1, 2, 3, 4, or 5) amino acid substitutions, and the cleavable moiety (CM) of illustrative antibody TY26294 as shown in Table 6B., or a variant thereof comprising up to 5 (e.g., 1, 2, 3, 4, or 5) amino acid substitutions, and (b) a target binding moiety (TBM) comprising the antibody heavy chain variable region (VH) and the antibody light chain variable region (VL) of illustrative antibody TY26294 as shown in Table 6B, or an amino acid sequence having at least 80% (e.g., at least 85%, 90%, 95%, 98%, or 99%; or 100%) sequence identity thereto; wherein the masking peptide is linked to the N terminus of the VL; and wherein the activatable antibody has a higher binding affinity to human CD47 in vitro upon cleavage of the CM than before the cleavage of the CM. In some embodiments, provided herein is an activatable antibody comprising: (a) the masking peptide of illustrative antibody TY26294 as shown in Table 6B, or a variant thereof comprising up to 5 (e.g., 1, 2, 3, 4, or 5) amino acid substitutions, and (b) a target binding moiety (TBM) comprising an antibody heavy chain variable region (VH) and an antibody light chain variable region (VL); wherein the masking peptide is linked to the N terminus of the VL; and wherein the activatable antibody comprises one, two, three, four, five, or six CDRs of illustrative antibody TY26294 as shown in Table 6B, or variants thereof comprising up to 5 (e.g., 1, 2, 3, 4, or 5) amino acid substitutions. In some embodiments, provided herein is an activatable antibody comprising: (a) the masking peptide of illustrative antibody TY26294 as shown in Table 6B, or a variant thereof comprising up to 5 (e.g., 1, 2, 3, 4, or 5) amino acid substitutions, and (b) a target binding moiety (TBM) comprising the antibody heavy chain variable region (VH) and the antibody light chain variable region (VL) of illustrative antibody TY26294 as shown in Table 6B, or an amino acid sequence having at least 80% (e.g., at least 85%, 90%, 95%, 98%, or 99%; or 100%) sequence identity thereto; wherein the masking peptide is linked to the N terminus of the VL. In some embodiments, provided herein is an activatable antibody comprising the heavy chain of illustrative antibody TY26294 as shown in Table 6B, or an amino acid sequence having at least 80% (e.g., at least 85%, 90%, 95%, 98%, or 99%; or 100%) sequence identity thereto; and the light chain of illustrative antibody TY26294 as shown in Table 6B, or an amino acid sequence having at least 80% (e.g., at least 85%, 90%, 95%, 98%, or 99%; or 100%) sequence identity thereto. In certain embodiments, the isolated anti-CD47 antibody is a multispecific (e.g., bispecific) antibody.

In some embodiments, provided herein is an activatable antibody comprising: (a) a masking peptide comprising, from N terminus to C terminus, the masking moiety (MM) of illustrative antibody TY26898 as shown in Table 6B, or a variant thereof comprising up to 5 (e.g., 1, 2, 3, 4, or 5) amino acid substitutions, and the cleavable moiety (CM) of illustrative antibody TY26898 as shown in Table 6B, or a variant thereof comprising up to 5 (e.g., 1, 2, 3, 4, or 5) amino acid substitutions, and (b) a target binding moiety (TBM) comprising an antibody heavy chain variable region (VH) and an antibody light chain variable region (VL); wherein the masking peptide is linked to the N terminus of the VL; and wherein the activatable antibody has a higher binding affinity to human CD47 in vitro upon cleavage of the CM than before the cleavage of the CM; wherein the activatable antibody comprises one, two, three, four, five, or six CDRs of illustrative antibody TY26898 as shown in Table 6B, or variants thereof comprising up to 5 (e.g., 1, 2, 3, 4, or 5) amino acid substitutions. In some embodiments, provided herein is an activatable antibody comprising: (a) a masking peptide comprising, from N terminus to C terminus, the masking moiety (MM) of illustrative antibody TY26898 as shown in Table 6B, or a variant thereof comprising up to 5 (e.g., 1, 2, 3, 4, or 5) amino acid substitutions, and the cleavable moiety (CM) of illustrative antibody TY26898 as shown in Table 6B, or a variant thereof comprising up to 5 (e.g., 1, 2, 3, 4, or 5) amino acid substitutions, and (b) a target binding moiety (TBM) comprising the antibody heavy chain variable region (VH) and the antibody light chain variable region (VL) of illustrative antibody TY26898 as shown in Table 6B, or an amino acid sequence having at least 80% (e.g., at least 85%, 90%, 95%, 98%, or 99%; or 100%) sequence identity thereto; wherein the masking peptide is linked to the N terminus of the VL; and wherein the activatable antibody has a higher binding affinity to human CD47 in vitro upon cleavage of the CM than before the cleavage of the CM. In some embodiments, provided herein is an activatable antibody comprising: (a) the masking peptide of illustrative antibody TY26898 as shown in Table 6B, or a variant thereof comprising up to 5 (e.g., 1, 2, 3, 4, or 5) amino acid substitutions, and (b) a target binding moiety (TBM) comprising an antibody heavy chain variable region (VH) and an antibody light chain variable region (VL); wherein the masking peptide is linked to the N terminus of the VL; and wherein the activatable antibody comprises one, two, three, four, five, or six CDRs of illustrative antibody TY26898 as shown in Table 6B, or variants thereof comprising up to 5 (e.g., 1, 2, 3, 4, or 5) amino acid substitutions. In some embodiments, provided herein is an activatable antibody comprising: (a) the masking peptide of illustrative antibody TY26898 as shown in Table 6B, or a variant thereof comprising up to 5 (e.g., 1, 2, 3, 4, or 5) amino acid substitutions, and (b) a target binding moiety (TBM) comprising the antibody heavy chain variable region (VH) and the antibody light chain variable region (VL) of illustrative antibody TY26898 as shown in Table 6B, or an amino acid sequence having at least 80% (e.g., at least 85%, 90%, 95%, 98%, or 99%; or 100%) sequence identity thereto; wherein the masking peptide is linked to the N terminus of the VL. In some embodiments, provided herein is an activatable antibody comprising the heavy chain of illustrative antibody TY26898 as shown in Table 6B, or an amino acid sequence having at least 80% (e.g., at least 85%, 90%, 95%, 98%, or 99%; or 100%) sequence identity thereto; and the light chain of illustrative antibody TY26898 as shown in Table 6B, or an amino acid sequence having at least 80% (e.g., at least 85%, 90%, 95%, 98%, or 99%; or 100%) sequence identity thereto. In certain embodiments, the isolated anti-CD47 antibody is a multispecific (e.g., bispecific) antibody.

In some embodiments, provided herein is an activatable antibody comprising: (a) a masking peptide comprising, from N terminus to C terminus, the masking moiety (MM) of illustrative antibody TY26899 as shown in Table 6B, or a variant thereof comprising up to 5 (e.g., 1, 2, 3, 4, or 5) amino acid substitutions, and the cleavable moiety (CM) of illustrative antibody TY26899 as shown in Table 6B, or a variant thereof comprising up to 5 (e.g., 1, 2, 3, 4, or 5) amino acid substitutions, and (b) a target binding moiety (TBM) comprising an antibody heavy chain variable region (VH) and an antibody light chain variable region (VL); wherein the masking peptide is linked to the N terminus of the VL; and wherein the activatable antibody has a higher binding affinity to human CD47 in vitro upon cleavage of the CM than before the cleavage of the CM; wherein the activatable antibody comprises one, two, three, four, five, or six CDRs of illustrative antibody TY26899 as shown in Table 6B, or variants thereof comprising up to 5 (e.g., 1, 2, 3, 4, or 5) amino acid substitutions. In some embodiments, provided herein is an activatable antibody comprising: (a) a masking peptide comprising, from N terminus to C terminus, the masking moiety (MM) of illustrative antibody TY26899 as shown in Table 6B, or a variant thereof comprising up to 5 (e.g., 1, 2, 3, 4, or 5) amino acid substitutions, and the cleavable moiety (CM) of illustrative antibody TY26899 as shown in Table 6B, or a variant thereof comprising up to 5 (e.g., 1, 2, 3, 4, or 5) amino acid substitutions, and (b) a target binding moiety (TBM) comprising the antibody heavy chain variable region (VH) and the antibody light chain variable region (VL) of illustrative antibody TY26899 as shown in Table 6B, or an amino acid sequence having at least 80% (e.g., at least 85%, 90%, 95%, 98%, or 99%; or 100%) sequence identity thereto; wherein the masking peptide is linked to the N terminus of the VL; and wherein the activatable antibody has a higher binding affinity to human CD47 in vitro upon cleavage of the CM than before the cleavage of the CM. In some embodiments, provided herein is an activatable antibody comprising: (a) the masking peptide of illustrative antibody TY26899 as shown in Table 6B, or a variant thereof comprising up to 5 (e.g., 1, 2, 3, 4, or 5) amino acid substitutions, and (b) a target binding moiety (TBM) comprising an antibody heavy chain variable region (VH) and an antibody light chain variable region (VL); wherein the masking peptide is linked to the N terminus of the VL; and wherein the activatable antibody comprises one, two, three, four, five, or six CDRs of illustrative antibody TY26899 as shown in Table 6B, or variants thereof comprising up to 5 (e.g., 1, 2, 3, 4, or 5) amino acid substitutions. In some embodiments, provided herein is an activatable antibody comprising: (a) the masking peptide of illustrative antibody TY26899 as shown in Table 6B, or a variant thereof comprising up to 5 (e.g., 1, 2, 3, 4, or 5) amino acid substitutions, and (b) a target binding moiety (TBM) comprising the antibody heavy chain variable region (VH) and the antibody light chain variable region (VL) of illustrative antibody TY26899 as shown in Table 6B, or an amino acid sequence having at least 80% (e.g., at least 85%, 90%, 95%, 98%, or 99%; or 100%) sequence identity thereto; wherein the masking peptide is linked to the N terminus of the VL. In some embodiments, provided herein is an activatable antibody comprising the heavy chain of illustrative antibody TY26899 as shown in Table 6B, or an amino acid sequence having at least 80% (e.g., at least 85%, 90%, 95%, 98%, or 99%; or 100%) sequence identity thereto; and the light chain of illustrative antibody TY26899 as shown in Table 6B, or an amino acid sequence having at least 80% (e.g., at least 85%, 90%, 95%, 98%, or 99%; or 100%) sequence identity thereto. In certain embodiments, the isolated anti-CD47 antibody is a multispecific (e.g., bispecific) antibody.

### B-6. Variants

In some embodiments, amino acid sequence variants of the anti-CD47 binding molecules (e.g., antibodies, antigen-binding fragments, and masked antibodies (e.g., activatable antibodies)) provided herein are contemplated. For example, it may be desirable to improve the binding affinity and/or other biological properties of the antibody, antigen-binding fragment, or masked antibody (e.g., activatable antibody). In some particular embodiments, the disclosure provides variant of an antibody or masked antibody (e.g., activatable antibody) selected from those listed in Tables 3A-6.

Amino acid sequence variants of an antibody, antigen-binding fragment, or masked antibody (e.g., activatable antibody) may be prepared by introducing appropriate modifications into the nucleotide sequence encoding the antibody or masked antibody (e.g., activatable antibody), or by peptide synthesis. Such modifications include, for example, deletions from, and/or insertions into and/or substitutions of residues within the amino acid sequences of the antibody or masked antibody (e.g., activatable antibody). Any combination of deletion, insertion, and substitution can be made to arrive at the final construct, provided that the final construct possesses the desired characteristics, e.g., antigen-binding.

In some embodiments, variants of the antibodies, antigen-binding fragments, and masked antibodies (e.g., activatable antibodies) described herein having one or more amino acid substitutions are provided. Sites of interest for substitutional mutagenesis include, but are not limited to, the hypervariable regions (HVRs), complementarity determining regions (CDRs), heavy chain variable regions (VHs), light chain variable regions (VLs), heavy chain constant regions (CHs), light chain constant regions (CLs), and fragment crystallizable regions (Fc regions). Amino acid substitutions may be introduced into an antibody, antigen-binding fragment, or masked antibody (e.g., activatable antibody) of interest and the products screened for a desired activity, e.g., retained/improved antigen binding, decreased immunogenicity, or improved antibody-dependent cellular cytotoxicity (ADCC) or complement dependent cytotoxicity (CDC).

Conservative substitutions are shown in Table 2 below.

TABLE 2
Conservative amino acid substitutions.

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:
a. hydrophobic: Norleucine, Met, Ala, Val, Leu, Ile;
b. neutral hydrophilic: Cys, Ser, Thr, Asn, Gln;
c. acidic: Asp, Glu;
d. basic: His, Lys, Arg;
c. residues that influence chain orientation: Gly, Pro;
f. aromatic: Trp, Tyr, Phc.

Non-conservative substitutions will entail exchanging a member of one of these classes for another class.

An exemplary substitutional variant is an affinity matured antibody moiety, which may be conveniently generated, e.g., using phage display-based affinity maturation techniques. Briefly, one or more CDR residues are mutated and the variant antibody moieties displayed on phage and screened for a particular biological activity (e.g., binding affinity). Alterations (e.g., substitutions) may be made in HVRs (e.g., within a CDR), e.g., to improve antibody moiety affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or specificity determining residues (SDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody moiety variants with the desired affinity.

Another method to introduce diversity involves HVR-directed or CDR-directed approaches, in which several HVR residues (e.g., 4-6 residues at a time) are randomized (e.g., within a CDR). HVR and/or CDR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs (e.g., within a CDR) so long as such alterations do not substantially reduce the ability of the antibody to bind antigen. For example, conservative alterations (e.g., conservative substitutions as provided herein) that do not substantially reduce binding affinity may be made in HVRs (e.g., within a CDR). Such alterations may be outside of HVR “hotspots” or SDRs. In some embodiments of the variant VH and VL sequences provided above, each HVR or CDR either is unaltered, or contains no more than one, two or three amino acid substitutions.

A useful method for identification of residues or regions of an antibody, antigen-binding fragment, or masked antibody (e.g., activatable antibody) that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody, antigen-binding fragment, or masked antibody (e.g., activatable antibody) with the antigen is affected. Further substitutions may be introduced at the amino acid locations demonstrating functional sensitivity to the initial substitutions.

Alternatively, or additionally, a crystal structure of an antigen-antibody complex can be determined to identify contact points between the antibody or masked antibody (e.g., activatable antibody) and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule or masked antibody (e.g., activatable antibody) molecule include the fusion to the N- or C-terminus of the antibody or masked antibody (e.g., activatable antibody) to an enzyme (e.g. for ADEPT) or a polypeptide which increases the serum half-life of the antibody or masked antibody (e.g., activatable antibody).

### Fc Region Variants

In some embodiments, one or more amino acid modifications may be introduced into the Fc region of an anti-CD47 antibody or masked antibody (e.g., activatable antibody) protein provided herein, thereby generating an Fc region variant. In some embodiments, the Fc region variant has enhanced ADCC effector function, often related to binding to Fc receptors (FcRs). In some embodiments, the Fc region variant has decreased ADCC effector function. There are many examples of changes or mutations to Fc sequences that can alter effector function. For example, WO 00/42072 and Shields et al. J Biol. Chem. 9 (2): 6591-6604 (2001) describe antibody variants with improved or diminished binding to FcRs. The contents of those publications are specifically incorporated herein by reference.

Antibody-Dependent Cell-Mediated Cytotoxicity (ADCC) is a mechanism of action of therapeutic antibodies against tumor cells. ADCC is a cell-mediated immune defense whereby an effector cell of the immune system actively lyses a target cell (e.g., a cancer cell), whose membrane-surface antigens have been bound by specific antibodies (e.g., an anti-CD47 antibody). The typical ADCC involves activation of NK cells by antibodies. An NK cell expresses CD16 which is an Fc receptor. This receptor recognizes, and binds to, the Fc portion of an antibody bound to the surface of a target cell. The most common Fc receptor on the surface of an NK cell is called CD16 or FcγRIII. Binding of the Fc receptor to the Fc region of an antibody results in NK cell activation, release of cytolytic granules and consequent target cell apoptosis. The contribution of ADCC to tumor cell killing can be measured, for example, with a specific test that uses NK-92 cells that have been transfected with a high-affinity FcR. Results are compared to wild-type NK-92 cells that do not express the FcR.

In some embodiments, the invention contemplates a variant anti-CD47 antibody or masked (e.g., activatable) antibody (e.g., an isolated anti-CD47 antibody variant or a masked (e.g., activatable) anti-CD47 antibody variant) comprising an Fc region that possesses some but not all effector functions, which makes it a desirable candidate for certain applications, for example, in which the half-life of the anti-CD47 antibody or masked antibody (e.g., activatable antibody) in vivo is important, yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody or masked antibody (e.g., activatable antibody) lacks FcγR binding (hence likely lacking ADCC activity), but retains FcRn binding ability. The primary cells for mediating ADCC, NK cells, express FcγRIII only, whereas monocytes express FcγRI, FcγRI I and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I. et al., Proc. Nat'l Acad. Sci. USA 82:1499-1502 (1985); U.S. Pat. No. 5,821,337 (see Bruggemann, M. et al, J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96TM non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). Clq binding assays may also be carried out to confirm that the antibody or masked antibody (e.g., activatable antibody) is unable to bind Clq and hence lacks CDC activity. Sec, e.g., Clq and C3c binding ELISA in WO 2006/029879 and WO 2005/100402. To assess complement activation, a CDC assay may be performed (see, for example, Gazzano-Santoro et al, J. Immunol. Methods 202:163 (1996); Cragg. M. S. el al, Blood 101:1045-1052 (2003); and Cragg, M. S. and M. J. Glennic, Blood 103:2738-2743 (2004)). FcRn binding and in vivo clearance/half-life determinations can also be performed using methods known in the art (see, e.g., Petkova, S. B. et al, Int'l. Immunol. 18 (12): 1759-1769 (2006)).

Antibodies (e.g., isolated antibodies and activatable antibodies) with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al, J. Biol. Chem. 9 (2): 6591-6604 (2001).)

In some embodiments, provided herein is a variant anti-CD47 antibody or masked (e.g., activatable) antibody (e.g., an isolated anti-CD47 antibody variant or a masked (e.g., activatable) anti-CD47 antibody variant) comprising a variant Fc region comprising one or more amino acid substitutions which improve ADCC. In some embodiments, the variant Fc region comprises one or more amino acid substitutions which improve ADCC, wherein the substitutions are at positions 298, 333, and/or 334 of the variant Fc region (EU numbering of residues). In some embodiments, the anti-CD47 antibody or masked antibody (e.g., activatable antibody) variant comprises the following amino acid substitution in its variant Fc region: S298A, E333 A, and/or K334A.

In some embodiments, alterations are made in the Fc region that result in altered (i.e., either improved or diminished) Clq binding and/or Complement Dependent Cytotoxicity (CDC), e.g., as described in U.S. Pat. No. 6,194,551, WO 99/51642, and Idusogie et al. J. Immunol. 164:4178-4184 (2000).

In some embodiments, provided herein is variant anti-CD47 antibody or masked (e.g., activatable) antibody (e.g., an isolated anti-CD47 antibody variant or a masked (e.g., activatable) anti-CD47 antibody variant) comprising a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

Variant anti-CD47 antibody or masked (e.g., activatable) antibody (e.g., an isolated anti-CD47 antibody variant or a masked (e.g., activatable) anti-CD47 antibody variant) comprising any of the Fc variants described herein, or combinations thereof, are contemplated.

### Glycosylation Variants

In some embodiments, an anti-CD47 antibody, antigen-binding fragment, or masked antibody (e.g., activatable antibody) provided herein is altered to increase or decrease the extent to which the anti-CD47 antibody, antigen-binding fragment, or masked antibody (e.g., activatable antibody) is glycosylated. Addition or deletion of glycosylation sites to an anti-CD47 antibody, antigen-binding fragment, or masked antibody (e.g., activatable antibody) may be conveniently accomplished by altering the amino acid sequence of the anti-CD47 antibody, antigen-binding fragment, or masked antibody (e.g., activatable antibody) or polypeptide portion thereof such that one or more glycosylation sites is created or removed.

Where the anti-CD47 antibody or masked antibody (e.g., activatable antibody) comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an anti-CD47 antibody, antigen-binding fragment, or masked antibody (e.g., activatable antibody) of the invention may be made in order to create anti-CD47 antibody, antigen-binding fragment, or masked antibody (e.g., activatable antibody) variants with certain improved properties.

The N-glycans attached to the CH2 domain of Fc is heterogeneous. Antibodies or Fc fusion proteins generated in CHO cells are fucosylated by fucosyltransferase activity. Sec Shoji-Hosaka et al., J. Biochem. 2006, 140:777-83. Normally, a small percentage of naturally occurring afucosylated IgGs may be detected in human serum. N-glycosylation of the Fc is important for binding to FcγR; and afucosylation of the N-glycan increases Fc's binding capacity to FcγRIIIa. Increased FcγRIIIa binding can enhance ADCC, which can be advantageous in certain antibody therapeutic applications in which cytotoxicity is desirable.

In some embodiments, an enhanced effector function can be detrimental when Fc-mediated cytotoxicity is undesirable. In some embodiments, the Fc fragment or CH2 domain is not glycosylated. In some embodiments, the N-glycosylation site in the CH2 domain is mutated to prevent from glycosylation.

In some embodiments, anti-CD47 antibody, antigen-binding fragment, or masked antibody (e.g., activatable antibody) variants are provided comprising an Fc region wherein a carbohydrate structure attached to the Fc region has reduced fucose or lacks fucose, which may improve ADCC function. Specifically, anti-CD47 antibodies, antigen-binding fragments, and masked antibodies (e.g., activatable antibodies) are contemplated herein that have reduced fucose relative to the amount of fucose on the same anti-CD47 antibody, antigen-binding fragment, or masked antibody (e.g., activatable antibody) produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In some embodiments, the anti-CD47 antibody, antigen-binding fragment, or masked antibody (e.g., activatable antibody) is one wherein less than about 50%, 40%, 30%, 20%, 10%, or 5% of the N-linked glycans thereon comprise fucose. For example, the amount of fucose in such an anti-CD47 antibody, antigen-binding fragment, or masked antibody (e.g., activatable antibody) may be from 1% to 80%, from 1% to 65%, from 5% to 65% or from 20% to 40%. In some embodiments, the anti-CD47 antibody, antigen-binding fragment, or masked antibody (e.g., activatable antibody) is one wherein none of the N-linked glycans thereon comprise fucose, i.e., wherein the anti-CD47 antibody, antigen-binding fragment, or masked antibody (e.g., activatable antibody) is completely without fucose, or has no fucose or is afucosylated. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. Sec, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such asa-1,6-fucosyltransferase gene, FUT8, knockout CHO cells (see, e.g., Yamane-Ohnuki et al. Biotech. Bioeng. 87:614 (2004); Kanda, Y. et al., Biotechnol. Bioeng. 94 (4): 680-688 (2006); and WO2003/085107).

Anti-CD47 antibody, antigen-binding fragment, or masked antibody (e.g., activatable antibody) variants are further provided with bisected oligosaccharides, e.g., in which a biantennary oligosaccharide attached to the Fc region of the anti-GPC3 construct is bisected by GlcNAc. Such anti-CD47 antibody, antigen-binding fragment, or masked antibody (e.g., activatable antibody) variants may have reduced fucosylation and/or improved ADCC function. Examples of such antibody variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93 (5): 851-861 (2006). Anti-CD47 antibody, antigen-binding fragment, or masked antibody (e.g., activatable antibody) variants with at least one galactose residue in the oligosaccharide attached to the Fc region are also provided. Such anti-CD47 antibody, antigen-binding fragment, or masked antibody (e.g., activatable antibody) variants may have improved CDC function. Such antibody variants are described, e.g., in WO 1997/30087 (Patel et al.); WO 1998/58964 (Raju. S.); and WO 1999/22764 (Raju, S.).

In some embodiments, the anti-CD47 antibody, antigen-binding fragment, or masked antibody (e.g., activatable antibody) variants comprising an Fc region are capable of binding to an FcγRIII. In some embodiments, the anti-CD47 antibody, antigen-binding fragment, or masked antibody (e.g., activatable antibody) variants comprising an Fc region have ADCC activity in the presence of human effector cells (e.g., T cell) or have increased ADCC activity in the presence of human effector cells compared to the otherwise same anti-CD47 antibody, antigen-binding fragment, or masked antibody (e.g., activatable antibody) comprising a human wild-type IgG1Fc region.

### Cysteine Engineered Variants

In some embodiments, it may be desirable to create cysteine engineered anti-CD47 antibody, antigen-binding fragment, or masked antibody (e.g., activatable antibody) variants in which one or more amino acid residues are substituted with cysteine residues. In some embodiments, the substituted residues occur at accessible sites of the anti-CD47 antibody, antigen-binding fragment, or masked antibody (e.g., activatable antibody). By substituting those residues with cysteine, reactive thiol groups are thereby positioned at accessible sites of the anti-CD47 antibody, antigen-binding fragment, or masked antibody (e.g., activatable antibody) and may be used to conjugate the anti-CD47 antibody, antigen-binding fragment, or masked antibody (e.g., activatable antibody) to other moieties, such as drug moieties or linker-drug moieties, to create an anti-CD47 immunoconjugate, as described further herein. Cysteine engineered anti-CD47 antibody, antigen-binding fragment, or masked antibody (e.g., activatable antibody) variants may be generated as described, e.g., in U.S. Pat. No. 7,521,541.

### Derivatives

In some embodiments, an anti-CD47 antibody, antigen-binding fragment, or masked antibody (e.g., activatable antibody) provided herein may be further modified to contain additional non-proteinaceous moieties that are known in the art and readily available. The moieties suitable for derivatization of the anti-CD47 antibody, antigen-binding fragment, or masked antibody (e.g., activatable antibody) include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, polypropylene oxide/ethylene oxide copolymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the anti-CD47 antibody, antigen-binding fragment, or masked antibody (e.g., activatable antibody) may vary, and if more than one polymer are attached, they can be the same or different molecules. In general, the number and/or type of polymers used for derivatization can be determined based on considerations including, but not limited to, the particular properties or functions of the anti-CD47 antibody, antigen-binding fragment, or masked antibody (e.g., activatable antibody) to be improved, whether the anti-CD47 antibody, antigen-binding fragment, or masked antibody (e.g., activatable antibody) derivative will be used in a therapy under defined conditions, etc.

In some embodiments, conjugates of an anti-CD47 antibody, antigen-binding fragment, or masked antibody (e.g., activatable antibody) and a non-proteinaccous moiety that may be selectively heated by exposure to radiation are provided. In some embodiments, the non-proteinaceous moiety is a carbon nanotube (Kam et al., Proc. Natl. Acad. Sci. USA 102:11600-11605 (2005)). The radiation may be of any wavelength, and includes, but is not limited to, wavelengths that do not harm ordinary cells, but which heat the non-proteinaccous moiety to a temperature at which cells proximal to the anti-CD47 antibody-, antigen-binding fragment-, or masked antibody-(e.g., activatable antibody-) non-proteinaccous moiety conjugate are killed.

### D. Polynucleotides, Vectors, Host Cells, and Recombinant Methods of Producing Anti-CD47 Antibodies and Masked Antibodies

Another aspect of the disclosure provides an isolated polynucleotide that comprises a nucleotide sequence encoding an amino acid sequence of a binding molecule (e.g., antibody, masked antibody (e.g., activatable antibody), or antigen-binding fragment) provided by the present disclosure. The amino acid sequence encoded by the nucleotide sequence may be any portion of an antibody, antigen-binding fragment, or masked antibody (e.g., activatable antibody), such as a CDR, a sequence comprising one, two, or three CDRs, a variable region of a heavy chain, variable region of a light chain, a masking peptide, a masking moiety (MM), a linkage moiety (LM), a cleavable moiety (CM), or a target-binding moiety (TBM), or may be a full-length heavy chain or full-length light chain. A polynucleotide of the disclosure can be, for example, DNA or RNA, and may or may not contain intronic sequences. Typically, the polynucleotide is a cDNA molecule.

In some embodiments, the disclosure provides an isolated polynucleotide that comprises or consists of a nucleotide sequence encoding an amino acid sequence selected from the group consisting of: (1) amino acid sequence of a CDR of an illustrative antibody, antigen-binding fragment, or masked antibody (e.g., activatable antibody); (2) a variable region of a heavy chain (VH) or variable region of a light chain (VL) of an illustrative antibody, antigen-binding fragment, or masked antibody (e.g., activatable antibody); or (3) a full length heavy chain or full length light chain of an illustrative antibody, antigen-binding fragment, or masked antibody (e.g., activatable antibody),.

Polynucleotides of the disclosure can be obtained using any suitable molecular biology techniques. For antibodies or masked antibodies (e.g., activatable antibodies) expressed by hybridomas, cDNAs encoding the light and heavy chains of the antibody or masked antibody (e.g., activatable antibody) made by the hybridoma can be obtained by PCR amplification or cDNA cloning techniques. For antibodies or masked antibodies (e.g., activatable antibodies) obtained from an immunoglobulin gene library (e.g., using phage display techniques), the polynucleotide encoding the antibody or masked antibody (e.g., activatable antibody) can be recovered from the library.

The isolated DNA encoding the VH region can be converted to a full-length heavy chain gene by operatively linking the VH-encoding DNA to another DNA molecule encoding an fragment crystallizable (Fc) region comprising heavy chain constant regions (CH1, CH2 and CH3). The sequences of human heavy chain constant region genes are known in the art (scc e.g., Kabat et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The Fc region can be an IgG1, IgG2, IgG3, IgG4, IgA, IgE, IgM or IgD Fc region, but most preferably is an IgG4 or IgG1 constant region. The IgG4 or IgG1 constant region sequence can be any of the various alleles or allotypes known to occur among different individuals. These allotypes represent naturally occurring amino acid substitution in the IgG4 and IgG1 constant regions. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

To create a scFv gene, the VH- and VL-encoding DNA fragments are operatively linked to another fragment encoding a flexible linker, e.g., encoding the amino acid sequence (Gly4-Ser) 3, such that the VH and VL sequences can be expressed as a contiguous single-chain protein, with the VL and VH regions joined by the flexible linker (see e.g., Bird et al., Science 242:423-426 (1988); Huston et al., Proc. Natl. Acad. Sci. USA 85:5879-5883 (1988); and McCafferty et al., Nature 348:552-554 (1990)).

The present disclosure further provides a vector that comprises a polynucleotide provided by the present disclosure. The polynucleotide may encode a portion of a light chain or heavy chain (such as a CDR or a HVR), a full-length light or heavy chain, polypeptide that comprises a portion or full-length of a heavy or light chain, or an amino acid sequence of an antibody or masked antibody (e.g., activatable antibody) derivative or antigen-binding fragment. In some embodiments, the vector is an expression vector useful for the expression of a binding molecule, such as an antibody, a masked antibody (e.g., activatable antibody), or an antigen binding fragment thereof. In some embodiments, provided herein are vectors, wherein a first vector comprises a polynucleotide sequence encoding a heavy chain variable region as described herein, and a second vector comprises a polynucleotide sequence encoding a light chain variable region as described herein. In some embodiments, a single vector comprises polynucleotides encoding a heavy chain variable region as described herein and a light chain variable region as described herein.

To express a binding molecule (e.g., antibody, masked antibody (e.g., activatable antibody), or antigen-binding fragment) of the disclosure, DNAs encoding partial or full-length light and heavy chains are inserted into expression vectors such that the DNA molecules are operatively linked to transcriptional and translational control sequences. In this context, the term “operatively linked” means that an antibody, masked antibody (e.g., activatable antibody), or antigen-binding fragment gene is ligated into a vector such that transcriptional and translational control sequences within the vector serve their intended function of regulating the transcription and translation of the DNA molecule. The expression vector and expression control sequences are chosen to be compatible with the expression host cell used. The antibody, masked antibody (e.g., activatable antibody), or antigen-binding fragment light chain gene and the antibody, masked antibody (e.g., activatable antibody), or antigen-binding fragment heavy chain gene can be inserted into separate vector or, more typically, both genes are inserted into the same expression vector. The antibody, masked antibody (e.g., activatable antibody), or antigen-binding fragment genes are inserted into the expression vector by any suitable methods (e.g., ligation of complementary restriction sites on the antibody, masked antibody (e.g., activatable antibody), or antigen-binding fragment gene fragment and vector, or homologous recombination-based DNA ligation). The light and heavy chain variable regions of the antibodies, antigen-binding fragments, and masked antibodies (e.g., activatable antibodies) described herein can be used to create full-length antibody or masked antibody (e.g., activatable antibody) genes of any antibody isotype and subclass by inserting them into expression vectors already encoding heavy chain constant and light chain constant regions of the desired isotype and subclass such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody or masked antibody (e.g., activatable antibody) chain from a host cell. The antibody or masked antibody (e.g., activatable antibody) chain gene can be cloned into the vector such that the signal peptide is linked in-frame to the amino terminus of the antibody or masked antibody (e.g., activatable antibody) chain gene. The signal peptide can be an immunoglobulin signal peptide or a heterologous signal peptide (i.e., a signal peptide from a non-immunoglobulin protein).

In addition to the antibody and masked antibody (e.g., activatable antibody) chain genes, the expression vectors of the disclosure typically carry regulatory sequences that control the expression of the antibody or masked antibody (e.g., activatable antibody) chain genes in a host cell. The term “regulatory sequence” is intended to include promoters, enhancers and other expression control elements (e.g., polyadenylation signals) that control the transcription or translation of the antibody or masked antibody (e.g., activatable antibody) chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Examples of regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and polyoma. Alternatively, nonviral regulatory sequences may be used, such as the ubiquitin promoter or B-globin promoter. Still further, regulatory elements composed of sequences from different sources, such as the SR promoter system, which contains sequences from the SV40 early promoter and the long terminal repeat of human T cell leukemia virus type 1 (Takebe, Y. et al. (1988) Mol. Cell. Biol. 8:466-472).

In addition to the antibody or masked antibody (e.g., activatable antibody) chain genes and regulatory sequences, the expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by any suitable techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies and masked antibodies (e.g., activatable antibodies) of the disclosure in either prokaryotic or eukaryotic host cells, expression of antibodies and masked antibodies (e.g., activatable antibodies) in eukaryotic cells, and typically mammalian host cells, is most typical.

The present disclosure further provides a host cell containing a polynucleotide provided by the present disclosure. The host cell can be virtually any cell for which expression vectors are available. It may be, for example, a higher eukaryotic host cell, such as a mammalian cell, a lower eukaryotic host cell, such as a yeast cell, and may be a prokaryotic cell, such as a bacterial cell. Introduction of the recombinant polynucleotide construct into the host cell can be effected by calcium phosphate transfection, DEAE, dextran mediated transfection, electroporation or phage infection.

Suitable prokaryotic hosts for transformation include E. coli, Bacillus subtilis, Salmonella typhimurium and various species within the genera Pseudomonas, Streptomyces, and Staphylococcus.

Mammalian host cells for expressing a binding molecule (e.g., antibody, masked antibody (e.g., activatable antibody), or antigen-binding fragment) of the disclosure include, for example, Chinese Hamster Ovary (CHO) cells (including dhfr-CHO cells, described in Urlaub and Chasin, Proc. Natl. Acad. Sci. USA 77:4216-4220 (1980), used with a DHFR selectable marker, e.g., as described in Kaufman and Sharp, J. Mol. Biol. 159:601-621 (1982), NSO myeloma cells, COS cells and Sp2 cells. In particular, for use with NSO myeloma or CHO cells, another expression system is the GS (glutamine synthetase) gene expression system disclosed in WO 87/04462, WO 89/01036 and EP 338,841. When expression vectors encoding antibody or masked antibody (e.g., activatable antibody) genes are introduced into mammalian host cells, the antibodies or masked antibodies (e.g., activatable antibodies) are produced by culturing the host cells for a period of time sufficient to allow for expression of the antibody or masked antibody (e.g., activatable antibody) in the host cells or secretion of the antibody or masked antibody (e.g., activatable antibody) into the culture medium in which the host cells are grown. Antibodies and masked antibodies (e.g., activatable antibodies) can be recovered from the culture medium using any suitable protein purification methods.

The present disclosure further provides a method of making an antibody, masked antibody (e.g., activatable antibody), or antigen-binding fragment comprising culturing a host cell comprising a vector comprising a polynucleotide encoding an antibody, masked antibody (e.g., activatable antibody), or antigen-binding fragment described herein under conditions suitable for producing the masked antibody (e.g., activatable antibody), or the antibody or antigen-binding fragment thereof. In some embodiments, the method further comprises recovering the masked antibody (e.g., activatable antibody), or the antibody or antigen-binding fragment thereof, produced by the cell.

### E. Compositions Comprising Anti-CD47 Antibodies and Masked Antibodies

In other aspects, the present disclosure provides a composition containing a binding molecule (e.g. antibody, masked antibody (e.g., activatable antibody), or antigen-binding fragment) provided by the disclosure. In one aspect, the composition is a pharmaceutical composition comprising an antibody, masked antibody (e.g., activatable antibody), or antigen-binding fragment and a pharmaceutically acceptable carrier. The compositions can be prepared by conventional methods known in the art.

The term “pharmaceutically acceptable carrier” refers to any inactive substance that is suitable for use in a formulation for the delivery of a binding molecule (e.g., antibody, masked antibody (e.g., activatable antibody), or antigen-binding fragment). A carrier may be an antiadherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like) dextrose, vegetable oils (such as olive oil), saline, buffer, buffered saline, and isotonic agents such as sugars, polyalcohols, sorbitol, and sodium chloride.

The compositions may be in any suitable forms, such as liquid, semi-solid, and solid dosage forms. Examples of liquid dosage forms include solution (e.g., injectable and infusible solutions), microemulsion, liposome, dispersion, or suspension. Examples of solid dosage forms include tablet, pill, capsule, microcapsule, and powder. A particular form of the composition suitable for delivering a binding molecule (e.g., antibody, masked antibody (e.g., activatable antibody), or antigen-binding fragment) is a sterile liquid, such as a solution, suspension, or dispersion, for injection or infusion. Sterile solutions can be prepared by incorporating the antibody, masked antibody (e.g., activatable antibody), or antigen-binding fragment in the required amount in an appropriate carrier, followed by sterilization microfiltration. Generally, dispersions are prepared by incorporating the antibody, masked antibody (e.g., activatable antibody), or antigen-binding fragment into a sterile vehicle that contains a basic dispersion medium and other carriers. In the case of sterile powders for the preparation of sterile liquid, methods of preparation include vacuum drying and freeze-drying (lyophilization) to yield a powder of the active ingredient plus any additional desired ingredient from a previously sterile-filtered solution thereof. The various dosage forms of the compositions can be prepared by conventional techniques known in the art.

The relative amount of a binding molecule (e.g., antibody, masked antibody (e.g., activatable antibody), or antigen-binding fragment) included in the composition will vary depending upon a number of factors, such as the specific binding molecule and carriers used, dosage form, and desired release and pharmacodynamic characteristics. The amount of an antibody, masked antibody (e.g., activatable antibody), or antigen-binding fragment in a single dosage form will generally be that amount which produces a therapeutic effect, but may also be a lesser amount. Generally, this amount will range from about 0.01 percent to about 99 percent, from about 0.1 percent to about 70 percent, or from about 1 percent to about 30 percent relative to the total weight of the dosage form.

In addition to the antibody, masked antibody (e.g., activatable antibody), or antigen-binding fragment, one or more additional therapeutic agents may be included in the composition. Examples of additional therapeutic agents are described herein below. The suitable amount of the additional therapeutic agent to be included in the composition can be readily selected by a person skilled in the art, and will vary depending on a number of factors, such as the particular agent and carriers used, dosage form, and desired release and pharmacodynamic characteristics. The amount of the additional therapeutic agent included in a single dosage form will generally be that amount of the agent which produces a therapeutic effect, but may be a lesser amount as well.

### F. Methods of Using Anti-CD47 Antibodies and Masked Antibodies

Binding molecules (e.g., antibodies, antigen-binding fragments, or masked antibodies (e.g., activatable antibodies)) and pharmaceutical compositions provided by the present disclosure are useful for therapeutic, diagnostic, or other purposes, such as modulating an immune response, treating cancer, and enhancing efficacy of other cancer therapy. Thus, in other aspects, the present disclosure provides methods of using the antibodies, masked antibodies (e.g., activatable antibodies), antigen-binding fragments, or pharmaceutical compositions thereof. In one aspect, the present disclosure provides a method of treating a disorder in a subject, which comprises administering to the subject in need of treatment a therapeutically effective amount of an antibody, masked antibody (e.g., activatable antibody), or antigen-binding fragment provided by the disclosure. In some embodiments, the subject is a human. In some embodiments, administration of an effective amount of the antibody, masked antibody (e.g., activatable antibody), or antigen-binding fragment does not cause hemagglutination (e.g., clustering) of red blood cells (RBCs) (e.g., human RBCs) in the subject. In certain embodiments, administration of an effective amount of the antibody, masked antibody (e.g., activatable antibody), or antigen-binding fragment does not cause anemia in the subject.

In some embodiments, there is provided a method of treating a cancer (e.g., a CD47-positive cancer) in a subject, comprising administering to the subject a therapeutically effective amount of an antibody, masked antibody (e.g., activatable antibody), or antigen-binding fragment provided by the disclosure. In some embodiments, there is proved a method of treating a cancer (e.g., a CD47-positive cancer) in a subject, comprising administering to the subject a therapeutically effective amount of a masked antibody, comprising: (a) a masking peptide comprising, from N terminus to C terminus, a masking moiety (MM) and a linkage moiety (LM); (b) a target binding moiety (TBM) comprising an antibody heavy chain variable region (VH) and an antibody light chain variable region (VL), wherein the TBM binds to human CD47; and (c) an IgG1 Fc region, or an IgG Fc region with enhanced antibody-dependent cellular cytotoxicity (ADCC) activity, wherein the masking peptide is linked to the N terminus of the VH or the VL, and wherein the MM competes with human CD47 to bind the TBM. In some embodiments, the TBM competitively binds to the same epitope as any one of the anti-CD47 antibodies described herein. In some embodiments, the antibody, masked antibody (e.g., activatable antibody), or antigen-binding fragment is a CD47 antibody, masked antibody (e.g., activatable antibody), or antigen-binding fragment thereof and the subject is a human. A variety of CD47-positive cancers (i.e., cancers where CD47 is implicated) whether malignant or benign and whether primary or secondary, may be treated or prevented with a method provided by the disclosure. Examples of such cancer types include, but are not limited to, lymphoma (e.g., diffuse large B-cell lymphoma (DLBCL), lymphoid neoplasm diffuse large B-cell lymphoma (DLBC), mantle cell lymphoma, marginal cell lymphoma, non-Hodgkin's lymphoma, multiple B cell non-Hodgkin lymphoma subtypes (NHL) (e.g., diffuse large B cell lymphoma (DLBCL), B cell chronic lymphocytic leukemia (B-CLL), mantle cell lymphoma (MCL), follicular lymphoma (FL), marginal zone lymphoma (MZL), and pre-B acute lymphoblastic leukemia (pre-B ALL))), leukemia (e.g., acute myeloid leukemia (AML), acute lymphoblastic leukemia, acute lymphocytic leukemia, chronic lymphocytic leukemia, and chronic myeloid leukemia (CML)), head and neck cancer (e.g., head and neck squamous cell carcinoma (HNSC)), gastric cancer (e.g., gastric carcinoma, Epstein-Barr virus-associated gastric carcinoma (EBVaGC), and Her2+ gastric-esophageal junction (GEJ) cancer), breast cancer (e.g., breast invasive carcinoma (BRCA), HER2+ breast cancer, hormone receptor positive breast cancer, triple negative breast cancer (TNBC)), cervical cancer (e.g., cervical squamous cell carcinoma and endocervical adenocarcinoma (CESC)), cholangiocarcinoma (CHOL), colon cancer (e.g., colon adenocarcinoma (COAD)), ovarian cancer (e.g., ovarian serous cystadenocarcinoma (OV), ovarian clear cell carcinoma, epithelial ovarian cancer), thyroid cancer (e.g., thyroid carconima (THCA)), uterine cancer (e.g., uterine corpus endometrial carcinoma (UCEC)), endometrial cancer, lung cancer (e.g., lung adenocarcinoma (LUAD), lung squamous cell carcinoma (LUSC), small cell lung cancer (SCLC), and non-small cell lung cancer (NSCLC)), mesothelioma, pancreatic cancer (e.g., pancreatic adenocarcinoma (PAAD) and pancreatic ductal adenocarcinoma (PDAC)), bladder cancer (e.g., muscle invasive bladder cancer (MIBC), non-muscle invasive bladder cancer (NMIBC)), brain cancer (e.g., glioma), esophageal cancer, kidney cancer, liver cancer (e.g., hepatocellular carcinoma (HCC)), melanoma, prostate cancer, myeloma (e.g., multiple myeloma), bone cancer (e.g., osteosarcoma), muscle cancer (e.g., leiomyosarcoma), and myelodysplastic syndrome (MDS).

In another aspect, the present disclosure provides a method of enhancing an immune response in a subject, which comprises administering to the subject a therapeutically effective amount of an antibody, masked antibody (e.g., activatable antibody), or antigen-binding fragment provided by the disclosure. In some embodiments, the antibody, masked antibody (e.g., activatable antibody), or antigen-binding fragment is a CD47 antibody, masked antibody (e.g., activatable antibody), or antigen-binding fragment thereof and the subject is a human. The term “enhancing immune response” or its grammatical variations, means stimulating, evoking, increasing, improving, or augmenting any response of a subject's immune system. The immune response may be a cellular response (i.e. cell-mediated, such as cytotoxic T lymphocyte mediated) or a humoral response (i.e. antibody mediated response), and may be a primary or secondary immune response. Examples of enhancement of immune response include increased CD4+ helper T cell activity and generation of cytolytic T cells. The enhancement of immune response can be assessed using a number of in vitro or in vivo measurements known to those skilled in the art, including, but not limited to, cytotoxic T lymphocyte assays, release of cytokines (for example IL-2 production), regression of tumors, survival of tumor bearing animals, antibody production, immune cell proliferation, expression of cell surface markers, and cytotoxicity (e.g., antibody-dependent cellular cytotoxicity (ADCC)). Typically, methods of the disclosure enhance the immune response by a subject when compared to the immune response by an untreated subject or a subject not treated using the claimed methods. In one embodiment, the antibody, masked antibody (e.g., activatable antibody), or antigen-binding fragment is used to enhance the immune response of a human to a microbial pathogen (such as a virus). In another embodiment, the antibody, masked antibody (e.g., activatable antibody), or antigen-binding fragment is used to enhance the immune response of a human to a vaccine. The antibody, masked antibody (e.g., activatable antibody), or antigen-binding fragment may be used to enhance the immune response of a human to a microbial pathogen (such as a virus) or to a vaccine.

In practicing the therapeutic methods, the antibody, masked antibody (e.g., activatable antibody), or antigen-binding fragments may be administered alone as monotherapy, or administered in combination with one or more additional therapeutic agents or therapies. Thus, in another aspect, the present disclosure provides a combination therapy, which comprises an antibody, masked antibody (e.g., activatable antibody), or antigen-binding fragment in combination with one or more additional therapies or therapeutic agents for separate, sequential or simultaneous administration. The term “additional therapy” refers to a therapy which does not employ an antibody, masked antibody (e.g., activatable antibody), or antigen-binding fragment provided by the disclosure as a therapeutic agent. The term “additional therapeutic agent” refers to any therapeutic agent other than an antibody, masked antibody (e.g., activatable antibody), or antigen-binding fragment provided by the disclosure. In one particular aspect, the present disclosure provides a combination therapy for treating cancer in a subject, which comprises administering to the subject a therapeutically effective amount of an antibody, masked antibody (e.g., activatable antibody), or antigen-binding fragment provided by the disclosure in combination with one or more additional therapeutic agents. In a further embodiment, the subject is a human.

A wide variety of cancer therapeutic agents may be used in combination with an antibody, masked antibody (e.g., activatable antibody), or antigen-binding fragment provided by the present disclosure. One of ordinary skill in the art will recognize the presence and development of other cancer therapies which can be used in combination with the methods and antibody, masked antibody (e.g., activatable antibody), or antigen-binding fragments of the present disclosure, and will not be restricted to those forms of therapy set forth herein. Examples of categories of additional therapeutic agents that may be used in the combination therapy for treating cancer include (1) chemotherapeutic agents, (2) immunotherapeutic agents, and (3) hormone therapeutic agents.

The term “chemotherapeutic agent” refers to a chemical or biological substance that can cause death of cancer cells, or interfere with growth, division, repair, and/or function of cancer cells. Examples of chemotherapeutic agents include those that are disclosed in WO 2006/129163, and US20060153808, the disclosures of which are incorporated herein by reference. Examples of particular chemotherapeutic agents include: (1) alkylating agents, such as chlorambucil (LEUKERAN), mcyclophosphamide (CYTOXAN), ifosfamide (IFEX), mechlorethamine hydrochloride (MUSTARGEN), thiotepa (THIOPLEX), streptozotocin (ZANOSAR), carmustine (BICNU, GLIADEL WAFER), lomustine (CEENU), and dacarbazine (DTIC-DOME); (2) alkaloids or plant vinca alkaloids, including cytotoxic antibiotics, such as doxorubicin (ADRIAMYCIN), epirubicin (ELLENCE, PHARMORUBICIN), daunorubicin (CERUBIDINE, DAUNOXOME), nemorubicin, idarubicin (IDAMYCIN PFS, ZAVEDOS), mitoxantrone (DHAD, NOVANTRONE). dactinomycin (actinomycin D, COSMEGEN), plicamycin (MITHRACIN), mitomycin (MUTAMYCIN), and bleomycin (BLENOXANE), vinorelbine tartrate (NAVELBINE)), vinblastine (VELBAN), vincristine (ONCOVIN), and vindesine (ELDISINE); (3) antimetabolites, such as capecitabine (XELODA), cytarabine (CYTOSAR-U), fludarabinc (FLUDARA), gemcitabine (GEMZAR), hydroxyurea (HYDRA), methotrexate (FOLEX, MEXATE, TREXALL), nelarabine (ARRANON), trimetrexate (NEUTREXIN), and pemetrexed (ALIMTA); (4) Pyrimidine antagonists, such as 5-fluorouracil (5-FU); capecitabine (XELODA), raltitrexed (TOMUDEX), tegafur-uracil (UFTORAL), and gemcitabine (GEMZAR); (5) taxanes, such as docetaxel (TAXOTERE), paclitaxel (TAXOL); (6) platinum drugs, such as cisplatin (PLATINOL) and carboplatin (PARAPLATIN), and oxaliplatin (ELOXATIN); (7) topoisomerase inhibitors, such as irinotecan (CAMPTOSAR), topotecan (HYCAMTIN), etoposide (ETOPOPHOS, VEPESSID, TOPOSAR), and teniposide (VUMON); (8) cpipodophyllotoxins (podophyllotoxin derivatives), such as etoposide (ETOPOPHOS, VEPESSID, TOPOSAR); (9) folic acid derivatives, such as leucovorin (WELLCOVORIN); (10) nitrosoureas, such as carmustinc (BiCNU), lomustine (CecNU); (11) inhibitors of receptor tyrosine kinase, including epidermal growth factor receptor (EGFR), vascular endothelial growth factor (VEGF), insulin receptor, insulin-like growth factor receptor (IGFR), hepatocyte growth factor receptor (HGFR), and platelet-derived growth factor receptor (PDGFR), such as gefitinib (IRESSA), erlotinib (TARCEVA), bortezomib (VELCADE), imatinib mesylate (GLEEVEC), genefitinib, lapatinib, sorafenib, thalidomide, sunitinib (SUTENT), axitinib, rituximab (RITUXAN, MABTHERA), trastuzumab (HERCEPTIN), cetuximab (ERBITUX), bevacizumab (AVASTIN), and ranibizumab (LUCENTIS), lym-1 (ONCOLYM), antibodies to insulin-like growth factor-1 receptor (IGF-1R) that are disclosed in WO2002/053596); (12) angiogenesis inhibitors, such as bevacizumab (AVASTIN), suramin (GERMANIN), angiostatin, SU5416, thalidomide, and matrix metalloproteinase inhibitors (such as batimastat and marimastat), and those that are disclosed in WO2002055106; and (13) proteasome inhibitors, such as bortezomib (VELCADE).

The term “immunotherapeutic agents” refers to a chemical or biological substance that can enhance an immune response of a subject. Examples of immunotherapeutic agents include: bacillus Calmette-Guerin (BCG); cytokines such as interferons; vaccines such as MyVax personalized immunotherapy, Onyvax-P. Oncophage, GRNVAC1, Favld, Provenge, GVAX, Lovaxin C, BiovaxID, GMXX, and NeuVax; and antibodies such as alemtuzumab (CAMPATH), bevacizumab (AVASTIN), cetuximab (ERBITUX), gemtuzunab ozogamicin (MYLOTARG), ibritumomab tiuxetan (ZEVALIN), panitumumab (VECTIBIX), rituximab (RITUXAN, MABTHERA), trastuzumab (HERCEPTIN), tositumomab (BEXXAR), ipilimumab (YERVOY) tremelimumab, CAT-3888, agonist antibodies to OX40 receptor (such as those disclosed in WO2009/079335), agonist antibodies to CD40 receptor (such as those disclosed in WO2003/040170, and TLR-9 agonists (such as those disclosed in WO2003/015711, WO2004/016805, and WO2009/022215).

The term “hormone therapeutic agent” refers to a chemical or biological substance that inhibits or eliminates the production of a hormone, or inhibits or counteracts the effect of a hormone on the growth and/or survival of cancerous cells. Examples of such agents suitable for the methods herein include those that are disclosed in US20070117809. Examples of particular hormone therapeutic agents include tamoxifen (NOLVADEX), toremifene (Fareston), fulvestrant (FASLODEX), anastrozole (ARIMIDEX), exemestane (AROMASIN), letrozole (FEMARA), megestrol acetate (MEGACE), goserelin (ZOLADEX), and leuprolide (LUPRON). The antibody, masked antibody (e.g., activatable antibody), or antigen-binding fragments of this disclosure may also be used in combination with non-drug hormone therapies such as (1) surgical methods that remove all or part of the organs or glands which participate in the production of the hormone, such as the ovaries, the testicles, the adrenal gland, and the pituitary gland, and (2) radiation treatment, in which the organs or glands of the patient are subjected to radiation in an amount sufficient to inhibit or eliminate the production of the targeted hormone.

The combination therapy for treating cancer also encompasses the combination of an antibody, masked antibody (e.g., activatable antibody), or antigen-binding fragment with surgery to remove a tumor. The antibody, masked antibody (e.g., activatable antibody), or antigen-binding fragment may be administered to the subject before, during, or after the surgery.

The combination therapy for treating cancer also encompasses combination of an antibody, masked antibody (e.g., activatable antibody), or antigen-binding fragment with radiation therapy, such as ionizing (electromagnetic) radiotherapy (e.g., X-rays or gamma rays) and particle beam radiation therapy (e.g., high linear energy radiation). The source of radiation can be external or internal to the subject. The antibody, masked antibody (e.g., activatable antibody), or antigen-binding fragment may be administered to the subject before, during, or after the radiation therapy.

The antibody, masked antibody (e.g., activatable antibody), or antigen-binding fragments and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumoral, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The antibodies, antigen-binding fragments, masked antibodies (e.g., activatable antibodies), and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific antibody, antigen-binding fragment, or masked antibody (e.g., activatable antibody) being used, the rate of absorption desired, specific formulation or dosage form used, type or severity of the disorder being treated, the specific site of action, and conditions of the patient, and can be readily selected by a person skilled in the art.

The term “therapeutically effective amount” of an antibody, masked antibody (e.g., activatable antibody), or antigen-binding fragment refers to an amount that is effective for an intended therapeutic purpose. For example, in the context of enhancing an immune response, a “therapeutically effective amount” is any amount that is effective in stimulating, evoking, increasing, improving, or augmenting any response of a subject's immune system. In the context of treating a disease, a “therapeutically effective amount” is any amount that is sufficient to cause any desirable or beneficial effect in the subject being treated. Specifically, in the treatment of cancer, examples of desirable or beneficial effects include inhibition of further growth or spread of cancer cells, death of cancer cells, inhibition of reoccurrence of cancer, reduction of pain associated with the cancer, or improved survival of the subject. The therapeutically effective amount of a CD47 antibody, antigen-binding fragment, or masked antibody (e.g., activatable antibody) usually ranges from about 0.001 to about 500 mg/kg, such as about 0.01 to about 100 mg/kg, of the body weight of the subject (e.g., a cynomolgus monkey). In some embodiments, the CD47 antibody, antigen-binding fragment, or masked antibody (e.g., activatable antibody) is administered at a dose of about 0.01 to 60 mg/kg of the subject (e.g., a cynomolgus monkey), such as about any one of 0.01-1 mg/kg, 1-10 mg/kg, or 10-60 mg/kg of the subject (e.g., a cynomolgus monkey). For example, the CD47 antibody, antigen-binding fragment, or masked antibody (e.g., activatable antibody) is administered at a dose of at least about any one of 0.01 mg/kg, 0.1 mg/kg, 0.6 mg/kg. 1 mg/kg, 5 mg/kg, 10 mg/kg, 15 mg/kg, 20 mg/kg, 25 mg/kg, or 30 mg/kg of body weight of the subject (e.g., a cynomolgus monkey). In some embodiments, the CD47 antibody, antigen-binding fragment, or masked antibody (e.g., activatable antibody) is administered at a dose of no more than about any one of 0.6 mg/kg, 1 mg/kg, 5 mg/kg, 10 mg/kg, 15 mg/kg, 20 mg/kg, 25 mg/kg. 30 mg/kg, 40 mg/kg, 50 mg/kg, or 60 mg/kg of body weight of the subject (e.g., a cynomolgus monkey). In some embodiments, the CD47 antibody, antigen-binding fragment, or masked antibody (e.g., activatable antibody) is administered at a dose of at least 0.6 mg/kg of the subject. The precise dosage level to be administered can be readily determined by a person skilled in the art and will depend on a number of factors, such as the type, and severity of the disorder to be treated, the particular antibody, masked antibody (e.g., activatable antibody), or antigen-binding fragment employed, the route of administration, the time of administration, the duration of the treatment, the particular additional therapy employed, the age, sex, weight, condition, general health and prior medical history of the patient being treated, and like factors well known in the medical arts.

An antibody, masked antibody (e.g., activatable antibody), or antigen-binding fragment or composition is usually administered on multiple occasions. Intervals between single doses can be, for example, weekly, monthly, every three months or yearly. An exemplary treatment regimen entails administration once per week, once every two weeks, once every three weeks, once every four weeks, once a month, once every three months or once every three to six months. Typical dosage regimens for a CD47 antibody, antigen-binding fragment, or masked antibody (e.g., activatable antibody) include 1 mg/kg body weight or 3 mg/kg body weight via intravenous administration, using one of the following dosing schedules: (i) every four weeks for six dosages, then every three months; (ii) every three weeks; (iii) 3 mg/kg body weight once followed by 1 mg/kg body weight every three weeks. In some embodiments, the antibody, masked antibody (e.g., activatable antibody), or antigen-binding fragment is administered at least once every three weeks. In certain embodiments, the antibody, masked antibody (e.g., activatable antibody), or antigen-binding fragment is administered at least once every two weeks. In some embodiments, a pharmaceutical composition comprising the antibody, masked antibody (e.g., activatable antibody), or antigen-binding fragment is administered at least once every three weeks. In certain embodiments, a pharmaceutical composition comprising the antibody, masked antibody (e.g., activatable antibody), or antigen-binding fragment is administered at least once every two weeks.

### G. Articles of Manufacture and Kits

In some embodiments of the invention, there is provided an article of manufacture containing materials useful for the treatment of a CD47-positive disease such as cancer, for delivering an anti-CD47 antibody, antigen-binding fragment, or masked antibody (e.g., activatable antibody) to a cell expressing CD47 on its surface. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein and may have a sterile access port (for example the container may be an intravenous solution bag or a vial having a stopper pierceable by a hypodermic injection needle). At least one active agent in the composition is an anti-CD47 antibody, antigen-binding fragment, or masked antibody (e.g., activatable antibody) construct of the invention. The label or package insert indicates that the composition is used for treating the particular condition. The label or package insert will further comprise instructions for administering the anti-CD47 antibody, antigen-binding fragment, or masked antibody (e.g., activatable antibody) composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating cancer.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of an CD47-positive disease or disorder described herein, for delivering an anti-CD47 antibody, antigen-binding fragment, or masked antibody (e.g., activatable antibody) to a cell expressing CD47 on its surface, optionally in combination with the articles of manufacture. Kits of the invention include one or more containers comprising an anti-CD47 antibody, antigen-binding fragment, or masked antibody (e.g., activatable antibody) composition (or unit dosage form and/or article of manufacture), and in some embodiments, further comprise another agent (such as the agents described herein) and/or instructions for use in accordance with any of the methods described herein. The kit may further comprise a description of selection of individuals suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition comprising an anti-CD47 antibody, antigen-binding fragment, or masked antibody (e.g., activatable antibody). In some embodiments, the kit comprises a) a composition comprising an anti-CD47 antibody, antigen-binding fragment, or masked antibody (e.g., activatable antibody), and b) an effective amount of at least one other agent, wherein the other agent enhances the effect (e.g., treatment effect) of the anti-CD47 antibody, antigen-binding fragment, or masked antibody (e.g., activatable antibody). In some embodiments, the kit comprises a) a composition comprising an anti-CD47 antibody, antigen-binding fragment, or masked antibody (e.g., activatable antibody), and b) instructions for administering the anti-CD47 antibody, antigen-binding fragment, or masked antibody (e.g., activatable antibody) composition to an individual for treatment of a CD47-positive disease, such as cancer. In some embodiments, the kit comprises a) a composition comprising an anti-CD47 antibody, antigen-binding fragment, or masked antibody (e.g., activatable antibody), b) an effective amount of at least one other agent, wherein the other agent enhances the effect (e.g., treatment effect) of the anti-CD47 antibody, antigen-binding fragment, or masked antibody (e.g., activatable antibody), and c) instructions for administering the anti-CD47 antibody, antigen-binding fragment, or masked antibody (e.g., activatable antibody) composition and the other agent(s) to an individual for treatment of an CD47-positive disease, such as cancer. The anti-CD47 antibody, antigen-binding fragment, or masked antibody (e.g., activatable antibody) and the other agent(s) can be present in separate containers or in a single container. For example, the kit may comprise one distinct composition or two or more compositions wherein one composition comprises an anti-CD47 antibody, antigen-binding fragment, or masked antibody (e.g., activatable antibody) and another composition comprises another agent.

In some embodiments, the kit comprises a nucleic acid (or set of nucleic acids) encoding an anti-CD47 antibody, antigen-binding fragment, or masked antibody (e.g., activatable antibody) or one or more polypeptide portions thereof (e.g., a CDR, a VH, a VR, a heavy chain, a light chain, a masking peptide, an Fc region, or the like). In some embodiments, the kit comprises a) a nucleic acid (or set of nucleic acids) encoding an anti-CD47 antibody, antigen-binding fragment, or masked antibody (e.g., activatable antibody) or one or more polypeptide portions thereof, and b) a host cell for expressing the nucleic acid (or set of nucleic acids). In some embodiments, the kit comprises a) a nucleic acid (or set of nucleic acids) encoding an anti-CD47 antibody, antigen-binding fragment, or masked antibody (e.g., activatable antibody) or more or more polypeptide portions thereof, and b) instructions for i) expressing the anti-CD47 antibody, antigen-binding fragment, or masked antibody (e.g., activatable antibody) in a host cell, ii) preparing a composition comprising the anti-CD47 antibody, antigen-binding fragment, or masked antibody (e.g., activatable antibody), or the host cell expressing the anti-CD47 antibody, antigen-binding fragment, or masked antibody (e.g., activatable antibody), and iii) administering the composition comprising the anti-CD47 antibody, antigen-binding fragment, or masked antibody (e.g., activatable antibody), or the host cell expressing the anti-CD47 antibody, antigen-binding fragment, or masked antibody (e.g., activatable antibody), to an individual for the treatment of an CD47-positive disease, such as cancer. In some embodiments, the host cell is derived from the individual. In some embodiments, the kit comprises a) a nucleic acid (or set of nucleic acids) encoding an anti-CD47 antibody, antigen-binding fragment, or masked antibody (e.g., activatable antibody) or one or more polypeptide portions thereof, b) a host cell for expressing the nucleic acid (or set of nucleic acids), and c) instructions for i) expressing the anti-CD47 antibody, antigen-binding fragment, or masked antibody (e.g., activatable antibody) in the host cell, ii) preparing a composition comprising the anti-CD47 antibody, antigen-binding fragment, or masked antibody (e.g., activatable antibody), or the host cell expressing the anti-CD47 antibody, antigen-binding fragment, or masked antibody (e.g., activatable antibody), and iii) administering the composition comprising the anti-CD47 antibody, antigen-binding fragment, or masked antibody (e.g., activatable antibody), or the host cell expressing the anti-CD47 antibody, antigen-binding fragment, or masked antibody (e.g., activatable antibody), to an individual for the treatment of an CD47-positive disease, such as cancer. In some embodiments, the expression of the anti-CD47 antibody, antigen-binding fragment, or masked antibody (e.g., activatable antibody), or one or more polypeptide portions thereof, is inducible. In some embodiments, the nucleic acid (or set of nucleic acids) encoding an anti-CD47 antibody, antigen-binding fragment, or masked antibody (e.g., activatable antibody), or one or more polypeptide portions thereof, are under control of inducible promoter(s).

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the anti-CD47 antibody, antigen-binding fragment, or masked antibody (e.g., activatable antibody) compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of an anti-CD47 antibody, antigen-binding fragment, or masked antibody (e.g., activatable antibody), as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the anti-CD47 antibody, antigen-binding fragment, or masked antibody (e.g., activatable antibody) and pharmaceutical compositions and instructions for use and packaged in quantities sufficient for storage and use in pharmacies, for example, hospital pharmacies and compounding pharmacies.

The present disclosure will be more fully understood by reference to the following examples. The examples should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims. The contents of all figures and all references, patents and published patent applications cited throughout this disclosure are expressly incorporated herein by reference in their entirety.

### EXAMPLES

### Example 1: Generation of Antibodies that Specifically Bind to Human CD47

Multiple proprietary phagemid libraries were employed to pan against human CD47-Fc antigen (Sinobiological). The antigen was either directly immobilized to Maxisorp microplates (Thermo Scientific 446469) or was biotinylated and captured by Dynabeads (M280, Streptavidin, Invitrogen #60210) for panning through KingFisher (Thermo Scientific) according to manufacturer's instructions. Standard phage panning protocols were employed in the process. A total of three rounds of panning were conducted against each antigen, and 10-100 fold excess of purified human Fc was included to reduce background binding. After the final round of panning, single-colony supernatant ELISA was performed to identify the primary hits that specifically recognize human CD47. The primary hits were defined as those whose ELISA signals were at least twice that of background. As shown in Table 7, a total of seventeen unique hits were identified, and the heavy chains and light chains of the Fabs were cloned into the mammalian expression vector pcDNA3.3 (Thermo Fisher Scientific) with IgG4 isotype. Pairs of plasmids were transiently transfected into HEK293 cells following manufacturer's instructions. Seven days later, the supernatants were harvested, cleared by centrifugation and filtration, and IgGs were purified with standard protein A affinity chromatography (MabSelect SuRe, GE Healthcare). The proteins were eluted and neutralized, and buffer exchanged into PB buffer (20 mM sodium phosphate, 150 mM NaCl, pH 7.0). Protein concentrations were determined by UV-spectrophotometry, and IgG purity was analyzed under denaturing, reducing and non-reducing conditions.

TABLE 7
Amino acid sequences of anti-CD47 antibodies from phage display screening.

| | Heavy Chain Variable Region | | | | Light Chain Variable Region | | | |
|---|---|---|---|---|---|---|---|---|
| | Amino Acid SEQ ID Nos | | | | Amino Acid SEQ ID Nos | | | |
| Ab ID | HCVR | HCDR1 | HCDR2 | HCDR3 | LCVR | LCDR1 | LCDR2 | LCDR3 |
| TY25029 | 1 | 35 | 52 | 69 | 2 | 86 | 103 | 120 |
| TY25030 | 3 | 36 | 53 | 70 | 4 | 87 | 104 | 121 |
| TY25031 | 5 | 37 | 54 | 71 | 6 | 88 | 105 | 122 |
| TY25032 | 7 | 38 | 55 | 72 | 8 | 89 | 106 | 123 |
| TY25033 | 9 | 39 | 56 | 73 | 10 | 90 | 107 | 124 |
| TY25034 | 11 | 40 | 57 | 74 | 12 | 91 | 108 | 125 |
| TY25035 | 13 | 41 | 58 | 75 | 14 | 92 | 109 | 126 |
| TY25036 | 15 | 42 | 59 | 76 | 16 | 93 | 110 | 127 |
| TY25037 | 17 | 43 | 60 | 77 | 18 | 94 | 111 | 128 |
| TY25038 | 19 | 44 | 61 | 78 | 20 | 95 | 112 | 129 |
| TY25039 | 21 | 45 | 62 | 79 | 22 | 96 | 113 | 130 |
| TY25040 | 23 | 46 | 63 | 80 | 24 | 97 | 114 | 131 |
| TY25041 | 25 | 47 | 64 | 81 | 26 | 98 | 115 | 132 |
| TY21446 | 27 | 48 | 65 | 82 | 28 | 99 | 116 | 133 |
| TY21447 | 29 | 49 | 66 | 83 | 30 | 100 | 117 | 134 |
| TY21449 | 31 | 50 | 67 | 84 | 32 | 101 | 118 | 135 |
| TY21451 | 33 | 51 | 68 | 85 | 34 | 102 | 119 | 136 |

### Example 2: In Vitro Screening of the Anti-CD47 Parental Antibodies

Binding affinity to human CD47 (hCD47) using ELISA

The 96-well plates were coated with 2 μg/mL hCD47-ECD/Fc protein (Sinobiological) at 4° C. overnight. The humanized anti-CD47 antibody TAC2204 (also known as 5F9; as described in Table 4B and U.S. Pat. No. 9,017,675) was used as a benchmark control. After blocking, the plates were washed three times and incubated with test antibodies with serial dilution at 37° C. for 1 hour. Then the plates were washed three times and incubated with HRP-conjugated anti-human Fab secondary antibody (Sigma) at 37° C. for 1 hour. After washing three times, the plates were incubated with TMB substrate for 15 minutes at room temperature. The reaction was stopped by the addition of H2SO4 stop solution. Absorbance at 450 nm was measured by a microplate reader. The data were analyzed by GraphPad Prism 7.0.

As shown in Table 8, all of the test antibodies except TY25032 and TY21451 bind to human CD47 extracellular domain with high affinity.

TABLE 8
Summary of EC50 values of ELISA binding to hCD47.
Duplicate values given for the same antibody represent
two independent measurements for that antibody.

| Ab ID | EC50 (nM) |
|---|---|
| TY25029 | 0.58 |
| TY25030 | 2.42 |
| TY25031 | 0.59 |
| TY25032 | ND |
| TY25033 | 6.11 |
| TY25034 | 0.47 |
| TY25035 | 2.55 |
| TY25036 | 1.71 |
| TY25037 | 0.95 |
| TY25038 | 0.78 |
| TY25039 | 3.66 |
| TY25040 | 0.46 |
| TY25041 | 1.17 |
| TY21446 | 0.41 |
| TY21447 | 0.33 |
| TY21451 | ND |
| TY25031 | 0.60 |
| TY25034 | 0.41 |
| TY25040 | 0.39 |
| TAC2204 | 0.34 |
| TAC2204 | 0.34 |

### Ligand Blocking

Recombinant hCD47-ECD/Fc (Sinobiological) was coated on 96-well plate (100 ng/well) at 4° C. overnight. After blocking, the plates were washed three times and incubated with the mixture of biotinylated hSIRPα-ECD/His (Sinobiological) of 0.5 g/mL and test antibodies with serial dilution at 37° C. for 1 hour. The plates were washed three times again and incubated with HRP-conjugated streptavidin at 37° C. for 1 hour. After washing, the plates were incubated with TMB substrate for 15 minutes at room temperature. The reaction was stopped by the addition of H2SO4 stop solution. Absorbance at 450 nm was measured by a microplate reader. The data was analyzed by GraphPad Prism 7.0.

As shown in Tables 9A-9B, TAC2204, TY25031, TY25034, TY25040, TY21446, TY21447 and TY21449 showed comparable ability to block hSIRPα/hCD47 interaction.

TABLE 9A
Summary of IC50 values of ligand
blocking; first independent experiment.

| Ab ID | IC50 (nM) |
|---|---|
| TY25029 | >100 |
| TY25030 | 21.68 |
| TY25031 | 4.55 |
| TY25033 | >100 |
| TY25034 | 3.53 |
| TY25035 | 60.56 |
| TY25036 | >100 |
| TY25037 | >100 |
| TY25038 | >100 |
| TY25039 | >100 |
| TY25040 | 3.30 |
| TY25041 | >100 |
| TAC2204 | 2.95 |

TABLE 9B
Summary of IC50 values of ligand blocking—second experiment.

| Ab ID | IC50 (nM) |
|---|---|
| TY21446 | 2.86 |
| TY21447 | 3.12 |
| TY21449 | 3.18 |
| TY21451 | >100 |
| TAC2204 | 3.12 |

### Affinity Ranking Study

The binding affinity between antibodies and human CD47 was measured through Octet® RED96 System (ForteBio). TAC2204, and the anti-CD47 antibody TAC2407 (also known as 13H3 and TJC4 (lemzoparlimab)); as described in Table 4B and U.S. Pat. Pub. No. US20210095019) were used as benchmark controls. The SA sensors (ForteBio) were loaded with biotinylated hCD47-ECD/Fc (Sinobiological). After washing, the sensors were dipped into solutions containing test antibodies for 300 seconds to monitor the association phase, and then were dipped into running buffer for 300 seconds to monitor the dissociation phase. The acquired ForteBio data were processed with Data Acquisition software 7.1, and kinetic data were fitted to a 1:1 Langmuir binding model.

As shown in Table 10, TY21446 and TAC2204 showed similar equilibrium dissociation constants.

TABLE 10
Affinity ranking study of antibody binding to hCD47 as
measured by ForteBio. The duplicate values given for
TY25034 represent two independent measurements.

| | K_{D} | Kₒₙ | K_{off} |
|---|---|---|---|
| Sample ID | (nM) | (1/Ms) | (1/s) |
| TAC2204 | 0.3475 | 6.76E+05 | 2.35E−04 |
| TAC2407 | 1.404 | 5.20E+05 | 7.29E−04 |
| TY25029 | 1.515 | 4.17E+05 | 6.31E−04 |
| TY25030 | 4.616 | 3.61E+05 | 1.66E−03 |
| TY25031 | 0.4834 | 1.68E+05 | 8.10E−05 |
| TY25032 | 2.729 | 2.95E+05 | 8.06E−04 |
| TY25033 | <0.001 | 5.89E+04 | <1.0E−07 |
| TY25034 | 0.8256 | 8.13E+05 | 6.71E−04 |
| TY25035 | <0.001 | 1.71E+05 | <1.0E−07 |
| TY25036 | <0.001 | 9.37E+04 | <1.0E−07 |
| TY25037 | 0.6109 | 5.60E+04 | 3.42E−05 |
| TY25038 | 4.467 | 2.49E+05 | 1.11E−03 |
| TY25039 | 0.1407 | 9.73E+04 | 1.37E−05 |
| TY25040 | 0.4739 | 4.22E+05 | 2.00E−04 |
| TY21446 | 0.1937 | 6.42E+05 | 1.24E−04 |
| TY25034 | 0.8697 | 8.09E+05 | 7.04E−04 |

### Example 3: Generation of Anti-CD47 Activatable Antibodies

The IgG isotype of TY21446 was further switched from human IgG4 to human IgG1, and IgG1 with S239D and 1332E substitutions, to produce TY26896 and TY26897, respectively, in order to induce ADCC and/or CDC effect against tumor cells. To achieve tumor-specific activation of immune cells by the anti-CD47 antibodies, the activatable antibody of TY21446, TY26896 and TY26897 were further developed. Using recombinant DNA technology, a masking moiety (MM) and a cleavable moiety (CM) linker containing an enzyme-specific cleavage site were tandemly connected to the N-terminus of the light chain of the parental antibodies TY21446, TY26896 and TY26897 to produce activatable antibodies TY26294, TY26898, and TY26899, respectively. Table 11 shows the identifiers (IDs) and IgG isotypes of the activatable and parental (non-activatable) antibodies.

TABLE 11
Anti-CD47 antibodies and activatable antibodies.

| Activatable Ab ID | Parental Ab ID | IgG |
|---|---|---|
| TY26294 | TY21446 | hIgG4 |
| TY26898 | TY26896 | hIgG1 |
| TY26899 | TY26897 | hIgG1 S239D and I332E |

Some proteases are specifically highly expressed in human tumors. The masking peptide on anti-CD47 activatable antibodies can be removed by MMP protease cleavage in the tumor microenvironment, which leads to the restoration of CD47 binding affinity of the antibodies and therefore tumor-specific activation of macrophages by blocking hSIRPα/hCD47 pathway. For TY26898 and TY26899, NK cells will also be recruited to kill tumor cells in a tumor-specific manner. The restricted activations of all anti-CD47 activatable antibodies were supposed to reduce on-target off-tumor toxicity in cancer patients, especially RBC depletion, and increase half-life due to decreased antigen sink effect.

### Example 4: Stability Studies of the Anti-CD47 Activatable Antibodies

Antibody stabilities were examined under different stress conditions.

As shown in Tables 12A-12C, TY26294, TY26898 and TY26899 at a concentration of 1 mg/mL remained stable after 6 cycles of freezing (−80° C.) and thawing (room temperature), as analyzed by SEC-HPLC. After 28 days at 4 or 40° C., or 7 days at 25° C., there was little change of HMW aggregate or low molecular weight (LMW) fragments for TY26294, TY26898 and TY26899. TY26294, TY26898 and TY26899 were also stable under pH 3.6 for 2 hours at room temperature, and under high salt buffer containing 300 mM NaCl for 24 hours at room temperature. In use stability studies showed that TY26294, TY26898 and TY26899 at 1 mg/kg were stable for 30 hours at room temperature. Taken together, these results indicated that even without formulation optimization. TY26294. TY26898 and TY26899 had excellent developability profiles.

TABLE 12A
Changes of HMW % in SEC-HPLC analysis under different stress conditions for TY26294.

| | | No-treatment | | | After-treatment | | |
|---|---|---|---|---|---|---|---|
| | | | Main | | | Main | |
| Conditions | Buffer | HMW % | peak % | LMW % | HMW % | peak % | LMW % |
| Freeze thaw 6 cycles | 20 mM Histidine, | 3.2% | 94.4% | 2.4% | 3.6% | 94.6% | 1.8% |
| | pH 5.5 | | | | | | |
| Storage stability | 20 mM Histidine, | 2.9% | 96.9% | 0.3% | 2.9% | 96.9% | 0.3% |
| at 4° C. for 28 days | pH 5.5 | | | | | | |
| Storage stability | 20 mM Histidine, | 3.2% | 94.4% | 2.4% | 3.9% | 93.4% | 2.7% |
| at 25° C. for 7 days | pH 5.5 | | | | | | |
| Storage stability | 20 mM Histidine, | 3.5% | 96.5% | 0.1% | 3.3% | 96.6% | 0.1% |
| at 40° C. for 28 days | pH 5.5 | | | | | | |
| pH stability at pH | 50 mM NaAc/Hac, | 1.4% | 97.5% | 1.1% | 1.2% | 96.9% | 1.9% |
| 3.6 for 2 hours at R.T. | pH 3.6 | | | | | | |
| High salt stability | 50 mM Histidine, | 2.7% | 97.3% | 0.0% | 1.6% | 98.4% | 0.0% |
| for 24 hours at R.T. | 300 mM NaCl, pH 7.0 | | | | | | |
| In use stability at | 0.9% NaCl | 2.8% | 97.2% | 0.0% | 2.8% | 97.0% | 0.2% |
| 1 mg/mL for 30 hours. | | | | | | | |

TABLE 12B
Changes of HMW % in SEC-HPLC analysis under different stress conditions for TY26898.

| | | No-treatment | | | After-treatment | | |
|---|---|---|---|---|---|---|---|
| | | | Main | | | Main | |
| Conditions | Buffer | HMW % | peak % | LMW % | HMW % | peak % | LMW % |
| Freeze thaw 6 cycles | 20 mM Histidine, | 3.0% | 95.9% | 1.1% | 3.3% | 96.1% | 0.7% |
| | pH 5.5 | | | | | | |
| Storage stability | 20 mM Histidine, | 3.2% | 96.0% | 0.7% | 1.7% | 97.6% | 0.8% |
| at 4° C. for 28 days | pH 5.5 | | | | | | |
| Storage stability | 20 mM Histidine, | 3.2% | 96.0% | 0.7% | 1.8% | 97.2% | 0.9% |
| at 25° C. for 7 days | pH 5.5 | | | | | | |
| Storage stability | 20 mM Histidine, | 3.2% | 96.0% | 0.7% | 1.3% | 96.9% | 1.7% |
| at 40° C. for 28 days | pH 5.5 | | | | | | |
| pH stability at pH | 50 mM NaAc/Hac, | 1.1% | 98.0% | 0.8% | 1.1% | 97.9% | 1.0% |
| 3.6 for 2 hours at R.T. | pH 3.6 | | | | | | |
| High salt stability | 50 mM Histidine, | 2.0% | 97.4% | 0.6% | 2.5% | 96.9% | 0.6% |
| for 24 hours at R.T. | 300 mM NaCl, pH 7.0 | | | | | | |
| In use stability at | 0.9% NaCl | 1.9% | 97.4% | 0.7% | 2.3% | 97.0% | 0.7% |
| 1 mg/mL for 30 hours. | | | | | | | |

TABLE 12C
Changes of HMW % in SEC-HPLC analysis under different stress conditions for TY26898.

| | | No-treatment | | | After-treatment | | |
|---|---|---|---|---|---|---|---|
| | | | Main | | | Main | |
| Conditions | Buffer | HMW % | peak % | LMW % | HMW % | peak % | LMW % |
| Freeze thaw 6 cycles | 20 mM Histidine, | 2.2% | 96.8% | 1.0% | 3.4% | 95.6% | 1.0% |
| | pH 5.5 | | | | | | |
| Storage stability | 20 mM Histidine, | 2.3% | 96.9% | 0.8% | 1.2% | 98.0% | 0.9% |
| at 4° C. for 28 days | pH 5.5 | | | | | | |
| Storage stability | 20 mM Histidine, | 2.3% | 96.9% | 0.8% | 1.3% | 97.6% | 1.1% |
| at 25° C. for 7 days | pH 5.5 | | | | | | |
| Storage stability | 20 mM Histidine, | 2.3% | 96.9% | 0.8% | 1.3% | 96.6% | 2.1% |
| at 40° C. for 28 days | pH 5.5 | | | | | | |
| pH stability at pH | 50 mM NaAc/Hac, | 1.4% | 97.6% | 1.0% | 1.3% | 97.8% | 1.0% |
| 3.6 for 2 hours at R.T. | pH 3.6 | | | | | | |
| High salt stability | 50 mM Histidine, | 1.6% | 97.7% | 0.7% | 1.8% | 97.5% | 0.7% |
| for 24 hours at R.T. | 300 mM NaCl, pH 7.0 | | | | | | |
| In use stability at | 0.9% NaCl | 1.7% | 97.4% | 0.9% | 2.0% | 97.2% | 0.8% |
| 1 mg/mL for 30 hours. | | | | | | | |

### Example 5: Anti-CD47 Antibody Binding Affinities to Human, Cynomolgus Monkey, and Mouse CD47

The binding affinities to human, cynomolgus monkey (cyno), and mouse (m) CD47 were measured by ELISA. The 96-well plates were coated with 100 ng/well hCD47-ECD/Fc or cynoCD47-ECD/His or mCD47-ECD/His proteins (Sinobiological) at 4° C. overnight. After blocking, the plates were washed three times and incubated with test antibodies with serial dilution at 37° C. for 1 hour. Then the plates were washed three times and incubated with HRP-conjugated anti-human Fab secondary antibody (Sigma) at 37° C. for 1 hour. After washing three times, the plates were incubated with TMB substrate for 15 minutes at room temperature. The reaction was stopped by the addition of H2SO4 stop solution. Absorbance at 450 nm was measured by a microplate reader. The data was analyzed by GraphPad Prism. The binding affinities to human CD47 were measured in three separate batches.

As shown in Table 13, the parental antibodies TY21446, TY26896 and TY26897 showed similar binding affinity to human and cynomolgus monkey CD47, but they did not bind to mouse CD47 as high as 8 μM. Masking efficiency of each activatable antibody was calculated by dividing EC50 value of the activatable antibody by EC50 value of parental antibody. The calculated masking efficiencies of TY26294, TY26898 and TY26899 by using human CD47 ranged from 190 to 696 fold when calculated for the masked antibody vs. the parental antibody, and from 266 to 572 fold when calculated for the uncleaved vs. cleaved masked antibody. However, the binding affinities of the activatable antibodies to human CD47 was fully recovered after cleavage by MMP9 enzyme in vitro.

TABLE 13
Summary of EC50 values of ELISA binding to human,
monkey and mouse CD47 proteins.

| | Human CD47 | | | Monkey | Mouse |
|---|---|---|---|---|---|
| | EC50 (nM) | | | CD47 | CD47 |
| Ab ID | Batch 1 | Batch 2 | Batch 3 | EC50 (nM) | EC50 (nM) |
| TAC2204 | 0.35 | NT | 0.54 | NT | NT |
| TY21446 | 0.23 | 0.35 | 0.62 | 0.46 | ND |
| TY26896 | 0.19 | 0.33 | 0.5315 | 0.43 | ND |
| TY26897 | 0.34 | 0.34 | NT | 0.58 | ND |
| TY26294 | 160.1 | 185.6 | 146.3 | * | ND |
| TY26898 | 42.8 | 158.1 | 202 | * | ND |
| TY26899 | 64.6 | 160.3 | NT | * | ND |
| TY26294 after | | | | | |
| cleavage | NT | 0.34 | 0.55 | NT | NT |
| TY26898 after | | | | | |
| cleavage | NT | 0.32 | 0.55 | NT | NT |
| TY26899 after | | | NT | | |
| cleavage | NT | 0.28 | | NT | NT |

ND: non-detectable
*: ambiguous
NT: not tested

### Example 6: Anti-CD47 Antibody Binding to CD47 on Tumor Cell Surface

The abilities of the activatable antibodies and the parental antibodies to bind to CD47+ tumor cell lines were tested by flow cytometry. Raji and CEM were B- and T-cell lymphoma cell lines, respectively, with varying CD47 expression levels on cell surface. Briefly, the test antibodies were serially diluted and were incubated with the tumor cells on ice. After washing, the cells were subsequently incubated with the APC-labelled anti-human-Fc secondary antibody on ice. The cells were then washed with PBS prior to analysis by the CytoFLEX flow cytometer (Beckman Coulter). The data were analyzed by FlowJo software 10. The binding affinities to CEM cells were measured in two separate batches (Table 14A), and the binding affinities to Raji cells were measured in three separate batches (Table 14B). The secondary antibody used in batch 2 for CEM cell binding and batch 3 for Raji cell binding is goat anti-human IgG (H+L) APC (Jackson Immunoresearch), while the secondary antibody used in batch 1 for CEM cell binding and batches 1 and 2 for Raji cell binding is APC anti-human IgG Fc (BioLegend).

As shown in Tables 14A and 14B, the parental antibodies TY21446, TY26896 and TY26897, as well as the benchmarks TAC2204 and TAC2407, showed high binding to Raji and CEM cells. The activatable antibodies TY26294, TY26898 and TY26899 showed lower binding than the parental antibodies. However, binding of the activatable antibodies TY26294. TY26898 and TY26899 were almost fully restored after MMP9 cleavage in vitro to remove the masking peptide. The calculated masking efficiencies of the activatable antibodies for binding to CEM cells ranged from 139 to 1507 fold when calculated for the masked antibody vs. the parental antibody, and from 174 to 341 fold when calculated for the uncleaved vs. cleaved masked antibody. Similarly, the calculated masking efficiencies of the activatable antibodies for binding to Raji cells ranged from 35 to 774 fold when calculated for the masked antibody vs. the parental antibody, and from 64 to 891 fold when calculated for the uncleaved vs. cleaved masked antibody.

TABLE 14A
Summary of EC50 values of the antibodies binding to
CD47 on CEM tumor cells.

| | Batch 1 | Batch 2 | |
|---|---|---|---|
| Ab ID | EC50 (nM) | EC50 (nM) | max MFI |
| TY21446 | 0.41 | 0.51 | 301403 |
| TY26896 | 0.27 | 0.52 | 333719 |
| TY26897 | 0.64 | 0.44 | 324966 |
| TY26294 | 520 | 71.2 | 303589 |
| TY26898 | 407 | 153.6 | 324074 |
| TY26899 | 446 | 106.1 | 387032 |
| TY26294 after cleavage | NT | 0.41 | 287019 |
| TY26898 after cleavage | NT | 0.45 | 305769 |
| TY26899 after cleavage | NT | 0.43 | 323020 |
| TAC2204 | 0.44 | 0.36 | 262989 |
| TAC2407 | NT | 1.18 | 224357 |
| hIgG1 isotype control | ND | ND | 2895 |

MFI = mean fluorescent intensity
ND: non-detectable
NT: not tested

TABLE 14B
Summary of EC50 values of the
antibodies binding to CD47 on Raji tumor cells.

| | Batch 1 | Batch 2 | Batch 3 | |
|---|---|---|---|---|
| | EC50 | EC50 | EC50 | max |
| Ab ID | (nM) | (nM) | (nM) | MFI |
| TY21446 | 0.48 | 0.48 | 0.24 | 144862 |
| TY26896 | 0.49 | 0.46 | 0.26 | 173834 |
| TY26897 | 0.39 | 0.33 | 0.42 | 212761 |
| TY26294 | 272 | 334.8 | 27.6 | 150303 |
| TY26898 | 274 | 356.2 | 31.1 | 118330 |
| TY26899 | 156 | 165.8 | 14.8 | 118354 |
| TY26294 after cleavage | NT | 0.48 | 0.26 | 156700 |
| TY26898 after cleavage | NT | 0.40 | 0.19 | 164413 |
| TY26899 after cleavage | NT | 0.34 | 0.23 | 190675 |
| TAC2204 | 0.29 | 0.33 | 0.14 | 116619 |
| TAC2407 | NT | NT | 1.97 | 65859 |
| iIgG1 isotype control | ND | ND | ND | 6411 |

MFI = mean fluorescent intensity
ND: non-detectable
NT: not tested

### Example 7: Anti-CD47 Antibody Binding to CD47 on RBCs

The abilities of the antibodies to bind to human red blood cells (RBCs), which express CD47 on the cell surface, were tested by flow cytometry. Human RBCs were collected from human peripheral blood by centrifugation and washed twice with DPBS/EDTA buffer, followed by resuspension with DPBS/EDTA buffer. The RBCs were then incubated with test antibodies with serial dilution at 37° C. for 1 hour. The RBCs were washed twice and further incubated with APC conjugated anti-human IgG Fc secondary antibody (BioLegend) at 37° C. for 30 minutes. After washing twice, the RBCs were analyzed on the CytoFLEX flow cytometer (Beckman Coulter) and the data were analyzed by FlowJo software 10. Binding to human RBCs was measured in two different batches corresponding to two different RBC donors.

A summary of the EC50 values of the antibodies binding to CD47 on human RBCs is shown in Table 15. Binding to RBCs is greatly reduced in the activatable antibodies. The calculated masking efficiencies of the activatable antibodies for binding to RBCs ranged from 344 to 2007 fold when calculated for the masked antibody vs. the parental antibody.

TABLE 15
Summary of EC50 values of the antibodies binding
to CD47-positive human RBCs.

| | Batch 1 | Batch 2 | |
|---|---|---|---|
| Ab ID | EC50 (nM) | EC50 (nM) | max MFI |
| TY21446 | 0.37 | 0.55 | 18016 |
| TY26896 | 0.20 | 0.44 | 14043 |
| TY26897 | 0.36 | 0.43 | 13110 |
| TY26294 | 482.4 | 1104 | 8257 |
| TY26898 | 185.1 | 653 | 2805 |
| TY26899 | 123.7 | 329 | 4862 |
| TAC2204 | 0.76 | 0.33 | 14564 |
| TAC2407 | NT | ND | 2219 |
| Isotype control | ND | ND | 105 |

MFI = mean fluorescent intensity
ND: non-detectable
NT: not tested

### Example 8: Study of RBC Hemagglutination by Anti-CD47 Antibodies

The candidate anti-CD47 antibodies were tested for their capacities to induce the agglutination of human RBCs and were compared to the benchmark antibodies. Human RBCs purified from human peripheral blood samples from two independent donors were incubated with a serial dilution of the test antibodies at 37° C. for 1 hour in U-bottom 96-well plates. After incubation, evidence of RBC agglutination was demonstrated by the appearance of a haze as opposed to a punctate red pellet of non-agglutinated RBCs.

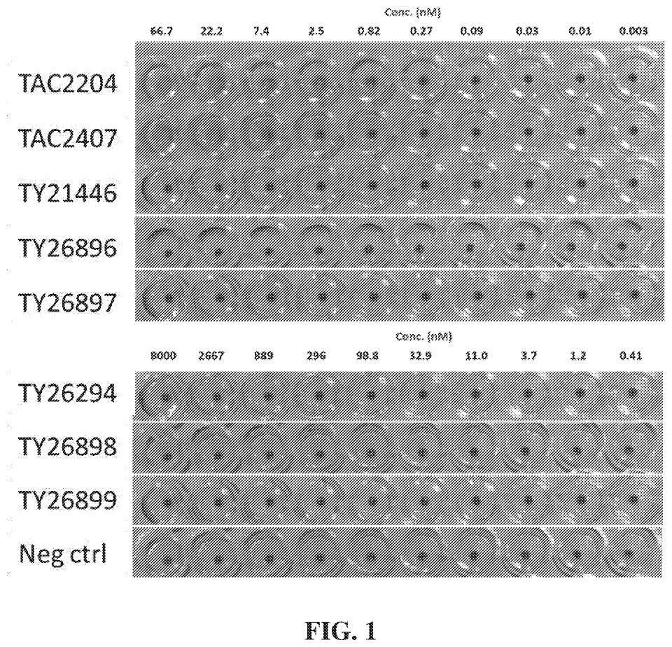

As shown FIG. 1, TAC2204 and TAC2407 caused human RBC hemagglutination, whereas TY21446, TY26896 and TY26897 did not (antibody concentrations from 0.003 to 66.7 nM). The activatable antibodies TY26294, TY26898 and TY26899 did not cause human RBC hemagglutination even as high as 8000 nM. FIG. 1 depicts hemagglutination of red blood cells from one of the two donors; similar hemagglutination was observed in red blood cells from the other donor.

### Example 9: Anti-CD47 Antibody Induction of Phagocytosis of CEM Cells by Human Macrophages

CD47 is considered as a “don't eat me” signal that prevents the phagocytosis of CD47-expressing cells by interacting with SIRPα on cells with phagocytosis ability such as macrophages. The ability of the test antibodies to increase the phagocytosis of tumor cells by macrophages was evaluated. Briefly, human macrophages were derived from peripheral monocytes purified from human PBMCs in the presence of CSF-1 (Sinobiological) for 8 days. The macrophages were re-plated to 24-well plates at a concentration of 1×10⁵ cells/well and allowed to adhere at 37° C. for 24 hours. CEM cells labeled with 5 (6)-carboxyfluorescein diacetate N-succinimidyl ester (CFSE, Sigma) were added to the macrophage culture at the concentration of 3.2×10⁵ cells/well. The test antibodies with serial dilution were added immediately upon mixture of target and effector cells and incubated at 37° C. for 2 hours. After incubation, non-phagocytosed CEM cells were removed by washing twice with PBS. The remaining cells were then trypsinized and labeled with APC conjugated anti-human CD206 antibody (BioLegend), which is a marker on human macrophage. The cells were analyzed on the CytoFLEX flow cytometer (Beckman Coulter) and the data were analyzed by FlowJo and GraphPad Prism 7.0 software.

As shown in Table 16, TY21446 showed comparable phagocytosis activity to TAC2204. However, TY26896 and TY26897 showed significantly increased potency and efficacy of phagocytosis, comparing to TY21446, TAC2204 and TAC2407. The phagocytosis activities of the activatable antibodies TY26294, TY26898 and TY26899 were in the baseline at 20 nM. However, the phagocytosis activities were fully recovered after the activatable antibodies were pre-treated with MMP9 cleavage in vitro.

TABLE 16
Summary of macrophage phagocytosis
studies of the anti-CD47 antibodies.

| | donor A | | donor B | |
|---|---|---|---|---|
| | | Max. | | Max. |
| | | Phago- | | Phago- |
| | EC50 | cytosis | EC50 | cytosis |
| Ab ID | (nM) | (%) | (nM) | (%) |
| TY21446 | 0.90 | 48.0% | 0.54 | 56.1% |
| TY26896 | 0.22 | 78.8% | 0.16 | 92.2% |
| TY26897 | 0.20 | 75.9% | 0.13 | 85.9% |
| TY26294 | NA | 15.2% | NA | 5.2% |
| TY26898 | NA | 21.2% | NA | 17.8% |
| TY26899 | NA | 18.6% | NA | 14.4% |
| TY26294 after cleavage | 1.04 | 55.6% | 0.79 | 57.0% |
| TY26898 after cleavage | 0.28 | 88.3% | 0.18 | 87.9% |
| TY26899 after cleavage | 0.17 | 87.3% | 0.16 | 85.3% |
| TAC2204 | 1.07 | 58.3% | 1.17 | 76.1% |
| TAC2407 | 4.40 | 29.7% | 0.83 | 29.0% |
| IgG1 isotype control | NA | 14.3% | NA | 2.8% |

### Example 10: ADCC Activity of the Antibodies

ADCC activity of the test antibodies was evaluated using NK cell as the effector cell and CD47+ CEM cell as the target cell. Human NK cells were purified from PBMCs using the Human NK Cell Isolation kit (StemCell). Calcein-AM labeled CEM cells of 1×10⁴ cells/well were incubated with test antibodies of different concentrations at 37° C. for 30 minutes. Human NK cells of 5×10⁴ cells/well (E/T ratio 5:1) were added and the mixture was further incubated at 37° C. for 2 hours. After incubation, the Calcein-AM in the supernatant was measured by SpectraMax i3x (Molecular Devices). The percent lysis was then calculated using the following formula: % Lysis= [(Experimental Release)-Ave (Target+NK)]/[Ave (Target Max)-Ave (Target only)]×100%.

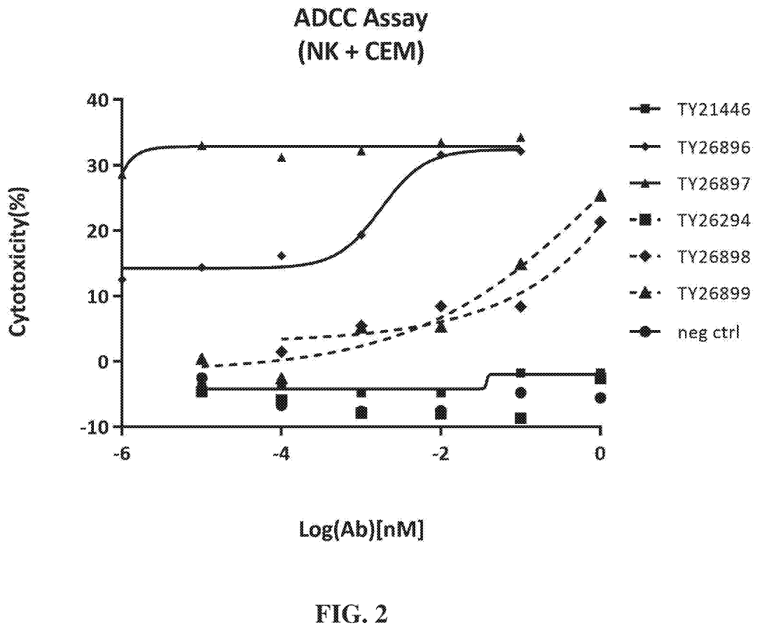

As shown in FIG. 2, TY21446 and TAC2204 did not show ADCC activity, whereas TY26896 and TY26897 showed ADCC activity. TY26897 showed stronger ADCC activity than TY26896 did.

### Example 11: In Vivo Anti-Tumor Efficacy of the Anti-CD47 Antibodies in Mouse Xenograft Models

### B-NDG/Raji-Luc Mouse Systemic Model

The in vivo anti-tumor efficacy of the antibodies was tested in the B-NDG mice (Biocytogen) inoculated with Raji cells expressing luciferase (Raji-Luc). B-NDG mice are generated by deleting the IL2rg gene from NOD-scid mice with severe immunodeficiency phenotype. The mice lack mature T cells, B cells and functional NK cells, but have deficient macrophages. Raji-Luc cells of 1×10⁵ cells per injection in PBS were intravenously injected into B-NDG mice. When the luminescent signal reached ˜1×10⁶ p/see, the mice were started to be intraperitoneally treated with test antibodies or control IgG (N=6 each group) twice per week for total 4 doses. Luminescent signal and body weight were measured twice per week until the study was finished. The mice were euthanized if movement disorder (e.g. hind-limb paralysis) was observed or weight loss exceeded 20%. Tumor growth inhibition was calculated using the formula: TGI %=(1-TRTV/CRTV)×100%. TRTV and CRTV represent relative tumor volumes of treatment group and control group, respectively, at a particular time point.

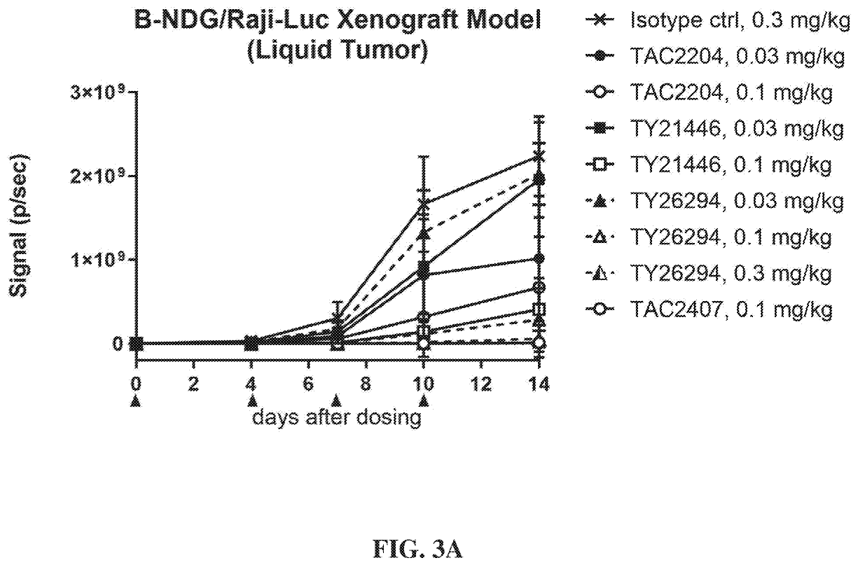

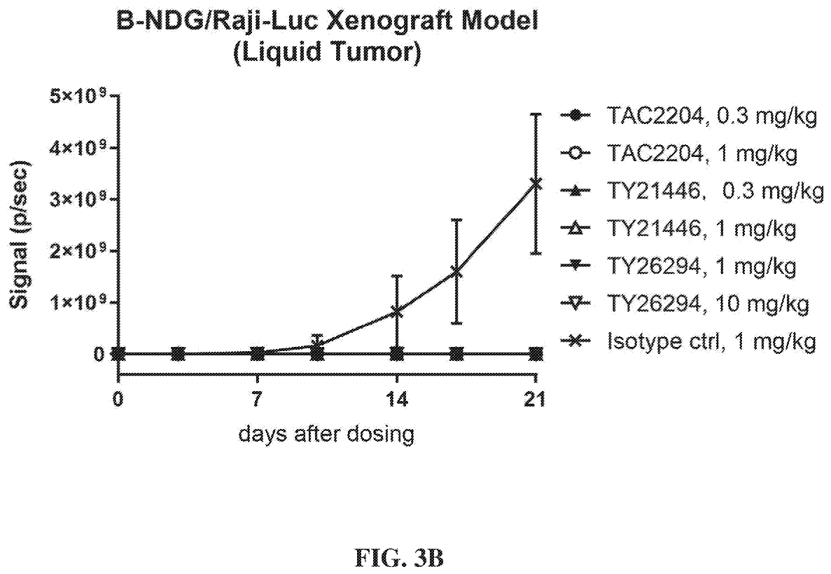

As shown in FIG. 3A and Table 17A, TAC2204 showed 99.6% tumor growth inhibition at 0.1 mg/kg dose in the B-NDG/Raji-Luc systemic mouse model. TY21446 showed 81.8% tumor growth inhibition at 0.1 mg/kg dose. The activatable antibody TY26294 showed 87.4% and 97.5% tumor growth inhibition at 0.1 and 0.3 mg/kg dose, respectively. Therefore, TAC2204, TY21446 and TY26294 showed comparable anti-tumor efficacy in the B-NDG/Raji-Luc systemic mouse model. In an independent study (FIG. 3B and Table 17B), TAC2204 and parental antibody TY21446 at 0.3 mg/kg, and the activatable antibody TY26294 at 1 mg/kg showed complete tumor inhibition.

TABLE 17A
Tumor growth inhibition of the antibodies at
day 14 in the B-NDG/Raji-Luc
systemic model; first independent experiment.

| | | | P value | | |
|---|---|---|---|---|---|
| | | | | vs | vs |
| | | | vs | TAC2204 | TAC2407 |
| | Dose | TGI | Isotype | (0.1 | (0.1 |
| Ab ID | (mg/kg) | (%) | ctrl | mg/kg) | mg/kg) |
| TY21446 | 0.03 | 12.5% | p < 0.01 | | |
| | 0.1 | 81.8% | p < 0.0001 | ns | ns |
| TY26294 | 0.03 | 9.6% | ns | | |
| | 0.1 | 87.4% | p < 0.0001 | ns | ns |
| | 0.3 | 97.5% | p < 0.0001 | | |
| TAC2204 | 0.03 | 54.6% | p < 0.01 | | |
| | 0.1 | 99.6% | p < 0.0001 | | ns |
| TAC2407 | 0.1 | 70.1% | p < 0.001 | ns | |
| Isotype ctrl | 0.3 | 0.0% | | | |

TABLE 17B
Tumor growth inhibition of the antibodies
at day 14 in the B-NDG/Raji-Luc systemic
model; second independent experiment.

| | | | P value |
|---|---|---|---|
| | Dose | TGI | vs Isotype |
| Ab ID | (mg/kg) | (%) | control |
| TY21446 | 0.3 | 100.0% | p < 0.0001 |
| | 1 | 100.0% | p < 0.0001 |
| TY26294 | 1 | 100.0% | p < 0.0001 |
| | 10 | 100.0% | p < 0.0001 |
| TAC2204 | 0.3 | 100.0% | p < 0.0001 |
| | 1 | 100.0% | p < 0.0001 |
| Isotype ctrl | 1 | 0.0% | |

### B-NDG/Raji Mouse Solid Tumor Model

The in vivo anti-tumor efficacy of the antibodies was tested in the B-NDG mice (Biocytogen) inoculating subcutaneously with Raji cells. Raji cells of 5×10⁵ cells per injection in PBS were subcutaneously injected into B-NDG mice. When the tumor volume reached ˜100 mm³, the mice were started to be intraperitoneally treated with test antibodies or control IgG (N=5 each group) twice per week for total 6 doses. Tumor volume and body weight were measured twice per week until the study was finished. The mice were euthanized if tumor volume was greater than 3000 mm³, movement disorder (e.g. hind-limb paralysis) was observed or weight loss exceeded 20%. Tumor growth inhibition was calculated using the formula: TG1%=(1-TRTV/CRTV)×100%. TRTV and CRTV represent relative tumor volumes of treatment group and control group, respectively, at a particular time point.

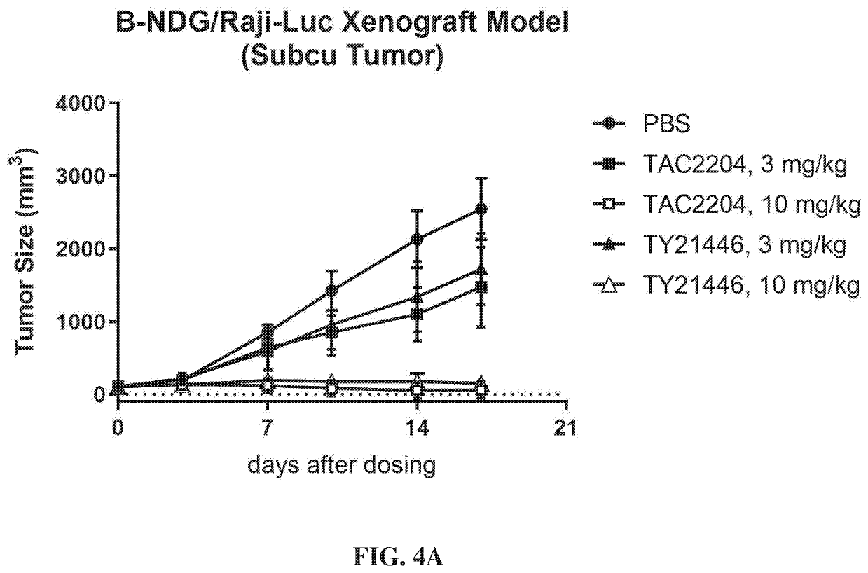

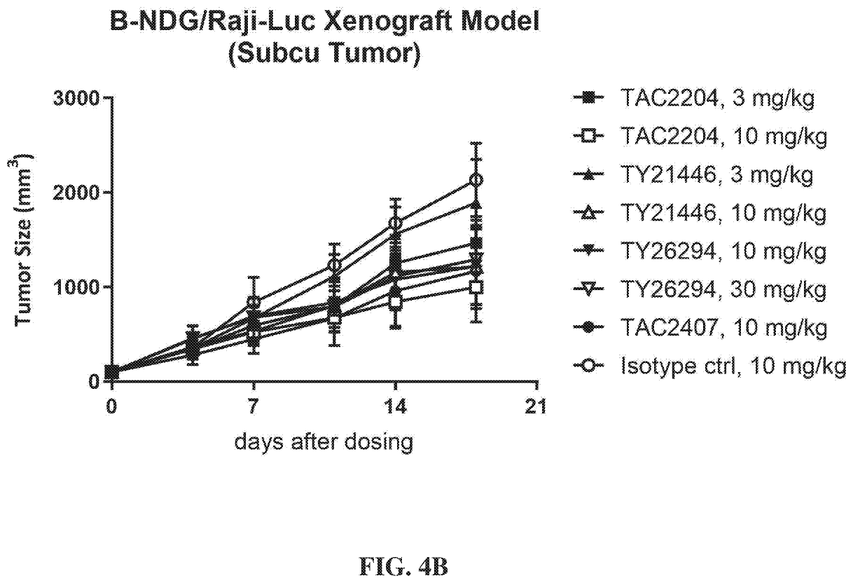

As shown in FIG. 4A and Table 18A, the parental antibody TY21446 and TAC2204 showed comparable anti-tumor efficacy in the mouse solid tumor model. Both antibodies partially inhibited tumor growth at 3 mg/kg and almost completely inhibited tumor growth at 10 mg/kg. The similar anti-tumor efficacy between TY21446 and TAC2204 was also observed in an independent study (FIG. 4B). As shown in Table 18B, the activatable antibody TY26294 at 10 and 30 mg/kg showed comparable tumor growth inhibition to TAC2204 and to its parental antibody TY21446 at 10 mg/kg.

TABLE 18A
Tumor growth inhibition of the antibodies at day 17 in the B-NDG/
Raji subcutaneous model; first independent experiment.

| | | | P value | | |
|---|---|---|---|---|---|
| | | | | vs | vs |
| | Dose | TGI | | TAC2204 | TAC2204 |
| Ab ID | (mg/kg) | (%) | vs PBS | (3 mg/kg) | (10 mg/kg) |
| TAC2204 | 3 | 44.2% | p < 0.01 | | |
| | 10 | 101.6% | p < 0.0001 | | |
| TY21446 | 3 | 25.0% | p < 0.05 | ns | |
| | 10 | 97.5% | p < 0.0001 | | ns |
| PBS | — | 0.0% | | | |

TABLE 18B
Tumor growth inhibition of the antibodies at day 17 in the B-NDG/
Raji-Luc subcutaneous model; second independent experiment.

| | | | P value | | | |
|---|---|---|---|---|---|---|
| | Dose | | vs | vs | vs | Vs |
| | (mg/ | TGI | Isotype | TAC2204 | TAC2204 | TAC2407 |
| Ab ID | kg) | (%) | ctrl | (3 mg/kg) | (10 mg/kg) | (10 mg/kg) |
| TAC2204 | 3 | 33.0% | p < 0.05 | | | |
| | 10 | 56.0% | p < 0.001 | | | ns |
| TY21446 | 3 | 24.0% | ns | p < 0.01 | | |
| | 10 | 45.0% | p < 0.001 | | ns | ns |
| TY26294 | 10 | 45.0% | p < 0.001 | | p < 0.05 | ns |
| | 30 | 42% | p < 0.01 | | | |
| TAC2407 | 10 | 48% | p < 0.001 | | ns | |
| Isotype | 10 | 0% | | | | |
| ctrl | | | | | | |

### SCID/Raji-Luc Mouse Systemic Model

The in vivo anti-tumor efficacy of the antibodies was tested in CB17 SCID mice (Biocytogen) inoculated with Raji cells expressing luciferase (Raji-Luc). CB17 SCID mice lack functional T and B cells, but have normal NK cells, macrophages and granulocytes. Raji-Luc cells of 5×10⁵ cells per injection in PBS were intravenously injected into CB17 SCID mice. When the luminescent signal reached ˜1×10⁶ p/see, the mice were started to be intraperitoneally treated with test antibodies or control IgG (N=8 each group) twice per week for total 4 doses. Luminescent signal and body weight were measured twice per week until the study was finished. The mice were euthanized when movement disorder (e.g., hind-limb paralysis) was observed or weight loss exceeded 20%. Tumor growth inhibition was calculated using the formula: TGI %=(1-TRTV/CRTV)×100%. TRTV and CRTV represent relative tumor volumes of treatment group and control group, respectively, at a particular time point.

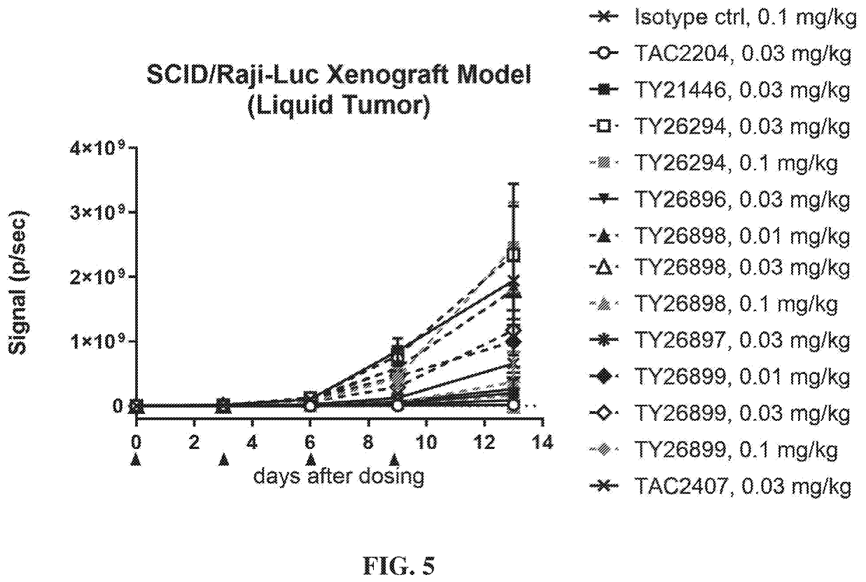

As shown in FIG. 5 and Table 19, the TAC2204 and the parental antibodies TY21446, TY26896 and TY26897 showed comparable anti-tumor efficacy at 0.03 mg/kg dose in the SCID/Raji-Luc systemic mouse model. The activatable antibodies TY26294 did not show efficacy in the 0.03˜0.1 mg/kg dose range. The activatable antibodies TY26898 and TY26899 showed 81.2% and 91.8% tumor growth inhibition at 0.1 mg/kg dose, respectively.

TABLE 19
Tumor growth inhibition of the antibodies at
day 13 in the SCID/Raji-Luc systemic model.

| | | | P value | | | |
|---|---|---|---|---|---|---|
| | | | vs | vs | vs | vs |
| | Dose | TGI | Isotype | TAC2204 | TY26294 | TY26294 |
| Ab ID | (mg/kg) | (%) | ctrl | (0.03 mg/kg) | (0.03 mg/kg) | (0.1 mg/kg) |
| TY21446 | 0.03 | 89.7% | p < 0.0001 | ns | | |
| TY26294 | 0.03 | −20.7% | ns | p < 0.0001 | | |
| | 0.1 | −26.7% | ns | p < 0.0001 | | |
| TY26896 | 0.03 | 95.6% | p < 0.0001 | ns | | |
| TY26898 | 0.01 | 7.6% | ns | | | |
| | 0.03 | 8% | ns | p < 0.0001 | ns | p < 0.0001 |
| | 0.1 | 81% | p < 0.0001 | ns | | |
| TY26897 | 0.03 | 87% | p < 0.0001 | ns | | |
| TY26899 | 0.01 | 48.60% | ns | | | |
| | 0.03 | 39.70% | ns | p < 0.0001 | p < 0.0001 | p < 0.0001 |
| | 0.1 | 91.80% | p < 0.0001 | ns | | |
| TAC2204 | 0.03 | 99.30% | p < 0.0001 | | | |
| TAC2407 | 0.03 | 66.30% | p < 0.01 | ns | | |
| Isotype ctrl | 0.3 | 0% | | | | |

### Example 12: Study of the Toxicity of the Anti-CD47 Antibodies in Mice

The toxicity of TY21446 was tested using B-hSIRPα/hCD47 humanized mice (Biocytogen). Female mice of 5-8 weeks were intraperitoneally injected with TAC2204, TY21446 or PBS (N=5 each group). RBC and hemoglobin level in peripheral blood were monitored at pre-dose, Day 1, Day 3, Day 5, Day 7, Day 9 and Day 11. The mice were euthanized if movement disorder (e.g., hind-limb paralysis) was observed or weight loss exceeded 20%.

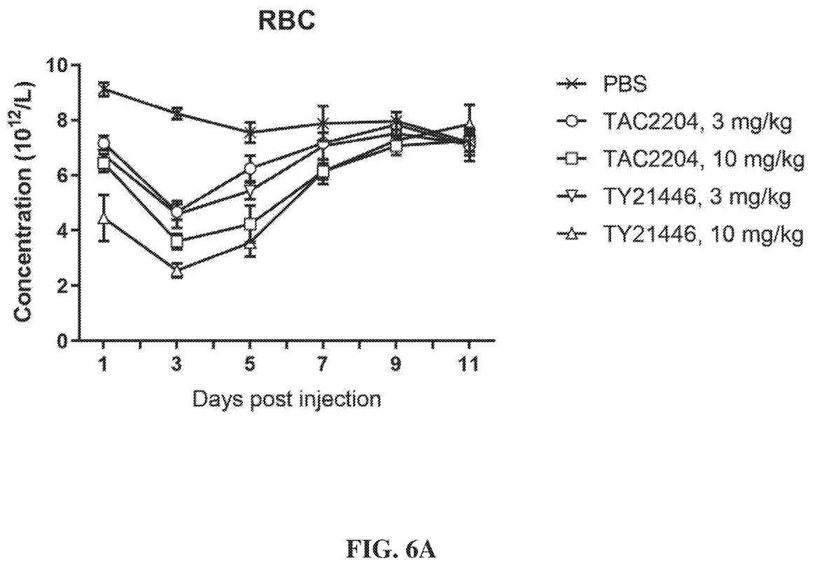

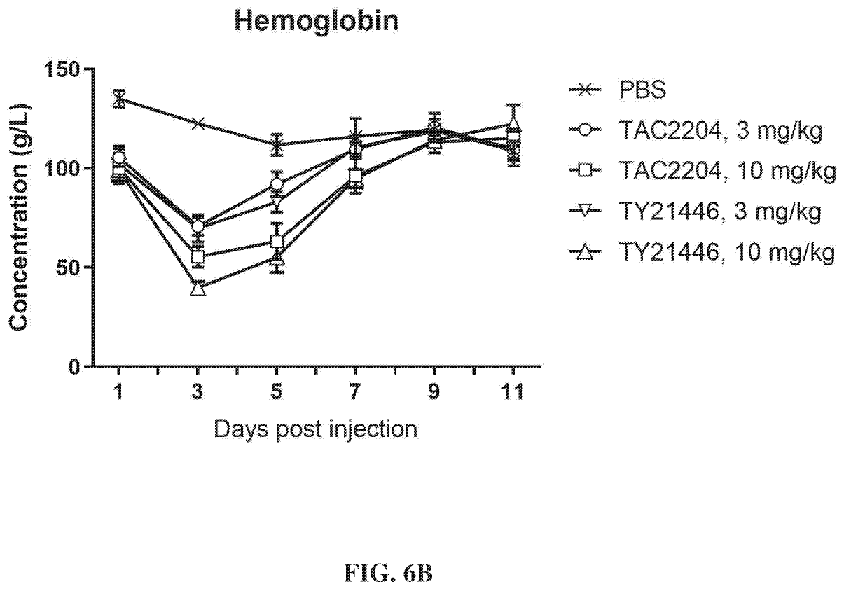

As shown in FIGS. 6A-6B, benchmark control TAC2204 and TY21446 showed similar decrease of RBC numbers (FIG. 6A) and hemoglobin concentrations (FIG. 6B) in peripheral blood at the same dosage levels.

### Example 13: Study of the Toxicity and Pharmacokinetics of the Anti-CD47 Antibodies

### Toxicity and Pharmacokinetics in Cynomolgus Monkey

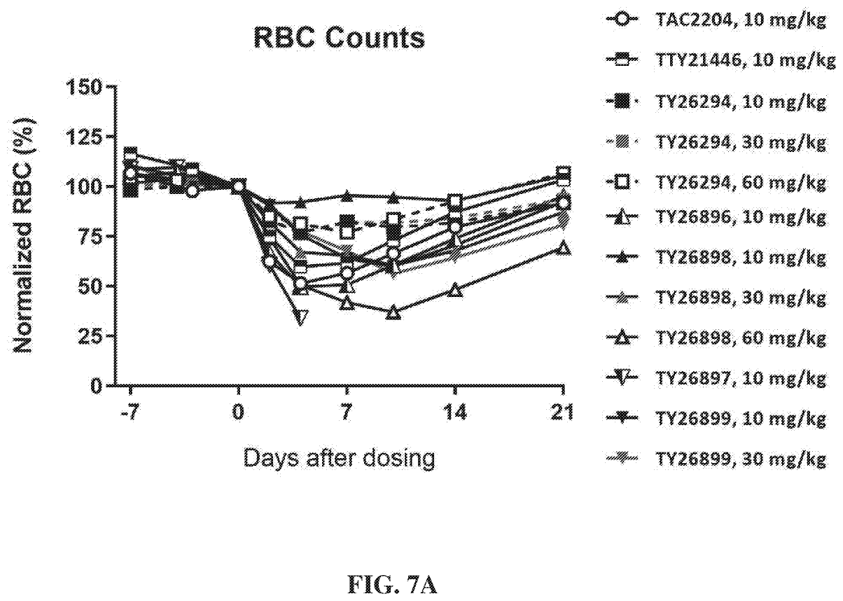

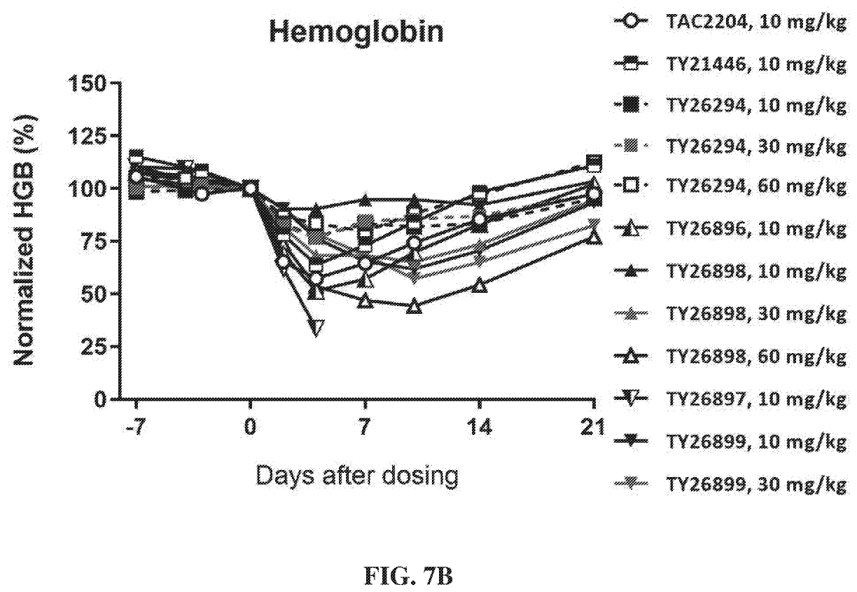

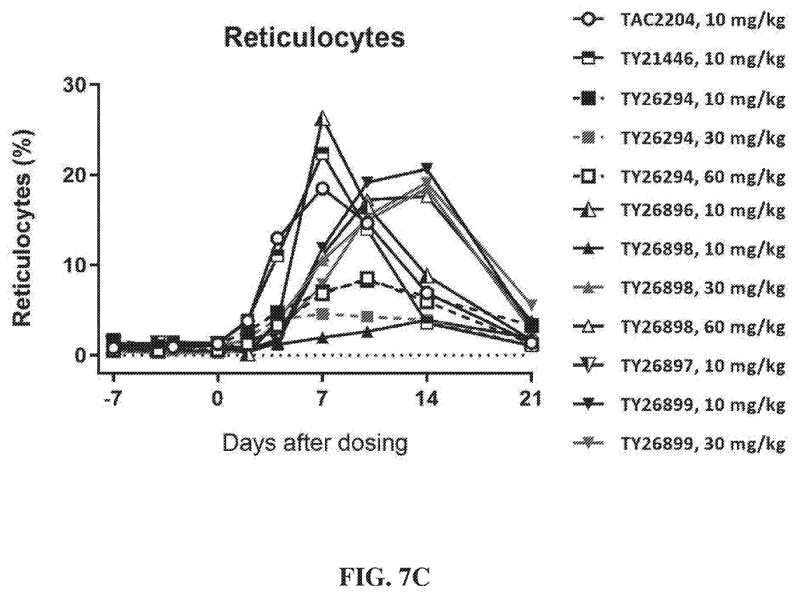

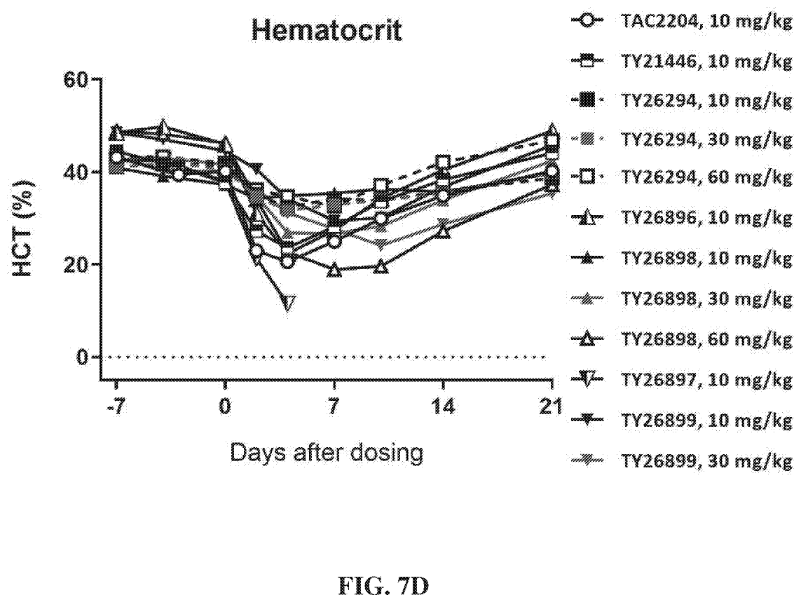

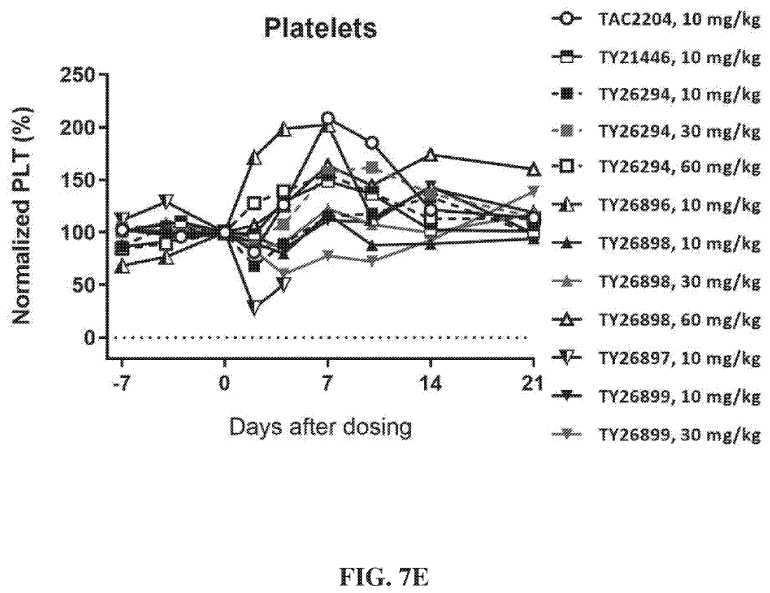

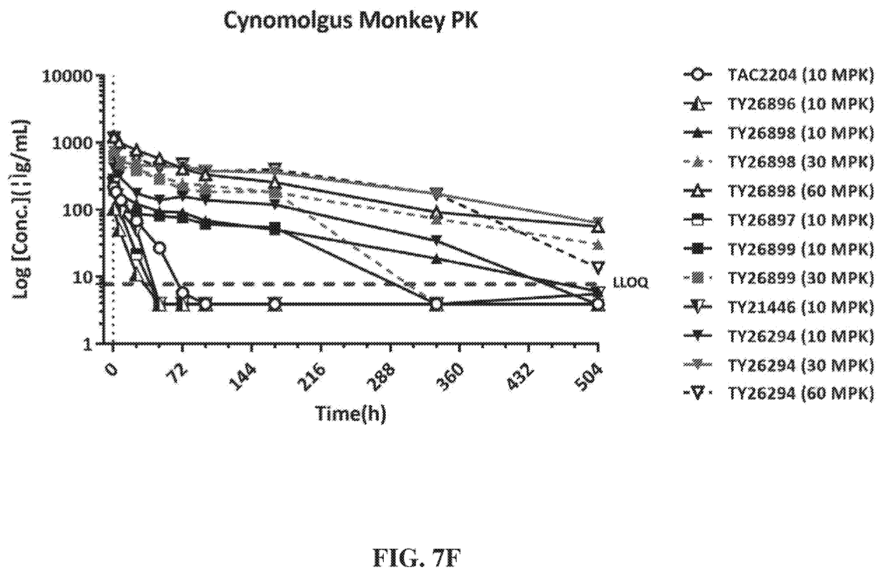

A single dose study in cynomolgus monkeys was conducted. Twelve male cynomolgus monkeys were divided into 12 groups (1 animal each group). Animals were intravenously administered with TAC2204 (10 mg/kg), TY21446 (10 mg/kg), TY26294 (10, 30 and 60 mg/kg), TY26896 (10 mg/kg), TY26898 (10, 30 and 60 mg/kg), TY26897 (10 mg/kg) or TY26899 (10 and 30 mg/kg) (Table 20A). Cage-side observations for general health and appearance were performed regularly. The blood samples for hematology and blood chemistry were collected at pre-dose (Day-7, Day-3 and Day 0), Day 2, Day 4, Day 7, Day 10, Day 14 and Day 21 for total drug. The plasma samples for pharmacokinetics were collected at pre-dose (Oh), 0.5 h, 3 h, 8 h, 24 h, Day 2, Day 3, Day 4, Day 7, Day 14 and Day 21. The antibodies were captured by goat anti-human IgG antibody (SouthernBiotech) and detected by HRP-conjugated goat-anti-human IgG (Fab specific) antibody (Sigma). The results of the study are shown in FIGS. 7A-7E.

TABLE 20A
Grouping and dosing of the single-dose
toxicity study in cynomolgus monkey.

| | Number of | Dose | Dosing |
|---|---|---|---|
| Ab ID | animals | Level | Strategy |
| TAC2204 | 1 male | 10 mg/kg | Single IV |
| TY21446 | 1 male | 10 mg/kg | Single IV |
| TY26294 | 1 male | 10 mg/kg | Single IV |
| TY26294 | 1 male | 30 mg/kg | Single IV |
| TY26294 | 1 male | 60 mg/kg | Single IV |
| TY26896 | 1 male | 10 mg/kg | Single IV |
| TY26898 | 1 male | 10 mg/kg | Single IV |
| TY26898 | 1 male | 30 mg/kg | Single IV |
| TY26898 | 1 male | 60 mg/kg | Single IV |
| TY26897 | 1 male | 10 mg/kg | Single IV |
| TY26899 | 1 male | 10 mg/kg | Single IV |
| TY26899 | 1 male | 30 mg/kg | Single IV |

After early antibody administration, all animals, except for the one dosed with 10 mg/kg TY26897, showed normal general health and appearance. The animal with 10 mg/kg TY26897 died 5 days after dosing, probably due to the severe loss of circulating RBCs and HGB as shown in FIGS. 7A-7B.

As shown in FIGS. 7A-7B, single administration of TAC2204, TY21446 and TY26896 at 10 mg/kg induced comparable reduction of RBC and HGB in cynomolgus monkeys. Single administration of TY26897 caused severe RBC and HGB reduction, and the animal died at day 5. Single administration of TY26294 at 10, 30 and 60 mg/kg showed less reduction of RBC and HGB compared to the benchmark TAC2204 and the parental antibody TY21446. TAC2204 at 10 mg/kg caused ˜49% maximum decrease in RBCs, TY26294 at 60 mg/kg showed ˜23% maximum decrease in RBCs (FIG. 7A). Single administration of TY26898 showed dose-dependent hematology toxicity. TY26898 at 10 and 30 mg/kg induced less reduction of RBC and HGB than TAC2204. TY21446 and TY26896 at the same dosage, whereas TY26898 at 60 mg/kg may cause stronger reduction of RBC and HGB than TAC2204, TY21446 and TY26896 at 10 mg/kg. Single administration of TY26899 at 10 and 30 mg/kg showed comparable reduction of RBC and HGB to TAC2204, TY21446 and TY26896 at 10 mg/kg. Taken together, the activatable antibodies TY26294, TY26898 and TY26899 showed better hematology safety profile than the benchmark TAC2204 and the parental antibodies.

The pharmacokinetics properties of the anti-CD47 antibodies in cynomolgus monkey are shown in FIG. 7F. Non-compartmental analysis (NCA) was conducted to assess the plasma PK properties of the tested molecules in cynomolgus monkeys. Greater than ˜ 10 fold increase in calculated half-lives (e.g., ≥5 days at doses ≥10 mg/kg) was generally seen across all three activatable anti-CD47 antibodies compared to their parental Abs (e.g., <1 day) after a single IV dose. TY26898 has >20 fold area under the curve (AUC) increase compared with TY26896 at 10 mg/kg while TY26294 has ˜3 fold AUC increase compared with TY21446 at 10 mg/kg. AUClast comparisons across activatable anti-CD47 antibodies showed that TY26294 has a slight AUC advantage (e.g., 70-80% higher AUC) at 10 and 30 mg/kg, but not at 60 mg/kg, when compared with TY26898. In addition, TAC2204 at 10 mg/kg had an estimated half-life of about 1 day. Compared to TAC2204, TY26898 has ˜5-6 fold and ˜2.5 fold AUC increase at 10 and 30 mg/kg, while TY26294 has ˜9-10 fold and ˜4.4 fold AUC increase at 10 and 30 mg/kg, respectively.

TABLE 20B
Half-life and AUC values for anti-CD47
antibodies administered at various doses.

| | Dose | Half-life | AUClast |
|---|---|---|---|
| Antibody | (mg/kg) | (days) | (hour*μg/mL) |
| TAC2204 | 10 | 0.6 | 4395.0 |
| TY21446 | 10 | 0.8 | 12067.0 |
| TY26294 | 10 | 4.7 | 38026.5 |
| | 30 | 6.4 | 131897.2 |
| | 60 | 8.2 | 120453.2 |

### Pharmacokinetics in Mouse

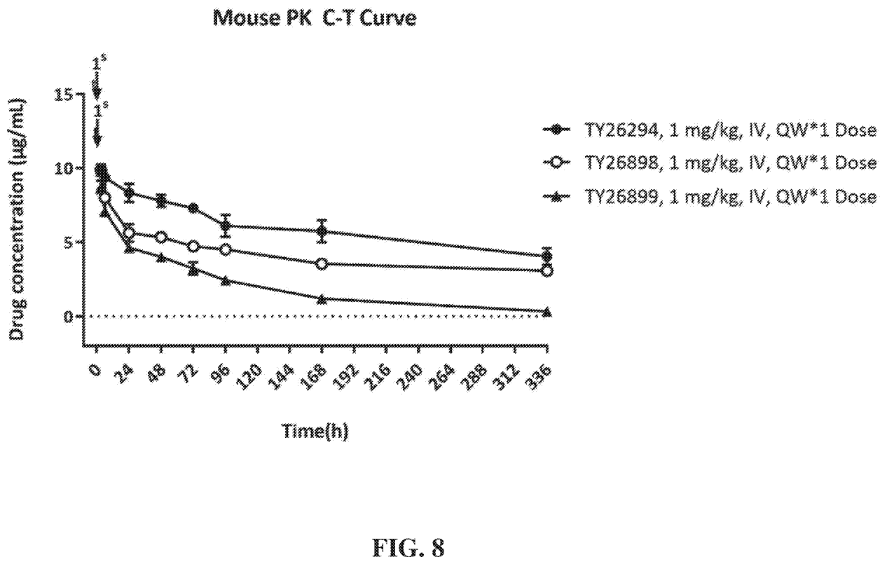

A single dose pharmacokinetics (PK) study was conducted in CB17 SCID mice (CB17 SCID, N=3 per molecule) and the plasma PK profiles (total drug concentrations) are shown in FIG. 8. The antibody concentration was determined by ELISA method for total drug. The antibodies were captured by goat anti-human IgG antibody (SouthernBiotech) and detected by HRP-conjugated goat-anti-human IgG (Fab specific) antibody (Sigma). Non-compartmental analysis (NCA) was conducted to assess the PK properties of the tested molecules in mice.

Based on mean AUClast, TY26898 accounts for ˜70% of that for TY26294, while the percent dropped to ˜33% for TY26899 compared to TY26294. This is also reflected from the Clast (336 hour data). Based on mean AUCinf, TY26898 accounts for ˜81% of that for TY26294, while the percent dropped to ˜17% for TY26899 compared to TY26294. For measured Cmax, 3-hour, there is no difference between TY26898 and TY26294, while the TY26899 has a slightly lower mean value compared with TY26294 (e.g., ˜ 89%). The mean (SD) terminal half-life of TY26898 (˜ 18+2 days) and TY26294 (˜ 15+4 days) are similar (P value=0.3), with some slight numerical differences. However, TY26899 had significantly decreased terminal half-life (˜ 3.4+0.5 days).

### Example 14: Cis/Trans Binding Studies of the Anti-CD47 Antibodies

The antibody candidates were tested for their preference to bind in cis or in trans to CD47 on the cell surface.

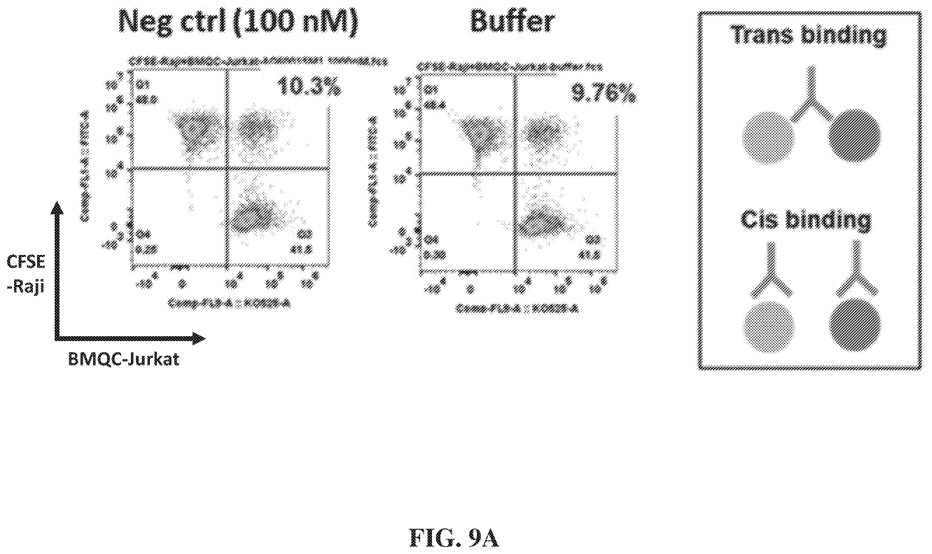

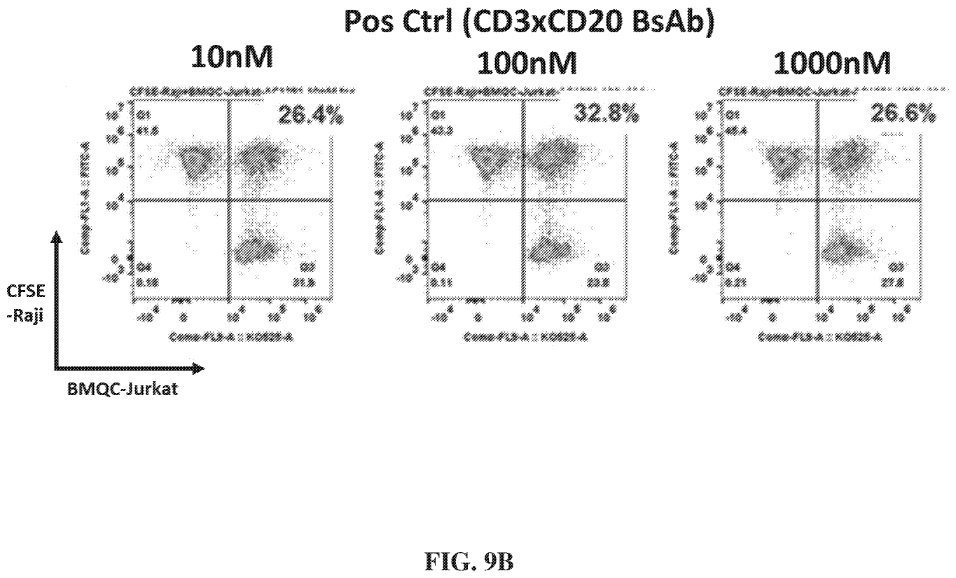

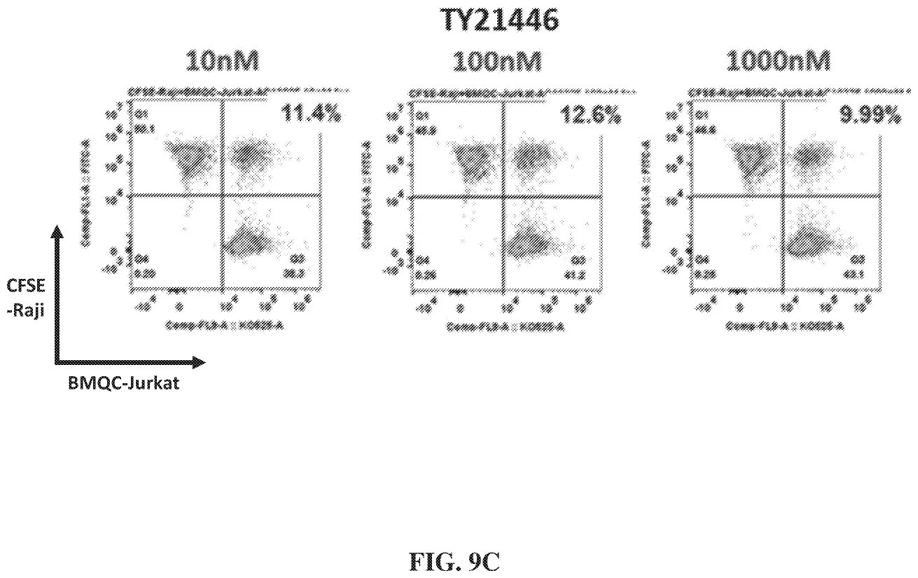

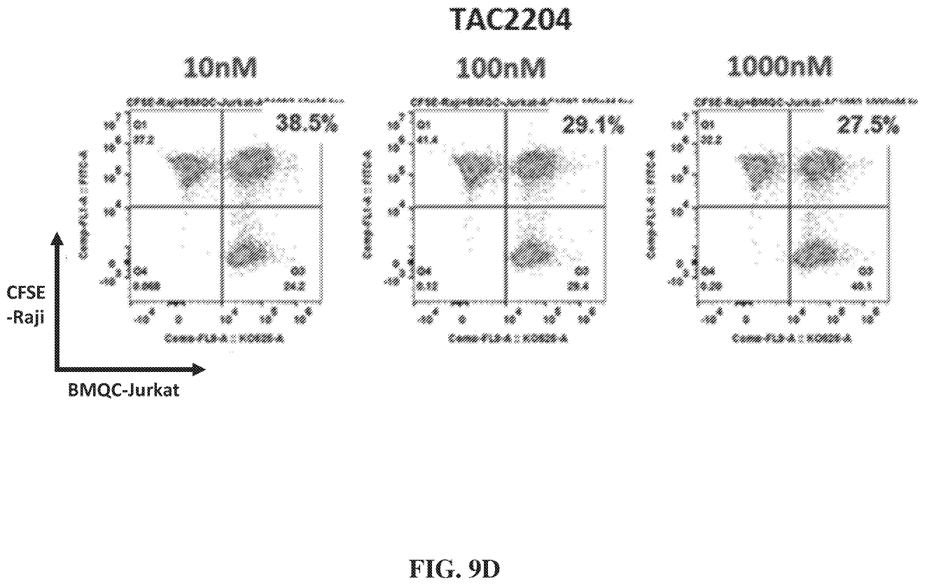

In one study, 2,3,6,7-tetrahydro-9-bromomethyl-1H,5H-quinolizino (9,1-gh) coumarin (BMQC)-labeled Jurkat cells and CFSE-labeled Raji cells were incubated with TAC2204, TY21446, negative control or positive control (CD3xCD20 BsAb) at 37° C. for 1 hour. Antibody mediated-crosslinking of the fluorescent dye-labeled Jurkat and Raji cells was detected by flow cytometry. The results of this study are shown in FIGS. 9A-9D.

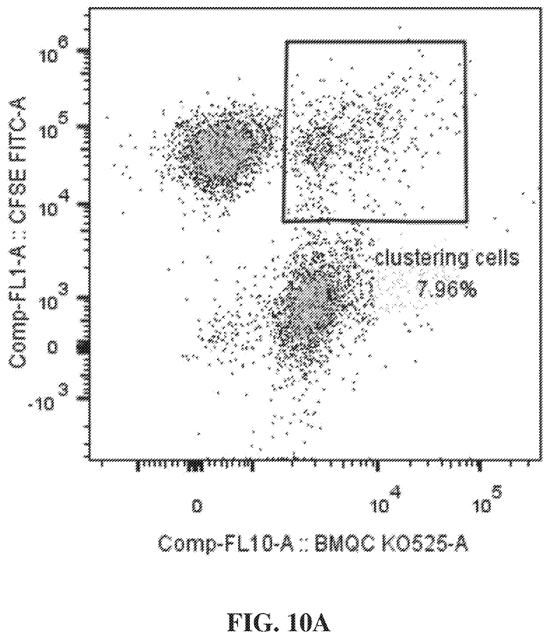

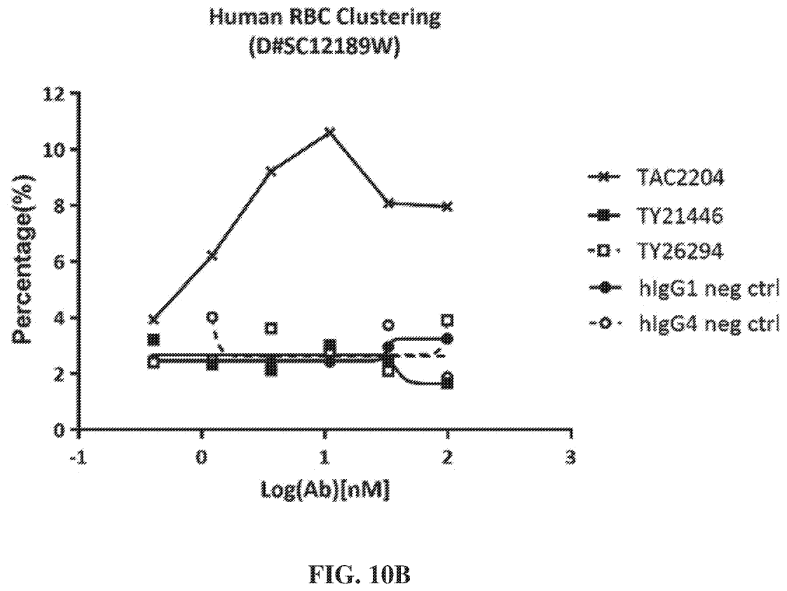

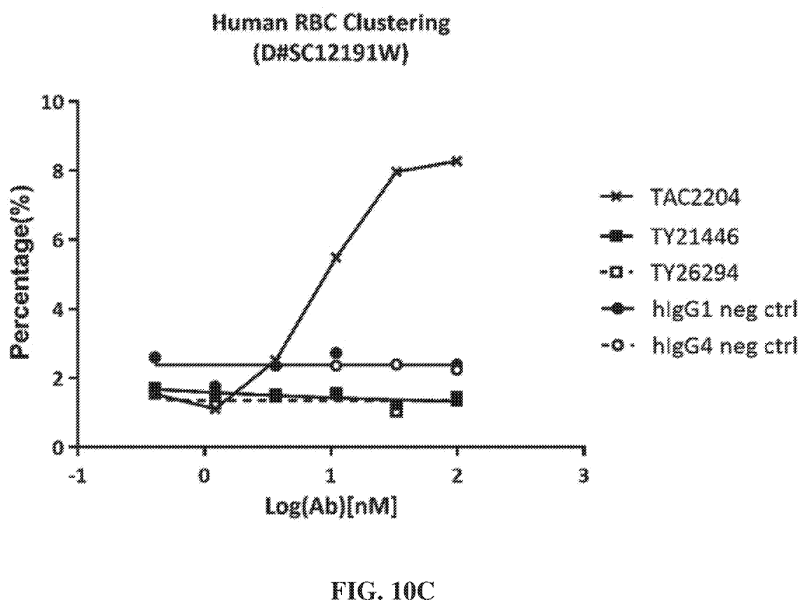

In a second study, human RBCs were labeled with BMQC or CFSE. BMQC-labeled RBCs and CFSE-labeled RBCs were mixed and incubated with TAC2204 or TY21446 at 37° C. for 4 hours, followed by flow cytometric analysis. The results of the study are shown in FIGS. 10A-10C.

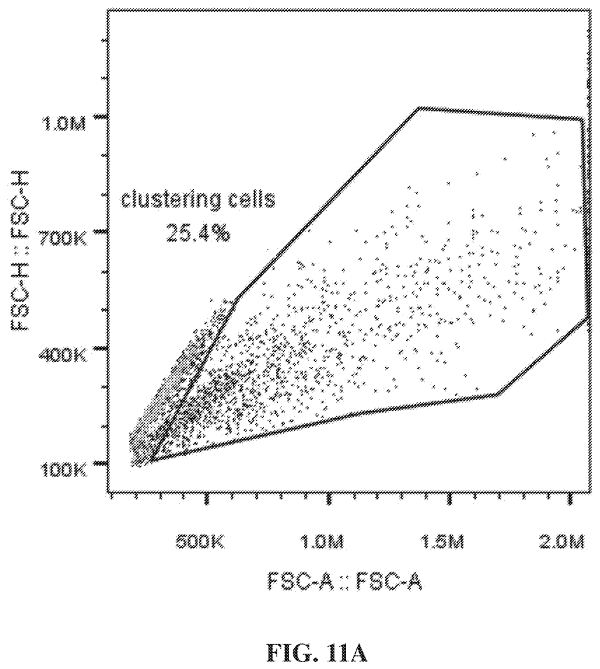

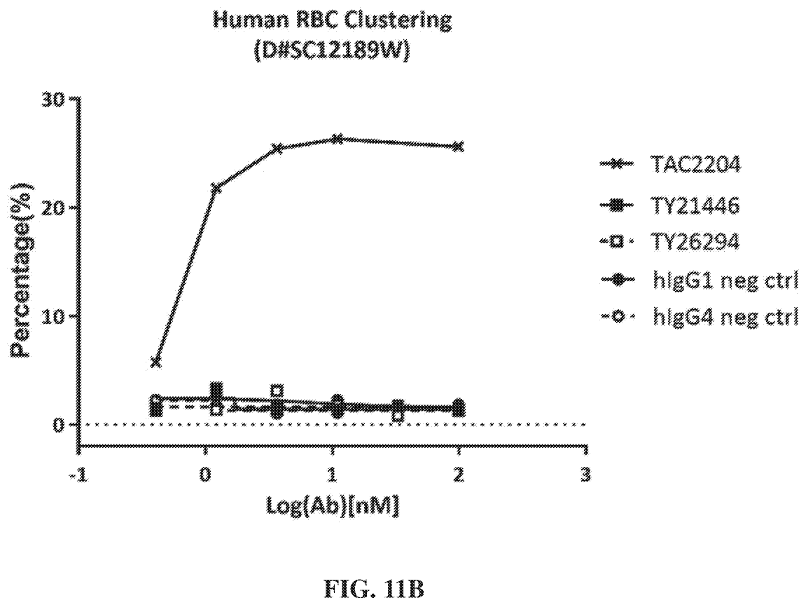

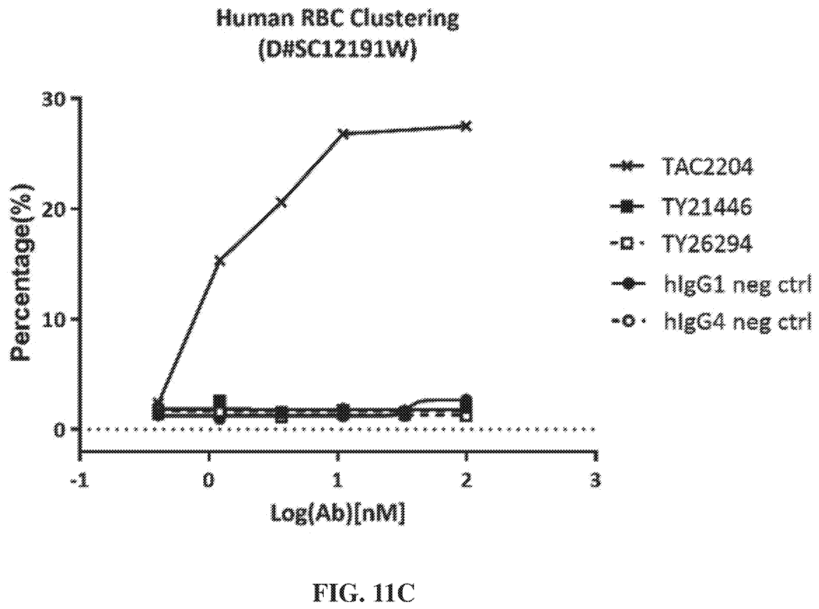

In a third study, human RBCs were incubated with TAC2204, TAC2407, TY21446, TY26294 at 37° C. for 4 hours, followed by flow cytometry analysis. Antibody mediated-crosslinking of the RBCs was measured by FSC-A/FSC-H gating strategy. The results of the study are shown in FIGS. 11A-11C.

As shown in FIGS. 9A-11C, TAC2204 increased cell clustering compared to the negative control on CD47-positive tumor cells (FIG. 9D) and human RBCs (FIGS. 10B-10C and FIGS. 11B-11C), indicating trans binding on cell surface. The parental antibody TY21446 (hlgG4 Fc format) did not show increased cell clustering on tumor cells (FIG. 9C) and human RBCs (FIGS. 10B-10C and FIGS. 11B-11C), indicating cis binding on the cell surface. The activatable antibody TY26294 (hlgG1 Fc format) did not show increased cell clustering on human RBCs (FIGS. 10B-10C and FIGS. 11B-11C), possibly due to the combination of the masking moiety and cis binding.

### Example 15: Anti-CD47 Antibody Binding to RBCs and CD47-Positive Raji Tumor Cells In Vivo

hSIRPα/hCD47 knock-in mice, injected with Raji tumor cells via tail vein and subcutaneously, were used to assess the binding of TY21446 parental antibody, TY26294 activatable antibody, and TAC2204 benchmark molecule to mouse blood cells and Raji tumor cells in vivo. In these knock-in mice, the extracellular domain (ECD) of human CD47 replaces that of mouse CD47; thus, the ECD of human CD47 should be expressed in all tissues in which the mouse CD47 is expressed, including mouse RBCs. Following intraperitoneal dosing of TAC2204 (0.5, 2.5, or 7.5 mg/kg), TY21446 (0.5, 2.5, or 7.5 mg/kg), or TY26294 (0.5, 2.5, or 15 mg/kg), the in vivo binding of antibodies to RBCs, blood cells other than RBCs (“WBC”), Raji tumor cells in the mouse bone marrow, and Raji tumor cells in the subcutaneous tumor were assessed using a receptor occupancy (RO) assay. Briefly, at 4, 24, or 72 h after dosing, mouse whole blood samples were harvested, and at 72 h after dosing, mouse bone marrow samples and subcutaneous tumors were harvested. Mouse whole blood was diluted, washed, and divided into 3 aliquots. Alternatively, mouse whole blood underwent RBC lysis (to prepare “WBC”), with the remaining cells being washed and divided into 3 aliquots. Mouse bone marrow cells were washed and divided into 3 aliquots. Single cell suspensions were prepared from subcutaneous tumors and divided into 3 aliquots. For each tissue type, the first aliquot was not stained with test antibodies, the second aliquot was incubated with 10 nM human IgG4 Isotype Control for 30 min at 37° C., and the third aliquot was incubated with 10 nM TY21446 or TAC2204 for 30 min at 37° C. The second and third aliquots were further incubated with mouse anti-hCD20 antibody and APC-conjugated anti-human-IgG secondary antibody. Samples were analyzed on the CytoFLEX flow cytometer (Beckman Coulter) and the data were analyzed by FlowJo software. The RO rate for each tissue cell type was calculated as mean fluorescence intensity (MFI) of the target population for the second aliquot minus MFI for the first aliquot, divided by MFI for the third aliquot minus MFI for the first aliquot.

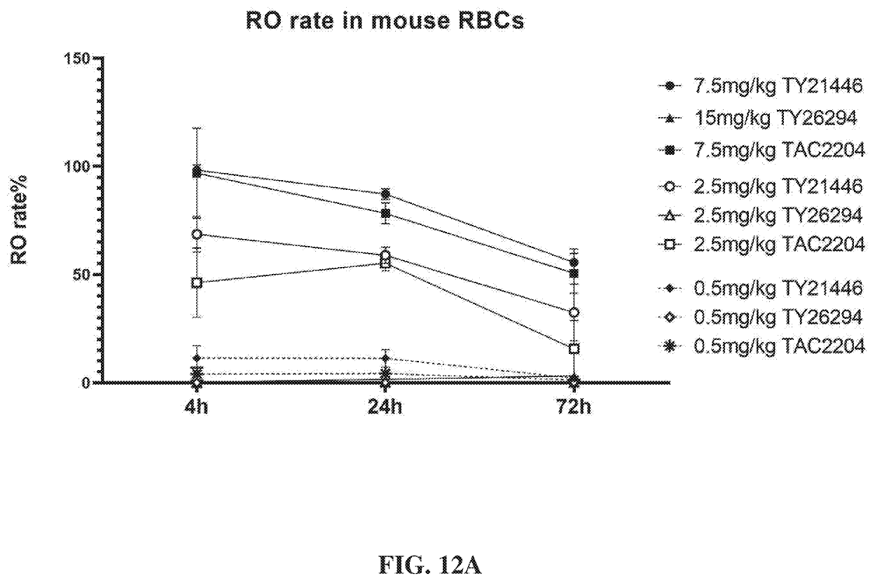

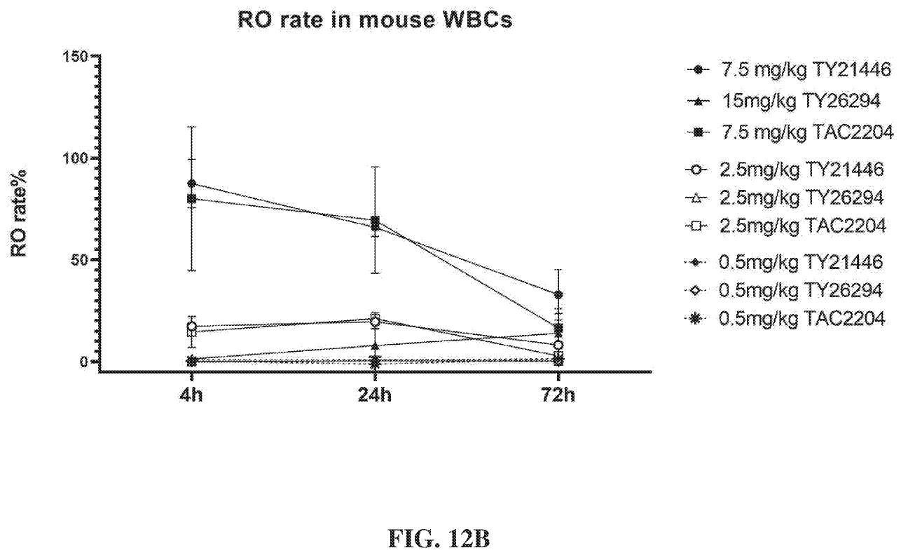

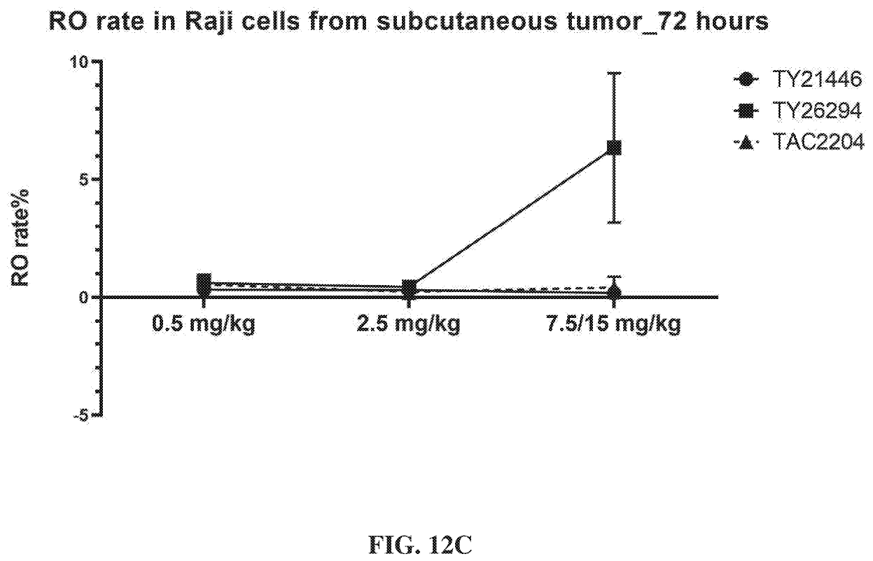

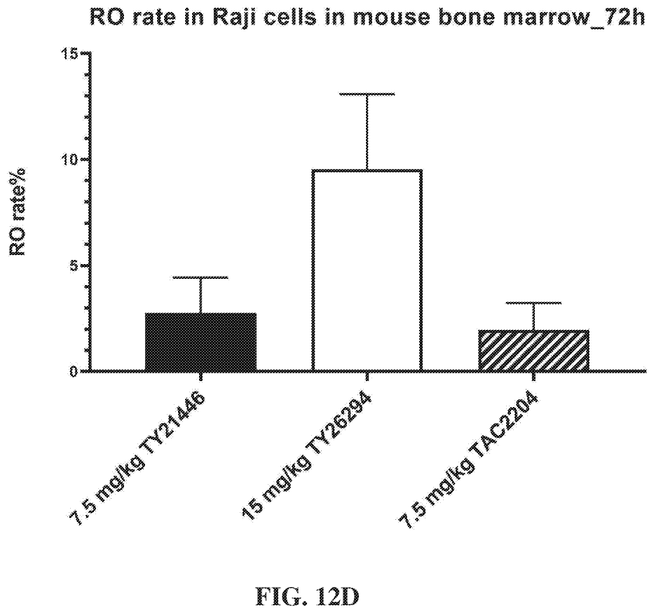

Binding of TAC2204 and TY21446 parental antibodies on RBCs from hCD47 knock-in mice was observed in a time- and dose-dependent manner, with RO approaching 100% at the highest doses tested (FIG. 12A). On the other hand, the TY26294 activatable antibody did not bind RBCs in vivo and RO was less than 5% at all doses and timepoints examined (FIG. 12A). Similarly, FIG. 12B shows a time- and dose-dependent increase in the binding of TAC2204 and TY21446 parental antibodies to mouse WBC, while the TY26294 activatable antibody showed only minimal binding to these mouse cells. RBC and blood cell viability numbers in TAC2204-treated and TY21446 parental-treated mice were significantly reduced compared with those in TY26294 activatable antibody-treated animals (FIG. 12E, Table 21), consistent with the notion that the binding of unmasked anti-CD47 antibodies to RBCs leads to decreased RBC counts. Interestingly, RO was higher for theTY26294 activatable antibody vs. TAC2204 and TY21446 parental antibodies on tumor cells in bone marrow and in subcutaneous tumors. FIGS. 12C and 12D show that theTY26294 demonstrated higher binding than TAC2204 and TY21446 parental antibodies to Raji cells in mouse bone marrow and Raji cells from subcutaneous tumors.

TABLE 21
Blood cell viability and total blood cell count in samples
from mice injected with the indicated anti-CD47 antibodies.

| Group | Blood cell viability (%) | | | Total blood cell count (×10¹²/L) | | |
|---|---|---|---|---|---|---|
| 7.5 mg/kg TY21446 | 81.5 | 85.4 | 86.0 | 2.70 | 5.18 | 3.85 |
| 15 mg/kg TY26294 | 93.3 | 94.7 | 94.2 | 11.05 | 10.48 | 11.80 |
| 7.5 mg/kg TAC2204 | 84.7 | 85.6 | 88.3 | 8.10 | 5.55 | 4.85 |

### Example 16: Preliminary Human Pharmacokinetic and Dose Predictions for the Anti-CD47 Antibodies

Human pharmacokinetic (PK) parameters (e.g., clearance, volume of distribution and half-life) were predicted from cynomolgus monkey PK (total PK only) using body weight-based allometric scaling methods (Mahmood, 2021. “A Single Animal Species-Based Prediction of Human Clearance and First-in-Human Dose of Monoclonal Antibodies: Beyond Monkey.” Antibodies 10.3:35.). Given that the terminal half-life of TY26898 or TY26294 is ≥5 days after a single IV dose at 10 to 60 mg/kg in cynomolgus monkey, a minimal dosing frequency of Q2W (once every two weeks) is predicted in patients while Q3W (once every three weeks) dosing may be achievable. Repeat dose PK will be completed in cynomolgus monkey, which can further elucidate any potential PK-based dosing advantages for TY26898 or TY26294 compared with other anti-CD47 molecules. Mechanism-based modeling and simulation (M&S) was conducted using various currently available in vitro (e.g., ADCC, ADCP) and in vivo (e.g., mouse efficacy) data, leveraging approaches (e.g., Receptor Occupancy based methods; PK-based method targeting specific concentrations from in vitro assays) utilized for other anti-CD47 drugs. Industry standard software such as Phoenix® WinNonlin®, NONMEM® 7.5 and GraphPad Prism were used for M&S. TY26898 has predicted potentially efficacious doses≤10 mg/kg (Q2W or Q3W dosing depending on human PK predictions), the safety of which is supported by initial single dose cynomolgus monkey toxicology data. First-in-human starting dose is likely minimally ˜0.6 mg/kg based on current toxicology data and will likely increase after the determination of no-observed-adverse-effect level (NOAEL) in future repeat dose toxicology studies in cynomolgus monkey. This will allow for rapid dose escalation (e.g., 2-3 cohorts) to reach potential efficacious dose ranges from the starting dose.

### Example 17: Epitope Mapping

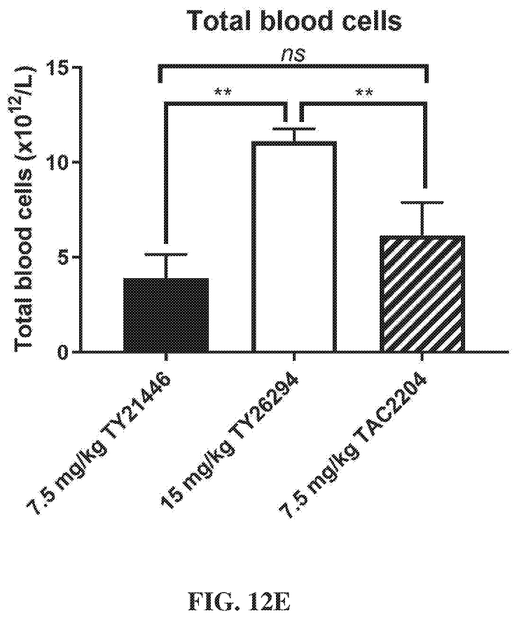

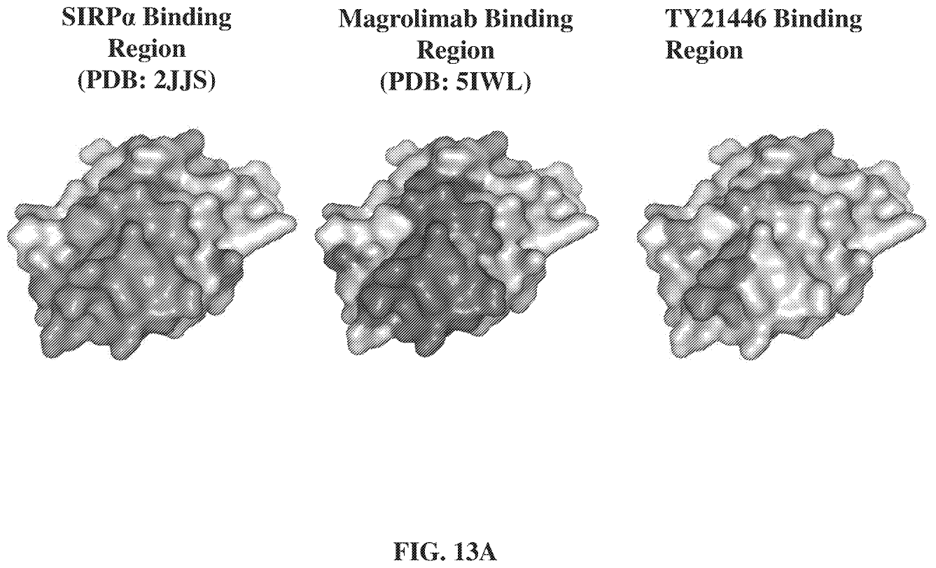

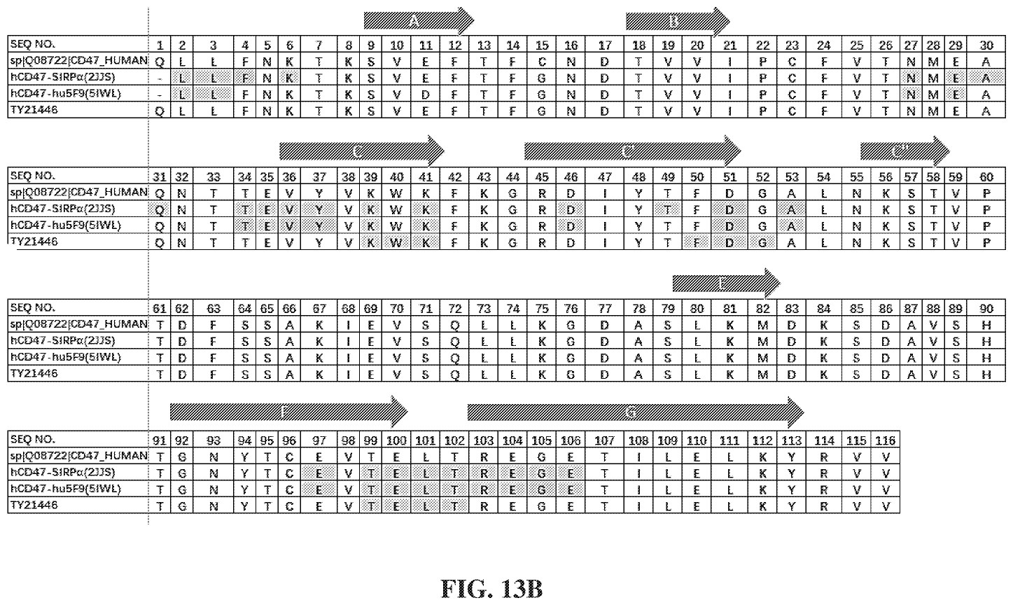

To determine the binding regions of TY21446 at the amino acid residue level, a series of mutants (Table 22) were made of human CD47. The binding of TY21446 to human CD47 mutants was assessed by flow cytometry analysis. The results are summarized in Table 22., The data show TY21446 directly binds on residues 39-41, 50-52, which are in region C to C′ (39K, 40W, 41K, 50F, 51D and 52G), and residues 99-102 in region FG (99T,100E, 101L and 102T) on human CD47 (Table 22). As shown in FIGS. 13A-13B, the binding regions of TY21446 on human CD47 were mapped on human CD47 crystal structure, also the contact regions of magrolimab and SIRPα with human CD47 were determined based on their complex crystal structure and mapped on human CD47. Compared with magrolimab, TY21446 binds to a distinct epitope. TY21446's binding epitope partially overlaps with that of SIRPα on the side, while magrolimab's epitope largely overlaps with that of SIRPα.

TABLE 21
TY21446 binding to mutants of human CD47;
“+” indicates binding, “−” indicates no binding.

| Mutations | TY21446 | Mutations | TY21446 |
|---|---|---|---|
| LL2AA | − | IY47AA | − |
| NK5AA | − | FD50AA | + |
| EF11AA | − | DG51AA | + |
| VT25AA | − | NK55AA | − |
| TN26AA | − | ST57AA | − |
| NME27AAA | − | TD61AA | − |
| ME28AA | − | FS63AA | − |
| QN31AA | − | LK74AA | − |
| TE34AA | − | TE99AA | + |
| KW39AA | + | EL100AA | + |
| WK40AA | + | LT101AA | + |
| FK42AA | − | RE103AA | − |
| RD45AA | − | ET106AA | − |

Since the binding epitope of magrolimab is similar to the binding epitope between SIRPα and CD47, it suggests that magrolimab is likely to bind in trans fashion to bring two anchored proteins on two different cells together, similar to the binding between SIRPα and

CD47 in trans form, while TY21446 is different and likely to be in cis form (see Example 14 for definition for trans and cis binding mode and diagram).

### Example 18: CD47 Receptor Occupancy (RO) in Normal Tissues and Tumors of MDA-MB-231 TNBC Xenograft Model in B-NDG hCD47/hSIRPα Transgenic Mice

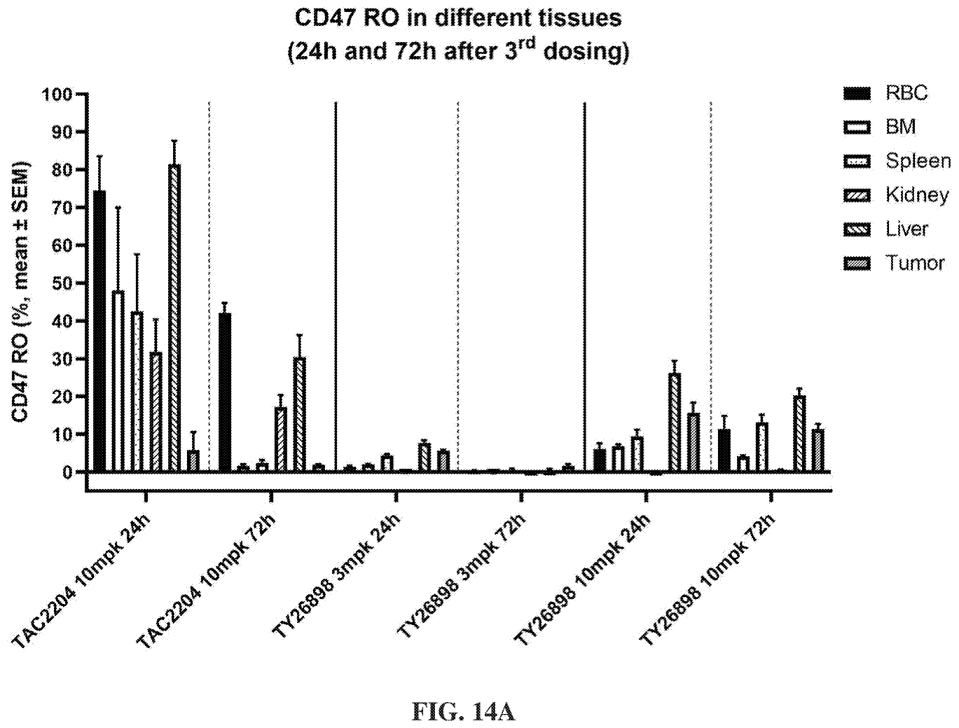

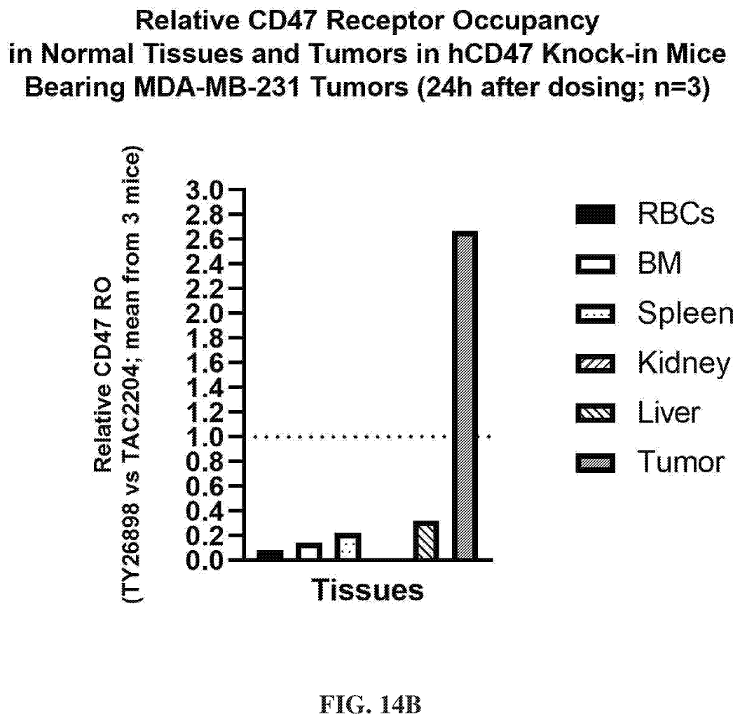

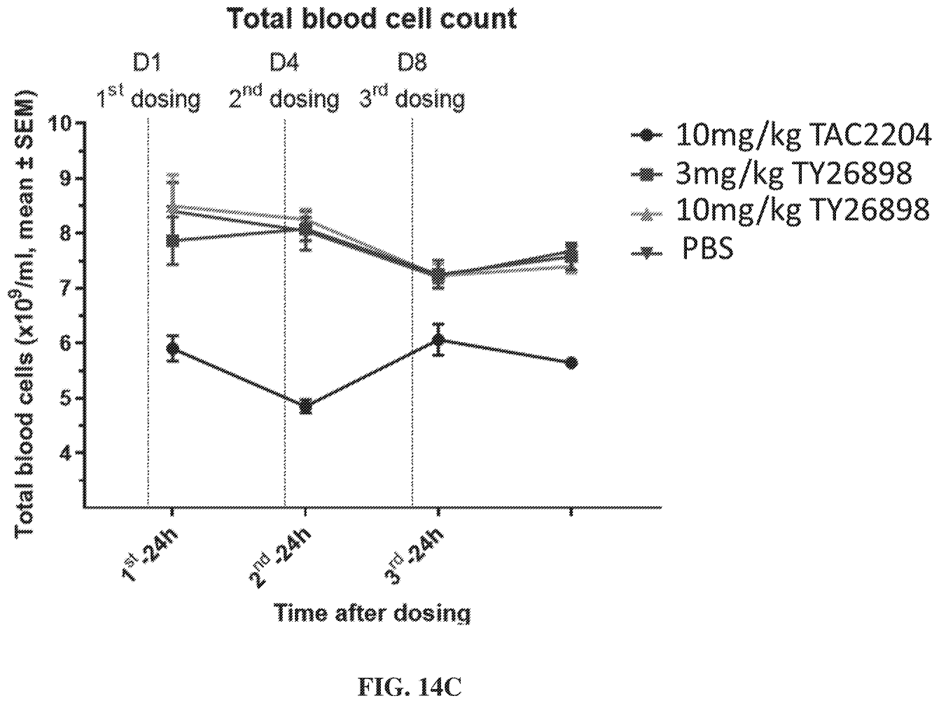

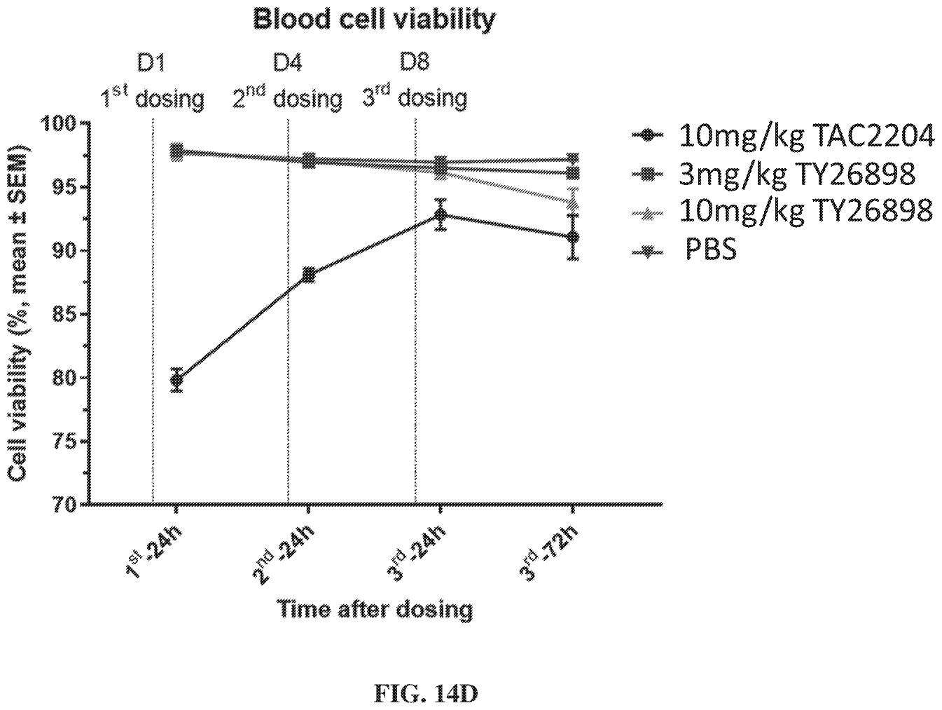

B-NDG hCD47/hSIRPα transgenic mice were acquired from Biocytogen. MDA-MB-231 triple negative breast cancer (TNBC) cells were subcutaneously inoculated in the right upper flank of the mice. When tumor volume reached about 200 mm³ on average, the mice were grouped and then given three doses of PBS, TY26898, or TAC2204. At 24 h after the first and second dosing, blood was collected for blood cell counting, viability and CD47 RO analysis. At 24 h and 72 h after the third dosing, mice were sacrificed and RBC, bone marrow, spleen, kidney, liver, and tumor were collected. The tissues were dissociated into single cells for CD47 RO analysis. As shown in FIGS. 14A-14B, the masked antibody TY26898 showed increased RO in the targeted tumor tissue and reduced RO in off-target tissues compared to TAC2204. Further, treatment with TY26898 resulted in decreased toxicity resulting from off-target binding as compared to TAC2204, as evidenced by increased total blood cell count and blood cell viability as compared to TAC2204 (FIGS. 14C-14D).

### Example 19: In Vivo Efficacy and Receptor Occupancy (RO) of TY26898 in Solid Tumor Xenograft Models

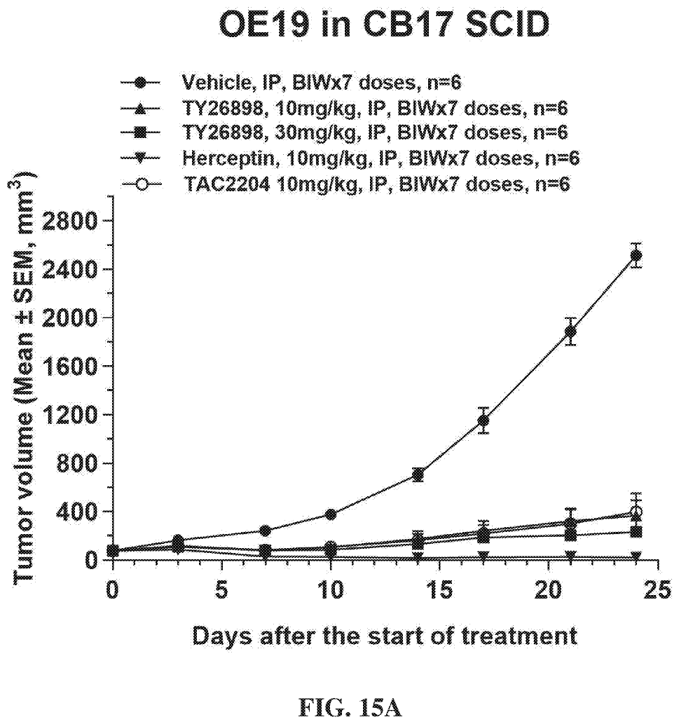

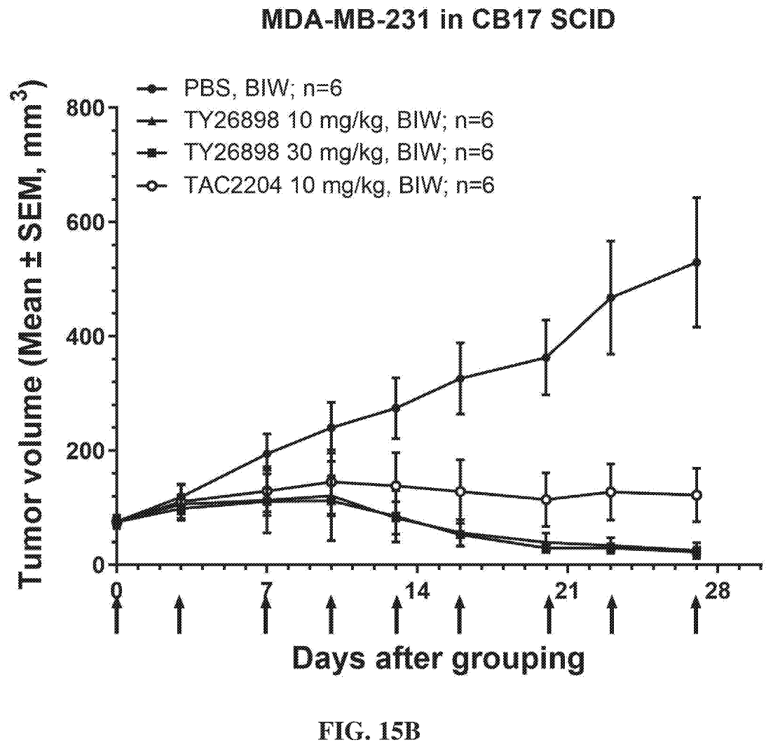

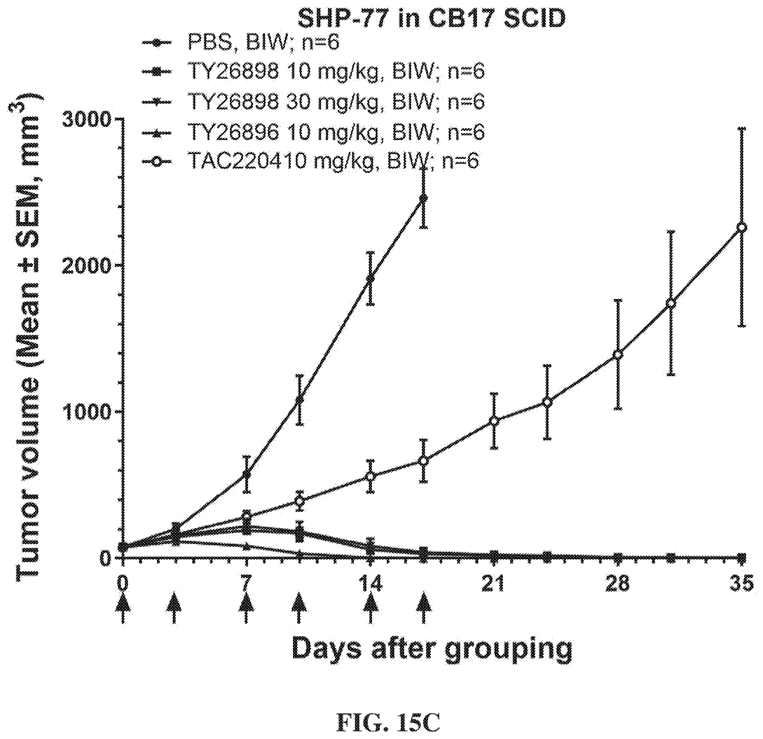

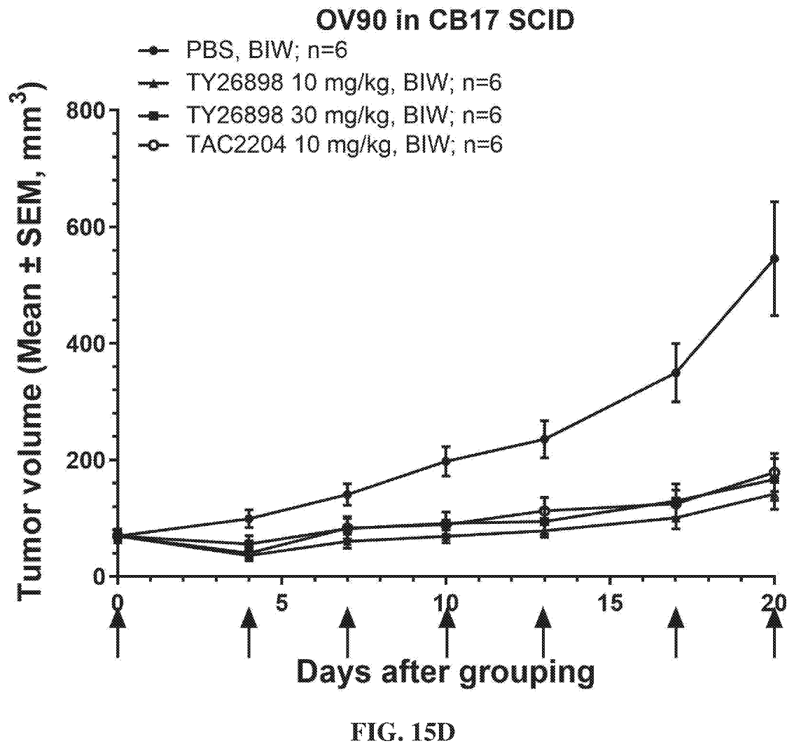

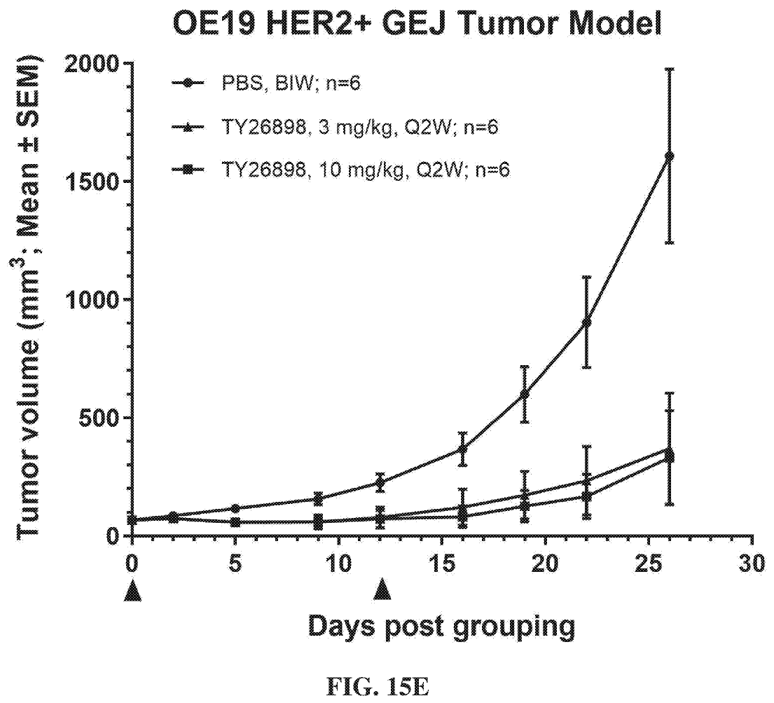

The in vivo anti-tumor activity of TY26898 was evaluated in OE19 HER2+ gastric-esophageal junction (GEJ) cancer, MDA-MB-231 TNBC, SHP-77 small cell lung cancer (SCLC), and OV90 ovarian cancer xenograft models, and OE19 HER2+ GEJ cancer xenograft model in CB17 SCID mice (FIGS. 15A-15E). The in vivo anti-tumor activity of TY26896 was also evaluated in the SHP-77 SCLC model (FIG. 15C). The treatment was given to mice intraperitoneally twice per week (BIW) when tumor volume reached on average 70-80 mm³. Tumor volume and body weight were measured twice per week until the study end. The mice were euthanized when tumor volume exceeded 2000 mm³ or body weight loss exceeded 20%. Treatment with TY26898 resulted in suppression of tumor growth in all cancer models evaluated (FIGS. 15A-15E). Further, TY26898 demonstrated a greater suppression of tumor growth compared to TAC2204 in the MDA-MB-231 TNBC and SHP-77 SCLC models (FIGS. 15B and 15C). TY26896 also showed significantly increased tumor growth suppression as compared to TAC2204 in the SCLC model (FIG. 15C).

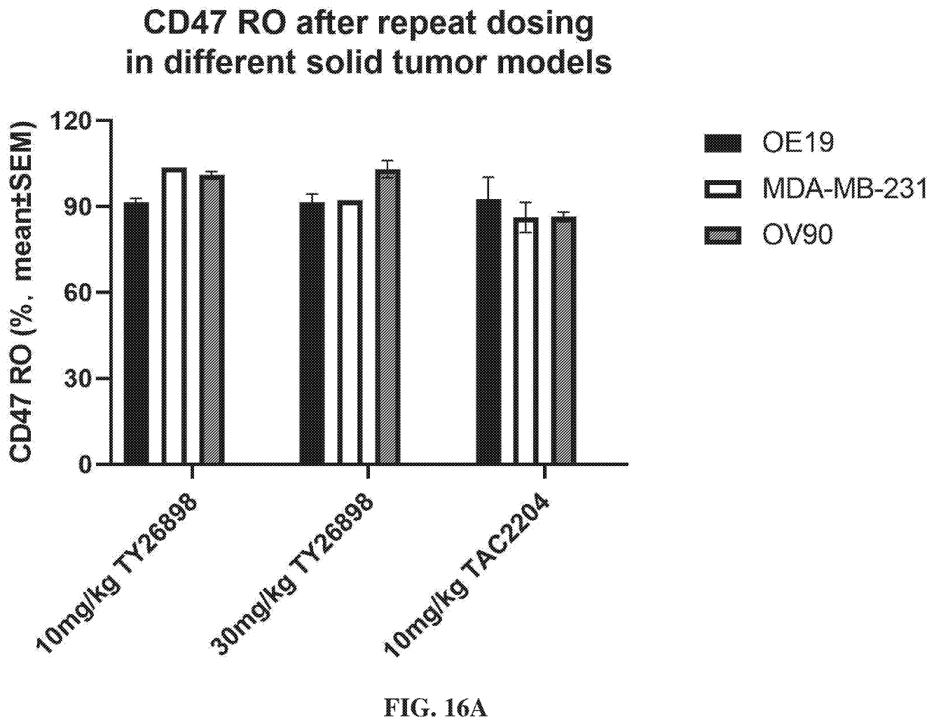

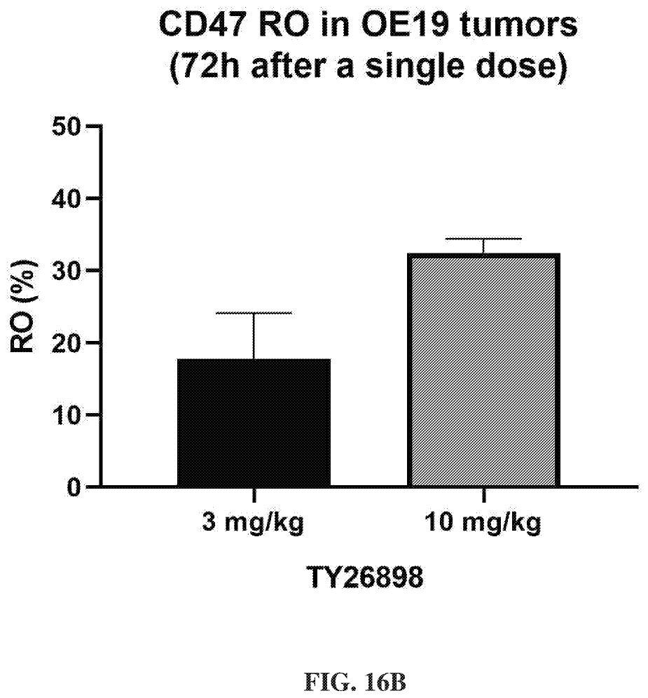

At 72 h after the last dosing in the efficacy studies in solid tumor models, tumors were collected and dissociated into single tumor cells. Single tumor cells were divided into two parts, then stained with either TY26898, TAC2204, or isotype antibody, followed by staining with APC-anti-human IgG secondary antibody. The stained samples were analyzed by flow cytometry, then the CD47 RO was calculated (FIGS. 16A-16B). As shown in FIG. 16A, TY26898 a comparable RO as TAC2204 in the OE19 model, in the MDA-MB-231 and OV90 models.

### Example 20: TY26898 Cleavage in Different Solid Tumor Xenograft Models

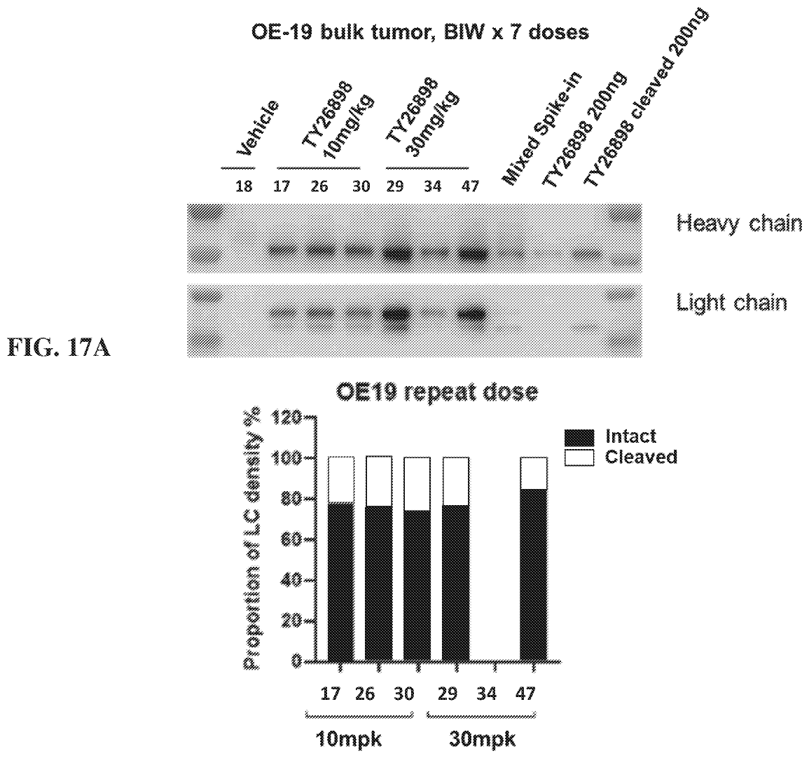

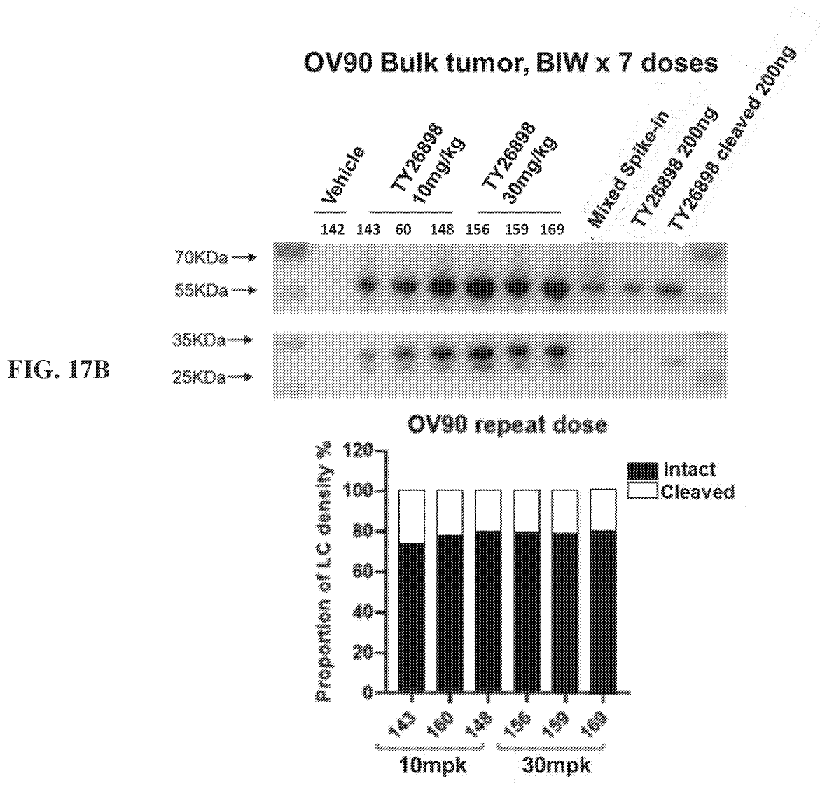

72 h after the last dose of the efficacy studies described in FIGS. 15A and 15D above, tumors were collected and stored frozen. Later, tumors were homogenized in RIPA buffer and proteins were extracted. 2 mg total protein was taken for human IgG enrichment by protein A. All the enriched protein samples were run in reduced SDS-PAGE gel. The heavy chain and light chain of human IgG were detected by corresponding primary and secondary antibodies. 200 ng intact or cleaved TY26898 were spiked into the blank tumor sample, processed in the same way as the tumor samples, and were run together with the test tumor samples as controls. As shown in FIGS. 17A-17B, about 15%-25% of TY26898 cleaved in the OE19 model, and about 20%-25% cleaved in the OV90 model.

What is claimed is:

1. A masked antibody, comprising:
(a) a masking peptide comprising, from N terminus to C terminus, a masking moiety (MM) and a linkage moiety (LM);
(b) a target binding moiety (TBM) comprising an antibody heavy chain variable region (VH) and an antibody light chain variable region (VL), wherein the TBM binds to human CD47; and
(c) a human IgG1 Fc region, or a human IgG Fc region with enhanced antibody-dependent cellular cytotoxicity (ADCC) activity,
wherein the masking peptide is linked to the N terminus of the VH or the VL, and
wherein the MM competes with human CD47 to bind the TBM.

2. The masked antibody of claim 1, wherein the masked antibody comprises a first polypeptide, a second polypeptide, a third polypeptide, and a fourth polypeptide, wherein:
a) the first polypeptide comprises a light chain comprising, from N terminus to C terminus, the masking peptide and the VL;
b) the second polypeptide comprises a heavy chain comprising, from N terminus to C terminus, the VH and a first human IgG1 Fc domain;
c) the third polypeptide comprises a light chain comprising, from N terminus to C terminus, the masking peptide and the VL; and
d) the fourth polypeptide comprises a heavy chain comprising, from N terminus to C terminus, the VH and a second human IgG1 Fc domain.

3. A masked antibody, comprising:
(a) a masking peptide comprising, from N terminus to C terminus, a masking moiety (MM) and a linkage moiety (LM), and
(b) a target binding moiety (TBM) comprising an antibody heavy chain variable region (VH) and an antibody light chain variable region (VL), wherein the TBM binds to human CD47;
wherein the masking peptide is linked to the N terminus of the VH or the VL; and
wherein the MM competes with human CD47 to bind the TBM;
wherein:
1) the VH comprises a first complementary-determining-region (CDR-H1), a second complementary-determining-region (CDR-H2), and a third complementary-determining-region (CDR-H3),
wherein the CDR-H1 comprises the amino acid sequence of SEQ ID NO: 182 or 183;
wherein the CDR-H2 comprises the amino acid sequence of SEQ ID NO: 184 or 185; and
wherein the CDR-H3 comprises an amino acid sequence selected from the group consisting of SEQ ID NOs: 186-189; and/or
2) the VL comprises a first complementary-determining-region (CDR-L1), a second complementary-determining-region (CDR-L2), and a third complementary-determining-region (CDR-L3),
wherein the CDR-L1 comprises an amino acid sequence selected from the group consisting of SEQ ID NOs: 190-193;
wherein the CDR-L2 comprises the amino acid sequence of SEQ ID NO: 194; and
wherein the CDR-L3 comprises the amino acid sequence of SEQ ID NO: 195 or 196.

4. The masked antibody of claim 1 or 3, wherein:
a) the masked antibody comprises a first polypeptide comprising, from N terminus to C terminus, the masking peptide and the VL, and a second polypeptide comprising the VH;
or
b) the masked antibody comprises a first polypeptide comprising, from N terminus to C terminus, the masking peptide and the VH, and a second polypeptide comprising the VL;
or
c) the masked antibody comprises a first polypeptide comprising, from N terminus to C terminus, the masking peptide, the VL, and the VH; or
d) the masked antibody comprises a first polypeptide comprising, from N terminus to C terminus the masking peptide, the VH, and the VL.

5. The masked antibody of claim 3 or 4, wherein:
a) the VH comprises
a CDR-H1 comprising the amino acid sequence of SEQ ID NO: 182,
a CDR-H2 comprising the amino acid sequence of SEQ ID NO: 185, and
a CDR-H3 comprising the amino acid sequence of SEQ ID NO: 188; and
b) the VL comprises
a CDR-L1 comprising the amino acid sequence of SEQ ID NO: 191,
a CDR-L2 comprising the amino acid sequence of SEQ ID NO: 194, and
a CDR-L3 comprising the amino acid sequence of SEQ ID NO: 195.

6. The masked antibody of claim 3 or 4, wherein:
(a) the VH comprises a CDR-H1 comprising an amino acid sequence selected from the group consisting of SEQ ID NOs: 35-37 and 39-50, a CDR-H2 comprising an amino acid sequence selected from the group consisting of SEQ ID NOs: 52-54 and 56-67, and a CDR-H3 comprising an amino acid sequence selected from the group consisting of SEQ ID NOS: 69-71 and 73-84; and
(b) the VL comprises a CDR-L1 comprising an amino acid sequence selected from the group consisting of SEQ ID NOs: 86-88 and 89-101, a CDR-L2 comprising an amino acid sequence selected from the group consisting of SEQ ID NOs: 103-105 and 106-118, and a CDR-L3 comprising an amino acid sequence selected from the group consisting of SEQ ID NOs: 120-122 and 124-135.

7. The masked antibody of claim 3 or 4, wherein the VH comprises an amino acid sequence selected from the group consisting of SEQ ID NOs: 1, 3, 5, 9, 11, 13, 15, 17, 19, 21, 23, 25, 27, 29, and 31; and/or wherein the VL comprises an amino acid sequence selected from the group consisting of SEQ ID NOs: 2, 4, 6, 10, 12, 14, 16, 18, 20, 22, 24, 26, 28, 30, and 32.

8. The masked antibody of claim 3 or 4, wherein
a) the VH comprises
a CDR-H1 comprising the amino acid sequence of SEQ ID NO: 48,
a CDR-H2 comprising the amino acid sequence of SEQ ID NO: 65, and
a CDR-H3 comprising the amino acid sequence of SEQ ID NO: 82; and
b) the VL comprises
a CDR-L1 comprising the amino acid sequence of SEQ ID NO: 99,
a CDR-L2 comprising the amino acid sequence of SEQ ID NO: 116, and
a CDR-L3 comprising the amino acid sequence of SEQ ID NO: 133.

9. The masked antibody of claim 8, wherein the VH comprises the amino acid sequence of SEQ ID NO: 27, and/or the VL comprises the amino acid sequence of SEQ ID NO: 28.

10. The masked antibody of claim 3 or 4, wherein
a) the VH comprises
a CDR-H1 comprising the amino acid sequence of SEQ ID NO: 49,
a CDR-H2 comprising the amino acid sequence of SEQ ID NO: 66, and
a CDR-H3 comprising the amino acid sequence of SEQ ID NO: 83; and
b) the VL comprises
a CDR-L1 comprising the amino acid sequence of SEQ ID NO: 100,
a CDR-L2 comprising the amino acid sequence of SEQ ID NO: 117, and
a CDR-L3 comprising the amino acid sequence of SEQ ID NO: 134.

11. The masked antibody of claim 10, wherein the VH comprises the amino acid sequence of SEQ ID NO: 29, and/or the VL comprises the amino acid sequence of SEQ ID NO: 30.

12. The masked antibody of claim 3 or 4, wherein
a) the VH comprises
a CDR-H1 comprising the amino acid sequence of SEQ ID NO: 50,
a CDR-H2 comprising the amino acid sequence of SEQ ID NO: 67, and
a CDR-H3 comprising the amino acid sequence of SEQ ID NO: 84; and
b) the VL comprises
a CDR-L1 comprising the amino acid sequence of SEQ ID NO: 101,
a CDR-L2 comprising the amino acid sequence of SEQ ID NO: 118, and
a CDR-L3 comprising the amino acid sequence of SEQ ID NO: 135.

13. The masked antibody of claim 12, wherein the VH comprises the amino acid sequence of SEQ ID NO: 31, and/or the VL comprises the amino acid sequence of SEQ ID NO: 32.

14. The masked antibody of claim 3 or 4, wherein
a) the VH comprises
a CDR-H1 comprising the amino acid sequence of SEQ ID NO: 35,
a CDR-H2 comprising the amino acid sequence of SEQ ID NO: 52, and
a CDR-H3 comprising the amino acid sequence of SEQ ID NO: 69; and
b) the VL comprises
a CDR-L1 comprising the amino acid sequence of SEQ ID NO: 86,
a CDR-L2 comprising the amino acid sequence of SEQ ID NO: 103, and
a CDR-L3 comprising the amino acid sequence of SEQ ID NO: 120.

15. The masked antibody of claim 14, wherein the VH comprises the amino acid sequence of SEQ ID NO: 1, and/or the VL comprises the amino acid sequence of SEQ ID NO: 2.

16. The masked antibody of claim 3 or 4, wherein
a) the VH comprises
a CDR-H1 comprising the amino acid sequence of SEQ ID NO: 36,
a CDR-H2 comprising the amino acid sequence of SEQ ID NO: 53, and
a CDR-H3 comprising the amino acid sequence of SEQ ID NO: 70; and
b) the VL comprises
a CDR-L1 comprising the amino acid sequence of SEQ ID NO: 87,
a CDR-L2 comprising the amino acid sequence of SEQ ID NO: 104, and
a CDR-L3 comprising the amino acid sequence of SEQ ID NO: 121.

17. The masked antibody of claim 16, wherein the VH comprises the amino acid sequence of SEQ ID NO: 3, and/or the VL comprises the amino acid sequence of SEQ ID NO: 4.

18. The masked antibody of claim 3 or 4, wherein
a) the VH comprises
a CDR-H1 comprising the amino acid sequence of SEQ ID NO: 37,
a CDR-H2 comprising the amino acid sequence of SEQ ID NO: 54, and
a CDR-H3 comprising the amino acid sequence of SEQ ID NO: 71; and
b) the VL comprises
a CDR-L1 comprising the amino acid sequence of SEQ ID NO: 88,
a CDR-L2 comprising the amino acid sequence of SEQ ID NO: 105, and
a CDR-L3 comprising the amino acid sequence of SEQ ID NO: 122.

19. The masked antibody of claim 18, wherein the VH comprises the amino acid sequence of SEQ ID NO: 5, and/or the VL comprises the amino acid sequence of SEQ ID NO: 6.

20. The masked antibody of claim 3 or 4, wherein
a) the VH comprises
a CDR-H1 comprising the amino acid sequence of SEQ ID NO: 39,
a CDR-H2 comprising the amino acid sequence of SEQ ID NO: 56, and
a CDR-H3 comprising the amino acid sequence of SEQ ID NO: 73; and
b) the VL comprises
a CDR-L1 comprising the amino acid sequence of SEQ ID NO: 90,
a CDR-L2 comprising the amino acid sequence of SEQ ID NO: 107, and
a CDR-L3 comprising the amino acid sequence of SEQ ID NO: 124.

21. The masked antibody of claim 20, wherein the VH comprises the amino acid sequence of SEQ ID NO: 9, and/or the VL comprises the amino acid sequence of SEQ ID NO: 10.

22. The masked antibody of claim 3 or 4, wherein
a) the VH comprises
a CDR-H1 comprising the amino acid sequence of SEQ ID NO: 40,
a CDR-H2 comprising the amino acid sequence of SEQ ID NO: 57, and
a CDR-H3 comprising the amino acid sequence of SEQ ID NO: 74; and
b) the VL comprises
a CDR-L1 comprising the amino acid sequence of SEQ ID NO: 91,
a CDR-L2 comprising the amino acid sequence of SEQ ID NO: 108, and
a CDR-L3 comprising the amino acid sequence of SEQ ID NO: 125.

23. The masked antibody of claim 22, wherein the VH comprises the amino acid sequence of SEQ ID NO: 11, and/or the VL comprises the amino acid sequence of SEQ ID NO: 12.

24. The masked antibody of claim 3 or 4, wherein
a) the VH comprises
a CDR-H1 comprising the amino acid sequence of SEQ ID NO: 41,
a CDR-H2 comprising the amino acid sequence of SEQ ID NO: 58, and
a CDR-H3 comprising the amino acid sequence of SEQ ID NO: 75; and
b) the VL comprises
a CDR-L1 comprising the amino acid sequence of SEQ ID NO: 92,
a CDR-L2 comprising the amino acid sequence of SEQ ID NO: 109, and
a CDR-L3 comprising the amino acid sequence of SEQ ID NO: 126.

25. The masked antibody of claim 24, wherein the VH comprises the amino acid sequence of SEQ ID NO: 13, and/or the VL comprises the amino acid sequence of SEQ ID NO: 14.

26. The masked antibody of claim 3 or 4, wherein
a) the VH comprises
a CDR-H1 comprising the amino acid sequence of SEQ ID NO: 42,
a CDR-H2 comprising the amino acid sequence of SEQ ID NO: 59, and
a CDR-H3 comprising the amino acid sequence of SEQ ID NO: 76; and
b) the VL comprises
a CDR-L1 comprising the amino acid sequence of SEQ ID NO: 93,
a CDR-L2 comprising the amino acid sequence of SEQ ID NO: 110, and
a CDR-L3 comprising the amino acid sequence of SEQ ID NO: 127.

27. The masked antibody of claim 26, wherein the VH comprises the amino acid sequence of SEQ ID NO: 15, and/or the VL comprises the amino acid sequence of SEQ ID NO: 16.

28. The masked antibody of claim 3 or 4, wherein
a) the VH comprises
a CDR-H1 comprising the amino acid sequence of SEQ ID NO: 43,
a CDR-H2 comprising the amino acid sequence of SEQ ID NO: 60, and
a CDR-H3 comprising the amino acid sequence of SEQ ID NO: 77; and
b) the VL comprises
a CDR-L1 comprising the amino acid sequence of SEQ ID NO: 94,
a CDR-L2 comprising the amino acid sequence of SEQ ID NO: 111, and
a CDR-L3 comprising the amino acid sequence of SEQ ID NO: 128.

29. The masked antibody of claim 28, wherein the VH comprises the amino acid sequence of SEQ ID NO: 17, and/or the VL comprises the amino acid sequence of SEQ ID NO: 18.

30. The masked antibody of claim 3 or 4, wherein
a) the VH comprises
a CDR-H1 comprising the amino acid sequence of SEQ ID NO: 44,
a CDR-H2 comprising the amino acid sequence of SEQ ID NO: 61, and
a CDR-H3 comprising the amino acid sequence of SEQ ID NO: 78; and
b) the VL comprises
a CDR-L1 comprising the amino acid sequence of SEQ ID NO: 95,
a CDR-L2 comprising the amino acid sequence of SEQ ID NO: 112, and
a CDR-L3 comprising the amino acid sequence of SEQ ID NO: 129.

31. The masked antibody of claim 30, wherein the VH comprises the amino acid sequence of SEQ ID NO: 19, and/or the VL comprises the amino acid sequence of SEQ ID NO: 20.

32. The masked antibody of claim 3 or 4, wherein
a) the VH comprises
a CDR-H1 comprising the amino acid sequence of SEQ ID NO: 45,
a CDR-H2 comprising the amino acid sequence of SEQ ID NO: 62, and
a CDR-H3 comprising the amino acid sequence of SEQ ID NO: 79; and
b) the VL comprises
a CDR-L1 comprising the amino acid sequence of SEQ ID NO: 96,
a CDR-L2 comprising the amino acid sequence of SEQ ID NO: 113, and
a CDR-L3 comprising the amino acid sequence of SEQ ID NO: 130.

33. The masked antibody of claim 32, wherein the VH comprises the amino acid sequence of SEQ ID NO: 21, and/or the VL comprises the amino acid sequence of SEQ ID NO: 22.

34. The masked antibody of claim 3 or 4, wherein
a) the VH comprises
a CDR-H1 comprising the amino acid sequence of SEQ ID NO: 46,
a CDR-H2 comprising the amino acid sequence of SEQ ID NO: 63, and
a CDR-H3 comprising the amino acid sequence of SEQ ID NO: 80; and
b) the VL comprises
a CDR-L1 comprising the amino acid sequence of SEQ ID NO: 97,
a CDR-L2 comprising the amino acid sequence of SEQ ID NO: 114, and
a CDR-L3 comprising the amino acid sequence of SEQ ID NO: 131.

35. The masked antibody of claim 34, wherein the VH comprises the amino acid sequence of SEQ ID NO: 23, and/or the VL comprises the amino acid sequence of SEQ ID NO: 24.

36. The masked antibody of claim 3 or 4, wherein
a) the VH comprises
a CDR-H1 comprising the amino acid sequence of SEQ ID NO: 47,
a CDR-H2 comprising the amino acid sequence of SEQ ID NO: 64, and
a CDR-H3 comprising the amino acid sequence of SEQ ID NO: 81; and
b) the VL comprises
a CDR-L1 comprising the amino acid sequence of SEQ ID NO: 98,
a CDR-L2 comprising the amino acid sequence of SEQ ID NO: 115, and
a CDR-L3 comprising the amino acid sequence of SEQ ID NO: 132.

37. The masked antibody of claim 36, wherein the VH comprises the amino acid sequence of SEQ ID NO: 25, and/or the VL comprises the amino acid sequence of SEQ ID NO: 26.

38. The masked antibody of any one of claims 1-37, wherein the MM comprises an amino acid sequence selected from the group consisting of SEQ ID NOs: 197-200.

39. The masked antibody of claim 38, wherein the MM comprises an amino acid sequence selected from the group consisting of SEQ ID NOs: 137 and 167-181.

40. The masked antibody of claim 39, wherein the MM comprises the amino acid sequence of SEQ ID NO: 199.

41. The masked antibody of claim 40, wherein the MM comprises the amino acid sequence of SEQ ID NO: 137.

42. The masked antibody of any one of claims 1-41, wherein the LM does not comprise a cleavable moiety (CM) comprising at least one cleavage site.

43. The masked antibody of any one of claims 1-42, wherein the masked antibody is an activatable antibody.

44. The masked antibody of claim 43, wherein the LM comprises a cleavable moiety (CM) comprising at least one cleavage site, and wherein the activatable antibody has a higher binding affinity to human CD47 in vitro upon cleavage of the CM than before the cleavage of the CM.

45. The masked antibody of claim 43 or 44, wherein the cleavage site is a protease cleavage site for a protease selected from the group consisting of urokinase-type plasminogen activator (uPA), matrix metalloproteinase-1 (MMP-1), MMP-2, MMP-3, MMP-8, MMP-9, MMP-14, Tobacco Etch Virus (TEV) protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE.

46. The masked antibody of claim 45, wherein the masking peptide comprises an amino acid sequence selected from the group consisting of SEQ ID NOs: 139 and 152-166.

47. The masked antibody of claim 45, wherein the CM comprises an MMP-9 cleavage site that is cleavable by MMP-9.

48. The masked antibody of claim 47, wherein the CM comprises the amino acid sequence of SEQ ID NO: 138.

49. The masked antibody of claim 48, wherein the masking peptide comprises the amino acid sequence of SEQ ID NO: 139.

50. The masked antibody of any one of claims 1-49, wherein the masked antibody comprises a first polypeptide comprising, from N terminus to C terminus the masking peptide and the VL; and a second polypeptide comprising the VH.

51. The masked antibody of claim 50, wherein the masked antibody comprises a human IgG4 fragment crystallizable (Fc) region.

52. The masked antibody of claim 51, wherein the masked antibody further comprises a third polypeptide and a fourth polypeptide, and wherein:
a) the first polypeptide comprises a light chain comprising, from N terminus to C terminus, the masking peptide and the VL;
b) the second polypeptide comprises a heavy chain comprising, from N terminus to C terminus, the VH and a human IgG4 Fc domain;
c) the third polypeptide comprises a light chain comprising, from N terminus to C terminus, the masking peptide and the VL; and
d) the fourth polypeptide comprises a heavy chain comprising, from N terminus to C terminus, the VH and a human IgG4 Fc domain.

53. The masked antibody of claim 51 or 52, wherein the first polypeptide comprises a light chain comprising, from N terminus to C terminus, the masking peptide and the VL, wherein the first polypeptide comprises the amino acid sequence of SEQ ID NO: 142; and wherein the second polypeptide comprises a heavy chain comprising, from N terminus to C terminus, the VH and a human IgG4 Fc domain, wherein the second polypeptide comprises the amino acid sequence of SEQ ID NO: 143.

54. The masked antibody of claim 50, wherein the masked antibody comprises a human IgG1 Fc region.

55. The masked antibody of claim 54, wherein the masked antibody further comprises a third polypeptide and a fourth polypeptide, and wherein:
a) the first polypeptide comprises a light chain comprising, from N terminus to C terminus, the masking peptide and the VL;
b) the second polypeptide comprises a heavy chain comprising, from N terminus to C terminus, the VH and a human IgG1 Fc domain;
c) the third polypeptide comprises a light chain comprising, from N terminus to C terminus, the masking peptide and the VL; and
d) the fourth polypeptide comprises a heavy chain comprising, from N terminus to C terminus, the VH and a human IgG1 Fc domain.

56. The masked antibody of claim 54 or 55, wherein the first polypeptide comprises a light chain comprising, from N terminus to C terminus, the masking peptide and the VL, wherein the first polypeptide comprises the amino acid sequence of SEQ ID NO: 148; and wherein the second polypeptide comprises a heavy chain comprising, from N terminus to C terminus, the VH and a human IgG1 Fc domain, wherein the second polypeptide comprises the amino acid sequence of SEQ ID NO: 149.

57. The masked antibody of claim 54, wherein the human IgG1 Fc region comprises two Fc domains each comprising a S239D substitution and/or an 1332E substitution.

58. The masked antibody of claim 57, wherein the masked antibody further comprises a third polypeptide and a fourth polypeptide, and wherein:
a) the first polypeptide comprises a light chain comprising, from N terminus to C terminus, the masking peptide and the VL;
b) the second polypeptide comprises a heavy chain comprising, from N terminus to C terminus, the VH and a human IgG1 Fc domain comprising a S239D substitution and/or an 1332E substitution;
c) the third polypeptide comprises a light chain comprising, from N terminus to C terminus, the masking peptide and the VL; and
d) the fourth polypeptide comprises a heavy chain comprising, from N terminus to C terminus, the VH and a human IgG1 Fc domain comprising a S239D substitution and/or an 1332E substitution.

59. The masked antibody of claim 57 or 58, wherein the first polypeptide comprises a light chain comprising, from N terminus to C terminus, the masking peptide and the VL, wherein the first polypeptide comprises the amino acid sequence of SEQ ID NO: 150; and wherein the second polypeptide comprises a heavy chain comprising, from N terminus to C terminus, the VH and a human IgG1 Fc domain, wherein the second polypeptide comprises the amino acid sequence of SEQ ID NO: 151.

60. The masked antibody of any one of claims 1-59, wherein the masking peptide has a masking efficiency of at least about 50 as determined by a Jurkat NFAT reporter assay.

61. The masked antibody of claim 60, wherein the masking peptide has a masking efficiency of at least about 100 as determined by a Jurkat NFAT reporter assay.

62. The masked antibody of any one of claims 1-61, wherein the masked antibody is cross-reactive with a CD47 polypeptide from at least one non-human species selected from the group consisting of cynomolgus monkey, rat, and dog.

63. The masked antibody of claim 62, wherein the masked antibody binds to cynomolgus monkey CD47.

64. The masked antibody of any one of claims 1-63, wherein the masked antibody binds to one or more amino acid residues of human CD47 selected from the group consisting of K39, W40, K41, F50, D51, G52, T99, E100, L101, and T102.

65. The masked anybody of any one of claims 1-64, wherein the masked antibody does not bind to one or more amino acid residues of human CD47 selected from the group consisting of L2, L3, F4, K6, N27, E29, A30, Q31, T34, E35, V36, Y37, D46, T49, A53, E97, R103, E104, G105, and E106.

66. The masked antibody of any one of claims 1-65, wherein the masked antibody binds to amino acid residues K39, W40, K41, F50, D51, G52, T99, E100, L101, and T102 of human CD47.

67. The masked antibody of any one of claims 1-66, wherein the masked antibody does not bind to amino acid residues L2, L3, F4, K6, N27, E29, A30, Q31, T34, E35, V36, Y37, D46, T49, A53, E97, R103, E104, G105, and E106 of human CD47.

68. An isolated antibody, or antigen-binding fragment thereof, that binds to human CD47, comprising a VH and a VL, wherein
1) the VH comprises a CDR-H1, a CDR-H2, and a CDR-H3,
wherein the CDR-H1 comprises the amino acid sequence of SEQ ID NO: 182 or 183;
wherein the CDR-H2 comprises the amino acid sequence of SEQ ID NO: 184 or 185; and
wherein the CDR-H3 comprises an amino acid sequence selected from the group consisting of SEQ ID NOs: 186-189; and/or
2) the VL comprises a CDR-L1, a CDR-L2, and a CDR-L3,
wherein the CDR-L1 comprises an amino acid sequence selected from the group consisting of SEQ ID NOs: 190-193;
wherein the CDR-L2 comprises the amino acid sequence of SEQ ID NO: 194; and
wherein the CDR-L3 comprises the amino acid sequence of SEQ ID NO: 195 or 196.

69. The isolated antibody or antigen-binding fragment of claim 68, wherein:
a) the VH comprises
a CDR-H1 comprising the amino acid sequence of SEQ ID NO: 182,
a CDR-H2 comprising the amino acid sequence of SEQ ID NO: 185, and
a CDR-H3 comprising the amino acid sequence of SEQ ID NO: 188; and
b) the VL comprises
a CDR-L1 comprising the amino acid sequence of SEQ ID NO: 191,
a CDR-L2 comprising the amino acid sequence of SEQ ID NO: 194, and
a CDR-L3 comprising the amino acid sequence of SEQ ID NO: 195.

70. The isolated antibody or antigen-binding fragment of claim 68, wherein
(a) the VH comprises a CDR-H1 comprising an amino acid sequence selected from the group consisting of SEQ ID NOs: 35-37 and 39-50, a CDR-H2 comprising an amino acid sequence selected from the group consisting of SEQ ID NOs: 52-54 and 56-67, and a CDR-H3 comprising an amino acid sequence selected from the group consisting of SEQ ID NOs: 69-71 and 73-84; and
(b) the VL comprises a CDR-L1 comprising an amino acid sequence selected from the group consisting of SEQ ID NOs: 86-88 and 90-101, a CDR-L2 comprising an amino acid sequence selected from the group consisting of SEQ ID NOs: 103-105 and 106-118, and a CDR-L3 comprising an amino acid sequence selected from the group consisting of SEQ ID NOs: 120-122 and 124-135.

71. The isolated antibody or antigen-binding fragment of claim 68, wherein the VH comprises an amino acid sequence selected from the group consisting of SEQ ID NOs: 1, 3, 5, 9, 11, 13, 15, 17, 19, 21, 23, 25, 27, 29, and 31; and/or wherein the VL comprises an amino acid sequence selected from the group consisting of SEQ ID NOs: 2, 4, 6, 10, 12, 14, 16, 18, 20, 22, 24, 26, 28, 30, and 32.

72. The isolated antibody or antigen-binding fragment of claim 68, wherein
a) the VH comprises
a CDR-H1 comprising the amino acid sequence of SEQ ID NO: 48,
a CDR-H2 comprising the amino acid sequence of SEQ ID NO: 65, and
a CDR-H3 comprising the amino acid sequence of SEQ ID NO: 82; and
b) the VL comprises
a CDR-L1 comprising the amino acid sequence of SEQ ID NO: 99,
a CDR-L2 comprising the amino acid sequence of SEQ ID NO: 116, and
a CDR-L3 comprising the amino acid sequence of SEQ ID NO: 133.

73. The isolated antibody or antigen-binding fragment of claim 72, wherein the VH comprises the amino acid sequence of SEQ ID NO: 27, and/or the VL comprises the amino acid sequence of SEQ ID NO: 28.

74. The isolated antibody or antigen-binding fragment of claim 68, wherein
a) the VH comprises
a CDR-H1 comprising the amino acid sequence of SEQ ID NO: 49,
a CDR-H2 comprising the amino acid sequence of SEQ ID NO: 66, and
a CDR-H3 comprising the amino acid sequence of SEQ ID NO: 83; and
a) the VL comprises
a CDR-L1 comprising the amino acid sequence of SEQ ID NO: 100,
a CDR-L2 comprising the amino acid sequence of SEQ ID NO: 117, and
a CDR-L3 comprising the amino acid sequence of SEQ ID NO: 134.

75. The isolated antibody or antigen-binding fragment of claim 74, wherein the VH comprises the amino acid sequence of SEQ ID NO: 29, and/or the VL comprises the amino acid sequence of SEQ ID NO: 30.

76. The isolated antibody or antigen-binding fragment of claim 68, wherein
a) the VH comprises
a CDR-H1 comprising the amino acid sequence of SEQ ID NO: 50,
a CDR-H2 comprising the amino acid sequence of SEQ ID NO: 67, and
a CDR-H3 comprising the amino acid sequence of SEQ ID NO: 84; and
a) the VL comprises
a CDR-L1 comprising the amino acid sequence of SEQ ID NO: 101,
a CDR-L2 comprising the amino acid sequence of SEQ ID NO: 118, and
a CDR-L3 comprising the amino acid sequence of SEQ ID NO: 135.

77. The isolated antibody or antigen-binding fragment of claim 76, wherein the VH comprises the amino acid sequence of SEQ ID NO: 31, and/or the VL comprises the amino acid sequence of SEQ ID NO: 32.

78. The isolated antibody or antigen-binding fragment of claim 68, wherein
a) the VH comprises
a CDR-H1 comprising the amino acid sequence of SEQ ID NO: 35,
a CDR-H2 comprising the amino acid sequence of SEQ ID NO: 52, and
a CDR-H3 comprising the amino acid sequence of SEQ ID NO: 69; and
a) the VL comprises
a CDR-L1 comprising the amino acid sequence of SEQ ID NO: 86,
a CDR-L2 comprising the amino acid sequence of SEQ ID NO: 103, and
a CDR-L3 comprising the amino acid sequence of SEQ ID NO: 120.

79. The isolated antibody or antigen-binding fragment of claim 78, wherein the VH comprises the amino acid sequence of SEQ ID NO: 1, and/or the VL comprises the amino acid sequence of SEQ ID NO: 2.

80. The isolated antibody or antigen-binding fragment of claim 68, wherein
a) the VH comprises
a CDR-H1 comprising the amino acid sequence of SEQ ID NO: 36,
a CDR-H2 comprising the amino acid sequence of SEQ ID NO: 53, and
a CDR-H3 comprising the amino acid sequence of SEQ ID NO: 70; and
a) the VL comprises
a CDR-L1 comprising the amino acid sequence of SEQ ID NO: 87,
a CDR-L2 comprising the amino acid sequence of SEQ ID NO: 104, and
a CDR-L3 comprising the amino acid sequence of SEQ ID NO: 121.

81. The isolated antibody or antigen-binding fragment of claim 81, wherein the VH comprises the amino acid sequence of SEQ ID NO: 3, and/or the VL comprises the amino acid sequence of SEQ ID NO: 4.

82. The isolated antibody or antigen-binding fragment of claim 68, wherein
a) the VH comprises
a CDR-H1 comprising the amino acid sequence of SEQ ID NO: 37,
a CDR-H2 comprising the amino acid sequence of SEQ ID NO: 54, and
a CDR-H3 comprising the amino acid sequence of SEQ ID NO: 71; and
a) the VL comprises
a CDR-L1 comprising the amino acid sequence of SEQ ID NO: 88,
a CDR-L2 comprising the amino acid sequence of SEQ ID NO: 105, and
a CDR-L3 comprising the amino acid sequence of SEQ ID NO: 122.

83. The isolated antibody or antigen-binding fragment of claim 82, wherein the VH comprises the amino acid sequence of SEQ ID NO: 5, and/or the VL comprises the amino acid sequence of SEQ ID NO: 6.

84. The isolated antibody or antigen-binding fragment of claim 68, wherein
a) the VH comprises
a CDR-H1 comprising the amino acid sequence of SEQ ID NO: 39,
a CDR-H2 comprising the amino acid sequence of SEQ ID NO: 56, and
a CDR-H3 comprising the amino acid sequence of SEQ ID NO: 73; and
a) the VL comprises
a CDR-L1 comprising the amino acid sequence of SEQ ID NO: 90,
a CDR-L2 comprising the amino acid sequence of SEQ ID NO: 107, and
a CDR-L3 comprising the amino acid sequence of SEQ ID NO: 124.

85. The isolated antibody or antigen-binding fragment of claim 84, wherein the VH comprises the amino acid sequence of SEQ ID NO: 9, and/or the VL comprises the amino acid sequence of SEQ ID NO: 10.

86. The isolated antibody or antigen-binding fragment of claim 68, wherein
a) the VH comprises
a CDR-H1 comprising the amino acid sequence of SEQ ID NO: 40,
a CDR-H2 comprising the amino acid sequence of SEQ ID NO: 57, and
a CDR-H3 comprising the amino acid sequence of SEQ ID NO: 74; and
a) the VL comprises
a CDR-L1 comprising the amino acid sequence of SEQ ID NO: 91,
a CDR-L2 comprising the amino acid sequence of SEQ ID NO: 108, and
a CDR-L3 comprising the amino acid sequence of SEQ ID NO: 125.

87. The isolated antibody or antigen-binding fragment of claim 86 wherein the VH comprises the amino acid sequence of SEQ ID NO: 11, and/or the VL comprises the amino acid sequence of SEQ ID NO: 12.

88. The isolated antibody or antigen-binding fragment of claim 68, wherein
a) the VH comprises
a CDR-H1 comprising the amino acid sequence of SEQ ID NO: 41,
a CDR-H2 comprising the amino acid sequence of SEQ ID NO: 58, and
a CDR-H3 comprising the amino acid sequence of SEQ ID NO: 75; and
a) the VL comprises
a CDR-L1 comprising the amino acid sequence of SEQ ID NO: 92,
a CDR-L2 comprising the amino acid sequence of SEQ ID NO: 109, and
a CDR-L3 comprising the amino acid sequence of SEQ ID NO: 126.

89. The isolated antibody or antigen-binding fragment of claim 88, wherein the VH comprises the amino acid sequence of SEQ ID NO: 13, and/or the VL comprises the amino acid sequence of SEQ ID NO: 14.

90. The isolated antibody or antigen-binding fragment of claim 68, wherein
a) the VH comprises
a CDR-H1 comprising the amino acid sequence of SEQ ID NO: 42,
a CDR-H2 comprising the amino acid sequence of SEQ ID NO: 59, and
a CDR-H3 comprising the amino acid sequence of SEQ ID NO: 76; and
a) the VL comprises
a CDR-L1 comprising the amino acid sequence of SEQ ID NO: 93,
a CDR-L2 comprising the amino acid sequence of SEQ ID NO: 110, and
a CDR-L3 comprising the amino acid sequence of SEQ ID NO: 127.

91. The isolated antibody or antigen-binding fragment of claim 90, wherein the VH comprises the amino acid sequence of SEQ ID NO: 15, and/or the VL comprises the amino acid sequence of SEQ ID NO: 16.

92. The isolated antibody or antigen-binding fragment of claim 68, wherein
a) the VH comprises
a CDR-H1 comprising the amino acid sequence of SEQ ID NO: 43,
a CDR-H2 comprising the amino acid sequence of SEQ ID NO: 60, and
a CDR-H3 comprising the amino acid sequence of SEQ ID NO: 77; and
a) the VL comprises
a CDR-L1 comprising the amino acid sequence of SEQ ID NO: 94,
a CDR-L2 comprising the amino acid sequence of SEQ ID NO: 111, and
a CDR-L3 comprising the amino acid sequence of SEQ ID NO: 128.

93. The isolated antibody or antigen-binding fragment of claim 92, wherein the VH comprises the amino acid sequence of SEQ ID NO: 17, and/or the VL comprises the amino acid sequence of SEQ ID NO: 18.

94. The isolated antibody or antigen-binding fragment of claim 68, wherein
a) the VH comprises
a CDR-H1 comprising the amino acid sequence of SEQ ID NO: 44,
a CDR-H2 comprising the amino acid sequence of SEQ ID NO: 61, and
a CDR-H3 comprising the amino acid sequence of SEQ ID NO: 78; and
a) the VL comprises
a CDR-L1 comprising the amino acid sequence of SEQ ID NO: 95,
a CDR-L2 comprising the amino acid sequence of SEQ ID NO: 112, and
a CDR-L3 comprising the amino acid sequence of SEQ ID NO: 129.

95. The isolated antibody or antigen-binding fragment of claim 94, wherein the VH comprises the amino acid sequence of SEQ ID NO: 19, and/or the VL comprises the amino acid sequence of SEQ ID NO: 20.

96. The isolated antibody or antigen-binding fragment of claim 68, wherein
a) the VH comprises
a CDR-H1 comprising the amino acid sequence of SEQ ID NO: 45,
a CDR-H2 comprising the amino acid sequence of SEQ ID NO: 62, and
a CDR-H3 comprising the amino acid sequence of SEQ ID NO: 79; and
a) the VL comprises
a CDR-L1 comprising the amino acid sequence of SEQ ID NO: 96,
a CDR-L2 comprising the amino acid sequence of SEQ ID NO: 113, and
a CDR-L3 comprising the amino acid sequence of SEQ ID NO: 130.

97. The isolated antibody or antigen-binding fragment of claim 96, wherein the VH comprises the amino acid sequence of SEQ ID NO: 21, and/or the VL comprises the amino acid sequence of SEQ ID NO: 22.

98. The isolated antibody or antigen-binding fragment of claim 68, wherein
a) the VH comprises
a CDR-H1 comprising the amino acid sequence of SEQ ID NO: 46,
a CDR-H2 comprising the amino acid sequence of SEQ ID NO: 63, and
a CDR-H3 comprising the amino acid sequence of SEQ ID NO: 80; and
a) the VL comprises
a CDR-L1 comprising the amino acid sequence of SEQ ID NO: 97,
a CDR-L2 comprising the amino acid sequence of SEQ ID NO: 114, and
a CDR-L3 comprising the amino acid sequence of SEQ ID NO: 131.

99. The isolated antibody or antigen-binding fragment of claim 98, wherein the VH comprises the amino acid sequence of SEQ ID NO: 23, and/or the VL comprises the amino acid sequence of SEQ ID NO: 24.

100. The isolated antibody or antigen-binding fragment of claim 68, wherein
a) the VH comprises
a CDR-H1 comprising the amino acid sequence of SEQ ID NO: 47,
a CDR-H2 comprising the amino acid sequence of SEQ ID NO: 64, and
a CDR-H3 comprising the amino acid sequence of SEQ ID NO: 81; and
a) the VL comprises
a CDR-L1 comprising the amino acid sequence of SEQ ID NO: 98,
a CDR-L2 comprising the amino acid sequence of SEQ ID NO: 115, and
a CDR-L3 comprising the amino acid sequence of SEQ ID NO: 132.

101. The isolated antibody or antigen-binding fragment of claim 100, wherein the VH comprises the amino acid sequence of SEQ ID NO: 25, and/or the VL comprises the amino acid sequence of SEQ ID NO: 26.

102. The isolated antibody or antigen-binding fragment of any one of claims 68-101, wherein the isolated antibody comprises a human IgG4 Fc region.

103. The isolated antibody or antigen-binding fragment of claim 102, wherein the isolated antibody comprises a first polypeptide comprising a light chain comprising the VL, wherein the first polypeptide comprises the amino acid sequence of SEQ ID NO: 140; and a second polypeptide comprising a heavy chain comprising, from N terminus to C terminus, the VH and a human IgG4 Fc domain, wherein the second polypeptide comprises the amino acid sequence of SEQ ID NO: 141.

104. The isolated antibody or antigen-binding fragment of any one of claims 68-101, wherein the isolated antibody comprises a human IgG1 Fc region.

105. The isolated antibody or antigen-binding fragment of claim 104, wherein the isolated antibody comprises a first polypeptide comprising a light chain comprising the VL, wherein the first polypeptide comprises the amino acid sequence of SEQ ID NO: 144; and a second polypeptide comprising a heavy chain comprising, from N terminus to C terminus, the VH and a human IgG1 Fc domain, wherein the second polypeptide comprises the amino acid sequence of SEQ ID NO: 145.

106. The isolated antibody or antigen-binding fragment of claim 104, wherein the human IgG1 Fc region comprises two Fc domains each comprising a S239D substitution and/or an 1332E substitution.

107. The isolated antibody or antigen-binding fragment of claim 106, wherein the isolated antibody comprises a first polypeptide comprising a light chain comprising the VL, wherein the first polypeptide comprises the amino acid sequence of SEQ ID NO: 146; and a second polypeptide comprising a heavy chain comprising, from N terminus to C terminus, the VH and a human IgG1 Fc domain, wherein the second polypeptide comprises the amino acid sequence of SEQ ID NO: 147.

108. The isolated antibody or antigen-binding fragment of any one of claims 68-101, wherein the antigen-binding fragment is selected from the group consisting of a Fab, a Fv, a scFab, and a scFv.

109. The isolated antibody or antigen-binding fragment of any one of claims 68-108, wherein the isolated antibody or antigen-binding fragment binds human CD47 with a Kp of about 50 nM or less.

110. The isolated antibody or antigen-binding fragment of claim 109, wherein the isolated antibody or antigen-binding fragment binds human CD47 with a Kp of about 10 nM or less.

111. The isolated antibody or antigen-binding fragment of any one of claims 68-110, wherein the isolated antibody or antigen-binding fragment has a half maximal inhibitory concentration (IC50) of about 100 nM or less for blocking binding of human CD47 to human SIRPα in vitro.

112. The isolated antibody or antigen-binding fragment of claim 111, wherein the isolated antibody or antigen-binding fragment has a half maximal inhibitory concentration (IC50) of about 10 nM or less for blocking binding of human CD47 to human SIRPα in vitro.

113. The isolated antibody or antigen-binding fragment of any one of claims 68-112, wherein the isolated antibody or antigen-binding fragment completely blocks binding of human CD47 to human SIRPα in vitro when the isolated antibody or antigen-binding fragment is provided at a concentration of about 1 μM or greater.

114. The isolated antibody or antigen-binding fragment of any one of claims 68-113, wherein the isolated antibody or antigen-binding fragment has a half maximal effective concentration (EC50) of about 10 nM or less for binding to tumor cells in vitro, wherein the tumor cells comprise a B cell lymphoma cell line, a T cell lymphoma cell line, or a combination thereof.

115. The isolated antibody or antigen-binding fragment of any one of claims 68-114, wherein the isolated antibody or antigen-binding fragment has a half maximal effective concentration (EC50) of about 10 nM or less for increasing macrophage phagocytosis of tumor cells in vitro, wherein the tumor cells comprise a B cell lymphoma cell line, a T cell lymphoma cell line, or a combination thereof.

116. The isolated antibody or antigen-binding fragment of claim 115, wherein the isolated antibody or antigen-binding fragment has a half maximal effective concentration (EC50) of about 1 nM or less for increasing macrophage phagocytosis of tumor cells in vitro, wherein the tumor cells comprise a B cell lymphoma cell line, a T cell lymphoma cell line, or a combination thereof.

117. The isolated antibody or antigen-binding fragment of any one of claims 68-116, wherein the isolated antibody or antigen-binding fragment is cross-reactive with a CD47 polypeptide from at least one non-human species selected from the group consisting of cynomolgus monkey, rat, and dog.

118. The isolated antibody or antigen-binding fragment of claim 117, wherein the isolated antibody or antigen-binding fragment binds to cynomolgus monkey CD47.

119. An isolated antibody or antigen-binding fragment binds to one or more amino acid residues of human CD47 selected from the group consisting of K39, W40, K41, F50, D51, G52, T99, E100, L101, and T102.

120. The isolated antibody or antigen-binding fragment of claim 119, wherein the isolated antibody or antigen-binding fragment does not bind to one or more amino acid residues of human CD47 selected from the group consisting of L2, L3, F4, K6, N27, E29, A30, Q31, T34, E35, V36, Y37, D46, T49, A53, E97, R103, E104, G105, and E106.

121. The isolated antibody or antigen-binding fragment of any one of claims 119-120, wherein the isolated antibody or antigen-binding fragment binds to amino acid residues K39, W40, K41, F50, D51, G52, T99, E100, L101, and T102 of human CD47.

122. The isolated antibody or antigen-binding fragment of any one of claims 119-121, wherein the isolated antibody or antigen-binding fragment does not bind to amino acid residues L2, L3, F4, K6, N27, E29, A30, Q31, T34, E35, V36, Y37, D46, T49, A53, E97, R103, E104, G105, and E106 of human CD47.

123. An isolated polynucleotide encoding one or more polypeptide chains of the masked antibody of any one of claims 1-67 or the isolated antibody or antigen-biding fragment of any one of claims 68-122.

124. A vector comprising the polynucleotide of claim 123.

125. The vector of claim 124, wherein the vector is an expression vector.

126. A host cell comprising the vector of claim 124 or 125.

127. A method of making a masked antibody, or an antibody or antigen-binding fragment thereof, comprising culturing the host cell of claim 126 under conditions suitable for producing the masked antibody, or the antibody or antigen-binding fragment thereof.

128. The method of claim 127, further comprising recovering the masked antibody, or the antibody or antigen-binding fragment thereof, produced by the cell.

129. A pharmaceutical composition comprising the masked antibody of any one of claims 1-67, or the antibody or antigen-binding fragment thereof of any one of claims 68-122, and a pharmaceutically acceptable carrier.

130. A method for treating a CD47-positive disease or condition in a subject in need thereof, comprising administering to the subject an effective amount of the pharmaceutical composition of claim 129.

131. The method of claim 130, wherein the administering does not cause anemia in the subject.

132. The method of claim 130 or 131, wherein the disease or condition is cancer.

133. The method of claim 132 wherein the cancer comprises B cell lymphoma, T cell lymphoma, or a combination thereof.

134. The method of claim 132, wherein the cancer is selected from the group consisting of lymphoma, leukemia, head and neck cancer, gastric cancer, esophageal cancer, breast cancer, cervical cancer, cholangiocarcinoma, colon cancer, ovarian cancer, thyroid cancer, uterine cancer, endometrial cancer, lung cancer, mesothelioma, and pancreatic cancer.

135. The method of claim 132, wherein the cancer is selected from the group consisting of triple negative breast cancer (TNBC), Her2+ gastric-esophageal junction (GEJ) cancer, small cell lung cancer (SCLC), diffuse large B-cell lymphoma (DLBCL), acute myeloid leukemia (AML), Head and neck squamous cell carcinoma (HNSC), gastric carcinoma (GC), breast invasive carcinoma (BRCA), cervical squamous cell carcinoma and endocervical adenocarcinoma (CESC), cholangiocarcinoma (CHOL), colon adenocarcinoma (COAD), ovarian serous cystadenocarcinoma (OV), thyroid carconima (THCA), uterine corpus endometrial carcinoma (UCEC), HER2+ breast cancer, hormone receptor positive breast cancer, lymphoid neoplasm diffuse large B-cell lymphoma (DLBC), lung adenocarcinoma (LUAD), lung squamous cell carcinoma (LUSC), mesothelioma (MESO), and pancreatic adenocarcinoma (PAAD).

136. The method of any one of claims 130-135, wherein the masked antibody, or the antibody or antigen-binding fragment thereof, is administered at a dose of at least about 0.6 mg/kg.

137. The method of any one of claims 130-134, wherein the pharmaceutical compositions is administered at a frequency of at least once every two weeks.

138. The method of any one of claims 130-134, wherein the pharmaceutical compositions is administered at a frequency of at least once every three weeks.

139. The method of any one of claims 130-138, further comprising administering to the subject an effective amount of one or more additional therapeutic agents.

140. The method of claim 139, wherein the one or more therapeutic agents comprise viral gene therapy, an immune checkpoint inhibitor, a target therapy, a radiation therapy, a chemotherapy, or any combination thereof.

141. The method of claim 139 or 140, wherein the one or more additional therapeutic agents comprise pomalyst, revlimid, lenalidomide, pomalidomide, thalidomide, a DNA-alkylating platinum-containing derivative, cisplatin, 5-fluorouracil, cyclophosphamide, an anti-CD47 antibody, an anti-CTLA4 antibody, an anti-PD-1 antibody, an anti-PD-L1 antibody, an anti-CD20 antibody, an anti-CD40 antibody, an anti-DR5 antibody, an anti-CD1d antibody, an anti-TIM3 antibody, an anti-SLAMF7 antibody, an anti-KIR receptor antibody, an anti-OX40 antibody, an anti-HER2 antibody, an anti-ErbB-2 antibody, an anti-EGFR antibody, cetuximab, rituximab, trastuzumab, pembrolizumab, radiotherapy, single dose radiation, fractionated radiation, focal radiation, whole organ radiation, IL-12, IFNa, GM-CSF, a chimeric antigen receptor, adoptively transferred T cells, an anti-cancer vaccine, an oncolytic virus, or any combination thereof.

142. A method of treating cancer comprising administering an anti-CD47 masked antibody, wherein the anti-CD47 masked antibody comprises:
(a) a masking peptide comprising, from N terminus to C terminus, a masking moiety (MM) and a linkage moiety (LM);
(b) a target binding moiety (TBM) comprising an antibody heavy chain variable region (VH) and an antibody light chain variable region (VL), wherein the TBM binds to human CD47; and
(c) a human IgG1 Fc region, or a human IgG Fc region with enhanced antibody-dependent cellular cytotoxicity (ADCC) activity,
wherein the masked antibody binds to human CD47 at the site of the cancer.

143. The method of claim 142, wherein the masking peptide does not comprise a cleavable moiety.

144. The method of claim 142, wherein the masked antibody is an activatable antibody, the LM comprises a cleavable moiety (CM) comprising at least one cleavage site, and the activatable antibody has a higher binding affinity to human CD47 when the CM is cleaved at the site of the cancer than when the CM is not cleaved.
